SEQUENCE LISTING (135 sequences)

SEQ ID NO: 1 - DNA, 20 bp, Artificial Sequence
  Primer
tttcaactaccaggggctgt

SEQ ID NO: 2 - DNA, 20 bp, Artificial Sequence
  Primer
gccagtctttccgttcagag

SEQ ID NO: 3 - DNA, 20 bp, Artificial Sequence
  Primer
tagtggagaccatccgttcc

SEQ ID NO: 4 - DNA, 20 bp, Artificial Sequence
  Primer
ccttctctggtcacgggata

SEQ ID NO: 5 - DNA, 20 bp, Artificial Sequence
  Primer
cagtacggtgtgtggagctg

SEQ ID NO: 6 - DNA, 20 bp, Artificial Sequence
  Primer
ggtgcaggttcttcatggat

SEQ ID NO: 7 - DNA, 20 bp, Artificial Sequence
  Primer
cctttccagagagccagaaa

SEQ ID NO: 8 - DNA, 20 bp, Artificial Sequence
  Primer
gcaaaacgtgacccagagac

SEQ ID NO: 9 - DNA, 20 bp, Artificial Sequence
  Primer
ttcaccagcactgtctccac

SEQ ID NO: 10 - DNA, 20 bp, Artificial Sequence
  Primer
ttcgattgattctgggctct

SEQ ID NO: 11 - DNA, 40 bp, Artificial Sequence
  Primer
ggcgcggatccgccgccaccatgtttggccgctcgcggag

SEQ ID NO: 12 - DNA, 73 bp, Artificial Sequence
  Primer
tgatagcggccgctcatcaagcgtaatctggaacatcgtatgggtactcgagtttgcgcttcctcagtatcag

SEQ ID NO: 13 - DNA, 40 bp, Artificial Sequence
  Primer
ggcgcggatccgccgccaccatggccgtgactgcctgtca

SEQ ID NO: 14 - DNA, 73 bp, Artificial Sequence
  Primer
gatagcggccgctcatcaagcgtaatctggaacatcgtatgggtactcgaggaggaactccacgtaattctca

SEQ ID NO: 15 - DNA, 42 bp, Artificial Sequence
  Primer
ggcgcttaattaagccgccaccatggcggccgagagggaacc

SEQ ID NO: 16 - DNA, 73 bp, Artificial Sequence
  Primer
tgatagcggccgctcatcaagcgtaatctggaacatcgtatgggtactcgagatccacttcgattgattctgg

SEQ ID NO: 17 - DNA, 40 bp, Artificial Sequence
  Primer
ggcgcggatccgccgccaccatgattttgaatagcctctc

SEQ ID NO: 18 - DNA, 74 bp, Artificial Sequence
  Primer
tgatagcggccgctcatcaagcgtaatctggaacatcgtatgggtactcgaggccattgtattgctgctggtag

SEQ ID NO: 19 - DNA, 20 bp, Artificial Sequence
  Primer
aaaacccacagcctcatgtc

SEQ ID NO: 20 - DNA, 20 bp, Artificial Sequence
  Primer
cacctggtccttgttctggt

SEQ ID NO: 21 - DNA, 20 bp, Artificial Sequence
  Primer
ggtttcccattatgccattg

SEQ ID NO: 22 - DNA, 20 bp, Artificial Sequence
  Primer
ttccaagacatgtgcagctc

SEQ ID NO: 23 - DNA, 20 bp, Artificial Sequence
  Primer
ccgacaggatgttgacaatg

SEQ ID NO: 24 - DNA, 20 bp, Artificial Sequence
  Primer
tcagagaggtcggcaaactt

SEQ ID NO: 25 - DNA, 20 bp, Artificial Sequence
  Primer
ggatgctgcctttaattgga

SEQ ID NO: 26 - DNA, 20 bp, Artificial Sequence
  Primer
cgcacccttgaagaagtagc

SEQ ID NO: 27 - DNA, 20 bp, Artificial Sequence
  Primer
caaactctacggcttctgcc

SEQ ID NO: 28 - DNA, 20 bp, Artificial Sequence
  Primer
tggcaccgatgaatgatcta

SEQ ID NO: 29 - DNA, 20 bp, Artificial Sequence
  Primer
aagcagttgcactgtgatgc

SEQ ID NO: 30 - DNA, 20 bp, Artificial Sequence
  Primer
gcagtgagggcaagaaaaag

SEQ ID NO: 31 - DNA, 20 bp, Artificial Sequence
  Primer
caaggccttcaactgcaaat

SEQ ID NO: 32 - DNA, 20 bp, Artificial Sequence
  Primer
aaggttcgggaacaggtctt

SEQ ID NO: 33 - DNA, 19 bp, Artificial Sequence
  Primer
ctgaagtagcttccccagg

SEQ ID NO: 34 - DNA, 21 bp, Artificial Sequence
  Primer
tgttgatgagtgagtccactg

SEQ ID NO: 35 - DNA, 19 bp, Artificial Sequence
  Primer
acacggatcccagagcagc

SEQ ID NO: 36 - DNA, 21 bp, Artificial Sequence
  Primer
tgcagcgataaaacaaaaggc

SEQ ID NO: 37 - DNA, 15 bp, Artificial Sequence
  Primer
gcccctgcaccgtgg

SEQ ID NO: 38 - DNA, 20 bp, Artificial Sequence
  Primer
tctctgaccctccagccaat

SEQ ID NO: 39 - DNA, 20 bp, Artificial Sequence
  Primer
gcgacggttctttctaggga

SEQ ID NO: 40 - DNA, 20 bp, Artificial Sequence
  Primer
tccccttgaggaaatgggag

SEQ ID NO: 41 - DNA, 17 bp, Artificial Sequence
  Primer
ccagggacagtcccccc

SEQ ID NO: 42 - DNA, 17 bp, Artificial Sequence
  Primer
gcgtcgggttccgagat

SEQ ID NO: 43 - DNA, 19 bp, Artificial Sequence
  Primer
ggtgggcatgagatgcact

SEQ ID NO: 44 - DNA, 20 bp, Artificial Sequence
  Primer
caccaccgccagtctgtctt

SEQ ID NO: 45 - DNA, 20 bp, Artificial Sequence
  Primer
gagggcctgtggatgaactg

SEQ ID NO: 46 - DNA, 21 bp, Artificial Sequence
  Primer
agtcgtacaccttgcactgca

SEQ ID NO: 47 - DNA, 21 bp, Artificial Sequence
  Primer
tccaccacctcgcatatctct

SEQ ID NO: 48 - DNA, 21 bp, Artificial Sequence
  Primer
gccatttagggcctcactgga

SEQ ID NO: 49 - DNA, 20 bp, Artificial Sequence
  Primer
ccagaaggttcctttgtgga

SEQ ID NO: 50 - DNA, 20 bp, Artificial Sequence
  Primer
ggctggtgtttgacttggtt

SEQ ID NO: 51 - DNA, 19 bp, Artificial Sequence
  Primer
ggtggccctgtccttaaag

SEQ ID NO: 52 - DNA, 19 bp, Artificial Sequence
  Primer
cgtacccgtcccttcctcc

SEQ ID NO: 53 - DNA, 20 bp, Artificial Sequence
  Primer
aagtgtgctctggggtcaag

SEQ ID NO: 54 - DNA, 20 bp, Artificial Sequence
  Primer
agcctttgtccgtgaggtaa

SEQ ID NO: 55 - DNA, 20 bp, Artificial Sequence
  Primer
agctcaactttctggcgaag

SEQ ID NO: 56 - DNA, 20 bp, Artificial Sequence
  Primer
cttcacgacgatgtcattgc

SEQ ID NO: 57 - DNA, 17 bp, Artificial Sequence
  Primer
ccatttaaagatctccg

SEQ ID NO: 58 - DNA, 19 bp, Artificial Sequence
  Primer
catttggaagtcatgttcg

SEQ ID NO: 59 - DNA, 21 bp, Artificial Sequence
  Primer
aggacgaggggagctatgacc

SEQ ID NO: 60 - DNA, 19 bp, Artificial Sequence
  Primer
gtgggggccttctgataag

SEQ ID NO: 61 - DNA, 20 bp, Artificial Sequence
  Primer
atcccagaggctccaaagat

SEQ ID NO: 62 - DNA, 20 bp, Artificial Sequence
  Primer
gctggagcttctctgctgtt

SEQ ID NO: 63 - DNA, 20 bp, Artificial Sequence
  Primer
gacctttgagtgtggggtgt

SEQ ID NO: 64 - DNA, 20 bp, Artificial Sequence
  Primer
tcttccgagcattcacactg

SEQ ID NO: 65 - DNA, 20 bp, Artificial Sequence
  Primer
acagtcccaagaaacggatg

SEQ ID NO: 66 - DNA, 20 bp, Artificial Sequence
  Primer
ccttcaccgtgtagcggtat

SEQ ID NO: 67 - DNA, 20 bp, Artificial Sequence
  Primer
aagcccatctccacacactc

SEQ ID NO: 68 - DNA, 20 bp, Artificial Sequence
  Primer
aggagaaggggctctcagtc

SEQ ID NO: 69 - DNA, 20 bp, Artificial Sequence
  Primer
tgagaccaggcagtgaacag

SEQ ID NO: 70 - DNA, 20 bp, Artificial Sequence
  Primer
ccgagaggtccatgaggtaa

SEQ ID NO: 71 - DNA, 20 bp, Artificial Sequence
  Primer
cgtgacttccgtcttggatt

SEQ ID NO: 72 - DNA, 20 bp, Artificial Sequence
  Primer
cctttctgggtggatgctaa

SEQ ID NO: 73 - DNA, 20 bp, Artificial Sequence
  Primer
atttggaaactgccacaagc

SEQ ID NO: 74 - DNA, 20 bp, Artificial Sequence
  Primer
atttggaaactgccacaagc

SEQ ID NO: 75 - DNA, 20 bp, Artificial Sequence
  Primer
catctaccacagcagctcca

SEQ ID NO: 76 - DNA, 20 bp, Artificial Sequence
  Primer
ctcctccccatggattacct

SEQ ID NO: 77 - DNA, 20 bp, Artificial Sequence
  Primer
gacgacacggaggactttgt

SEQ ID NO: 78 - DNA, 20 bp, Artificial Sequence
  Primer
tgtctgagccattgaggatg

SEQ ID NO: 79 - DNA, 20 bp, Artificial Sequence
  Primer
agtggagctgtggttttgct

SEQ ID NO: 80 - DNA, 20 bp, Artificial Sequence
  Primer
agaccttccccgtcaaaaat

SEQ ID NO: 81 - DNA, 20 bp, Artificial Sequence
  Primer
tccaggtggagcttcttttg

SEQ ID NO: 82 - DNA, 22 bp, Artificial Sequence
  Primer
ttcttagagtgacctggagacc

SEQ ID NO: 83 - DNA, 20 bp, Artificial Sequence
  Primer
aacatcatccctgcttccac

SEQ ID NO: 84 - DNA, 20 bp, Artificial Sequence
  Primer
gaccacctggtcctcagtgt

SEQ ID NO: 85 - DNA, 20 bp, Artificial Sequence
  Primer
acagtggccacctacaaagg

SEQ ID NO: 86 - DNA, 20 bp, Artificial Sequence
  Primer
ccgagatggggttgataatg

SEQ ID NO: 87 - DNA, 19 bp, Artificial Sequence
  Primer
aaaatggcagtgcgtttag

SEQ ID NO: 88 - DNA, 20 bp, Artificial Sequence
  Primer
tttgaaggcagtctgtcgta

SEQ ID NO: 89 - DNA, 20 bp, Artificial Sequence
  Primer
cgtggctacatctcccattt

SEQ ID NO: 90 - DNA, 20 bp, Artificial Sequence
  Primer
tccctcatgaccaggatctc

SEQ ID NO: 91 - DNA, 14 bp, Artificial Sequence
  Primer
gaccccttcattga

SEQ ID NO: 92 - DNA, 14 bp, Artificial Sequence
  Primer
cttctccatggtgg

SEQ ID NO: 93 - DNA, 6891 bp, Homo sapiens
aactgcatttcccagcgccccacgcggcggcggccgtaaagcgcggcggtcgaacggccggttccggctgaatgtcagtgctgggctgtgggccggggaggaaggcggctcgcggttcctccaccgcctccgccgccgcatcctccgcttgtgctaccgccgcgggcgctgggccgctctgctggtccggcatgagaccgtgagacgagagacgggtcggggccgccgacatgtttggccgctcgcggagctgggtgggcgggggccatggcaagacttcccgcaacatccactccttggaccacctcaagtatctgtaccacgttttgaccaaaaacaccacagtcacagaacagaaccggaacctgctagtggagaccatccgttccatcactgagatcctgatctggggagatcaaaatgacagctctgtatttgacttcttcctggagaagaatatgtttgttttcttcttgaacatcttgcggcaaaagtcgggccgttacgtgtgcgttcagctgctgcagaccttgaacatcctctttgagaacatcagtcacgagacctcactttattatttgctctcaaataactacgtaaattctatcatcgttcataaatttgacttttctgatgaggagattatggcctattatatatcgttcctgaaaacactttcgttaaaactcaacaaccacactgtccatttcttttataatgagcacaccaatgactttgccctgtacacagaagccatcaagtttttcaaccaccctgaaagcatggttagaattgctgtaagaaccataactttgaatgtctataaagtgtcattggataaccaggccatgctgcactacatccgagataaaactgctgttccttacttctccaatttggtctggttcattgggagccatgtgatcgaactcgatgactgcgtgcagactgatgaggagcatcggaatcggggtaaactgagtgatctggtggcagagcacctagaccacctgcactatctcaatgacatcctgatcatcaactgtgagttcctcaacgatgtgctcactgaccacctgctcaacaggctcttcctgcccctctacgtgtactcactggagaaccaggacaagggaggagaacggccgaaaattagcctgccggtgtctctttatcttctgtcacaggtcttcttaattatacatcatgcaccgctggtgaactcgttagctgaagtcattctgaatggtgatctgtctgagatgtacgctaagactgaacaggatattcagagaagttctgccaagcccagcattcggtgcttcattaaacccaccgagacactcgagcggtcccttgagatgaacaagcacaagggcaagaggcgggtgcaaaagagacccaactacaaaaacgttggggaagaagaagatgaggagaaagggcccaccgaggatgcccaagaagacgccgagaaggctaaaggtacagagggtggttcaaaaggcatcaagacgagtggggagagtgaagagatcgagatggtgatcatggagcgtagcaagctctcagagctggccgccagcacctccgtgcaggagcagaacaccacggacgaggagaaaagcgccgccgccacctgctctgagagcacgcaatggagcagacccttcctggatatggtgtaccacgcgctggacagcccggatgatgattaccatgccctgttcgtgctctgcctcctctatgccatgtctcataataaaggcatggatcctgaaaaattagagcgaatccagctccccgtgccaaatgcggccgagaagaccacctacaaccacccgctagctgaaagactcatcaggatcatgaacaacgctgcccagccagatgggaagatccggctggcgacgctggagctgagctgcctgcttctgaagcagcaagtcctgatgagtgctggctgcatcatgaaggacgtgcacctggcctgcctggagggtgcgagagaagaaagtgttcaccttgtacgacatttttataagggagaagacatttttttggacatgtttgaagatgagtataggagcatgacaatgaagcccatgaacgtggaatatctcatgatggacgcctccatcctgctgcccccaacaggcacgccactgacgggcattgacttcgtgaagcggctgccgtgtggcgatgtggagaagacccggcgggccatccgggtgttcttcatgctgcgttccctgtcactgcaattgcgaggggagcctgagacacagttgccgctgactcgggaggaggacctgatcaagactgatgatgtcctggatctgaataacagcgacttgattgcatgtacagtgatcaccaaggatggcggcatggtccagcgattcctggctgtggatatttaccagatgagtttggtggagcctgatgtgtccaggcttggctggggagtggtcaagtttgcaggcctattgcaggacatgcaggtgactggcgtggaggacgacagccgtgccctgaacatcaccatccacaagcctgcgtccagcccccattccaagcccttccccatcctccaggccaccttcatcttctcagaccacatccgctgcatcatcgccaagcagcgcctggccaaaggccgcatccaggcaaggcgcatgaagatgcagagaatagctgccctcctggacctcccaatccagcccaccactgaagtcctggggtttggactcggctcctccacctccactcagcacctgcctttccgcttctacgaccaggggcgccggggcagcagcgaccccacagtgcagcgctccgtgtttgcatcggtggacaaggtgccaggcttcgccgtggcccagtgcataaaccagcacagctccccgtccctgtcctcacagtcgccaccctccgccagcgggagccccagcggcagcgggagcaccagccactgcgactctggaggcaccagctcgtcctccaccccctccacagcccagagtccagcagatgcccccatgagtccagaactgcctaagcctcaccttcctgaccagttggtaatcgtcaacgaaacggaagcagactctaagcccagcaagaacgtggccaggagcgcagccgtggagacagccagcctgtcccccagcctcgtccctgcccggcagcccaccatttccctgctctgcgaggacacggctgacacgctgagcgtcgaatcgctgacccttgtccccccagttgacccccacagcctccgcagcctcaccggcatgcccccgctgtccacgccggctgccgcctgcacagagcccgtgggcgaagaggctgcatgtgctgagcctgtgggcaccgctgaggactgagtcagtgccggggcctccctttgtgtgtgtggccccgctggtagggaccccagtgccgctgactggcaagacacactgggagcacccaccattctgtgcggcccccagcagccatctcaaccacctatccctgcgctcccttgaatgggaagaagccccacgttgtccttgaattcctttttcactttgcatctcttcacgtgcaggctgggaccagcggagacaccgcggcgaatgcagatgactgcaccggccactcagggagctgcctgggctccgtgtctctgagccccgggtggcaggacccaccggcacctctttcttcctctgtcatatggctcctctgtcaccagccccagtgtgcacagaagaattggaccaggtcactgtacgtagaaatttgtagaaaagcagacttagataaacatctcctttggatatttatttccgcttttggcagcaggtgaacatttatttttaaaacttctatttaaaagaagtccaaaaacatcaacactaaggtttgatgtcatgtgaaaagtgtaataataacagttaagatttcatgatcattttcactggacctttcctgatattttgtttcagagttcttagtgtggctttttccatttatttaagtgattctttgttactcactaactctgcaagcctgtggaataatgaagtaccttcctggaaagtttggattattttttaaacaaaaacaagggagatacatgtattctcaggtacacacagagctgagagggctgaatggttttctgctatagcagccgagaggcctcccatcatggaaagatttctccaggaaaaggaggaatgtagccagctccccactcaggacgcttcctcatttctcttcaccaaaaccaaacagagacagcttccagcaccttcttcagtgttaccatctctaagaaggaaccagttgggaccgtgaagactcccgaccctgtggccatgatggaaatcaaaggaagacaccctctacgtcacctgccctcgactgtgtgtgcccacatgtgccgagagatggcccagagccagttcccctccagctgcaagggcatggtgtccccagagctctgagtctgtcactctccctctgctactgctgctgatctgaatatggaaaccccatggttcccttccccattcggactgggtgtgtacaagcaaggacccagatgcatcagacacagcccccaagatgttcctttctactcggccagctcgggagccagacacagcactcacagcccaggccgtgatccaccctccccaagtccaccagggccagcggcccctcacctctctggtcactggtgagaccttccacaactttcctccagacctgccagcagatgtgcccaccaggggcattaggtatccgccggagcctggccatagggtagtctcgggagccgcgctgagatcttttgccacctgcattttagaagaacatggtctctgtctcctcggcccagccagctgtcccggcaaggcctgccgagggcagttttcaacctcatgaaggaaacacagtcctgccaaggagggggagtggcgcccatggggacaggcctcagtccttagaagccctctgggtagctgtgcccacccagccttcatggctgcaggtacaaggacctttgcttccatagagaaaacgcacagctcagaaagggggccacatgggcagaaacccaaaggaaggacaaaccacgaccaccgtggccatctgcagaatccctggaagagaaggaaggcagggtggagcggggggaagaccatcatggagagaaggaccacagcatcaggagacgggacacgccacacccagcaggcagcctgtgtgttgcttaattttttaagagcaagaggggtagagaggatcaagctggccctggctggagatggctagcccctgagacatgcacttctggttttgaaatgactctgtctgtggggcagcagaaactagagaaggcaagtggctgccccaccccaaggcgtgaccaggaggaacagcctgcagctcactccatgccacacgggtgggccaccagcctgctgtcagaagtctctgggctccaactggtcttgtaaccactgagcactgaaggagagaggtcttggtcagggctggacagcatgcccgggaggaccagcagaggattaaaggtgactgggaggaccagcggaggataaaagacactgctcagggcagggcttctaccctgcatccctggccaagaaaagggcagtccccatgtgggcttgcagggtcactctcaggggcctctttcagctggggctggcaacttgcgtctgggggacacctccaggtgtgtggggtgaggatttcctataaccagggctcccagaagctttgcttatgtaaggaggtctgggagccagcccattggaggccaccagccattttggcttcaaaggaccccacctcacccaggtctcagcggcagtgggcacagctatgtcttcaggagctcccgtcaaacctcatagctggggcgctcccagacaggccagtccagacaggacacgctgggcccctggcatccagaggaagagccaggagtgtgggaaggcccacagtgggggctgtggcttctgacactcaggtcatagcctcagaggtctgaggtcagcccccacagacccatccggcccgccccccaagtccctgcagagagcacttagagttatggcccaggccctggtccacccttcccctgtgcacctccggctgggtttgccaagtcagggagcagggctggccgcaggaactcccaaaccttggctttgaatattgttgtggaggtgtgctcgtccctttctggacgtgcaaggtacctgtcccagcaggtcagatggggccagctgaggcgctcccccaggcaggaagggccagccttcaccatcgcgtgggattgggaggaggggcctccgtgagcagcccctcctctgccgctgtcccagcccagtccctctcccggagccttggcagcctcccacaacccagacacttgcgttcacaagcaacctaaggggcaggtgaagaagcgcagccctgccagacgcgctagattcctctaaggtctctgagatgcaccgttttttaaaaaggcgtggggtgaactgattttgatcttcttgtctagatgcaataaataaatctgaagcatttaatgtagtcatcttgacattgggcctacactgtacgagttccttatgtttccttgagctaaaaatatgtaaataatttttgtcccagtgagaaccgagggttagaaaacctcgatgcctctgagcctcgggaccgctctagggaagtacctgctttcgccagcatgactcatgcttcgtgggtactgaacacgagggtggaaatgaaaactggaacttccttgtaaatttaaacttggcaataaaagagaaaaaaagttaccaagaa

SEQ ID NO: 94 - protein, 1053 aa, Homo sapiens
MFGRSRSWVGGGHGKTSRNIHSLDHLKYLYHVLTKNTTVTEQNRNLLVETIRSITEILIWGDQNDSSVFDFFLEKNMFVFFLNILRQKSGRYVCVQLLQTLNILFENISHETSLYYLLSNNYVNSIIVHKFDFSDEEIMAYYISFLKTLSLKLNNHTVHFFYNEHTNDFALYTEAIKFFNHPESMVRIAVRTITLNVYKVSLDNQAMLHYIRDKTAVPYFSNLVWFIGSHVIELDDCVQTDEEHRNRGKLSDLVAEHLDHLHYLNDILIINCEFLNDVLTDHLLNRLFLPLYVYSLENQDKGGERPKISLPVSLYLLSQVFLIIHHAPLVNSLAEVILNGDLSEMYAKTEQDIQRSSAKPSIRCFIKPTETLERSLEMNKHKGKRRVQKRPNYKNVGEEEDEEKGPTEDAQEDAEKAKGTEGGSKGIKTSGESEEIEMVIMERSKLSELAASTSVQEQNTTDEEKSAAATCSESTQWSRPFLDMVYHALDSPDDDYHALFVLCLLYAMSHNKGMDPEKLERIQLPVPNAAEKTTYNHPLAERLIRIMNNAAQPDGKIRLATLELSCLLLKQQVLMSAGCIMKDVHLACLEGAREESVHLVRHFYKGEDIFLDMFEDEYRSMTMKPMNVEYLMMDASILLPPTGTPLTGIDFVKRLPCGDVEKTRRAIRVFFMLRSLSLQLRGEPETQLPLTREEDLIKTDDVLDLNNSDLIACTVITKDGGMVQRFLAVDIYQMSLVEPDVSRLGWGVVKFAGLLQDMQVTGVEDDSRALNITIHKPASSPHSKPFPILQATFIFSDHIRCIIAKQRLAKGRIQARRMKMQRIAALLDLPIQPTTEVLGFGLGSSTSTQHLPFRFYDQGRRGSSDPTVQRSVFASVDKVPGFAVAQCINQHSSPSLSSQSPPSASGSPSGSGSTSHCDSGGTSSSSTPSTAQSPADAPMSPELPKPHLPDQLVIVNETEADSKPSKNVARSAAVETASLSPSLVPARQPTISLLCEDTADTLSVESLTLVPPVDPHSLRSLTGMPPLSTPAAACTEPVGEEAACAEPVGTAED

SEQ ID NO: 95 - DNA, 5197 bp, Homo sapiens
ggcgggatcggggaaggaggggccccgccgcctagagggtggagggagggcgcgcagtcccagcccagagcttcaaaacagcccggcggcctcgcctcgcacccccagccagtccgtcgatccagctgccagcgcagccgccagcgccggcacatcccgctctgggctttaaacgtgacccctcgcctcgactcgccctgccctgtgaaaatgttggtgcttcttgctttcatcatcgccttccacatcacctctgcagccttgctgttcattgccaccgtcgacaatgcctggtgggtaggagatgagttttttgcagatgtctggagaatatgtaccaacaacacgaattgcacagtcatcaatgacagctttcaagagtactccacgctgcaggcggtccaggccaccatgatcctctccaccattctctgctgcatcgccttcttcatcttcgtgctccagctcttccgcctgaagcagggagagaggtttgtcctaacctccatcatccagctaatgtcatgtctgtgtgtcatgattgcggcctccatttatacagacaggcgtgaagacattcacgacaaaaacgcgaaattctatcccgtgaccagagaaggcagctacggctactcctacatcctggcgtgggtggccttcgcctgcaccttcatcagcggcatgatgtacctgatactgaggaagcgcaaatagagttccggagctgggttgcttctgctgcagtacagaatccacattcagataaccattttgtatataatcattattttttgaggtttttctagcaaacgtattgtttcctttaaaagccaaaaaaaaaaaaaaaaaaaaaaaaaaaagaaaaaagaaaaaaaaaatccaaaagagagaagagtttttgcattcttgagatcagagaatagactatgaaggctggtattcagaactgctgcccactcaaaagtctcaacaagacacaagcaaaaatccagcaatgctcaaatccaaaagcactcggcaggacatttcttaaccatggggctgtgatgggaggagaggagaggctgggaaagccgggtctctggggacgtgcttcctatgggtttcagctggcccaagcccctcccgaatctctctgctagtggtgggtggaagagggtgaggtggggtataggagaagaatgacagcttcctgagaggtttcacccaagttccaagtgagaagcaggtgtagtccctggcattctgtctgtatccaaaccagagcccagccatccctccggtatcggggtgggtcagaaaaagtctcacctcaatttgccgacagtgtcacctgcttgccttaggaatggtcatccttaacctgcgtgccagatttagactcgtctttaggcaaaacctacagcgccccccccctcaccccagacctacagaatcagagtcttcaagggatggggccagggaatctgcatttctaacgcgctccctgggcaacgcttcagatgcgttgaagttggggaccacggtgcctgggccaggtcagcagagctgcctcgtaaatgctggggtatcgtcatgtggagatggggaggtgaatgcaacccccacagcaggccaaaaccttggcctccatcgccacagctgtctacatctagggccccaaaactccattcctgagccatgtgaactcatagacaccttcagggtgtggggtacagcctccttcccatcttatcccagaaggcctctcccttcttgtccagcccttcatgctacacctggctggcctctcacccctatttctagagcctcagaggacccatccaccattcattcattcattcattcattcattcattcattcattcatcaacataaatcataacttgcatgcatgtgccaggcacaggggataccctctagagacaatctcctcctagggctcatggcctagtggaggagacagattaaaacttaattagaaaaactggctgggtacagtggctcatgcttgtaatcccagcactttgggaggctgaggcgggtggatcacctgaggtcaggagttcaagaccagcctggccaaaatggtaaaacctgtctctactaaaaatacaaaaatgagctgggcgtggtggtgcatgcctgtaatcccagctatcaggtggctgaggcaggagaatcacttgaaatgggaggtggaggttgcagtgagccgagaccgtgccactgcactccagcctgggtgacagagtgagactccatctcaaaaaaagaaaaaaaagaaaagaaactaattacacactgtgatggaggctgcaaagaacaccactaagaattcaaaatcagctgggtgcggtggctcacacctgtaatcccagcactttgggaggctgaggcaggtggatcacaaggtcaggagttcaagaccagcctggccaacatggtgaaaccccgtctctaccgaaaatacaacaaaattagcccggtgtggtggcaggtgcctgtaatcccagctacttaggaggctgaggcaggagaatcgcttgaaactgggaggcggaggtcgcagtgagccgagattcaccactgcactccagcccaggcgacagtctgagactccgtctcaaaaataaaacgattcaaaatcgaggcctgtggcatggtagggaggctgctttacgcgtgcctattattaaatgctcctggaggcatttaggtatttagatcagtctaaatatagctccattcagttcgtgcagatgacagttattgggcagtacctgtctgtgtaacacccagaaaacatgtctgtggaggggcccatggtcccgacagtaaatgcggtgagagggtcccatagagctggagttttcaagctttaggggttcccgtgctgcttgggacaggctgattcagagggtctgggtgaatgatttccaggtgattttaagactgtgctgagaaatagggcttttggggccttgtccttcaggatcaaagcatgatgctgtgtggcaatgcagaccacccaggaaccatcccaggagataagctctttgcacctcattgtctttttctgcttatgttggagcaggatgctgggggctgtcctgggatggggtgtgggacctcgtgctatttaaatacttttgcacttgaccttctgctgagtggagtggtggtttgccatcagctcagttccagtggagctgaagagacatctggtttgagtagttttagggccaccatggatatctcttcaatgcaggattggctctttccatctgctctttcattcatttgtttttgacagatagtattaaatgtttaccatgttccaggcactgtgtgaggctctgaaaatacaggggtgagcaaatccagatatcctccctgccatcatgaagtttggagtctatgagataggaccccctccctatggagaagccaccaatgcagtacagggtgacctggggccagagacaggacaaatgtcacctcctgcctccatgagatactctcactagtcatattgtgggcaagaatgtggcttacacccctagggttaacaggatgctacccaagctcatggaggaagttgaatcttaagttcccttgaaactttctaccttggtggcttttctataattttcttttttctttttcttttttttttttttttttgagactgagttttgctcttgttgcccaggctggagtgcagtggcaccatcttggctcaccgcaacctctgcctcctgggttcaagtgattctcctgcctcagcctcccgagtagctgggattacaggcatgtcccaccatgcccagctaatttttgtatttttagtagagatggggtttctccatgttggtcaggctggtttcgaactcccaacctcaggtgatccgcccacctcagccttccaaagtgctgggattacaggcatgagccactgcgtctggccttctataattttctggtagtcacgatggaaacaaacaaaacaccttagaaccagagatcgaccccctcaagcaatacatcaattcccttcacaagaaacgtcggggctacatgagtatctgtgttgaatgcggtctgaaatgatcctatggattttcccggctggttgccactgctgtacaacattcagtgcccacatccacctgtgccattaagcttttttgagacatgagagatgcctcttccctgctgtatgacatgcatttgggaagttggaaagaaatgacaaaatcagggagaaaacatccaagcttcttacctgtagatagaatcagccctcacttggtgcttattaccagttattcaagaacaataacaacaacaaaattagtagacatccaagaagcacatattaggaccaaagatagcatcaactgtatttgaaggaactgtagtttgcgcattttatgacatttttataaagtactgtaattctttcattgaggggctatgtgatggagacagactaactcattttgttatttgcattaaaattattttgggtctctgttcaaatgagtttggagaatgcttgacttgttggtctgtgtgaatgtgtatatatatatacctgaatacaggaacatcggagacctattcactcccacacactctgctatagtttgcgtgcttttgtggacacccctcatgaacaggctggcgctctaggacgctctgtgttcactgatgatgaagaaacctagaactccaagcctgtttgtaaacacactaaacacagtggcctagatagaaactgtatcgtagtttaaaatctgcctcgcgggatgttactaaactcgctaatagtttaaaggttacttacaatagagcaagttggacaattttgtggtgttggggaaatgttagggcaaggcctagaggttcattttgaatcttggtttgtgactttagggtagttagaaactttctacttaatgtacctttaaaatagtccattttctatgttttgtataatctgaaactgtacatggaaaataaagtttaaaaccagattgcccagagcaagactctaatgttcccaacggtgatgacatctagggcagaatgctgccattttgaggggcagggggtcagctgatttctcatcaagataataatgtatggtttttacactaagcaactgataaatggacaatttatcactgga

SEQ ID NO: 96 - protein, 167 aa, Homo sapiens
MLVLLAFIIAFHITSAALLFIATVDNAWWVGDEFFADVWRICTNNTNCTVINDSFQEYSTLQAVQATMILSTILCCIAFFIFVLQLFRLKQGERFVLTSIIQLMSCLCVMIAASIYTDRREDIHDKNAKFYPVTREGSYGYSYILAWVAFACTFISGMMYLILRKRK

SEQ ID NO: 97 - DNA, 1521 bp, Homo sapiens
atgtttggccgctcgcggagctgggtgggcgggggccatggcaagacttcccgcaacatccactccttggaccacctcaagtatctgtaccacgttttgaccaaaaacaccacagtcacagaacagaaccggaacctgctagtggagaccatccgttccatcactgagatcctgatctggggagatcaaaatgacagctctgtatttgacttcttcctggagaagaatatgtttgttttcttcttgaacatcttgcggcaaaagtcgggccgttacgtgtgcgttcagctgctgcagaccttgaacatcctctttgagaacatcagtcacgagacctcactttattatttgctctcaaataactacgtaaattctatcatcgttcataaatttgacttttctgatgaggagattatggcctattatatatcgttcctgaaaacactttcgttaaaactcaacaaccacactgtccatttcttttataatgagcacaccaatgactttgccctgtacacagaagccatcaagtttttcaaccaccctgaaagcatggttagaattgctgtaagaaccataactttgaatgtctataaagtgtcattggataaccaggccatgctgcactacatccgagataaaactgctgttccttacttctccaatttggtctggttcattgggagccatgtgatcgaactcgatgactgcgtgcagactgatgaggagcatcggaatcggggtaaactgagtgatctggtggcagagcacctagaccacctgcactatctcaatgacatcctgatcatcaactgtgagttcctcaacgatgtgctcactgaccacctgctcaacaggctcttcctgcccctctacgtgtactcactggagaaccaggacaagggaggagaacggccgaaaattagcctgccggtgtctctttatcttctgtcacagcacatcccgctctgggctttaaacgtgacccctcgcctcgactcgccctgccctgtgaaaatgttggtgcttcttgctttcatcatcgccttccacatcacctctgcagccttgctgttcattgccaccgtcgacaatgcctggtgggtaggagatgagttttttgcagatgtctggagaatatgtaccaacaacacgaattgcacagtcatcaatgacagctttcaagagtactccacgctgcaggcggtccaggccaccatgatcctctccaccattctctgctgcatcgccttcttcatcttcgtgctccagctcttccgcctgaagcagggagagaggtttgtcctaacctccatcatccagctaatgtcatgtctgtgtgtcatgattgcggcctccatttatacagacaggcgtgaagacattcacgacaaaaacgcgaaattctatcccgtgaccagagaaggcagctacggctactcctacatcctggcgtgggtggccttcgcctgcaccttcatcagcggcatgatgtacctgatactgaggaagcgcaaatag

SEQ ID NO: 98 - protein, 506 aa, Homo sapiens
MFGRSRSWVGGGHGKTSRNIHSLDHLKYLYHVLTKNTTVTEQNRNLLVETIRSITEILIWGDQNDSSVFDFFLEKNMFVFFLNILRQKSGRYVCVQLLQTLNILFENISHETSLYYLLSNNYVNSIIVHKFDFSDEEIMAYYISFLKTLSLKLNNHTVHFFYNEHTNDFALYTEAIKFFNHPESMVRIAVRTITLNVYKVSLDNQAMLHYIRDKTAVPYFSNLVWFIGSHVIELDDCVQTDEEHRNRGKLSDLVAEHLDHLHYLNDILIINCEFLNDVLTDHLLNRLFLPLYVYSLENQDKGGERPKISLPVSLYLLSQHIPLWALNVTPRLDSPCPVKMLVLLAFIIAFHITSAALLFIATVDNAWWVGDEFFADVWRICTNNTNCTVINDSFQEYSTLQAVQATMILSTILCCIAFFIFVLQLFRLKQGERFVLTSIIQLMSCLCVMIAASIYTDRREDIHDKNAKFYPVTREGSYGYSYILAWVAFACTFISGMMYLILRKRK

SEQ ID NO: 99 - DNA, 1056 bp, Homo sapiens
atgtttggccgctcgcggagctgggtgggcgggggccatggcaagacttcccgcaacatccactccttggaccacctcaagtatctgtaccacgttttgaccaaaaacaccacagtcacagaacagaaccggaacctgctagtggagaccatccgttccatcactgagatcctgatctggggagatcaaaatgacagctctgtatttgacttcttcctggagaagaatatgtttgttttcttcttgaacatcttgcggcaaaagtcgggccgttacgtgtgcgttcagctgctgcagaccttgaacatcctctttgagaacatcagtcacgagacctcactttattatttgctctcaaataactacgtaaattctatcatcgttcataaatttgacttttctgatgaggagattatggcctattatatatcgttcctgaaaacactttcgttaaaactcaacaaccacactgtccatttcttttataatgagcacatcccgctctgggctttaaacgtgacccctcgcctcgactcgccctgccctgtgaaaatgttggtgcttcttgctttcatcatcgccttccacatcacctctgcagccttgctgttcattgccaccgtcgacaatgcctggtgggtaggagatgagttttttgcagatgtctggagaatatgtaccaacaacacgaattgcacagtcatcaatgacagctttcaagagtactccacgctgcaggcggtccaggccaccatgatcctctccaccattctctgctgcatcgccttcttcatcttcgtgctccagctcttccgcctgaagcagggagagaggtttgtcctaacctccatcatccagctaatgtcatgtctgtgtgtcatgattgcggcctccatttatacagacaggcgtgaagacattcacgacaaaaacgcgaaattctatcccgtgaccagagaaggcagctacggctactcctacatcctggcgtgggtggccttcgcctgcaccttcatcagcggcatgatgtacctgatactgaggaagcgcaaatag

SEQ ID NO: 100 - protein, 351 aa, Homo sapiens
MFGRSRSWVGGGHGKTSRNIHSLDHLKYLYHVLTKNTTVTEQNRNLLVETIRSITEILIWGDQNDSSVFDFFLEKNMFVFFLNILRQKSGRYVCVQLLQTLNILFENISHETSLYYLLSNNYVNSIIVHKFDFSDEEIMAYYISFLKTLSLKLNNHTVHFFYNEHIPLWALNVTPRLDSPCPVKMLVLLAFIIAFHITSAALLFIATVDNAWWVGDEFFADVWRICTNNTNCTVINDSFQEYSTLQAVQATMILSTILCCIAFFIFVLQLFRLKQGERFVLTSIIQLMSCLCVMIAASIYTDRREDIHDKNAKFYPVTREGSYGYSYILAWVAFACTFISGMMYLILRKRK

SEQ ID NO: 101 - DNA, 1635 bp, Homo sapiens
atgtttggccgctcgcggagctgggtgggcgggggccatggcaagacttcccgcaacatccactccttggaccacctcaagtatctgtaccacgttttgaccaaaaacaccacagtcacagaacagaaccggaacctgctagtggagaccatccgttccatcactgagatcctgatctggggagatcaaaatgacagctctgtatttgacttcttcctggagaagaatatgtttgttttcttcttgaacatcttgcggcaaaagtcgggccgttacgtgtgcgttcagctgctgcagaccttgaacatcctctttgagaacatcagtcacgagacctcactttattatttgctctcaaataactacgtaaattctatcatcgttcataaatttgacttttctgatgaggagattatggcctattatatatcgttcctgaaaacactttcgttaaaactcaacaaccacactgtccatttcttttataatgagcacaccaatgactttgccctgtacacagaagccatcaagtttttcaaccaccctgaaagcatggttagaattgctgtaagaaccataactttgaatgtctataaagtgtcattggataaccaggccatgctgcactacatccgagataaaactgctgttccttacttctccaatttggtctggttcattgggagccatgtgatcgaactcgatgactgcgtgcagactgatgaggagcatcggaatcggggtaaactgagtgatctggtggcagagcacctagaccacctgcactatctcaatgacatcctgatcatcaactgtgagttcctcaacgatgtgctcactgaccacctgctcaacaggctcttcctgcccctctacgtgtactcactggagaaccaggacaagggaggagaacggccgaaaattagcctgccggtgtctctttatcttctgtcacaggtcttcttaattatacatcatgcaccgctggtgaactcgttagctgaagtcattctgaatggtgatctgtctgagatgtacgctaagactgaacaggatattcagagaagttctcacatcccgctctgggctttaaacgtgacccctcgcctcgactcgccctgccctgtgaaaatgttggtgcttcttgctttcatcatcgccttccacatcacctctgcagccttgctgttcattgccaccgtcgacaatgcctggtgggtaggagatgagttttttgcagatgtctggagaatatgtaccaacaacacgaattgcacagtcatcaatgacagctttcaagagtactccacgctgcaggcggtccaggccaccatgatcctctccaccattctctgctgcatcgccttcttcatcttcgtgctccagctcttccgcctgaagcagggagagaggtttgtcctaacctccatcatccagctaatgtcatgtctgtgtgtcatgattgcggcctccatttatacagacaggcgtgaagacattcacgacaaaaacgcgaaattctatcccgtgaccagagaaggcagctacggctactcctacatcctggcgtgggtggccttcgcctgcaccttcatcagcggcatgatgtacctgatactgaggaagcgcaaatag

SEQ ID NO: 102 - protein, 544 aa, Homo sapiens
MFGRSRSWVGGGHGKTSRNIHSLDHLKYLYHVLTKNTTVTEQNRNLLVETIRSITEILIWGDQNDSSVFDFFLEKNMFVFFLNILRQKSGRYVCVQLLQTLNILFENISHETSLYYLLSNNYVNSIIVHKFDFSDEEIMAYYISFLKTLSLKLNNHTVHFFYNEHTNDFALYTEAIKFFNHPESMVRIAVRTITLNVYKVSLDNQAMLHYIRDKTAVPYFSNLVWFIGSHVIELDDCVQTDEEHRNRGKLSDLVAEHLDHLHYLNDILIINCEFLNDVLTDHLLNRLFLPLYVYSLENQDKGGERPKISLPVSLYLLSQVFLIIHHAPLVNSLAEVILNGDLSEMYAKTEQDIQRSSHIPLWALNVTPRLDSPCPVKMLVLLAFIIAFHITSAALLFIATVDNAWWVGDEFFADVWRICTNNTNCTVINDSFQEYSTLQAVQATMILSTILCCIAFFIFVLQLFRLKQGERFVLTSIIQLMSCLCVMIAASIYTDRREDIHDKNAKFYPVTREGSYGYSYILAWVAFACTFISGMMYLILRKRK

SEQ ID NO: 103 - DNA, 3431 bp, Homo sapiens
aaccgcctccattacatggtccgttcctgacgtgtacaccagcctctcagagaaaactccatccctacactcggtagtctcagaattgcgctgtccacttgtcgtgtggctctgtgtcgacactgtgcgccaccatggccgtgactgcctgtcagggcttggggttcgtggtttcactgattgggattgcgggcatcattgctgccacctgcatggaccagtggagcacccaagacttgtacaacaaccccgtaacagctgttttcaactaccaggggctgtggcgctcctgtgtccgagagagctctggcttcaccgagtgccggggctacttcaccctgctggggctgccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtgtaatgctctaagacctctcagcacgggcggaagaaactcccggagagctcacccaaaaaacaaggagatcccatctagatttcttcttgcttttgactcacagctggaagttagaaaagcctcgatttcatctttggagaggccaaatggtcttagcctcagtctctgtctctaaatattccaccataaaacagctgagttatttatgaattagaggctatagctcacattttcaatcctctatttctttttttaaatataactttctactctgatgagagaatgtggttttaatctctctctcacattttgatgatttagacagactccccctcttcctcctagtcaataaacccattgatgatctatttcccagcttatccccaagaaaacttttgaaaggaaagagtagacccaaagatgttattttctgctgtttgaattttgtctccccacccccaacttggctagtaataaacacttactgaagaagaagcaataagagaaagatatttgtaatctctccagcccatgatctcggttttcttacactgtgatcttaaaagttaccaaaccaaagtcattttcagtttgaggcaaccaaacctttctactgctgttgacatcttcttattacagcaacaccattctaggagtttcctgagctctccactggagtcctctttctgtcgcgggtcagaaattgtccctagatgaatgagaaaattattttttttaatttaagtcctaaatatagttaaaataaataatgttttagtaaaatgatacactatctctgtgaaatagcctcacccctacatgtggatagaaggaaatgaaaaaataattgctttgacattgtctatatggtactttgtaaagtcatgcttaagtacaaattccatgaaaagctcactgatcctaattctttccctttgaggtctctatggctctgattgtacatgatagtaagtgtaagccatgtaaaaagtaaataatgtctgggcacagtggctcacgcctgtaatcctagcactttgggaggctgaggaggaaggatcacttgagcccagaagttcgagactagcctgggcaacatggagaagccctgtctctacaaaatacagagagaaaaaatcagccagtcatggtggcctacacctgtagtcccagcattccgggaggctgaggtgggaggatcacttgagcccagggaggttggggctgcagtgagccatgatcacaccactgcactccagccaggtgacatagcgagatcctgtctaaaaaaataaaaaataaataatggaacacagcaagtcctaggaagtaggttaaaactaattctttaaaaaaaaaaaaaagttgagcctgaattaaatgtaatgtttccaagtgacaggtatccacatttgcatggttacaagccactgccagttagcagtagcactttcctggcactgtggtcggttttgttttgttttgctttgtttagagacggggtctcactttccaggctggcctcaaactcctgcactcaagcaattcttctaccctggcctcccaagtagctggaattacaggtgtgcgccatcacaactagctggtggtcagttttgttactctgagagctgttcacttctctgaattcacctagagtggttggaccatcagatgtttgggcaaaactgaaagctctttgcaaccacacaccttccctgagcttacatcactgcccttttgagcagaaagtctaaattccttccaagacagtagaattccatcccagtaccaaagccagataggccccctaggaaactgaggtaagagcagtctctaaaaactacccacagcagcattggtgcaggggaacttggccattaggttattatttgagaggaaagtcctcacatcaatagtacatatgaaagtgacctccaaggggattggtgaatactcataaggatcttcaggctgaacagactatgtctggggaaagaacggattatgccccattaaataacaagttgtgttcaagagtcagagcagtgagctcagaggcccttctcactgagacagcaacatttaaaccaaaccagaggaagtatttgtggaactcactgcctcagtttgggtaaaggatgagcagacaagtcaactaaagaaaaaagaaaagcaaggaggagggttgagcaatctagagcatggagtttgttaagtgctctctggatttgagttgaagagcatccatttgagttgaaggccacagggcacaatgagctctcccttctaccaccagaaagtccctggtcaggtctcaggtagtgcggtgtggctcagctgggtttttaattagcgcattctctatccaacatttaattgtttgaaagcctccatatagttagattgtgctttgtaattttgttgttgttgctctatcttattgtatatgcattgagtattaacctgaatgttttgttacttaaatattaaaaacactgttatcctacagtt

SEQ ID NO: 104 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 105 - DNA, 6862 bp, Homo sapiens
ggcggggcggccgaggctgctgtgagagggcgctcgaggctgccgagagctagctagcgaaggaggcggggaggcggcgtctgcactcgctcgcccgctcgctcgcttcccggcgccgctgcgggtccgcgctgcgtttcctgctcgcgatccgctccgttgcccgcgcccggaacagcagcacctcggccgggtccgagctcggttcgggagtcttgcgcgccggcggacaccgcgcgcggagtgagccagcgccacacctgtggagccggcggccgtcgggggagccggccggggtcccgccgcgtgagtgctctgggcggcgggcggcccgggccccggcggaggcgcgccccccggctgggcgccgcgcgcaccatggggctcccagcgctcgagttcagcgactgctgcctcgatagtccgcacttccgagagacgctcaagtcgcacgaagcagagctggacaagaccaacaaattcatcaaggagctcatcaaggacgggaagtcactcataagcgcgctcaagaatttgtcttcagcgaagcggaagtttgcagattccttaaatgaatttaaatttcagtgcataggagatgcagaaacagatgatgagatgtgtatagcaagatctttgcaggagtttgccactgtcctcaggaatcttgaagatgaacggatacggatgattgagaatgccagcgaggtgctcatcactcccttggagaagtttcgaaaggaacagatcggggctgccaaggaagccaaaaagaagtatgacaaagagacagaaaagtattgtggcatcttagaaaaacacttgaatttgtcttccaaaaagaaagaatctcagcttcaggaggcagacagccaagtggacctggtccggcagcatttctatgaagtatccctggaatatgtcttcaaggtgcaggaagtccaagagagaaagatgtttgagtttgtggagcctctgctggccttcctgcaaggactcttcactttctatcaccatggttacgaactggccaaggatttcggggacttcaagacacagttaaccattagcatacagaacacaagaaatcgctttgaaggcactagatcagaagtggaatcactgatgaaaaagatgaaggagaatccccttgagcacaagaccatcagtccctacaccatggagggatacctctacgtgcaggagaaacgtcactttggaacttcttgggtgaagcactactgtacatatcaacgggattccaaacaaatcaccatggtaccatttgaccaaaagtcaggaggaaaagggggagaagatgaatcagttatcctcaaatcctgcacacggcggaaaacagactccattgagaagaggttttgctttgatgtggaagcagtagacaggccaggggttatcaccatgcaagctttgtcggaagaggaccggaggctctggatggaagccatggatggccgggaacctgtctacaactcgaacaaagacagccagagtgaagggactgcgcagttggacagcattggcttcagcataatcaggaaatgcatccatgctgtggaaaccagagggatcaacgagcaagggctgtatcgaattgtgggtgtcaactccagagtgcagaagttgctgagtgtcctgatggaccccaagactgcttctgagacagaaacagatatctgtgctgaatgggagataaagaccatcactagtgctctgaagacctacctaagaatgcttccaggaccactcatgatgtaccagtttcaaagaagtttcatcaaagcagcaaaactggagaaccaggagtctcgggtctctgaaatccacagccttgttcatcggctcccagagaaaaatcggcagatgttacagctgctcatgaaccacttggcaaatgttgctaacaaccacaagcagaatttgatgacggtggcaaaccttggtgtggtgtttggacccactctgctgaggcctcaggaagaaacagtagcagccatcatggacatcaaatttcagaacattgtcattgagatcctaatagaaaaccacgaaaagatatttaacaccgtgcccgatatgcctctcaccaatgcccagctgcacctgtctcggaagaagagcagtgactccaagcccccgtcctgcagcgagaggcccctgacgctcttccacaccgttcagtcaacagagaaacaggaacaaaggaacagcatcatcaactccagtttggaatctgtctcatcaaatccaaacagcatccttaattccagcagcagcttacagcccaacatgaactccagtgacccagacctggctgtggtcaaacccacccggcccaactcactccccccgaatccaagcccaacttcacccctctcgccatcttggcccatgttctcggcgccatccagccctatgcccacctcatccacgtccagcgactcatcccccgtcaggtctgttgcagggtttgtttggttttctgttgctgccgttgttctctcattggctcggtcctctcttcatgcagtgttcagcctcctcgtcaactttgttccctgccatccaaacctgcacttgctttttgacaggccagaagaagcggtacatgaagactccagcacaccgttccggaaggcaaaagccttgtatgcctgcaaagctgaacatgactcagaactttcgttcacagcaggcacggtcttcgataacgttcacccatctcaggagcctggctggttggaggggactctgaacggaaagactggcctcatccctgagaattacgtggagttcctctaaccgtgggccccagcagaactgctgagctttacatggtatccatgacaactgctgattccagtgtcgaggccatttctctttgccactgagaaatgcagcgtgactgactctgttgctacctgtcaacatgaatgtttctgtgagctctggtgtcactcatctccatgatcatctcagccaacatgcatcagtactgcaagaaaagaagtcaatcagcagaggagagcatttgataactaagaggaagacttgcaaagccgttttctcatgagtaccctgaatagggggcactcattttgtttcaacggtccaaacgcccaaccttcagaaagaggaagtcagatagaaatagtccctgagagcacactgtgtagctaagcctgctggggctgggtgaagaaattggcgctgagatccaggctggatccattgcttttgtttacaataggcactctctctaccccacctctcagtacttgagacttaaagtgctacaggcagctggatctgtttgcatgcaggatgaagagggttaaaacactgtttatataagatccaatctctcaccatctctaaagcagccgttggcctgtcatcagtgagatacaatccagtcttctcatgcacgggaacacacacaccctgcgtttctccctcccaggctaggaacctctctgccaccaagggctgccatccatcgcctagtaaccacggcaacccaacctactctaaaaccaaaccaaaaaaataaaataacacatcctctttgcatgacacattttttttctcccctttttggtacactttttttgaatggttttctaacaacttgaagcacaggatcaaggaattagggtggtctacttgaggcagatgggatagtagctgggaactgttccctttctgattaatttcagcagcatcggaatatatttggagcacaccctagtaacctcttgagattaaattacatagtcttaatatttctgttcctccatgcaactgatgtttgttttttaaagggtaagatgctgcctcccaatgggtgatgccatctgactggtttccccatgtcctcccattcacccatctctgctcccacccttgcctgcctctaacccaccactggccagcccccttgccctactctgggctgctgaacactggtgctgtggtggttttcaaggttaattcctaggctaaccgtatggcctatagtttaaaagcacatctatgttcactgccactctgaaaaagggaattatttctcagtctttcaaggcttgagactaatataggccattgtgattcaggaagaaacccaaggttggagggtgggatgagtaccctctgaaaaagggaatttgctggtgaaaagaggctggatcttgtggaagactgtcttggatggggaagtactacctggagatttcaaattcacttggcctgcaaacaacagagttatccgtatcttccacatgtgaatgtcattgcaagggtgactctagacaaactacaaaccgatggaccgtcaagctccccaggagccccttggatggcagcgttgcttcagagtgtttcctgtttctggaattccttgttagggaactttaaagaagaaaagaaaaacttgaattgtgttgaattactgtatcttttacttttttttttttgaaaagataaacttgtaaatagagtgatttgaaatactatatggcaaagttttatatttgatattctttaagttagttgctcacacacttaggctttgattgctgaagaagtatgtttaagagggagagaggggaggcaaagctgaagagagtcaaggtcactgtccccgcttcggcctgaaggaaagagaagacatttctatggccttgctctctgctgtcctgttggtgggcacgacacatcagtggtgttcagtctttatgtgtttttaagcatcccttgggctttggatttggagatgggaagagcatctccaggcaatgagtttttcaaagaatgcctacttagtagtaagatgaagctcaggatttaaataagtggggtcaggcattcgagtttttgtctttcttctcaggtgtatttcttggtacccccaagatatcaggccagaaagagatgagtcagttgctgtgctctttacttctttttctccacatcttctgaggctttagaaatgtggacaagctagttttcaaattttgtgtgcgtctgtaagttcttaaagaaccagcttcttagaatgttcagttctcaatgtgctgctgctttcccttctcctaaacattttaaaactcttccctttcacctccaattcccgtgatcccaaaagaagaggaagactccaggaggggtatagattgtgccgtcatagctttacaggtggttttaaagttaacaggggtttgtcatggtgattcactactcagtttatcagctcaaggattatacagctcttttccgggaactcacccaggagcaagcgagacactaccattgaatcagggaatgagaattaagaatggacaggaccaagacagaactcaagaaagccactggggaaaactcgagaagaaagggagtatactagtaggttagatctgtgaacctgaggacaagaagaccttgggaaatggaggcctcaggggatgtgcattcacatactattacgcttctcaaagagagaccaacatcatgcttttaacacatttgatgaggttttttatttgtgtttttgtttgttttttgagatggagtctcactctgtggcccaggctggagtgcagtggcgcaatcttggctcactgcaacctccacctcccaggttcaagtgattctcctgtctcagcctcccaagtagctgggactacaggcatgagccatcacacccagctagttttttgtatttttagtaaagatggggttttgccatgtttgccaggctgatctcgaactcctgacctcaagtgatctgcccacttcagacccccaaagtgctgggattccaggtgtgagccgctgcggccgaccacatttgatgtttgaagttgtaatctgtcccatcataaacttacctggagctcatgtggaggaacagaaggccaagatccttgctttgggggtgcctcacgaagcatccctgtagacatttggccccagcttcactgcttggaagcatgtccctccctcttgagttggctctgatttgaaatcgggagaaacagagctgctgccaatgggatcttttaggtaactccctccctagcttccgtgtgtctgtgcagtgcccatgagctgctgccaatgggatctttcaggtaccccctccccagcttccctgtggctgtgcggtgcccttgacagatggcttctctgtttccctttgcccagccaggctcccctccttcctattagctacaaaactggataaacttcagaatatgagccaatgagtaggaaggaacttgaagactaaagattttactctctcccctatccatgccccctacctctgactctctctgtgtgaacaggaaactttagggcagatgaggagaatgaattggttatcagagtggaagaccatggcccaggatccctgagctttcccagtagcctccagtttcctttgtaagacccagggatcacttagccatagcctgaatcttttaggggtattaaggtcagcctctcactcttccttcaggttactaacaaaatttcgtagctaaagaatgccatggccgggtgcagtggctcacgcctataatcccagcactttgggaggccgaggcgggcggatcacgaggtcaggagattgagaccatcctggctacgacggtgaaaccccgtctctactaaaaatacaaaaaattagccgggtgtggtggcgggcgcctgtagtcccagctactctggaggctgaggcaggagaatggcatgaacccaggaggcagagattgcagtgagccaagatcacgcccctgcactccagcctgggtgacagagccagactccgtctcaaagg

SEQ ID NO: 106 - protein, 814 aa, Homo sapiens
MGLPALEFSDCCLDSPHFRETLKSHEAELDKTNKFIKELIKDGKSLISALKNLSSAKRKFADSLNEFKFQCIGDAETDDEMCIARSLQEFATVLRNLEDERIRMIENASEVLITPLEKFRKEQIGAAKEAKKKYDKETEKYCGILEKHLNLSSKKKESQLQEADSQVDLVRQHFYEVSLEYVFKVQEVQERKMFEFVEPLLAFLQGLFTFYHHGYELAKDFGDFKTQLTISIQNTRNRFEGTRSEVESLMKKMKENPLEHKTISPYTMEGYLYVQEKRHFGTSWVKHYCTYQRDSKQITMVPFDQKSGGKGGEDESVILKSCTRRKTDSIEKRFCFDVEAVDRPGVITMQALSEEDRRLWMEAMDGREPVYNSNKDSQSEGTAQLDSIGFSIIRKCIHAVETRGINEQGLYRIVGVNSRVQKLLSVLMDPKTASETETDICAEWEIKTITSALKTYLRMLPGPLMMYQFQRSFIKAAKLENQESRVSEIHSLVHRLPEKNRQMLQLLMNHLANVANNHKQNLMTVANLGVVFGPTLLRPQEETVAAIMDIKFQNIVIEILIENHEKIFNTVPDMPLTNAQLHLSRKKSSDSKPPSCSERPLTLFHTVQSTEKQEQRNSIINSSLESVSSNPNSILNSSSSLQPNMNSSDPDLAVVKPTRPNSLPPNPSPTSPLSPSWPMFSAPSSPMPTSSTSSDSSPVRSVAGFVWFSVAAVVLSLARSSLHAVFSLLVNFVPCHPNLHLLFDRPEEAVHEDSSTPFRKAKALYACKAEHDSELSFTAGTVFDNVHPSQEPGWLEGTLNGKTGLIPENYVEFL

SEQ ID NO: 107 - DNA, 2088 bp, Homo sapiens
atggccgtgactgcctgtcagggcttggggttcgtggtttcactgattgggattgcgggcatcattgctgccacctgcatggaccagtggagcacccaagacttgtacaacaaccccgtaacagctgttttcaactaccaggggctgtggcgctcctgtgtccgagagagctctggcttcaccgagtgccggggctacttcaccctgctggggctgccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtctacaactcgaacaaagacagccagagtgaagggactgcgcagttggacagcattggcttcagcataatcaggaaatgcatccatgctgtggaaaccagagggatcaacgagcaagggctgtatcgaattgtgggtgtcaactccagagtgcagaagttgctgagtgtcctgatggaccccaagactgcttctgagacagaaacagatatctgtgctgaatgggagataaagaccatcactagtgctctgaagacctacctaagaatgcttccaggaccactcatgatgtaccagtttcaaagaagtttcatcaaagcagcaaaactggagaaccaggagtctcgggtctctgaaatccacagccttgttcatcggctcccagagaaaaatcggcagatgttacagctgctcatgaaccacttggcaaatgttgctaacaaccacaagcagaatttgatgacggtggcaaaccttggtgtggtgtttggacccactctgctgaggcctcaggaagaaacagtagcagccatcatggacatcaaatttcagaacattgtcattgagatcctaatagaaaaccacgaaaagatatttaacaccgtgcccgatatgcctctcaccaatgcccagctgcacctgtctcggaagaagagcagtgactccaagcccccgtcctgcagcgagaggcccctgacgctcttccacaccgttcagtcaacagagaaacaggaacaaaggaacagcatcatcaactccagtttggaatctgtctcatcaaatccaaacagcatccttaattccagcagcagcttacagcccaacatgaactccagtgacccagacctggctgtggtcaaacccacccggcccaactcactccccccgaatccaagcccaacttcacccctctcgccatcttggcccatgttctcggcgccatccagccctatgcccacctcatccacgtccagcgactcatcccccgtcaggtctgttgcagggtttgtttggttttctgttgctgccgttgttctctcattggctcggtcctctcttcatgcagtgttcagcctcctcgtcaactttgttccctgccatccaaacctgcacttgctttttgacaggccagaagaagcggtacatgaagactccagcacaccgttccggaaggcaaaagccttgtatgcctgcaaagctgaacatgactcagaactttcgttcacagcaggcacggtcttcgataacgttcacccatctcaggagcctggctggttggaggggactctgaacggaaagactggcctcatccctgagaattacgtggagttcctctaa

SEQ ID NO: 108 - protein, 695 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVYNSNKDSQSEGTAQLDSIGFSIIRKCIHAVETRGINEQGLYRIVGVNSRVQKLLSVLMDPKTASETETDICAEWEIKTITSALKTYLRMLPGPLMMYQFQRSFIKAAKLENQESRVSEIHSLVHRLPEKNRQMLQLLMNHLANVANNHKQNLMTVANLGVVFGPTLLRPQEETVAAIMDIKFQNIVIEILIENHEKIFNTVPDMPLTNAQLHLSRKKSSDSKPPSCSERPLTLFHTVQSTEKQEQRNSIINSSLESVSSNPNSILNSSSSLQPNMNSSDPDLAVVKPTRPNSLPPNPSPTSPLSPSWPMFSAPSSPMPTSSTSSDSSPVRSVAGFVWFSVAAVVLSLARSSLHAVFSLLVNFVPCHPNLHLLFDRPEEAVHEDSSTPFRKAKALYACKAEHDSELSFTAGTVFDNVHPSQEPGWLEGTLNGKTGLIPENYVEFL

SEQ ID NO: 109 - DNA, 2128 bp, Homo sapiens
aggccggccgggggcggggaggctggcgggtcggcgcgggcccagccgtgcgtgctcacgtgacgggtccgcgaggcccagctcgcgcagtcgttcgggtgagcgaagatggcggccgagagggaacctcctccgctgggggacgggaagcccaccgactttgaggatctggaggacggagaggacctgttcaccagcactgtctccaccctagagtcaagtccatcatctccagaaccagctagtcttcctgcagaagatattagtgcaaactccaatggcccaaaacccacagaagttgtattagatgatgacagagaagatctttttgcagaagccacagaagaagtttctttggacagccctgaaagggaacctatcctatcctcggaaccttctcctgcagtcacacctgtcactcctactacactcattgctcctagaattgaatcaaagagtatgtctgctcccgtgatctttgatagatccagggaagagattgaagaagaagcaaatggagacatttttgacatagaaattggtgtatcagatccagaaaaagttggtgatggcatgaatgcctatatggcatatagagtaacaacaaagacatctctttccatgttcagtaagagtgaattttcagtgaaaagaagattcagcgactttcttggtttgcacagcaaattagcaagcaaatatttacatgttggttatattgtgccaccagctccagaaaagagtatagtagggatgaccaaggtcaaagtgggtaaagaagactcatcatccactgagtttgtagaaaaacggagagcagctcttgaaaggtatcttcaaagaacagtaaaacatccaactttactacaggatcctgatttaaggcagttcttggaaagttcagagctgcctagagcagttaatacacaggctctgagtggagcaggaatattgaggatggtgaacaaggctgccgacgctgtcaacaaaatgacaatcaagatgaatgaatcggatgcatggtttgaagaaaagcagcagcaatttgagaatctggatcagcaacttaggaaacttcatgtcagtgttgaagccttggtctgtcatagaaaagaactttcagccaacacagctgcctttgctaaaagtgctgccatgttaggtaattctgaggatcatactgctttatctagagctttgtctcagcttgcagaggttgaggagaagatagaccagttacatcaagaacaagcttttgctgacttttatatgttttcagaactacttagtgactacattcgtcttattgctgcagtgaaaggtgtgtttgaccatcgaatgaagtgctggcagaaatgggaagatgctcaaattactttgctcaaaaaacgtgaagctgaagcaaaaatgatggttgctaacaaaccagataaaatacagcaagctaaaaatgaaataagagagtgggaggcgaaagtgcaacaaggggaaagagattttgaacagatatctaaaacgattcgaaaagaagtgggaagatttgagaaagaacgagtgaaggattttaaaaccgttatcatcaagtacttagaatcactagttcaaacacaacaacagctgataaaatactgggaagcattcctacctgaagccaaagccattgcctagcaataagattgttgccgttaagaagaccttggatgttgttccagttatgctggattccacagtgaaatcatttaaaaccatctaaataaaccactatatattttatgaattacatgtggttttatatacacacacacacacacacacacacacacacacacactctgacattttattacaagctgcatgtcctgaccctctttgaattaagtggactgtggcatgacattctgcaatactttgctgaattgaacactattgtgtcttaaatacttgcactaaatagtgcactgcaagaccagaaaattttacaatattttttctttacaatatgttctgtagtatgtttaccctctttatgaagtgaattaccaatgctttgaataatgttcacttatacattcctgtacagaaattacgattttgtgattacagtaataaaatgatattccttgtgaaa

SEQ ID NO: 110 - protein, 519 aa, Homo sapiens
MAAEREPPPLGDGKPTDFEDLEDGEDLFTSTVSTLESSPSSPEPASLPAEDISANSNGPKPTEVVLDDDREDLFAEATEEVSLDSPEREPILSSEPSPAVTPVTPTTLIAPRIESKSMSAPVIFDRSREEIEEEANGDIFDIEIGVSDPEKVGDGMNAYMAYRVTTKTSLSMFSKSEFSVKRRFSDFLGLHSKLASKYLHVGYIVPPAPEKSIVGMTKVKVGKEDSSSTEFVEKRRAALERYLQRTVKHPTLLQDPDLRQFLESSELPRAVNTQALSGAGILRMVNKAADAVNKMTIKMNESDAWFEEKQQQFENLDQQLRKLHVSVEALVCHRKELSANTAAFAKSAAMLGNSEDHTALSRALSQLAEVEEKIDQLHQEQAFADFYMFSELLSDYIRLIAAVKGVFDHRMKCWQKWEDAQITLLKKREAEAKMMVANKPDKIQQAKNEIREWEAKVQQGERDFEQISKTIRKEVGRFEKERVKDFKTVIIKYLESLVQTQQQLIKYWEAFLPEAKAIA

SEQ ID NO: 111 - DNA, 3052 bp, Homo sapiens
ctctctcacacacacacacacacacacacacacacacacacacacacacacacacacacacacacacacactcactctattttgtgctgtcgtaaaacccacgtgtccagccgggaagctgccagagcgtggaaccaaggagccaggacgcggcagcggccaagcgcagcagcccacggcggttgagtcgggcgcccaggtccgtccgcactctcgcgccctccgcgggcctcccaattttctcgcttgcaggtcgggaggtttccgggcggcacaatctctaggactctcctcccgcgctgctcaggggcatgtagcgcacgcagggcgcacactctcgcgcacccgcacgctcaccgagacacccgcacgcacccaccggcagcaccgagttttcagttcgaggcgccggacatgctgaagcccggagaccccggcggttcggccttcctcaaagtggacccagcctacctgcagcactggcagcaactcttccctcacggaggcgcaggcccgctcaagggcagcggcgccgcgggtctcctgagcgcgccgcagcctcttcagccgccgccgccgcccccgcccccggagcgcgctgagcctccgccggacagcctgcgcccgcggcccgcctctctctcctccgcctcgtccacgccggcttcctcttccacctccgcctcctccgcctcctcctgcgctgctgcggccgctgccgccgcgctggctggtctctcggccctgccggtgtcgcagctgccggtgttcgcgcctctagccgccgctgccgtcgccgccgagccgctgccccccaaggaactgtgcctcggcgccacctccggccccgggcccgtcaagtgcggtggtggtggcggcggcggcggggagggtcgcggcgccccgcgcttccgctgcagcgcagaggagctggactattacctgtatggccagcagcgcatggagatcatcccgctcaaccagcacaccagcgaccccaacaaccgttgcgacatgtgcgcggacaaccgcaacggcgagtgccctatgcatgggccactgcactcgctgcgccggcttgtgggcaccagcagcgctgcggccgccgcgcccccgccggagctgccggagtggctgcgggacctgcctcgcgaggtgtgcctctgcaccagtactgtgcccggcctggcctacggcatctgcgcggcgcagaggatccagcaaggcacctggattggacctttccaaggcgtgcttctgcccccagagaaggtgcaggcaggcgccgtgaggaacacgcagcatctctgggagatatatgaccaggatgggacactacagcactttattgatggtggggaacctagtaagtcgagctggatgaggtatatccgatgtgcaaggcactgcggagaacagaatctaacagtagttcagtacaggtcgaatatattctaccgagcctgtatagatatccctaggggcaccgagcttctggtgtggtacaatgacagctatacgtctttctttgggatccccttacaatgcattgcccaggatgaaaacttaaatgtcccttcaacggtaatggaagccatgtgcagacaagacgccctgcagcccttcaacaaaagcagcaaactcgcccctaccacccagcagcgctccgttgttttcccccagactccgtgcagcaggaacttctctcttctggataagtctgggcccattgaatcaggatttaatcaaatcaacgtgaaaaaccagcgagtcctggcaagcccaacttccacaagccagctccactcggagttcagtgactggcatctttggaaatgtgggcagtgctttaagactttcacccagcggatcctcttacagatgcacgtgtgcacgcagaaccccgacagaccctaccaatgcggccactgctcccagtccttttcccagccttcagaactgaggaaccacgtggtcactcactctagtgaccggcctttcaagtgcggctactgtggtcgtgcctttgccggggccaccaccctcaacaaccacatccgaacccacactggagaaaagcccttcaagtgcgagaggtgtgagaggagcttcacgcaggccacccagctgagccgacaccagcggatgcccaatgagtgcaagccaataactgagagcccagaatcaatcgaagtggattaacggattgactggttggaattaaactgcaaggaaagtcatgattaaatgtcacggacacttaagcaaaaccaaagatttcctctgagcaactttcaatcagtcccagaaaaccaaaagcagtaataaaataagtaagatgttaagagatattgatcctggcatggaagtcagaccaggaaagagattatttatttatgacttagggatgagacttatttcagtggacaactaacctgggatggttaacatttccagtcccaccatgtattttgctttgtttctaaaaagctttttaaaaactgttatttaataccaaagggaggaatcgtatgggttcttctgcccaccgttgtgactaagaatgcacagggacttggttctcgttgcacctttttttagtaacatgtttcatggggacccactgtacagcccttcattctgctgtgtcagtttggcctggcctgacactggctgccccagcggggaccacggaagcagagtgagagccttcgctgagtcaatgctaccttcagccccagacgcatcccatttccatgtcttccatgctcactgctcatgcactttttacacggtttcttccaaacagcccggtcttgatgcaggagagtctggaaaaggaagaaaatggtttcagtttcaaaattcaaaggaaaaagttgaggacttattttgtcctgtcaagattgcaagaacatgtaaaatgtacggagcttcataatacgttatattgttccgaagcagctcgttgagaaacatttgttttcaataacattttagcttaaaaaaaaaaaaagaaaatgaaaataaagttctttggtttaaggctgga

SEQ ID NO: 112 - protein, 595 aa, Homo sapiens
MLKPGDPGGSAFLKVDPAYLQHWQQLFPHGGAGPLKGSGAAGLLSAPQPLQPPPPPPPPERAEPPPDSLRPRPASLSSASSTPASSSTSASSASSCAAAAAAAALAGLSALPVSQLPVFAPLAAAAVAAEPLPPKELCLGATSGPGPVKCGGGGGGGGEGRGAPRFRCSAEELDYYLYGQQRMEIIPLNQHTSDPNNRCDMCADNRNGECPMHGPLHSLRRLVGTSSAAAAAPPPELPEWLRDLPREVCLCTSTVPGLAYGICAAQRIQQGTWIGPFQGVLLPPEKVQAGAVRNTQHLWEIYDQDGTLQHFIDGGEPSKSSWMRYIRCARHCGEQNLTVVQYRSNIFYRACIDIPRGTELLVWYNDSYTSFFGIPLQCIAQDENLNVPSTVMEAMCRQDALQPFNKSSKLAPTTQQRSVVFPQTPCSRNFSLLDKSGPIESGFNQINVKNQRVLASPTSTSQLHSEFSDWHLWKCGQCFKTFTQRILLQMHVCTQNPDRPYQCGHCSQSFSQPSELRNHVVTHSSDRPFKCGYCGRAFAGATTLNNHIRTHTGEKPFKCERCERSFTQATQLSRHQRMPNECKPITESPESIEVD

SEQ ID NO: 113 - DNA, 2244 bp, Homo sapiens
atggcggccgagagggaacctcctccgctgggggacgggaagcccaccgactttgaggatctggaggacggagaggacctgttcaccagcactgtctccaccctagagtcaagtccatcatctccagaaccagctagtcttcctgcagaagatattagtgcaaactccaatggcccaaaacccacagaagttgtattagatgatgacagagaagatctttttgcagaagccacagaagaagtttctttggacagccctgaaagggaacctatcctatcctcggaaccttctcctgcagtcacacctgtcactcctactacactcattgctcctagaattgaatcaaagagtatgtctgctcccgtgatctttgatagatccagggaagagattgaagaagaagcaaatggagacatttttgacatagaaattggtgtatcagatccagaaaaagttggtgatggcatgaatgcctatatggcatatagagtaacaacaaagacatctctttccatgttcagtaagagtgaattttcagtgaaaagaagattcagcgactttcttggtttgcacagcaaattagcaagcaaatatttacatgttggttatattgtgccaccagctccagaaaagagtatagtagggatgaccaaggtcaaagtgggtaaagaagactcatcatccactgagtttgtagaaaaacggagagcagctcttgaaaggtatcttcaaagaacagtaaaacatccaactttactacaggatcctgatttaaggcagttcttggaaagttcagagctgcctagagcagttaatacacaggctctgagtggagcaggaatattgaggatggtgaacaaggctgccgacgctgtcaacaaaatgacaatcaagatgaatgaatcggatgcatggtttgaagaaaagcagcagcaatttgagaatctggatcagcaacttaggaaacttcatgtcagtgttgaagccttggtctgtcatagaaaagaactttcagccaacacagctgcctttgctaaaagtgctgccatgttaggtaattctgaggatcatactgctttatctagagctttgtctcagcttgcagaggttgaggagaagatagaccagttacatcaagaacaagcttttgctgacttttatatgttttcagaactacttagtgactacattcgtcttattgctgcagtgaaaggtgtgtttgaccatcgaatgaagtgctggcagaaatgggaagatgctcaaattactttgctcaaaaaacgtgaagctgaagcaaaaatgatggttgctaacaaaccagataaaatacagcaagctaaaaatgaaataagagagatatatgaccaggatgggacactacagcactttattgatggtggggaacctagtaagtcgagctggatgaggtatatccgatgtgcaaggcactgcggagaacagaatctaacagtagttcagtacaggtcgaatatattctaccgagcctgtatagatatccctaggggcaccgagcttctggtgtggtacaatgacagctatacgtctttctttgggatccccttacaatgcattgcccaggatgaaaacttaaatgtcccttcaacggtaatggaagccatgtgcagacaagacgccctgcagcccttcaacaaaagcagcaaactcgcccctaccacccagcagcgctccgttgttttcccccagactccgtgcagcaggaacttctctcttctggataagtctgggcccattgaatcaggatttaatcaaatcaacgtgaaaaaccagcgagtcctggcaagcccaacttccacaagccagctccactcggagttcagtgactggcatctttggaaatgtgggcagtgctttaagactttcacccagcggatcctcttacagatgcacgtgtgcacgcagaaccccgacagaccctaccaatgcggccactgctcccagtccttttcccagccttcagaactgaggaaccacgtggtcactcactctagtgaccggcctttcaagtgcggctactgtggtcgtgcctttgccggggccaccaccctcaacaaccacatccgaacccacactggagaaaagcccttcaagtgcgagaggtgtgagaggagcttcacgcaggccacccagctgagccgacaccagcggatgcccaatgagtgcaagccaataactgagagcccagaatcaatcgaagtggattaa

SEQ ID NO: 114 - protein, 747 aa, Homo sapiens
MAAEREPPPLGDGKPTDFEDLEDGEDLFTSTVSTLESSPSSPEPASLPAEDISANSNGPKPTEVVLDDDREDLFAEATEEVSLDSPEREPILSSEPSPAVTPVTPTTLIAPRIESKSMSAPVIFDRSREEIEEEANGDIFDIEIGVSDPEKVGDGMNAYMAYRVTTKTSLSMFSKSEFSVKRRFSDFLGLHSKLASKYLHVGYIVPPAPEKSIVGMTKVKVGKEDSSSTEFVEKRRAALERYLQRTVKHPTLLQDPDLRQFLESSELPRAVNTQALSGAGILRMVNKAADAVNKMTIKMNESDAWFEEKQQQFENLDQQLRKLHVSVEALVCHRKELSANTAAFAKSAAMLGNSEDHTALSRALSQLAEVEEKIDQLHQEQAFADFYMFSELLSDYIRLIAAVKGVFDHRMKCWQKWEDAQITLLKKREAEAKMMVANKPDKIQQAKNEIREIYDQDGTLQHFIDGGEPSKSSWMRYIRCARHCGEQNLTVVQYRSNIFYRACIDIPRGTELLVWYNDSYTSFFGIPLQCIAQDENLNVPSTVMEAMCRQDALQPFNKSSKLAPTTQQRSVVFPQTPCSRNFSLLDKSGPIESGFNQINVKNQRVLASPTSTSQLHSEFSDWHLWKCGQCFKTFTQRILLQMHVCTQNPDRPYQCGHCSQSFSQPSELRNHVVTHSSDRPFKCGYCGRAFAGATTLNNHIRTHTGEKPFKCERCERSFTQATQLSRHQRMPNECKPITESPESIEVD

SEQ ID NO: 115 - DNA, 518 bp, Homo sapiens
atggcggccgagagggaacctcctccgctgggggacgggaagcccaccgactttgaggatctggaggacggagaggacctgttcaccagcactgtctccaccctagagtcaagtccatcatctccagaaccagctagtcttcctgcagaagatattagtgcaaactccaatggcccaaaacccacagaagttgtattagatgatgacagagaagatctttttgcagaccctaccaatgcggccactgctcccagtccttttcccagccttcagaactgaggaaccacgtggtcactcactctagtgaccggcctttcaagtgcggctactgtggtcgtgcctttgccggggccaccaccctcaacaaccacatccgaacccacactggagaaaagcccttcaagtgcgagaggtgtgagaggagcttcacgcaggccacccagctgagccgacaccagcggatgcccaatgagtgcaagccaataactgagagcccagaatcaatcgaagtggattaa

SEQ ID NO: 116 - protein, 172 aa, Homo sapiens
MAAEREPPPLGDGKPTDFEDLEDGEDLFTSTVSTLESSPSSPEPASLPAEDISANSNGPKPTEVVLDDDREDLFAEPYQCGHCSQSFSQPSELRNHVVTHSSDRPFKCGYCGRAFAGATTLNNHIRTHTGEKPFKCERCERSFTQATQLSRHQRMPNECKPITESPESIEVD

SEQ ID NO: 117 - DNA, 16862 bp, Homo sapiens
gaggtgcgcgcgcccgcgccgatgtgtgtgagtgcgtgtcctgctcgctccatgttgccgcctctcccggtacctgctgctgctcccggggctgcgggaaatgcgagaggctgagccggggaggaggaacccgagcagcagcggcggcggcggcggccgcggcggcgggagccccccaggaggaggaccgggatccatgtgtctttcctggtgactaggatgtcgtcggaggaggacaagagcgtggagcagccgcagccgccgccaccaccccccgaggagcctggagccccggccccgagccccgcagccgcagacaaaagacctcggggccggcctcgcaaagatggcgcttcccctttccagagagccagaaagaaacctcgaagtagggggaaaactgcagtggaagatgaggacagcatggatgggctggagacaacagaaacagaaacgattgtggaaacagaaatcaaagaacaatctgcagaagaggatgctgaagcagaagtggataacagcaaacagctaattccaactcttcagcgatctgtgtctgaggaatcggcaaactccctggtctctgttggtgtagaagccaaaatcagtgaacagctctgcgctttttgttactgtggggaaaaaagttccttaggacaaggagacttaaaacaattcagaataacgcctggatttatcttgccatggagaaaccaaccttctaacaagaaggacattgatgacaacagcaatggaacctatgagaaaatgcaaaactcagcaccacgaaaacaaagaggacagagaaaagaacgatctcctcagcagaatatagtatcttgtgtaagtgtaagcacccagacagcttcagatgatcaagctggtaaactgtgggatgaactcagtctggttgggcttccagatgccattgatatccaagccttatttgattctacaggcacttgttgggctcatcaccgttgtgtggagtggtcactaggagtatgccagatggaagaaccattgttagtgaacgtggacaaagctgttgtctcagggagcacagaacgatgtgcattttgtaagcaccttggagccactatcaaatgctgtgaagagaaatgtacccagatgtatcattatccttgtgctgcaggagccggcacctttcaggatttcagtcacatcttcctgctttgtccagaacacattgaccaagctcctgaaagatcgaaggaagatgcaaactgtgcagtgtgcgacagcccgggagacctcttagatcagttcttttgtactacttgtggtcagcactatcatggaatgtgcctggatatagcggttactccattaaaacgtgcaggttggcaatgtcctgagtgcaaagtgtgccagaactgcaaacaatcgggagaagatagcaagatgctagtgtgtgatacgtgtgacaaagggtatcatactttttgtcttcaaccagttatgaaatcagtaccaaccaatggctggaaatgcaaaaattgcagaatatgtatagagtgtggcacacggtctagttctcagtggcaccacaattgcctgatatgtgacaattgttaccaacagcaggataacttatgtcccttctgtgggaagtgttatcatccagaattgcagaaagacatgcttcattgtaatatgtgcaaaaggtgggttcacctagagtgtgacaaaccaacagatcatgaactggatactcagctcaaagaagagtatatctgcatgtattgtaaacacctgggagctgagatggatcgtttacagccaggtgaggaagtggagatagctgagctcactacagattataacaatgaaatggaagttgaaggccctgaagatcaaatggtattctcagagcaggcagctaataaagatgtcaacggtcaggagtccactcctggaattgttccagatgcggttcaagtccacactgaagagcaacagaagagtcatccctcagaaagtcttgacacagatagtcttcttattgctgtatcatcccaacatacagtgaatactgaattggaaaaacagatttctaatgaagttgatagtgaagacctgaaaatgtcttctgaagtgaagcatatttgtggcgaagatcaaattgaagataaaatggaagtgacagaaaacattgaagtcgttacacaccagatcactgtgcagcaagaacaactgcagttgttagaggaacctgaaacagtggtatccagagaagaatcaaggcctccaaaattagtcatggaatctgtcactcttccactagaaaccttagtgtccccacatgaggaaagtatttcattatgtcctgaggaacagttggttatagaaaggctacaaggagaaaaggaacagaaagaaaattctgaactttctactggattgatggactctgaaatgactcctacaattgagggttgtgtgaaagatgtttcataccaaggaggcaaatctataaagttatcatctgagacagagtcatcattttcatcatcagcagacataagcaaggcagatgtgtcttcctccccaacaccttcttcagacttgccttcgcatgacatgctgcataattacccttcagctcttagttcctctgctggaaacatcatgccaacaacttacatctcagtcactccaaaaattggcatgggtaaaccagctattactaagagaaaattttctcctggtagacctcggtccaaacagggggcttggagtacccataatacagtgagcccaccttcctggtccccagacatttcagaaggtcgggaaatttttaaacccaggcagcttcctggcagtgccatttggagcatcaaagtgggccgtgggtctggatttccaggaaagcggagacctcgaggtgcaggactgtcggggcgaggtggccgaggcaggtcaaagctgaaaagtggaatcggagctgttgtattacctggggtgtctactgcagatatttcatcaaataaggatgatgaagaaaactctatgcacaatacagttgtgttgttttctagcagtgacaagttcactttgaatcaggatatgtgtgtagtttgtggcagttttggccaaggagcagaaggaagattacttgcctgttctcagtgtggtcagtgttaccatccatactgtgtcagtattaagatcactaaagtggttcttagcaaaggttggaggtgtcttgagtgcactgtgtgtgaggcctgtgggaaggcaactgacccaggaagactcctgctgtgtgatgactgtgacataagttatcacacctactgcctagaccctccattgcagacagttcccaaaggaggctggaagtgcaaatggtgtgtttggtgcagacactgtggagcaacatctgcaggtctaagatgtgaatggcagaacaattacacacagtgcgctccttgtgcaagcttatcttcctgtccagtctgctatcgaaactatagagaagaagatcttattctgcaatgtagacaatgtgatagatggatgcatgcagtttgtcagaacttaaatactgaggaagaagtggaaaatgtagcagacattggttttgattgtagcatgtgcagaccctatatgcctgcgtctaatgtgccttcctcagactgctgtgaatcttcacttgtagcacaaattgtcacaaaagtaaaagagctagacccacccaagacttatacccaggatggtgtgtgtttgactgaatcagggatgactcagttacagagcctcacagttacagttccaagaagaaaacggtcaaaaccaaaattgaaattgaagattataaatcagaatagcgtggccgtccttcagacccctccagacatccaatcagagcattcaagggatggtgaaatggatgatagtcgagaaggagaacttatggattgtgatggaaaatcagaatctagtcctgagcgggaagctgtggatgatgaaactaagggagtggaaggaacagatggtgtcaaaaagagaaaaaggaaaccatacagaccaggtattggtggatttatggtgcggcaaagaagtcgaactgggcaagggaaaaccaaaagatctgtgatcagaaaagattcctcaggctctatttccgagcagttaccttgcagagatgatggctggagtgagcagttaccagatactttagttgatgaatctgtttctgttactgaaagcactgaaaaaataaagaagagataccgaaaaaggaaaaataagcttgaagaaactttccctgcctatttacaagaagctttctttggaaaagatcttctagatacaagtagacaaagcaagataagtttagataatctgtcagaagatggagctcagcttttatataaaacaaacatgaacacaggtttcttggatccttccttagatccactacttagttcatcctcggctccaacaaaatctggaactcacggtcctgctgatgacccattagctgatatttctgaagttttaaacacagatgatgacattcttggaataatttcagatgatctagcaaaatcagttgatcattcagatattggtcctgtcactgatgatccttcctctttgcctcagccaaatgtcaatcagagttcacgaccattaagtgaagaacagctagatgggatcctcagtcctgaactagacaaaatggtcacagatggagcaattcttggaaaattatataaaattccagagcttggcggaaaagatgttgaagacttatttacagctgtacttagtcctgcgaacactcagccaactccattgccacagcctcccccaccaacacagctgttgccaatacacaatcaggatgctttttcacggatgcctctcatgaatggccttattggatccagtcctcatctcccacataattctttgccacctggaagcggactgggaactttctctgcaattgcacaatcctcttatcctgatgccagggataaaaattcagcctttaatccaatggcaagtgatcctaacaactcttggacatcatcagctcccactgtggaaggagaaaatgacacaatgtcgaatgcccagagaagcacgcttaagtgggagaaagaggaggctctgggtgaaatggcaactgttgccccagttctctacaccaatattaatttccccaacttaaaggaagaattccctgattggactactagagtgaagcaaattgccaaattgtggagaaaagcaagctcacaagaaagagcaccatatgtgcaaaaagccagagataacagagctgctttacgcattaataaagtacagatgtcaaatgattccatgaaaaggcagcaacagcaagatagcattgatcccagctctcgtattgattcggagctttttaaagatcctttaaagcaaagagaatcagaacatgaacaggaatggaaatttagacagcaaatgcgtcagaaaagtaagcagcaagctaaaattgaagccacacagaaacttgaacaggtgaaaaatgagcagcagcagcagcaacaacagcaatttggttctcagcatcttctggtgcagtctggttcagatacaccaagtagtgggatacagagtcccttgacacctcagcctggcaatggaaatatgtctcctgcacagtcattccataaagaactgtttacaaaacagccacccagtacccctacgtctacatcttcagatgatgtgtttgtaaagccacaagctccacctcctcctccagccccatcccggattcccatccaggatagtctttctcaggctcagacttctcagccaccctcaccgcaagtgttttcacctgggtcctctaactcacgaccaccatctccaatggatccatatgcaaaaatggttggtacccctcgaccacctcctgtgggccatagtttttccagaagaaattctgctgcaccagtggaaaactgtacacctttatcatcggtatctaggccccttcaaatgaatgagacaacagcaaataggccatcccctgtcagagatttatgttcttcttccacgacaaataatgacccctatgcaaaacctccagacacacctaggcctgtgatgacagatcaatttcccaaatccttgggcctatcccggtctcctgtagtttcagaacaaactgcaaaaggccctatagcagctggaaccagtgatcactttactaaaccatctcctagggcagatgtgtttcaaagacaaaggatacctgactcatatgcacgacccttgttgacacctgcacctcttgatagtggtcctggaccttttaagactccaatgcaacctcctccatcctctcaggatccttatggatcagtgtcacaggcatcaaggcgattgtctgttgacccttatgaaaggcctgctttgacaccaagacctatagataatttttctcataatcagtcaaatgatccatatagtcagcctccccttaccccacatccagcagtgaatgaatcttttgcccatccttcaagggctttttcccagcctggaaccatatcaaggccaacatctcaggacccatactcccaacccccaggaactccacgacctgttgtagattcttattcccaatcttcaggaacagctaggtccaatacagacccttactctcaacctcctggaactccccggcctactactgttgacccatatagtcagcagccccaaaccccaagaccatctacacaaactgacttgtttgttacacctgtaacaaatcagaggcattctgatccatatgctcatcctcctggaacaccaagacctggaatttctgtcccttactctcagccaccagcaacaccaaggccaaggatttcagagggttttactaggtcctcaatgacaagaccagtcctcatgccaaatcaggatcctttcctgcaagcagcacaaaaccgaggaccagctttacctggcccgttggtaaggccacctgatacatgttcccagacacctaggccccctggacctggtctttcagacacatttagccgtgtttccccatctgctgcccgtgatccctatgatcagtctccaatgactccaagatctcagtctgactcttttggaacaagtcaaactgcccatgatgttgctgatcagccaaggcctggatcagaggggagcttctgtgcatcttcaaactctccaatgcactcccaaggccagcagttctctggtgtctcccaacttcctggacctgtgccaacttcaggagtaactgatacacagaatactgtaaatatggcccaagcagatacagagaaattgagacagcggcagaagttacgtgaaatcattctccagcagcaacagcagaagaagattgcaggtcgacaggagaaggggtcacaggactcacccgcagtgcctcatccagggcctcttcaacactggcaaccagagaatgttaaccaggctttcaccagacccccacctccctatcctgggaacattaggtctcctgttgcccctcctttaggacctagatatgctgttttcccaaaagatcagcgtggaccctatcctcctgatgttgctagtatggggatgagacctcatggatttagatttggatttccaggaggtagtcatggtaccatgccgagtcaagagcgcttccttgtgcctcctcagcaaatacagggatctggagtttctccacagctaagaagatcagtatctgtagatatgcctaggcctttaaataactcacaaatgaataatccagttggacttcctcagcatttttcaccacagagcttgccagttcagcagcacaacatactgggccaagcatatattgaactgagacatagggctcctgacggaaggcaacggctgcctttcagtgctccacctggcagcgttgtagaggcatcttctaatctgagacatggaaacttcattccccggccagactttccgggccctagacacacagaccccatgcgacgacctccccagggtctacctaatcagctacctgtgcacccagatttggaacaagtgccaccatctcaacaagagcaaggtcattctgtccattcatcttctatggtcatgaggactctgaaccatccactaggtggtgaattttcagaagctcctttgtcaacatctgtaccgtctgaaacaacgtctgataatttacagataaccacccagccttctgatggtctagaggaaaaacttgattctgatgacccttctgtgaaggaactggatgttaaagaccttgagggggttgaagtcaaagacttagatgatgaagatcttgaaaacttaaatttagatacagaggatggcaaggtagttgaattggatactttagataatttggaaactaatgatcccaacctggatgacctcttaaggtcaggagagtttgatatcattgcatatacagatccagaacttgacatgggagataagaaaagcatgtttaatgaggaactagaccttccaattgatgataagttagataatcagtgtgtatctgttgaaccaaaaaaaaaggaacaagaaaacaaaactctggttctctctgataaacattcaccacagaaaaaatccactgttaccaatgaggtaaaaacggaagtactgtctccaaattctaaggtggaatccaaatgtgaaactgaaaaaaatgatgagaataaagataatgttgacactccttgctcacaggcttctgctcactcagacctaaatgatggagaaaagacttctttgcatccttgtgatccagatctatttgagaaaagaaccaatcgagaaactgctggccccagtgcaaatgtcattcaggcatccactcaactacctgctcaagatgtaataaactcttgtggcataactggatcaactccagttctctcaagtttacttgctaatgagaaatctgataattcagacattaggccatcggggtctccaccaccaccaactctgccggcctccccatccaatcatgtgtcaagtttgcctcctttcatagcaccgcctggccgtgttttggataatgccatgaattctaatgtgacagtagtctctagggtaaaccatgttttttctcagggtgtgcaggtaaacccagggctcattccaggtcaatcaacagttaaccacagtctggggacaggaaaacctgcaactcaaactgggcctcaaacaagtcagtctggtaccagtagcatgtctggaccccaacagctaatgattcctcaaacattagcacagcagaatagagagaggccccttcttctagaagaacagcctctacttctacaggatcttttggatcaagaaaggcaagaacagcagcagcaaagacagatgcaagccatgattcgtcagcgatcagaaccgttcttccctaatattgattttgatgcaattacagatcctataatgaaagccaaaatggtggcccttaaaggtataaataaagtgatggcacaaaacaatctgggcatgccaccaatggtgatgagcaggttcccttttatgggccaggtggtaactggaacacagaacagtgaaggacagaaccttggaccacaggccattcctcaggatggcagtataacacatcagatttctaggcctaatcctccaaattttggtccaggctttgtcaatgattcacagcgtaagcagtatgaagagtggctccaggagacccaacagctgcttcaaatgcagcagaagtatcttgaagaacaaattggtgctcacagaaaatctaagaaggccctttcagctaaacaacgtactgccaagaaagctgggcgtgaatttccagaggaagatgcagaacaactcaagcatgttactgaacagcaaagcatggttcagaaacagctagaacagattcgtaaacaacagaaagaacatgctgaattgattgaagattatcggatcaaacagcagcagcaatgtgcaatggccccacctaccatgatgcccagtgtccagccccagccacccctaattccaggtgccactccacccaccatgagccaacccacctttcccatggtgccacagcagcttcagcaccagcagcacacaacagttatttctggccatactagccctgttagaatgcccagtttacctggatggcaacccaacagtgctcctgcccacctgcccctcaatcctcctagaattcagcccccaattgcccagttaccaataaaaacttgtacaccagccccagggacagtctcaaatgcaaatccacagagtggaccaccacctcgggtagaatttgatgacaacaatccctttagtgaaagttttcaagaacgggaacgtaaggaacgtttacgagaacagcaagagagacaacggatccaactcatgcaggaggtagatagacaaagagctttgcagcagaggatggaaatggagcagcatggtatggtgggctctgagataagtagtagtaggacatctgtgtcccagattcccttctacagttccgacttaccttgtgattttatgcaacctctaggaccccttcagcagtctccacaacaccaacagcaaatggggcaggttttacagcagcagaatatacaacaaggatcaattaattcaccctccacccaaactttcatgcagactaatgagcgaaggcaggtaggccctccttcatttgttcctgattcaccatcaatccctgttggaagcccaaatttttcttctgtgaagcagggacatggaaatctttctgggaccagcttccagcagtccccagtgaggccttcttttacacctgctttaccagcagcacctccagtagctaatagcagtctcccatgtggccaagattctactataacccatggacacagttatccgggatcaacccaatcgctcattcagttgtattctgatataatcccagaggaaaaagggaaaaagaaaagaacaagaaagaagaaaagagatgatgatgcagaatccaccaaggctccatcaactccccattcagatataactgccccaccgactccaggcatctcagaaactacctctactcctgcagtgagcacacccagtgagcttcctcaacaagccgaccaagagtcggtggaaccagtcggcccatccactcccaatatggcagcaggccagctatgtacagaattagagaacaaactgcccaatagtgatttctcacaagcaactccaaatcaacagacgtatgcaaattcagaagtagacaagctctccatggaaacccctgccaaaacagaagagataaaactggaaaaggctgagacagagtcctgcccaggccaagaggagcctaaattggaggaacagaatggtagtaaggtagaaggaaacgctgtagcctgtcctgtctcctcagcacagagtcctccccattctgctggggcccctgctgccaaaggagactcagggaatgaacttctgaaacacttgttgaaaaataaaaagtcatcttctcttttgaatcaaaaacctgagggcagtatttgttcagaagatgactgtacaaaggataataaactagttgagaagcagaacccagctgaaggactgcaaactttgggggctcaaatgcaaggtggttttggatgtggcaaccagttgccaaaaacagatggaggaagtgaaaccaagaaacagcgaagcaaacggactcagaggacgggtgagaaagcagcacctcgctcaaagaaaaggaaaaaggacgaagaggagaaacaagctatgtactctagcactgacacgtttacccacttgaaacagcagaataatttaagtaatcctccaacaccccctgcctctcttcctcctacaccacctcctatggcttgtcagaagatggccaatggttttgcaacaactgaagaacttgctggaaaagccggagtgttagtgagccatgaagttaccaaaactctaggacctaaaccatttcagctgcccttcagaccccaggacgacttgttggcccgagctcttgctcagggccccaagacagttgatgtgccagcctccctcccaacaccacctcataacaatcaggaagaattaaggatacaggatcactgtggtgatcgagatactcctgacagttttgttccctcatcctctcctgagagtgtggttggggtagaagtgagcaggtatccagatctgtcattggtcaaggaggagcctccagaaccggtgccgtcccccatcattccaattcttcctagcactgctgggaaaagttcagaatcaagaaggaatgacatcaaaactgagccaggcactttatattttgcgtcaccttttggtccttccccaaatggtcccagatcaggtcttatatctgtagcaattactctgcatcctacagctgctgagaacattagcagtgttgtggctgcattttccgaccttcttcacgtccgaatccctaacagctatgaggttagcagtgctccagatgtcccatccatgggtttggtcagtagccacagaatcaacccgggtttggagtatcgacagcatttacttctccgtgggcctccgccaggatctgcaaaccctcccagattagtgagctcttaccggctgaagcagcctaatgtaccatttcctccaacaagcaatggtctttctggatataaggattctagtcatggtattgcagaaagcgcagcactcagaccacagtggtgttgtcattgtaaagtggttattcttggaagtggtgtgcggaaatctttcaaagatctgacccttttgaacaaggattcccgagaaagcaccaagagggtagagaaggacattgtcttctgtagtaataactgctttattctttattcatcaactgcacaagcgaaaaactcagaaaacaaggaatccattccttcattgccacaatcacctatgagagaaacgccttccaaagcatttcatcagtacagcaacaacatctccactttggatgtgcactgtctcccccagctcccagagaaagcttctccccctgcctcaccacccatcgccttccctcctgcttttgaagcagcccaagtcgaggccaagccagatgagctgaaggtgacagtcaagctgaagcctcggctaagagctgtccatggtgggtttgaagattgcaggccgctcaataaaaaatggagaggaatgaaatggaagaagtggagcattcatattgtaatccctaaggggacatttaaaccaccttgtgaggatgaaatagatgaatttctaaagaaattgggcacttcccttaaacctgatcctgtgcccaaagactatcggaaatgttgcttttgtcatgaagaaggtgatggattgacagatggaccagcaaggctactcaaccttgacttggatctgtgggtccacttgaactgcgctctgtggtccacggaggtctatgagactcaggctggtgccttaataaatgtggagctagctctgaggagaggcctacaaatgaaatgtgtcttctgtcacaagacgggtgccactagtggatgccacagatttcgatgcaccaacatttatcacttcacttgcgccattaaagcacaatgcatgttttttaaggacaaaactatgctttgccccatgcacaaaccaaagggaattcatgagcaagaattaagttactttgcagtcttcaggagggtctatgttcagcgtgatgaggtgcgacagattgctagcatcgtgcaacgaggagaacgggaccatacctttcgcgtgggtagcctcatcttccacacaattggtcagctgcttccacagcagatgcaagcattccattctcctaaagcactcttccctgtgggctatgaagccagccggctgtactggagcactcgctatgccaataggcgctgccgctacctgtgctccattgaggagaaggatgggcgcccagtgtttgtcatcaggattgtggaacaaggccatgaagacctggttctaagtgacatctcacctaaaggtgtctgggataagattttggagcctgtggcatgtgtgagaaaaaagtctgaaatgctccagcttttcccagcgtatttaaaaggagaggatctgtttggcctgaccgtctctgcagtggcacgcatagcggaatcacttcctggggttgaggcatgtgaaaattataccttccgatacggccgaaatcctctcatggaacttcctcttgccgttaaccccacaggttgtgcccgttctgaacctaaaatgagtgcccatgtcaagaggtttgtgttaaggcctcacaccttaaacagcaccagcacctcaaagtcatttcagagcacagtcactggagaactgaacgcaccttatagtaaacagtttgttcactccaagtcatcgcagtaccggaagatgaaaactgaatggaaatccaatgtgtatctggcacggtctcggattcaggggctgggcctgtatgctgctcgagacattgagaaacacaccatggtcattgagtacatcgggactatcattcgaaacgaagtagccaacaggaaagagaagctttatgagtctcagaaccgtggtgtgtacatgttccgcatggataacgaccatgtgattgacgcgacgctcacaggagggcccgcaaggtatatcaaccattcgtgtgcacctaattgtgtggctgaagtggtgacttttgagagaggacacaaaattatcatcagctccagtcggagaatccagaaaggagaagagctctgctatgactataagtttgactttgaagatgaccagcacaagattccgtgtcactgtggagctgtgaactgccggaagtggatgaactgaaatgcattccttgctagctcagcgggcggcttgtccctaggaagaggcgattcaacacaccattggaattttgcagacagaaagagatttttgttttctgttttatgactttttgaaaaagcttctgggagttctgatttcctcagtcctttaggttaaagcagcgccaggaggaagctgacagaagcagcgttcctgaagtggccgaggttaaacggaatcacagaatggtccagcacttttgcttttttttcttttccttttcttttttttttgtttgttttttgttttgtttttcccttgtgggtgggtttcattgttttggttttctagtctcactaaggagaaacttttactggggcaaagagccgatggctgccctgccccgggcaggggccttcctatgaatgtaagactgaaatcaccagcgagggggacagagagtgctggccacggccttattaaaaaggggcaggccctctaacttcaaaatgtttttaaataaagtagacaccactgaacaaggaatgtactgaaatgacttccttagggatagagctaagggataataacttgcactaaatacatttaaatacttgattccatgagtcagtttattgtagtttttgatttctgtaaaataagagaaacttttgtatttattattgaataagtgaatgaagctatttttaaataaagttagaagaaagccaagctgctgctgttacctgcagaactaacaaaccctgttactttgtacagatatgtaaatattttgagaaaaaatacagtataaaaatagttattgaccaaatgctaccaggctctgcagcagctcgggggcttataaaatgttcatagggatgttacaatataattttgtgttataaaatatgccattataattatgtaataaccaaaatttcaacctagagtgttgggggttttttggaaaccgcagtctattagtactcaatggttttatacaccttacttctgacagagcggggcgtatgctacgactacaacttttatagctgttttggtaatttaaactaattttttcatattatattgttgcatccctacttcttcagtcaggtttttttgtgcttacaatttgtgataactgtgaataactgcttaaaaatacacccaaatggaggctgaattttttcttcagcaaaagtagttttgattagaactttgtttcagccacagagaatcatgtaaacgtaataggatcatgtagcagaaacttaaatctaaccctttagccttctatttaacacaaaaatttgaaaaagttaaaaaaaaaaaggagatgtgattatgcttacagctgcaggactctggcaatagggtttttggaagatgtaattttaaaatgtgtttgtatgaactgtttgtttacatttctttaataaaaaaaacactgttttgtgtttgcttgtagaaacttaatcagcattttgaaccaggttagctttttattttgtacttaaaattctggtactgacacttcacaggctaagtataaaatgaagttttgtgtgcacaattcaagtggactgtaaactgttggtatattcagtgatgcagttctgaacttgtatatggcatgatgtatttttatcttacagaataaatcaattgtatatatttttctcttgataaatagctgtatgaaatttgtttcctgaatatttttcttctcttgtacaatatcctgacatcctaccagtatttgtcctaccgggtttttgttgttttctgttctgtataatagtatctaatgttggcaaaaattgaattttttgaagtatacagagtgttatgggttttggaatttgtggacacagatttagaagatcaccatttacaaataaaatattttacatctataa

SEQ ID NO: 118 - protein, 4911 aa, Homo sapiens
MSSEEDKSVEQPQPPPPPPEEPGAPAPSPAAADKRPRGRPRKDGASPFQRARKKPRSRGKTAVEDEDSMDGLETTETETIVETEIKEQSAEEDAEAEVDNSKQLIPTLQRSVSEESANSLVSVGVEAKISEQLCAFCYCGEKSSLGQGDLKQFRITPGFILPWRNQPSNKKDIDDNSNGTYEKMQNSAPRKQRGQRKERSPQQNIVSCVSVSTQTASDDQAGKLWDELSLVGLPDAIDIQALFDSTGTCWAHHRCVEWSLGVCQMEEPLLVNVDKAVVSGSTERCAFCKHLGATIKCCEEKCTQMYHYPCAAGAGTFQDFSHIFLLCPEHIDQAPERSKEDANCAVCDSPGDLLDQFFCTTCGQHYHGMCLDIAVTPLKRAGWQCPECKVCQNCKQSGEDSKMLVCDTCDKGYHTFCLQPVMKSVPTNGWKCKNCRICIECGTRSSSQWHHNCLICDNCYQQQDNLCPFCGKCYHPELQKDMLHCNMCKRWVHLECDKPTDHELDTQLKEEYICMYCKHLGAEMDRLQPGEEVEIAELTTDYNNEMEVEGPEDQMVFSEQAANKDVNGQESTPGIVPDAVQVHTEEQQKSHPSESLDTDSLLIAVSSQHTVNTELEKQISNEVDSEDLKMSSEVKHICGEDQIEDKMEVTENIEVVTHQITVQQEQLQLLEEPETVVSREESRPPKLVMESVTLPLETLVSPHEESISLCPEEQLVIERLQGEKEQKENSELSTGLMDSEMTPTIEGCVKDVSYQGGKSIKLSSETESSFSSSADISKADVSSSPTPSSDLPSHDMLHNYPSALSSSAGNIMPTTYISVTPKIGMGKPAITKRKFSPGRPRSKQGAWSTHNTVSPPSWSPDISEGREIFKPRQLPGSAIWSIKVGRGSGFPGKRRPRGAGLSGRGGRGRSKLKSGIGAVVLPGVSTADISSNKDDEENSMHNTVVLFSSSDKFTLNQDMCVVCGSFGQGAEGRLLACSQCGQCYHPYCVSIKITKVVLSKGWRCLECTVCEACGKATDPGRLLLCDDCDISYHTYCLDPPLQTVPKGGWKCKWCVWCRHCGATSAGLRCEWQNNYTQCAPCASLSSCPVCYRNYREEDLILQCRQCDRWMHAVCQNLNTEEEVENVADIGFDCSMCRPYMPASNVPSSDCCESSLVAQIVTKVKELDPPKTYTQDGVCLTESGMTQLQSLTVTVPRRKRSKPKLKLKIINQNSVAVLQTPPDIQSEHSRDGEMDDSREGELMDCDGKSESSPEREAVDDETKGVEGTDGVKKRKRKPYRPGIGGFMVRQRSRTGQGKTKRSVIRKDSSGSISEQLPCRDDGWSEQLPDTLVDESVSVTESTEKIKKRYRKRKNKLEETFPAYLQEAFFGKDLLDTSRQSKISLDNLSEDGAQLLYKTNMNTGFLDPSLDPLLSSSSAPTKSGTHGPADDPLADISEVLNTDDDILGIISDDLAKSVDHSDIGPVTDDPSSLPQPNVNQSSRPLSEEQLDGILSPELDKMVTDGAILGKLYKIPELGGKDVEDLFTAVLSPANTQPTPLPQPPPPTQLLPIHNQDAFSRMPLMNGLIGSSPHLPHNSLPPGSGLGTFSAIAQSSYPDARDKNSAFNPMASDPNNSWTSSAPTVEGENDTMSNAQRSTLKWEKEEALGEMATVAPVLYTNINFPNLKEEFPDWTTRVKQIAKLWRKASSQERAPYVQKARDNRAALRINKVQMSNDSMKRQQQQDSIDPSSRIDSELFKDPLKQRESEHEQEWKFRQQMRQKSKQQAKIEATQKLEQVKNEQQQQQQQQFGSQHLLVQSGSDTPSSGIQSPLTPQPGNGNMSPAQSFHKELFTKQPPSTPTSTSSDDVFVKPQAPPPPPAPSRIPIQDSLSQAQTSQPPSPQVFSPGSSNSRPPSPMDPYAKMVGTPRPPPVGHSFSRRNSAAPVENCTPLSSVSRPLQMNETTANRPSPVRDLCSSSTTNNDPYAKPPDTPRPVMTDQFPKSLGLSRSPVVSEQTAKGPIAAGTSDHFTKPSPRADVFQRQRIPDSYARPLLTPAPLDSGPGPFKTPMQPPPSSQDPYGSVSQASRRLSVDPYERPALTPRPIDNFSHNQSNDPYSQPPLTPHPAVNESFAHPSRAFSQPGTISRPTSQDPYSQPPGTPRPVVDSYSQSSGTARSNTDPYSQPPGTPRPTTVDPYSQQPQTPRPSTQTDLFVTPVTNQRHSDPYAHPPGTPRPGISVPYSQPPATPRPRISEGFTRSSMTRPVLMPNQDPFLQAAQNRGPALPGPLVRPPDTCSQTPRPPGPGLSDTFSRVSPSAARDPYDQSPMTPRSQSDSFGTSQTAHDVADQPRPGSEGSFCASSNSPMHSQGQQFSGVSQLPGPVPTSGVTDTQNTVNMAQADTEKLRQRQKLREIILQQQQQKKIAGRQEKGSQDSPAVPHPGPLQHWQPENVNQAFTRPPPPYPGNIRSPVAPPLGPRYAVFPKDQRGPYPPDVASMGMRPHGFRFGFPGGSHGTMPSQERFLVPPQQIQGSGVSPQLRRSVSVDMPRPLNNSQMNNPVGLPQHFSPQSLPVQQHNILGQAYIELRHRAPDGRQRLPFSAPPGSVVEASSNLRHGNFIPRPDFPGPRHTDPMRRPPQGLPNQLPVHPDLEQVPPSQQEQGHSVHSSSMVMRTLNHPLGGEFSEAPLSTSVPSETTSDNLQITTQPSDGLEEKLDSDDPSVKELDVKDLEGVEVKDLDDEDLENLNLDTEDGKVVELDTLDNLETNDPNLDDLLRSGEFDIIAYTDPELDMGDKKSMFNEELDLPIDDKLDNQCVSVEPKKKEQENKTLVLSDKHSPQKKSTVTNEVKTEVLSPNSKVESKCETEKNDENKDNVDTPCSQASAHSDLNDGEKTSLHPCDPDLFEKRTNRETAGPSANVIQASTQLPAQDVINSCGITGSTPVLSSLLANEKSDNSDIRPSGSPPPPTLPASPSNHVSSLPPFIAPPGRVLDNAMNSNVTVVSRVNHVFSQGVQVNPGLIPGQSTVNHSLGTGKPATQTGPQTSQSGTSSMSGPQQLMIPQTLAQQNRERPLLLEEQPLLLQDLLDQERQEQQQQRQMQAMIRQRSEPFFPNIDFDAITDPIMKAKMVALKGINKVMAQNNLGMPPMVMSRFPFMGQVVTGTQNSEGQNLGPQAIPQDGSITHQISRPNPPNFGPGFVNDSQRKQYEEWLQETQQLLQMQQKYLEEQIGAHRKSKKALSAKQRTAKKAGREFPEEDAEQLKHVTEQQSMVQKQLEQIRKQQKEHAELIEDYRIKQQQQCAMAPPTMMPSVQPQPPLIPGATPPTMSQPTFPMVPQQLQHQQHTTVISGHTSPVRMPSLPGWQPNSAPAHLPLNPPRIQPPIAQLPIKTCTPAPGTVSNANPQSGPPPRVEFDDNNPFSESFQERERKERLREQQERQRIQLMQEVDRQRALQQRMEMEQHGMVGSEISSSRTSVSQIPFYSSDLPCDFMQPLGPLQQSPQHQQQMGQVLQQQNIQQGSINSPSTQTFMQTNERRQVGPPSFVPDSPSIPVGSPNFSSVKQGHGNLSGTSFQQSPVRPSFTPALPAAPPVANSSLPCGQDSTITHGHSYPGSTQSLIQLYSDIIPEEKGKKKRTRKKKRDDDAESTKAPSTPHSDITAPPTPGISETTSTPAVSTPSELPQQADQESVEPVGPSTPNMAAGQLCTELENKLPNSDFSQATPNQQTYANSEVDKLSMETPAKTEEIKLEKAETESCPGQEEPKLEEQNGSKVEGNAVACPVSSAQSPPHSAGAPAAKGDSGNELLKHLLKNKKSSSLLNQKPEGSICSEDDCTKDNKLVEKQNPAEGLQTLGAQMQGGFGCGNQLPKTDGGSETKKQRSKRTQRTGEKAAPRSKKRKKDEEEKQAMYSSTDTFTHLKQQNNLSNPPTPPASLPPTPPPMACQKMANGFATTEELAGKAGVLVSHEVTKTLGPKPFQLPFRPQDDLLARALAQGPKTVDVPASLPTPPHNNQEELRIQDHCGDRDTPDSFVPSSSPESVVGVEVSRYPDLSLVKEEPPEPVPSPIIPILPSTAGKSSESRRNDIKTEPGTLYFASPFGPSPNGPRSGLISVAITLHPTAAENISSVVAAFSDLLHVRIPNSYEVSSAPDVPSMGLVSSHRINPGLEYRQHLLLRGPPPGSANPPRLVSSYRLKQPNVPFPPTSNGLSGYKDSSHGIAESAALRPQWCCHCKVVILGSGVRKSFKDLTLLNKDSRESTKRVEKDIVFCSNNCFILYSSTAQAKNSENKESIPSLPQSPMRETPSKAFHQYSNNISTLDVHCLPQLPEKASPPASPPIAFPPAFEAAQVEAKPDELKVTVKLKPRLRAVHGGFEDCRPLNKKWRGMKWKKWSIHIVIPKGTFKPPCEDEIDEFLKKLGTSLKPDPVPKDYRKCCFCHEEGDGLTDGPARLLNLDLDLWVHLNCALWSTEVYETQAGALINVELALRRGLQMKCVFCHKTGATSGCHRFRCTNIYHFTCAIKAQCMFFKDKTMLCPMHKPKGIHEQELSYFAVFRRVYVQRDEVRQIASIVQRGERDHTFRVGSLIFHTIGQLLPQQMQAFHSPKALFPVGYEASRLYWSTRYANRRCRYLCSIEEKDGRPVFVIRIVEQGHEDLVLSDISPKGVWDKILEPVACVRKKSEMLQLFPAYLKGEDLFGLTVSAVARIAESLPGVEACENYTFRYGRNPLMELPLAVNPTGCARSEPKMSAHVKRFVLRPHTLNSTSTSKSFQSTVTGELNAPYSKQFVHSKSSQYRKMKTEWKSNVYLARSRIQGLGLYAARDIEKHTMVIEYIGTIIRNEVANRKEKLYESQNRGVYMFRMDNDHVIDATLTGGPARYINHSCAPNCVAEVVTFERGHKIIISSSRRIQKGEELCYDYKFDFEDDQHKIPCHCGAVNCRKWMN

SEQ ID NO: 119 - DNA, 3282 bp, Homo sapiens
gagctggtttattctgcggccgaggattacatttatgcacgaacgggcttactggttccagattccccacttgggcacaggcataggaggcttgttttccaaattgctggttttaattgcacctgcctttcagattacctctgggaatctgtgggaggagccgagagggtggaaaatgtttcttagctttgcaaaaggaagaaaactttgtcacccagcgggagacctcagccacgagtaacccggggagacaccagaaccgggacgggctttgactgatttgcctacgagggttccgtaggaaaggacgcttgaattcggcgcttcggcggcggcggcggccgcgcgagttccctgctcaccctccctctccgcggaagtccccacgaggtggcttcagggtgtaacagagcgcgcggctccagtccgaaggcagcggccgggggagggaaggaggggaccgaacccccgaggagtttcgcagaatcaacttctggttagagttatgggaagcgcggttatggacaccaagaagaaaaaagatgtttccagccccggcgggagcggcggcaagaaaaatgccagccagaagaggcgttcgctgcgcgtgcacattccggacctgagctccttcgccatgccgctcctggacggagacctggagggttccggaaagcattcctctcgaaaggtggacagccccttcggcccgggcagcccctccaaagggttcttctccagaggcccccagccccggccctccagccccatgtctgcacctgtgaggcccaagaccagccccggctctcccaaaaccgtgttcccgttctcctaccaggagtccccgccacgctcccctcgacgcatgagcttcagtgggatcttccgctcctcctccaaagagtcttcccccaactccaaccctgctacctcgcccgggggcatcaggtttttctcccgctccagaaaaacctccggcctctcctcctctccgtcaacacccacccaagtgaccaagcagcacacgtttcccctggaatcctataagcacgagcctgaacggttagagaatcgcatctatgcctcgtcttcccccccggacacagggcagaggttctgcccgtcttccttccagagcccgaccaggcctccactggcatcaccgacacactatgctccctccaaagccgcggcgctggcggcggccctgggacccgcggaagccggcatgctggagaagctggagttcgaggacgaagcagtagaagactcagaaagtggtgtttacatgcgattcatgaggtcacacaagtgttatgacatcgttccaaccagttcaaagcttgttgtctttgatactacattacaagttaaaaaggccttctttgctttggtagccaacggtgtccgagcagcgccactgtgggagagtaaaaaacaaagttttgtaggaatgctaacaattacagatttcataaatatactacatagatactataaatcacctatggtacagatttatgaattagaggaacataaaattgaaacatggagggagctttatttacaagaaacatttaagcctttagtgaatatatctccagatgcaagcctcttcgatgctgtatactccttgatcaaaaataaaatccacagattgcccgttattgaccctatcagtgggaatgcactttatatacttacccacaaaagaatcctcaagttcctccagctttttatgtctgatatgccaaagcctgccttcatgaagcagaacctggatgagcttggaataggaacgtaccacaacattgccttcatacatccagacactcccatcatcaaagccttgaacatatttgtggaaagacgaatatcagctctgcctgttgtggatgagtcaggaaaagttgtagatatttattccaaatttgatgtaattaatcttgctgctgagaaaacatacaataacctagatatcacggtgacccaggcccttcagcaccgttcacagtattttgaaggtgttgtgaagtgcaataagctggaaatactggagaccatcgtggacagaatagtaagagctgaggtccatcggctggtggtggtaaatgaagcagatagtattgtgggtattatttccctgtcggacattctgcaagccctgatcctcacaccagcaggtgccaaacaaaaggagacagaaacggagtgaccgccgtgaatgtagacgccctaggaggagaacttgaacaaagtctctgggtcacgttttgcctcatgaacactggctgcaagtggttaagaatgtatatcagggtttaacaataggtatttcttccagtgatgttgaaattaagcttaaaaaagaaagattttatgtgcttgaagattcaggcttgcattaaaagactgttttcagacctttgtctgaaggattttaaatgctgtatgtcattaaagtgcactgtgtcctgaagttttcattatttttcatttcaaagaattcactggtatggaacaggtgatgtggcataaggtgagtgcacggtatgttcagatcacagtgccttatgtccgaatacagcaatatgtcaccgccgcagccggggcgcacgcgtgtgaaacaacaccgagcttgaatgtggaagtctttgaaccttttaccaaatcagtttgttttctttagatttgtcaaaaagttgtaatttgaatataaataattactttaaaattgtaatgacacttttacacgtaagtgttttgttctgggctaccgtgtcaacgaggctgctttacaacagctttatttatttttactttcatgcaatttttttacacatcttttggtggagtaaacttcaccacatccatgaataaactctcagttattttgaaatggcaaatttctcattatttaagtttggatctggaaaggacatgacttctgaaatagccgctgctgggttttaaaagctgaggtctctcaaagtgtggaggagacgttgccgtcaggcgggagccaagtgccgggaagatgtctattttttttcttgtgtattgaaatgtaaaatcatgatgtttgttatgactgctgatgcgattgtttttgtaaattttattgtggcatatacagtattgtcatacagttgaagagaaacaatgtttcctaatgtaagtgctctgaaaatgttgacactgtatatatatatatgaggatagtttgttttttttttgttttgggtttttttttttcagattgaaaaattaaaatagatccta

SEQ ID NO: 120 - protein, 569 aa, Homo sapiens
MGSAVMDTKKKKDVSSPGGSGGKKNASQKRRSLRVHIPDLSSFAMPLLDGDLEGSGKHSSRKVDSPFGPGSPSKGFFSRGPQPRPSSPMSAPVRPKTSPGSPKTVFPFSYQESPPRSPRRMSFSGIFRSSSKESSPNSNPATSPGGIRFFSRSRKTSGLSSSPSTPTQVTKQHTFPLESYKHEPERLENRIYASSSPPDTGQRFCPSSFQSPTRPPLASPTHYAPSKAAALAAALGPAEAGMLEKLEFEDEAVEDSESGVYMRFMRSHKCYDIVPTSSKLVVFDTTLQVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPDTPIIKALNIFVERRISALPVVDESGKVVDIYSKFDVINLAAEKTYNNLDITVTQALQHRSQYFEGVVKCNKLEILETIVDRIVRAEVHRLVVVNEADSIVGIISLSDILQALILTPAGAKQKETETE

SEQ ID NO: 121 - DNA, 2325 bp, Homo sapiens
atgtcgtcggaggaggacaagagcgtggagcagccgcagccgccgccaccaccccccgaggagcctggagccccggccccgagccccgcagccgcagacaaaagacctcggggccggcctcgcaaagatggcgcttcccctttccagagagccagaaagaaacctcgaagtagggggaaaactgcagtggaagatgaggacagcatggatgggctggagacaacagaaacagaaacgattgtggaaacagaaatcaaagaacaatctgcagaagaggatgctgaagcagaagtggataacagcaaacagctaattccaactcttcagcgatctgtgtctgaggaatcggcaaactccctggtctctgttggtgtagaagccaaaatcagtgaacagctctgcgctttttgttactgtggggaaaaaagttccttaggacaaggagacttaaaacaattcagaataacgcctggatttatcttgccatggagaaaccaaccttctaacaagaaggacattgatgacaacagcaatggaacctatgagaaaatgcaaaactcagcaccacgaaaacaaagaggacagagaaaagaacgatctcctcagcagaatatagtatcttgtgtaagtgtaagcacccagacagcttcagatgatcaagctggtaaactgtgggatgaactcagtctggttgggcttccagatgccattgatatccaagccttatttgattctacaggcacttgttgggctcatcaccgttgtgtggagtggtcactaggagtatgccagatggaagaaccattgttagtgaacgtggacaaagctgttgtctcagggagcacagaacgatgtgcattttgtaagcaccttggagccactatcaaatgctgtgaagagaaatgtacccagatgtatcattatccttgtgctgcaggagccggcacctttcaggatttcagtcacatcttcctgctttgtccagaacacattgaccaagctcctgaaagatcgaaggaagatgcaaactgtgcagtgtgcgacagcccgggagacctcttagatcagttcttttgtactacttgtggtcagcactatcatggaatgtgcctggatatagcggttactccattaaaacgtgcaggttggcaatgtcctgagtgcaaagtgtgccagaactgcaaacaatcgggagaagatagcaagatgctagtgtgtgatacgtgtgacaaagggtatcatactttttgtcttcaaccagttatgaaatcagtaccaaccaatggctggaaatgcaaagcggcgctggcggcggccctgggacccgcggaagccggcatgctggagaagctggagttcgaggacgaagcagtagaagactcagaaagtggtgtttacatgcgattcatgaggtcacacaagtgttatgacatcgttccaaccagttcaaagcttgttgtctttgatactacattacaagttaaaaaggccttctttgctttggtagccaacggtgtccgagcagcgccactgtgggagagtaaaaaacaaagttttgtaggaatgctaacaattacagatttcataaatatactacatagatactataaatcacctatggtacagatttatgaattagaggaacataaaattgaaacatggagggagctttatttacaagaaacatttaagcctttagtgaatatatctccagatgcaagcctcttcgatgctgtatactccttgatcaaaaataaaatccacagattgcccgttattgaccctatcagtgggaatgcactttatatacttacccacaaaagaatcctcaagttcctccagctttttatgtctgatatgccaaagcctgccttcatgaagcagaacctggatgagcttggaataggaacgtaccacaacattgccttcatacatccagacactcccatcatcaaagccttgaacatatttgtggaaagacgaatatcagctctgcctgttgtggatgagtcaggaaaagttgtagatatttattccaaatttgatgtaattaatcttgctgctgagaaaacatacaataacctagatatcacggtgacccaggcccttcagcaccgttcacagtattttgaaggtgttgtgaagtgcaataagctggaaatactggagaccatcgtggacagaatagtaagagctgaggtccatcggctggtggtggtaaatgaagcagatagtattgtgggtattatttccctgtcggacattctgcaagccctgatcctcacaccagcaggtgccaaacaaaaggagacagaaacggagtga

SEQ ID NO: 122 - protein, 774 aa, Homo sapiens
MSSEEDKSVEQPQPPPPPPEEPGAPAPSPAAADKRPRGRPRKDGASPFQRARKKPRSRGKTAVEDEDSMDGLETTETETIVETEIKEQSAEEDAEAEVDNSKQLIPTLQRSVSEESANSLVSVGVEAKISEQLCAFCYCGEKSSLGQGDLKQFRITPGFILPWRNQPSNKKDIDDNSNGTYEKMQNSAPRKQRGQRKERSPQQNIVSCVSVSTQTASDDQAGKLWDELSLVGLPDAIDIQALFDSTGTCWAHHRCVEWSLGVCQMEEPLLVNVDKAVVSGSTERCAFCKHLGATIKCCEEKCTQMYHYPCAAGAGTFQDFSHIFLLCPEHIDQAPERSKEDANCAVCDSPGDLLDQFFCTTCGQHYHGMCLDIAVTPLKRAGWQCPECKVCQNCKQSGEDSKMLVCDTCDKGYHTFCLQPVMKSVPTNGWKCKAALAAALGPAEAGMLEKLEFEDEAVEDSESGVYMRFMRSHKCYDIVPTSSKLVVFDTTLQVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPDTPIIKALNIFVERRISALPVVDESGKVVDIYSKFDVINLAAEKTYNNLDITVTQALQHRSQYFEGVVKCNKLEILETIVDRIVRAEVHRLVVVNEADSIVGIISLSDILQALILTPAGAKQKETETE

SEQ ID NO: 123 - DNA, 1695 bp, Homo sapiens
atgtcgtcggaggaggacaagagcgtggagcagccgcagccgccgccaccaccccccgaggagcctggagccccggccccgagccccgcagccgcagacaaaagacctcggggccggcctcgcaaagatggcgcttcccctttccagagagccagaaagaaacctcgaagtagggggaaaactgcagtggaagatgaggacagcatggatgggctggagacaacagaaacagaaacgattgtggaaacagaaatcaaagaacaatctgcagaagaggatgctgaagcagaagtggataacagcaaacagctaattccaactcttcagcgatctgtgtctgaggaatcggcaaactccctggtctctgttggtgtagaagccaaaatcagtgaacagctctgcgctttttgttactgtggggaaaaaagttccttaggacaaggagacttaaaacaattcagaataacgcctggatttatcttgccatggagaaaccaaccttctaacaagaaggacattgatgacaacagcaatggaacctatgagaaaatgcaaaactcagcaccacgaaaacaaagaggacagagaaaagaacgatctcctcagcagaatatagtatcttgtgtaagtgtaagcacccagacagcttcagatgatcaagctggtaaactgtgggatgaactcagtctggttgggcttccagatgccattgatatccaagccttatttgattctacaggcacttgttgggctcatcaccgttgtgtggagtggtcactaggagtatgccagatggaagaaccattgttagtgaacgtggacaaagctgttgtctcagggagcacagaagttaaaaaggccttctttgctttggtagccaacggtgtccgagcagcgccactgtgggagagtaaaaaacaaagttttgtaggaatgctaacaattacagatttcataaatatactacatagatactataaatcacctatggtacagatttatgaattagaggaacataaaattgaaacatggagggagctttatttacaagaaacatttaagcctttagtgaatatatctccagatgcaagcctcttcgatgctgtatactccttgatcaaaaataaaatccacagattgcccgttattgaccctatcagtgggaatgcactttatatacttacccacaaaagaatcctcaagttcctccagctttttatgtctgatatgccaaagcctgccttcatgaagcagaacctggatgagcttggaataggaacgtaccacaacattgccttcatacatccagacactcccatcatcaaagccttgaacatatttgtggaaagacgaatatcagctctgcctgttgtggatgagtcaggaaaagttgtagatatttattccaaatttgatgtaattaatcttgctgctgagaaaacatacaataacctagatatcacggtgacccaggcccttcagcaccgttcacagtattttgaaggtgttgtgaagtgcaataagctggaaatactggagaccatcgtggacagaatagtaagagctgaggtccatcggctggtggtggtaaatgaagcagatagtattgtgggtattatttccctgtcggacattctgcaagccctgatcctcacaccagcaggtgccaaacaaaaggagacagaaacggagtga

SEQ ID NO: 124 - protein, 566 aa, Homo sapiens
MSSEEDKSVEQPQPPPPPPEEPGAPAPSPAAADKRPRGRPRKDGASPFQRARKKPRSRGKTAVEDEDSMETDGLETTETETIVETEIKEQSAEEDAEAEVDNSKQLIPTLQRSVSEESANSLVSVGVEAKISEQLCAFCYCGEKSSLGQGDLKQFRITPGFILPWRNQPSNKKDIDDNSNGTYEKMQNSAPRKQRGQRKERSPQQNIVSCVSVSTQTASDDQAGKLWDELSLVGLPDAIDIQALFDSTGTCWAHHRCVEWSLGVCQMEEPLLVNVDKAVVSGSTEVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPDTPIIKALNIFVERRISALPVVDESGKVVDIYSKFDVINLAAEKTYNNLDITVTQALQHRSQYFEGVVKCNKLEILETIVDRIVRAEVHRLVVVNEADSIVGIISLSDILQALILTPAGAKQKETETE

SEQ ID NO: 125 - DNA, 4668 bp, Homo sapiens
atgtcgtcggaggaggacaagagcgtggagcagccgcagccgccgccaccaccccccgaggagcctggagccccggccccgagccccgcagccgcagacaaaagacctcggggccggcctcgcaaagatggcgcttcccctttccagagagccagaaagaaacctcgaagtagggggaaaactgcagtggaagatgaggacagcatggatgggctggagacaacagaaacagaaacgattgtggaaacagaaatcaaagaacaatctgcagaagaggatgctgaagcagaagtggataacagcaaacagctaattccaactcttcagcgatctgtgtctgaggaatcggcaaactccctggtctctgttggtgtagaagccaaaatcagtgaacagctctgcgctttttgttactgtggggaaaaaagttccttaggacaaggagacttaaaacaattcagaataacgcctggatttatcttgccatggagaaaccaaccttctaacaagaaggacattgatgacaacagcaatggaacctatgagaaaatgcaaaactcagcaccacgaaaacaaagaggacagagaaaagaacgatctcctcagcagaatatagtatcttgtgtaagtgtaagcacccagacagcttcagatgatcaagctggtaaactgtgggatgaactcagtctggttgggcttccagatgccattgatatccaagccttatttgattctacaggcacttgttgggctcatcaccgttgtgtggagtggtcactaggagtatgccagatggaagaaccattgttagtgaacgtggacaaagctgttgtctcagggagcacagaacgatgtgcattttgtaagcaccttggagccactatcaaatgctgtgaagagaaatgtacccagatgtatcattatccttgtgctgcaggagccggcacctttcaggatttcagtcacatcttcctgctttgtccagaacacattgaccaagctcctgaaagatcgaaggaagatgcaaactgtgcagtgtgcgacagcccgggagacctcttagatcagttcttttgtactacttgtggtcagcactatcatggaatgtgcctggatatagcggttactccattaaaacgtgcaggttggcaatgtcctgagtgcaaagtgtgccagaactgcaaacaatcgggagaagatagcaagatgctagtgtgtgatacgtgtgacaaagggtatcatactttttgtcttcaaccagttatgaaatcagtaccaaccaatggctggaaatgcaaaaattgcagaatatgtatagagtgtggcacacggtctagttctcagtggcaccacaattgcctgatatgtgacaattgttaccaacagcaggataacttatgtcccttctgtgggaagtgttatcatccagaattgcagaaagacatgcttcattgtaatatgtgcaaaaggtgggttcacctagagtgtgacaaaccaacagatcatgaactggatactcagctcaaagaagagtatatctgcatgtattgtaaacacctgggagctgagatggatcgtttacagccaggtgaggaagtggagatagctgagctcactacagattataacaatgaaatggaagttgaaggccctgaagatcaaatggtattctcagagcaggcagctaataaagatgtcaacggtcaggagtccactcctggaattgttccagatgcggttcaagtccacactgaagagcaacagaagagtcatccctcagaaagtcttgacacagatagtcttcttattgctgtatcatcccaacatacagtgaatactgaattggaaaaacagatttctaatgaagttgatagtgaagacctgaaaatgtcttctgaagtgaagcatatttgtggcgaagatcaaattgaagataaaatggaagtgacagaaaacattgaagtcgttacacaccagatcactgtgcagcaagaacaactgcagttgttagaggaacctgaaacagtggtatccagagaagaatcaaggcctccaaaattagtcatggaatctgtcactcttccactagaaaccttagtgtccccacatgaggaaagtatttcattatgtcctgaggaacagttggttatagaaaggctacaaggagaaaaggaacagaaagaaaattctgaactttctactggattgatggactctgaaatgactcctacaattgagggttgtgtgaaagatgtttcataccaaggaggcaaatctataaagttatcatctgagacagagtcatcattttcatcatcagcagacataagcaaggcagatgtgtcttcctccccaacaccttcttcagacttgccttcgcatgacatgctgcataattacccttcagctcttagttcctctgctggaaacatcatgccaacaacttacatctcagtcactccaaaaattggcatgggtaaaccagctattactaagagaaaattttctcctggtagacctcggtccaaacagggggcttggagtacccataatacagtgagcccaccttcctggtccccagacatttcagaaggtcgggaaatttttaaacccaggcagcttcctggcagtgccatttggagcatcaaagtgggccgtgggtctggatttccaggaaagcggagacctcgaggtgcaggactgtcggggcgaggtggccgaggcaggtcaaagctgaaaagtggaatcggagctgttgtattacctggggtgtctactgcagatatttcatcaaataaggatgatgaagaaaactctatgcacaatacagttgtgttgttttctagcagtgacaagttcactttgaatcaggatatgtgtgtagtttgtggcagttttggccaaggagcagaaggaagattacttgcctgttctcagtgtggtcagtgttaccatccatactgtgtcagtattaagatcactaaagtggttcttagcaaaggttggaggtgtcttgagtgcactgtgtgtgaggcctgtgggaaggcaactgacccaggaagactcctgctgtgtgatgactgtgacataagttatcacacctactgcctagaccctccattgcagacagttcccaaaggaggctggaagtgcaaatggtgtgtttggtgcagacactgtggagcaacatctgcaggtctaagatgtgaatggcagaacaattacacacagtgcgctccttgtgcaagcttatcttcctgtccagtctgctatcgaaactatagagaagaagatcttattctgcaatgtagacaatgtgatagatggatgcatgcagtttgtcagaacttaaatactgaggaagaagtggaaaatgtagcagacattggttttgattgtagcatgtgcagaccctatatgcctgcgtctaatgtgccttcctcagactgctgtgaatcttcacttgtagcacaaattgtcacaaaagtaaaagagctagacccacccaagacttatacccaggatggtgtgtgtttgactgaatcagggatgactcagttacagagcctcacagttacagttccaagaagaaaacggtcaaaaccaaaattgaaattgaagattataaatcagaatagcgtggccgtccttcagacccctccagacatccaatcagagcattcaagggatggtgaaatggatgatagtcgagcagtagaagactcagaaagtggtgtttacatgcgattcatgaggtcacacaagtgttatgacatcgttccaaccagttcaaagcttgttgtctttgatactacattacaagttaaaaaggccttctttgctttggtagccaacggtgtccgagcagcgccactgtgggagagtaaaaaacaaagttttgtaggaatgctaacaattacagatttcataaatatactacatagatactataaatcacctatggtacagatttatgaattagaggaacataaaattgaaacatggagggagctttatttacaagaaacatttaagcctttagtgaatatatctccagatgcaagcctcttcgatgctgtatactccttgatcaaaaataaaatccacagattgcccgttattgaccctatcagtgggaatgcactttatatacttacccacaaaagaatcctcaagttcctccagctttttatgtctgatatgccaaagcctgccttcatgaagcagaacctggatgagcttggaataggaacgtaccacaacattgccttcatacatccagacactcccatcatcaaagccttgaacatatttgtggaaagacgaatatcagctctgcctgttgtggatgagtcaggaaaagttgtagatatttattccaaatttgatgtaattaatcttgctgctgagaaaacatacaataacctagatatcacggtgacccaggcccttcagcaccgttcacagtattttgaaggtgttgtgaagtgcaataagctggaaatactggagaccatcgtggacagaatagtaagagctgaggtccatcggctggtggtggtaaatgaagcagatagtattgtgggtattatttccctgtcggacattctgcaagccctgatcctcacaccagcaggtgccaaacaaaaggagacagaaacggagtga

SEQ ID NO: 126 - protein, 1557 aa, Homo sapiens
MSSEEDKSVEQPQPPPPPPEEPGAPAPSPAAADKRPRGRPRKDGASPFQRARKKPRSRGKTAVEDEDSMDGLETTETETIVETEIKEQSAEEDAEAEVDNSKQLIPTLQRSVSEESANSLVSVGVEAKISEQLCAFCYCGEKSSLGQGDLKQFRITPGFILPWRNQPSNKKDIDDNSNGTYEKMQNSAPRKQRGQRKERSPQQNIVSCVSVSTQTASDDQAGKLWDELSLVGLPDAIDIQALFDSTGTCWAHHRCVEWSLGVCQMEEPLLVNVDKAVVSGSTERCAFCKHLGATIKCCEEKCTQMYHYPCAAGAGTFQDFSHIFLLCPEHIDQAPERSKEDANCAVCDSPGDLLDQFFCTTCGQHYHGMCLDIAVTPLKRAGWQCPECKVCQNCKQSGEDSKMLVCDTCDKGYHTFCLQPVMKSVPTNGWKCKNCRICIECGTRSSSQWHHNCLICDNCYQQQDNLCPFCGKCYHPELQKDMLHCNMCKRWVHLECDKPTDHELDTQLKEEYICMYCKHLGAEMDRLQPGEEVEIAELTTDYNNEMEVEGPEDQMETVFSEQAANKDVNGQESTPGIVPDAVQVHTEEQQKSHPSESLDTDSLLIAVSSQHTVNTELEKQISNEVDSEDLKMSSEVKHICGEDQIEDKMEVTENIEVVTHQITVQQEQLQLLEEPETVVSREESRPPKLVMESVTLPLETLVSPHEESISLCPEEQLVIERLQGEKEQKENSELSTGLMDSEMTPTIEGCVKDVSYQGGKSIKLSSETESSFSSSADISKADVSSSPTPSSDLPSHDMLHNYPSALSSSAGNIMPTTYISVTPKIGMGKPAITKRKFSPGRPRSKQGAWSTHNTVSPPSWSPDISEGREIFKPRQLPGSAIWSIKVGRGSGFPGKRRPRGAGLSGRGGRGRSKLKSGIGAVVLPGVSTADISSNKDDEENSMHNTVVLFSSSDKFTLNQDMCVVCGSFGQGAEGRLLACSQCGQCYHPYCVSIKITKVVLSKGWRCLECTVCEACGKATDPGRLLLCDDCDISYHTYCLDPPLQTVPKGGWKCKWCVWCRHCGATSAGLRCEWQNNYTQCAPCASLSSCPVCYRNYREEDLILQCRQCDRWMHAVCQNLNTEEEVENVADIGFDCSMCRPYMPASNVPSSDCCESSLVAQIVTKVKELDPPKTYTQDGVCLTESGMTQLQSLTVTVPRRKRSKPKLKLKIINQNSVAVLQTPPDIQSEHSRDGEMDDSRAVEDSESGVYMRFMRSHKCYDIVPTSSKLVVFDTTLQVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPDTPIIKALNIFVERRISALPVVDESGKVVDIYSKFDVINLAAEKTYNNLDITVTQALQHRSQYFEGVVKCNKLEILETIVDRIVRAEVHRLVVVNEADSIVGIISLSDILQALILTPAGAKQKETETE

SEQ ID NO: 127 - DNA, 2310 bp, Homo sapiens
tgaggcgcgccggctggttcaactccggccgccgcgccgaaaccagcagcggtccgggtcgaaccagcaccggcctcgggaggttccgccgcctgctctgccgctgttccaactgccgctgtagagccactgggatgcgcaccaccggcaggggttcgtcgggactgcggaccgtgaggccccgtcgcggcgccaggagcaaccgagtcacgagggaaaagagccgcaccggccgcgttagagccatgtttcccttagtgcgggagaagcgcacatcagtgacgtcacggacgcgccgcgacctcgcgtacggtggctggcgaggctcagtacggtgtgtggagctggagcaccgtgaggaagaagcgaggttctttttaagagttcagctgcgagatatcaaacaaagaattactctgtacaaagccagaacacatatatcaaagtaatcctgaagtatcagaacaaaataataggctgtaacagaggaggaaatgattttgaatagcctctctctgtgttaccataataagctaatcctggccccaatggttcgggtagggactcttccaatgaggctgctggccctggattatggagcggacattgtttactgtgaggagctgatcgacctcaagatgattcagtgcaagagagttgttaatgaggtgctcagcacagtggactttgtcgcccctgatgatcgagttgtcttccgcacctgtgaaagagagcagaacagggtggtcttccagatggggacttcagacgcagagcgagcccttgctgtggccaggcttgtagaaaatgatgtggctggtattgatgtcaacatgggctgtccaaaacaatattccaccaagggaggaatgggagctgccctgctgtcagaccctgacaagattgagaagatcctcagcactcttgttaaagggacacgcagacctgtgacctgcaagattcgcatcctgccatcgctagaagataccctgagccttgtgaagcggatagagaggactggcattgctgccatcgcagttcatgggaggaagcgggaggagcgacctcagcatcctgtcagctgtgaagtcatcaaagccattgctgataccctctccattcctgtcatagccaacggaggatctcatgaccacatccaacagtattcggacatagaggactttcgacaagccacggcagcctcttccgtgatggtggcccgagcagccatgtggaacccatctatcttcctcaaggagggtctgcggcccctggaggaggtcatgcagaaatacatcagatacgcggtgcagtatgacaaccactacaccaacaccaagtactgcttgtgccagatgctacgagaacagctggagtcgccccagggaaggttgctccatgctgcccagtcttcccgggaaatttgtgaggcctttggccttggtgccttctatgaggagaccacacaggagctggatgcccagcaggccaggctctcagccaagacttcagagcagacaggggagccagctgaagatacctctggtgtcattaagatggctgtcaagtttgaccggagagcatacccagcccagatcacccctaagatgtgcctactagagtggtgccggagggagaagttggcacagcctgtgtatgaaacggttcaacgccctctagatcgcctgttctcctctattgtcaccgttgctgaacaaaagtatcagtctaccttgtgggacaagtccaagaaactggcggagcaggctgcagccatcgtctgtctgcggagccagggcctccctgagggtcggctgggtgaggagagcccttccttgcacaagcgaaagagggaggctcctgaccaagaccctgggggccccagagctcaggagctagcacaacctggggatctgtgcaagaagccctttgtggccttgggaagtggtgaagaaagccccctggaaggctggtgactactcttcctgccttagtcacccctccatgggcctggtgctaaggtggctgtggatgccacagcatgaaccagatgccgttgaacagtttgctggtcttgcctggcagaagttagatgtcctggcaggggccatcagcctagagcatggaccaggggccgcccaggggtggatcctggcccctttggtggatctgagtgacagggtcaagttctctttgaaaacaggagcttttcaggtggtaactccccaacctgacattggtactgtgcaataaagacaccccctaccctcacccacggctggctgcttcagccttgggcatcttcataaa

SEQ ID NO: 128 - protein, 493 aa, Homo sapiens
MILNSLSLCYHNKLILAPMVRVGTLPMRLLALDYGADIVYCEELIDLKMIQCKRVVNEVLSTVDFVAPDDRVVFRTCEREQNRVVFQMGTSDAERALAVARLVENDVAGIDVNMGCPKQYSTKGGMGAALLSDPDKIEKILSTLVKGTRRPVTCKIRILPSLEDTLSLVKRIERTGIAAIAVHGRKREERPQHPVSCEVIKAIADTLSIPVIANGGSHDHIQQYSDIEDFRQATAASSVMVARAAMWNPSIFLKEGLRPLEEVMQKYIRYAVQYDNHYTNTKYCLCQMLREQLESPQGRLLHAAQSSREICEAFGLGAFYEETTQELDAQQARLSAKTSEQTGEPAEDTSGVIKMAVKFDRRAYPAQITPKMCLLEWCRREKLAQPVYETVQRPLDRLFSSIVTVAEQKYQSTLWDKSKKLAEQAAAIVCLRSQGLPEGRLGEESPSLHKRKREAPDQDPGGPRAQELAQPGDLCKKPFVALGSGEESPLEGW

SEQ ID NO: 129 - DNA, 3760 bp, Homo sapiens
gagaatggcggcggcggcggcggcggcggcggccgctgccattgcccggagatggccggcagagccgccgagacgccgaagagcccgccgcccgcgcgaggtgtagacggggcactgccttcagagcaggtcctgccagcctcgctggagaggatgccctcgtgtccgtgatgggctgtgggacaagcaaggtccttcccgagccacccaaggatgtccagctggatctggtcaagaaggtggagcccttcagtggcactaagagtgacgtgtacaagcacttcatcacagaggtggacagtgttggccctgtcaaagccgggttcccagcagcaagtcagtatgcacacccctgccccggtcccccgactgctggccacacggagcctccctcagaaccaccacgcagggccagggtagctaagtacagggccaagtttgacccacgtgttacagctaagtatgacatcaaggccctaattggccgaggcagcttcagccgagtggtacgtgtagagcaccgggcaacccggcagccgtatgccatcaagatgattgagaccaagtaccgggaggggcgggaggtgtgtgagtcggagctgcgtgtgctgcgtcgggtgcgtcatgccaacatcatccagctggtggaggtgttcgagacacaggagcgggtgtacatggtgatggagctggccactggtggagagctctttgaccgcatcattgccaagggctccttcaccgagcgtgacgccacgcgggtgctgcagatggtgctggatggcgtccggtatctgcatgcactgggcatcacacaccgagacctcaaacctgagaatctgctctactaccatccgggcactgactccaagatcatcatcaccgacttcggcctggccagtgctcgcaagaagggtgatgactgcttgatgaagaccacctgtggcacgcctgagtacattgccccagaagtcctggtccgcaagccatacaccaactcagtggacatgtgggcgctgggcgtcattgcctacatcctactcagtggcaccatgccgtttgaggatgacaaccgtacccggctgtaccggcagatcctcaggggcaagtacagttactctggggagccctggcctagtgtgtccaacctggccaaggacttcattgaccgcctgctgacagtggaccctggagcccgtatgactgcactgcaggccctgaggcacccgtgggtggtgagcatggctgcctcttcatccatgaagaacctgcaccgctccatatcccagaacctccttaaacgtgcctcctcgcgctgccagagcaccaaatctgcccagtccacgcgttccagccgctccacacgctccaataagtcacgccgtgtgcgggaacgggagctgcgggagctcaacctgcgctaccagcagcaatacaatggctgagccgcctggctgtgcacacatgcagcacgacccagcctggccacacactgtggtgccatctgggtccgatgccctctctggagataggcctatgtggcccacagtaggtgaagaatgtctggctccagccctttctctgtgccttcagcagcccctgtcctcaccatgggcctgggccaggtgtgacagagtagaggtagcacagggggctgtgactccccctgaactgggagcctggcctggcactgatacccctcttggtgggcagctgctctggtggagttgggaagggataggacctggccttcactgtctcccttgccctttgacttttccccaatcaaagggaactgcagtgctgggtggagtgtcctgtggcctcaggaccctttgggacagttacttctgggaccccctttcctccacagagcccttctccctggtttcacacattcccatgcatcctgatccttaagattatgctccagtgggagaccctggtaggcacaaagcttgtgccttgactggacccgtagcccctggctaggtcgaaacagccctccacctcccagccaagatctgtcttccttcatggtgcctccagggagccttcctggtcccaggacctctggtggagggccatggcgtggaccttcacccttctggactgtgtggccatgctggtcatcggcttgcccaggctccagcctctccagattctgaggggtctcagcccaccgcccttggtgccttctttgtagagcccaccgctacctccctctccccgttggatgtccattccattccccaggtgcctccttcccaactgggggtggttaaagggagccccactgctgctacctggggaatggggcacctgggggccaaggcagagggaagggggtcctcccgattagggtcgagtgtcagcctgggttctatcctttggtgcagccccattgccttttcccttcaggctctgttgctccctcctctgcagctgcacgaaggcgccatctggtgtctgcatgggtgttggcagcctgggagtgatcactgcacgcccatcgtgcacacctgcccatcgtgcacacccacccatggtgcacacctgtagtcctccatgaggacatgggaaggtaggagttgccgccctgggggagggtcccgggctgctcacctctccccttctgctgagcttctgcgcacccctccctggaacttagccatactgtgtgacctgcctctgaaaccagggtgccaggggcactgccttctcacagctggccttgccccgtccaccctgtgctgcttcccttcacagcattaaccttccagtctgggtcccactgagcctcaagctggaaggagcccctgcgggaggtgggtggggttgggtggctgctttcccagaggcctgagccagaaccatccccatttcttttgtggtatctccccctaccacaaaccaggctggaacccaagccccttcctccacagctgccttcagtgggtagaatggggccagggcccagctttggccttagcttgacggcagggcccctgccattgcaggagggtttggttcccactcagcttctgccggtcggcagcctgggccaggcccttttcctgcatgtgccacctccagtgggaaacaaaactaaagagaccactctgtgccaagtcgactatgccttagacacatcctcctaccgtccccaatgccccctgggcaggaggcagtggagaaccaagccccatggcctcagaatttccccccagttccccaagtgtctctggggacctgaagccctggggcttacgttctctcttgcccagggtgggcctggtcctgagggcaggacagggggtttggagatgtgggcctttgatagacccacttgggccttcatgccatggcctgtggatggagaatgtgcagttatttattatgcgtattcagtttgtaaacgtatcctctgtattcagtaaacaggctgcctctccagggagggctgccattcattccaacagttctggcttcttgctgtaggaccaaggggttgccctggaggaggggtgggggccccggcctcggcatggctactctaggaagagccactgctactcaaggagtcactcagccccttctgtgccagaagtccaagtagggagtcggaccctcaacagcctcttctttctcctgagccaggaagacagacatgaatgcatgatgggacagggcctgggtctttaatgggttgagctggggagggcctgtggtgagctcagttgtaggctatgacctggtt

SEQ ID NO: 130 - protein, 424 aa, Homo sapiens
MGCGTSKVLPEPPKDVQLDLVKKVEPFSGTKSDVYKHFITEVDSVGPVKAGFPAASQYAHPCPGPPTAGHTEPPSEPPRRARVAKYRAKFDPRVTAKYDIKALIGRGSFSRVVRVEHRATRQPYAIKMIETKYREGREVCESELRVLRRVRHANIIQLVEVFETQERVYMVMELATGGELFDRIIAKGSFTERDATRVLQMVLDGVRYLHALGITHRDLKPENLLYYHPGTDSKIIITDFGLASARKKGDDCLMKTTCGTPEYIAPEVLVRKPYTNSVDMWALGVIAYILLSGTMPFEDDNRTRLYRQILRGKYSYSGEPWPSVSNLAKDFIDRLLTVDPGARMTALQALRHPWVVSMAASSSMKNLHRSISQNLLKRASSRCQSTKSAQSTRSSRSTRSNKSRRVRERELRELNLRYQQQYNG

SEQ ID NO: 131 - DNA, 1899 bp, Homo sapiens
atgattttgaatagcctctctctgtgttaccataataagctaatcctggccccaatggttcgggtagggactcttccaatgaggctgctggccctggattatggagcggacattgtttactgtgaggagctgatcgacctcaagatgattcagtgcaagagagttgttaatgaggtgctcagcacagtggactttgtcgcccctgatgatcgagttgtcttccgcacctgtgaaagagagcagaacagggtggtcttccagatggggacttcagacgcagagcgagcccttgctgtggccaggcttgtagaaaatgatgtggctggtattgatgtcaacatgggctgtccaaaacaatattccaccaagggaggaatgggagctgccctgctgtcagaccctgacaagattgagaagatcctcagcactcttgttaaagggacacgcagacctgtgacctgcaagattcgcatcctgccatcgctagaagataccctgagccttgtgaagcggatagagaggactggcattgctgccatcgcagttcatgggaggtgtagacggggcactgccttcagagcaggtcctgccagcctcgctggagaggatgccctcgtgtccgtgatgggctgtgggacaagcaaggtccttcccgagccacccaaggatgtccagctggatctggtcaagaaggtggagcccttcagtggcactaagagtgacgtgtacaagcacttcatcacagaggtggacagtgttggccctgtcaaagccgggttcccagcagcaagtcagtatgcacacccctgccccggtcccccgactgctggccacacggagcctccctcagaaccaccacgcagggccagggtagctaagtacagggccaagtttgacccacgtgttacagctaagtatgacatcaaggccctaattggccgaggcagcttcagccgagtggtacgtgtagagcaccgggcaacccggcagccgtatgccatcaagatgattgagaccaagtaccgggaggggcgggaggtgtgtgagtcggagctgcgtgtgctgcgtcgggtgcgtcatgccaacatcatccagctggtggaggtgttcgagacacaggagcgggtgtacatggtgatggagctggccactggtggagagctctttgaccgcatcattgccaagggctccttcaccgagcgtgacgccacgcgggtgctgcagatggtgctggatggcgtccggtatctgcatgcactgggcatcacacaccgagacctcaaacctgagaatctgctctactaccatccgggcactgactccaagatcatcatcaccgacttcggcctggccagtgctcgcaagaagggtgatgactgcttgatgaagaccacctgtggcacgcctgagtacattgccccagaagtcctggtccgcaagccatacaccaactcagtggacatgtgggcgctgggcgtcattgcctacatcctactcagtggcaccatgccgtttgaggatgacaaccgtacccggctgtaccggcagatcctcaggggcaagtacagttactctggggagccctggcctagtgtgtccaacctggccaaggacttcattgaccgcctgctgacagtggaccctggagcccgtatgactgcactgcaggccctgaggcacccgtgggtggtgagcatggctgcctcttcatccatgaagaacctgcaccgctccatatcccagaacctccttaaacgtgcctcctcgcgctgccagagcaccaaatctgcccagtccacgcgttccagccgctccacacgctccaataagtcacgccgtgtgcgggaacgggagctgcgggagctcaacctgcgctaccagcagcaatacaatggctga

SEQ ID NO: 132 - protein, 632 aa, Homo sapiens
MILNSLSLCYHNKLILAPMVRVGTLPMRLLALDYGADIVYCEELIDLKMIQCKRVVNEVLSTVDFVAPDDRVVFRTCEREQNRVVFQMGTSDAERALAVARLVENDVAGIDVNMGCPKQYSTKGGMGAALLSDPDKIEKILSTLVKGTRRPVTCKIRILPSLEDTLSLVKRIERTGIAAIAVHGRCRRGTAFRAGPASLAGEDALVSVMGCGTSKVLPEPPKDVQLDLVKKVEPFSGTKSDVYKHFITEVDSVGPVKAGFPAASQYAHPCPGPPTAGHTEPPSEPPRRARVAKYRAKFDPRVTAKYDIKALIGRGSFSRVVRVEHRATRQPYAIKMIETKYREGREVCESELRVLRRVRHANIIQLVEVFETQERVYMVMELATGGELFDRIIAKGSFTERDATRVLQMVLDGVRYLHALGITHRDLKPENLLYYHPGTDSKIIITDFGLASARKKGDDCLMKTTCGTPEYIAPEVLVRKPYTNSVDMWALGVIAYILLSGTMPFEDDNRTRLYRQILRGKYSYSGEPWPSVSNLAKDFIDRLLTVDPGARMTALQALRHPWVVSMAASSSMKNLHRSISQNLLKRASSRCQSTKSAQSTRSSRSTRSNKSRRVRERELRELNLRYQQQYNG

SEQ ID NO: 133 - DNA, 1609 bp, Homo sapiens
atgattttgaatagcctctctctgtgttaccataataagctaatcctggccccaatggttcgggtagggactcttccaatgaggctgctggccctggattatggagcggacattgtttactgtgaggagctgatcgacctcaagatgattcagtgcaagagagttgttaatgaggtgctcagcacagtggactttgtcgcccctgatgatcgagttgtcttccgcacctgtgaaagagagcagaacagggtggtcttccagatggtgtagacggggcactgccttcagagcaggtcctgccagcctcgctggagaggatgccctcgtgtccgtgatgggctgtgggacaagcaaggtccttcccgagccacccaaggatgtccagctggatctggtcaagaaggtggagcccttcagtggcactaagagtgacgtgtacaagcacttcatcacagaggtggacagtgttggccctgtcaaagccgggttcccagcagcaagtcagtatgcacacccctgccccggtcccccgactgctggccacacggagcctccctcagaaccaccacgcagggccagggtagctaagtacagggccaagtttgacccacgtgttacagctaagtatgacatcaaggccctaattggccgaggcagcttcagccgagtggtacgtgtagagcaccgggcaacccggcagccgtatgccatcaagatgattgagaccaagtaccgggaggggcgggaggtgtgtgagtcggagctgcgtgtgctgcgtcgggtgcgtcatgccaacatcatccagctggtggaggtgttcgagacacaggagcgggtgtacatggtgatggagctggccactggtggagagctctttgaccgcatcattgccaagggctccttcaccgagcgtgacgccacgcgggtgctgcagatggtgctggatggcgtccggtatctgcatgcactgggcatcacacaccgagacctcaaacctgagaatctgctctactaccatccgggcactgactccaagatcatcatcaccgacttcggcctggccagtgctcgcaagaagggtgatgactgcttgatgaagaccacctgtggcacgcctgagtacattgccccagaagtcctggtccgcaagccatacaccaactcagtggacatgtgggcgctgggcgtcattgcctacatcctactcagtggcaccatgccgtttgaggatgacaaccgtacccggctgtaccggcagatcctcaggggcaagtacagttactctggggagccctggcctagtgtgtccaacctggccaaggacttcattgaccgcctgctgacagtggaccctggagcccgtatgactgcactgcaggccctgaggcacccgtgggtggtgagcatggctgcctcttcatccatgaagaacctgcaccgctccatatcccagaacctccttaaacgtgcctcctcgcgctgccagagcaccaaatctgcccagtccacgcgttccagccgctccacacgctccaataagtcacgccgtgtgcgggaacgggagctgcgggagctcaacctgcgctaccagcagcaatacaatggctga

SEQ ID NO: 134 - protein, 89 aa, Homo sapiens
MILNSLSLCYHNKLILAPMVRVGTLPMRLLALDYGADIVYCEELIDLKMIQCKRVVNEVLSTVDFVAPDDRVVFRTCEREQNRVVFQMV

SEQ ID NO: 135 - DNA, 2277 bp, Homo sapiens
atggggctcccagcgctcgagttcagcgactgctgcctcgatagtccgcacttccgagagacgctcaagtcgcacgaagcagagctggacaagaccaacaaattcatcaaggagctcatcaaggacgggaagtcactcataagcgcgctcaagaatttgtcttcagcgaagcggaagtttgcagattccttaaatgaatttaaatttcagtgcataggagatgcagaaacagatgatgagatgtgtatagcaagatctttgcaggagtttgccactgtcctcaggaatcttgaagatgaacggatacggatgattgagaatgccagcgaggtgctcatcactcccttggagaagtttcgaaaggaacagatcggggctgccaaggaagccaaaaagaagtatgacaaagagacagaaaagtattgtggcatcttagaaaaacacttgaatttgtcttccaaaaagaaagaatctcagcttcaggaggcagacagccaagtggacctggtccggcagcatttctatgaagtatccctggaatatgtcttcaaggtgcaggaagtccaagagagaaagatgtttgagtttgtggagcctctgctggccttcctgcaaggactcttcactttctatcaccatggttacgaactggccaaggatttcggggacttcaagacacagttaaccattagcatacagaacacaagaaatcgctttgaaggcactagatcagaagtggaatcactgatgaaaaagatgaaggagaatccccttgagcacaagaccatcagtccctacaccatggagggatacctctacgtgcaggagaaacgtcactttggaacttcttgggtgaagcactactgtacatatcaacgggattccaaacaaatcaccatggtaccatttgaccaaaagtcaggaggaaaagggggagaagatgaatcagttatcctcaaatcctgcacacggcggaaaacagactccattgagaagaggttttgctttgatgtggaagcagtagacaggccaggggttatcaccatgcaagctttgtcggaagaggaccggaggctctggatggaagccatggatggccgggaacctgtctacaactcgaacaaagacagccagagtgaagggactgcgcagttggacagcattggcttcagcataatcaggaaatgcatccatgctgtggaaaccagagggatcaacgagcaagggctgtatcgaattgtgggtgtcaactccagagtgcagaagttgctgagtgtcctgatggaccccaagactgcttctgagacagaaacagatatctgtgctgaatgggagataaagaccatcactagtgctctgaagacctacctaagaatgcttccaggaccactcatgatgtaccagtttcaaagaagtttcatcaaagcagcaaaactggagaaccaggagtctcgggtctctgaaatccacagccttgttcatcggctcccagagaaaaatcggcagatgttacagctgctcatgaaccacttggcaaatgttgctaacaaccacaagcagaatttgatgacggtggcaaaccttggtgtggtgtttggacccactctgctgaggcctcaggaagaaacagtagcagccatcatggacatcaaatttcagaacattgtcattgagatcctaatagaaaaccacgaaaagatatttaacaccgtgcccgatatgcctctcaccaatgcccagctgcacctgtctcggaagaagagcagtgactccaagcccccgtcctgcagcgagaggcccctgacgctcttccacaccgttcagtcaacagagaaacaggaacaaaggaacagcatcatcaactccagtttggaatctgtctcatcaaatccaaacagcatccttaattccagcagcagcttacagcccaacatgaactccagtgacccagacctggctgtggtcaaacccacccggcccaactcactccccccgaatccaagcccaacttcacccctctcgccatcttggcccatgttctcggcgccatccagccctatgcccacctcatccacgtccagcgactcatcccccgtcagcacaccgttccggaaggcaaaagccttgtatgcctgcaaagctgaacatgactcagaactttcgttcacagcaggcacggtcttcgataacgttcacccatctcaggagcctggctggttggaggggactctgaacggaaagactggcctcatccctgagaattacgtggagttcctc

(19) United States
(12) Patent Application Publication
(10) Pub. No.: US 2017/0081723 A1
(43) Pub. Date: Mar. 23, 2017

(54) Fusion Genes in Cancer

(71) Applicants: Agency for Science, Technology and Research, Singapore (SG); National University of Singapore, Singapore (SG)

(72) Inventors: Axel Hillmer, Singapore (SG); Yijun Ruan, Singapore (SG); Fei Yao, Singapore (SG); Patrick Tan, Singapore (SG); Khay Guan Yeoh, Singapore (SG); Walter Hunziker, Singapore (SG); Audrey S M Teo, Singapore (SG); Yee Yen Sia, Singapore (SG)

(73) Assignees: Agency for Science, Technology and Research, Singapore (SG); National University of Singapore, Singapore (SG)

(21) Appl. No.: 15/122,554

(22) PCT Filed: Mar. 23, 2015
(86) PCT No.: PCT/SG2015/050047

§ 371 Date: Aug. 30, 2016

(30) Foreign Application Priority Data
Mar. 21, 2014 (SG) 10201400876T

(51) Int. Cl. C12Q 1/68

(52) U.S. Cl. CPC C12Q 1/6886; C12Q 2600/118; C12Q 2600/106

(57) ABSTRACT

The present invention relates to a method for determining or making of a prognosis if a patient has cancer or is at an increased risk of having cancer, the method comprising testing for the presence of one or more cancer-associated fusion genes, or proteins derived thereof, in a sample obtained from a patient. More specifically, the present invention relates to fusion genes CLEC16A-EMP2, SNX2-PRDM6, MLL3-PRKAG2, DUS2L-PSKH1 and CLDN18-ARHGAP26 in gastric cancer. Use of the method and a kit when used in the method are also provided.

### CROSS-REFERENCE TO RELATED APPLICATIONS

This application claims the benefit of priority of Singapore application No. 10201400876T, filed 21 Mar. 2014, the contents of it being hereby incorporated by reference in its entirety for all purposes.

### FIELD OF THE INVENTION

The present invention is in the field of cancer biomarkers, in particular fusion genes as prognostic biomarkers for cancer.

### BACKGROUND OF THE INVENTION

Cancer is a class of diseases characterized by a group of cells that has lost its normal control mechanisms resulting in unregulated growth. Cancerous cells are also called malignant cells and can develop from any tissue within any organ. As cancerous cells grow and multiply, they form a tumour that invades and destroys normal adjacent tissues. Cancerous cells from the primary site can also spread throughout the body.

An example of a cancer is gastric cancer (GC). Most GCs are diagnosed at an advanced stage, which limits the current treatment strategies with the overall 5-year survival rate for distant or metastatic disease of ˜3%.

On the molecular level, GC is heterogeneous and currently the only therapeutic target is the amplified receptor tyrosine-protein kinase ERBB2.

While recent whole-genome and exome sequencing studies have identified recurrently mutated genes genome rearrangements in GC have not been studied in great detail. Genomic rearrangements, can have dramatic impact on gene function by amplification, deletion and gene disruption, and can create fusion genes with new functions.

Therefore, there is a need to identify the prognostic factors and markers that can be used to reliably determine the prognosis of patients suffering from cancer, such as gastric cancer, to allow identification of high risk and low risk cancer patients to allow different treatment approaches.

### SUMMARY OF THE INVENTION

In one aspect, there is provided a method of determining or making of a prognosis if a patient has cancer or is at an increased risk of having cancer, the method comprising testing for the presence of one or more cancer-associated fusion genes, or proteins derived thereof, in a sample obtained from a patient, wherein said presence of one or more cancer-associated fusion genes in the sample indicates that said patient has cancer, or is at an increased risk of cancer, wherein the cancer-associated fusion genes are selected from the group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH1 (SEQ ID NO.: 131 or 133), or wherein the cancer-associated fusion genes are selected from the group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH1 (SEQ ID NO.: 131 or 133) in combination with CLDN18-ARHGAP26 (SEQ ID NO: 107).

In one aspect, there is provided a method of determining if a patient has cancer or is at an increased risk of having cancer, the method comprising testing for the presence of one or more cancer-associated fusion genes, or proteins derived thereof, in a sample obtained from a patient, wherein said presence of one or more cancer-associated fusion genes in the sample is indicative of cancer, or an increased risk of cancer, in said patient, wherein the cancer-associated fusion genes are selected from a group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125), DUS2L-PSKH1 (SEQ ID NO.: 131 or 133) and CLDN18-ARHGAP26 (SEQ ID NO: 107).

In one aspect, there is provided a method of determining if a patient has cancer or is at increased risk of developing cancer, wherein said method comprises detecting one or more cancer-associated fusion genes selected from the group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH1 (SEQ ID NO.: 131 or 133) in a sample obtained from a patient, or detecting one or more cancer-associated fusion genes selected from the group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH (SEQ ID NO.: 131 or 133) in combination with CLDN18-ARHGAP26 (SEQ ID NO: 107), wherein the presence of one or more cancer-associated fusion genes in the sample indicates that the patient has cancer or is at an increased risk of developing cancer.

In one aspect, there is provided a method of determining if a patient has cancer or is at increased risk of developing cancer, wherein said method comprises detecting one or more cancer-associated fusion genes selected from a group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125), DUS2L-PSKH1 (SEQ ID NO.: 131 or 133) and CLDN18-ARHGAP26 (SEQ ID NO: 107) in a sample obtained from a patient, wherein the presence of one or more cancer-associated fusion genes in the sample indicates that the patient has cancer or is at an increased risk of developing cancer.

In one aspect, there is provided an expression vector comprising a nucleic acid sequence encoding any one of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125), DUS2L-PSKH1 (SEQ ID NO.: 131 or 133) or CLDN18-ARHGAP26 (SEQ ID NO: 107).

In one aspect, there is provided a cell transformed with the expression vector as disclosed herein.

In one aspect, there is provided a method for producing a polypeptide, comprising culturing the transformed cell as disclosed herein under conditions suitable for polypeptide expression and collecting the amount of said polypeptide from the cell.

In one aspect, there is provided a use of a cancer-associated fusion gene in the determination or prognosis of cancer in a patient, wherein the presence of one or more cancer-associated fusion genes in a sample obtained from the patient indicates that the patient has cancer or is at an increased risk of developing cancer, wherein the cancer-associated fusion genes are selected from a group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH1 (SEQ ID NO.: 131 or 133), or wherein the cancer-associated fusion genes selected from the group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH1 (SEQ ID NO.: 131 or 133) in combination with CLDN18-ARHGAP26 (SEQ ID NO: 107).

In one aspect, there is provided a use of a cancer-associated fusion gene in determining if a patient has cancer or is at an increased risk of cancer, wherein the presence of one or more cancer-associated fusion genes is in a sample obtained from the patient indicates that the patient has cancer or is at an increased risk of developing cancer, wherein the cancer-associated fusion genes are selected from a group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH1 (SEQ ID NO.: 131 or 133), or wherein the cancer-associated fusion genes selected from the group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH1 (SEQ ID NO.: 131 or 133) in combination with CLDN18-ARHGAP26 (SEQ ID NO: 107).

In one aspect, there is provided a kit when used in the method as disclosed herein comprising:
a) a first primer selected from the group consisting of SEQ ID NO. 1, SEQ ID NO. 3, SEQ ID NO. 5, SEQ ID NO. 7 and SEQ ID NO. 9;
b) a second primer selected from the group consisting of SEQ ID NO. 2, SEQ ID NO. 4, SEQ ID NO. 6, SEQ ID NO. 8 and SEQ ID NO. 10; optionally together with instructions for use.

### BRIEF DESCRIPTION OF THE DRAWINGS

The invention will be better understood with reference to the detailed description when considered in conjunction with the non-limiting examples and the accompanying drawings, in which:

FIG. 1. Characteristics of somatic SVs identified by DNA-PET in GC. (A) SV filtering procedure for GC patient 125 is shown. SVs are plotted by Circos across the human genome arranged as a circle with the copy number alterations in the outer ring, followed by deletion, tandem duplications, inversions/unpaired inversions, and in the inner ring inter-chromosomal isolated translocations. SVs identified in the blood of patient 125 (top right) were subtracted from SVs identified in gastric tumor of patient 125 (top left), resulting in the somatically acquired SVs specific for the tumor (bottom). (B) Distribution of somatic and germline SVs of 15 GCs. (C) Proportion of somatic SVs and germline SVs in 15 GCs. SV counts shown on top. (D) Composition of somatic SVs in GC compared with germline SVs. SV counts shown on top. (E) Comparison of somatic SV compositions of GC with reported somatic SVs for pancreatic cancer, breast cancer, and prostate cancer. SVs were reduced to four categories to allow comparison.

FIG. 2. Breakpoint features of somatic SVs provide mechanistic insights. (A-C) Characterization of breakpoint locations of somatic SVs in GC. Coordinates of repeats and genes were downloaded from UCSC genome browser and open chromatin regions were compiled from Encyclopedia of DNA Elements (ENCODE). (D) Gene involving rearrangements can have insertions of small DNA fragments originating from one of the SV break points. Arrows represent genomic fragments. Breakpoint coordinates are indicated and micro-homologies are shown above breakpoint pairs. (E) Example of an overlap of a somatic tandem duplication and a chromatin interaction. Coordinates of chromosome 4 and enlarged locus are shown on top. The PET mapping coordinates of a somatic 59 kb tandem duplication of GC tumor 100 are shown with the upstream mapping region on the left and the downstream mapping region on the right. Number in brackets indicates number of non-redundant PET reads connecting the two regions (cluster size). Bottom: chromatin interaction identified by ChIA-PET in cell line MCF-7 shows an interaction between the two breakpoint regions indicated by an arch.

FIG. 3. Correlation between SVs identified in 15 GCs and chromatin interactions identified by ChIA-PET sequencing. (A) Overlap of somatic SVs identified by DNA-PET in breast cancer (BC, n=1,935) and GC (n=1,945) and germline SVs in GC patients (n=1,667) with long range chromatin interactions bound to RNA polymerase II in breast cancer cell line MCF-7 (n=87,253). Absolute numbers are shown above bars. Fraction of SVs overlapping with ChIA-PET interactions is calculated relative the total number of SVs of each data set (e.g. GC SVs). All SV/chromatin interaction overlaps are significantly higher than expected by chance (P<0.001, permutation based). (B) Overlap of somatic SVs identified by DNA-PET in chronic myeloid leukemia (CML, n=189) and GC (n=1,945) and germline SVs in GC patients (n=1,667) with long range chromatin interactions bound to RNA polymerase II in CML cell line K562 (n=154,130). All SV/chromatin interaction overlaps are significantly higher than expected by chance (P<0.001, permutation based). (C, E and G) Overlap characteristics between 1,667 non-redundant germline SVs identified in paired normal tissue of GC patients and 87,253 RNA polymerase II chromatin interactions identified by ChIA-PET of MCF-7 are shown. (D, F and H) Overlap characteristics between 1,945 somatic SVs identified in 15 GC with the same MCF-7 chromatin interactions as in C, E and G are shown. (C) and (D) Venn diagrams illustrating the proportion of overlap between SVs and chromatin interactions showing small overlap which is, however, significantly more than expected by chance (P<0.001, permutation based). (E) and (F) comparison of the cluster size distribution of SVs which overlap (common) or do not overlap (unique) with chromatin interaction sites, respectively. (G) and (H) show the distribution of the distance between SVs and chromatin interaction sites.

FIG. 4. Recurrent CLDN18-ARHGAP26 in-frame fusions in GC have a pro-proliferative effect in HGC27. (A) RefSeq gene track (top), copy number of tumor 136 by DNA-PET sequencing (middle), and PET mapping of a somatic balanced translocation with breakpoints in CLDN18 and ARHGAP26 in tumor 136 (bottom). Numbers of fused exons are shown in red. Mapping regions of DNA-PET clusters are shown by red and gray arrow heads with cluster size in brackets, dashed lines at Sanger sequencing validated breakpoint coordinates in squared brackets. Location of genomic breakpoints of tumor 07K611T (chr3:139,237,526 and chr5:142,309,897) are indicated by vertical arrows. (B) Validation of genomic rearrangement by FISH of tumor 136. (C) RT-PCRs of tumor/normal pairs of two gastric cancers with CLDN18-ARHGAP26 fusions. RT-PCRs for β-actin serve as positive control. N, normal gastric tissue; T, gastric tumor; M, marker. (D) Cryptic splice site in the coding region of exon 5 of CLDN18 results in the extension of the open reading frame into ARHGAP26. Sequences of the fusion transcript are highlighted in bold and are connected by a vertical line. (E) Protein domain ideogram of CLDN18-ARHGAP26. (F) Sanger sequencing chromatogram of RT-PCR of CLDN18-ARHGAP26 of tumor 136. Fusion point between CLDN18 and ARHGAP26 is indicated by vertical dashed line. (G) qRT-PCR for the CLDN18-ARHGAP26 fusion transcript in HGC27 parental cells and stable cell lines with empty and CLDN18-ARHGAP26 expressing vector. (H) Proliferation assay of HGC27 cells stably expressing CLDN18-ARHGAP26. Assay is done in quadruplicates. Error bars represent standard deviation. OD450, optical density at 450 nm. See FIG. 5 to 8 and Example 12 for characterization of MLL3-PRKAG2, DUS2L-PSKH1, CLEC16A-EMP2, and SNX2-PRDM6.

FIG. 5. Recurrent MLL3-PRKAG2 in-frame fusions in GC have a pro-proliferative effect in TMK1. (A) RefSeq gene track downloaded from UCSC (top) physical coverage by DNA-PET sequencing of TMK1 (middle) and PET mapping of a somatic deletion with breakpoints in MLL3 and PRKAG2 (bottom). (B) Gene structures of MLL3 and PRKAG2 as downloaded from Ensembl (www.ensembl.org). Exon-exon fusions on the transcript level are indicated by diagonal lines with exon numbers shown above and below the genes, respectively. Numbers in along the diagonal lines indicate the number of observations of each fusion. (C) RT-PCRs of tumor/normal pairs of three gastric cancers with MLL3-PRKAG2 fusions. RT-PCRs for β-actin serve as positive control. M, marker; N, normal gastric tissue; T, gastric tumor. (D) Sanger sequencing chromatogram of RT-PCR of MLL3-PRKAG2 fusion of TMK1. Fusion point between MLL3 and PRKAG2 is indicated by vertical dashed line. (E) Quantitative RT-PCR (qRT-PCR) for endogenous MLL3 and PRKAG2 and the fusion transcript after knock down in TMK1 cells with siRNAs A and B specific for the fusion point. Experiments were performed in triplicates. Error bars represent standard deviation of triplicates. (F) Proliferation assay of TMK1 cells with siRNA-A targeting the MLL3-PRKAG2 fusion. FGFR4 is positive control for negative proliferative effect after knock down. Assay is done in quadruplicates. Error bars represent standard deviation. OD450, optical density at 450 nm, the colorimetric read out of WST-1 assay.

FIG. 6. Identification of recurrent in-frame fusion gene DUS2L-PSKH1 and proliferation analysis of TMK1 after fusion knock down. (A) Chromosome ideogram (top) with enlarged region (bottom) highlighted by vertical boxes. Enlarged genomic view shows genomic coordinates on top, UCSC gene track below. Gene GFOD2, RANBP10, NUTF2, NRN1L, DPEP2/3, DDX28, DUS2L, and NFATC3 are implicated in cancer based on multiple entries in Catalogue Of Somatic Mutations In Cancer (COSMIC). Copy number and SV tracks of TMK1 are shown below gene tracks with physical coverage shown as smoothened or unsmoothened lines and the PET mapping is shown as left arrows for 5′ mapping region and right arrows for 3′ mapping region. The reconstructed genomic structure based on a tandem duplication of TMK1 is shown at the bottom. (B) RT-PCRs of tumor/normal pairs of two gastric cancers with DUS2L-PSKH1 gene fusion. RT-PCRs for β-actin serve as positive control. M, marker; N, normal gastric tissue; T, gastric tumor. (C) Sanger sequencing chromatogram of RT-PCR of DUS2L-PSKH1 fusion of TMK1. Fusion point between DUS2L and PSKH1 is indicated by vertical dashed line. (D) Four siRNAs targeting the fusion point of the DUS2L-PSKH1 transcript were used to knock down the expression of the fusion gene in TMK1. Experiments were performed in triplicates. One representative of two experiments. Error bars represent standard deviation of triplicates. (E) siRNAs A and C against DUS2L-PSKH1 were used to compare impact of knock down of the fusion gene on proliferation properties. TMK1 cells were transiently transfected with siRNAs and proliferation was estimated by colorimetric assay using WST-1 reagent. FGFR4 was used as positive control. Experiments were performed in triplicates. Error bars represent standard deviation of triplicates. Note inconsistent results for siRNA A and C. One representative of two experiments.

FIG. 7. Identification of recurrent in-frame fusion gene CLEC16A-EMP2 and proliferation analysis of HGC27 stably expressing CLEC16A-EMP2. (A) Unpaired inversion in tumor 133 identified by DNA-PET resulting in fusion of CLEC16A and EMP2. Chromosome ideogram, gene track, copy number and SV representations are as described for FIG. 6 with EMP2, TEKT5, NUBP1, FAM18A, CIITA and CLEC16A implicated in cancer. (B) Sanger sequencing chromatogram of fusion CLEC16A-EMP2 of tumor 06/0159. Fusion point between CLEC16A and EMP2 is indicated by vertical dashed line. (C) RT-PCRs of tumor/normal pairs of two gastric cancers with CLEC16A-EMP2 gene fusion. RT-PCRs for β-actin serve as positive control. M, marker; N, normal gastric tissue; T, gastric tumor. (D) qPCR analysis of HGC27 cells stably expressing CLEC16A-EMP2 fusion gene. Fold changes were calculated relative to parental cell line and cells stably transfected with empty vector. Error bars represent standard deviation of triplicates. (E) Proliferation assay of HGC27 cells stably expressing CLEC16A-EMP2. Assay was done in quadruplicates. Error bars represent standard deviation. OD450, optical density at 450 nm, the colorimetric read out of WST-1 assay.

FIG. 8. Identification of recurrent in-frame fusion gene SNX2-PRDM6 and proliferation analysis of HGC27 stably expressing SNX2-PRDM6. (A) Deletion in tumor 125 identified by DNA-PET resulting in fusion of SNX2 and PRDM6. Chromosome ideogram, gene track, copy number and SV representations are as described for FIG. 6. (B) RT-PCRs of Tumor 160 and paired normal tissue for SNX2-PRDM6 gene fusion. RT-PCRs for β-actin serve as positive control. M, marker; N, normal gastric tissue; T, gastric tumor. (C) Sanger sequencing chromatogram of fusion SNX2-PRDM6 of Tumor 125. Fusion point between SNX2 and PRDM6 is indicated by vertical dashed line. (D) qPCR analysis of HGC27 cells stably expressing SNX2-PRDM6 fusion gene. Fold changes were calculated relative to parental cell line and cells stably transfected with empty vector. Error bars represent standard deviation of triplicates. (E) Proliferation assay of HGC27 cells stably expressing SNX2-PRDM6. Assay was done in quadruplicates. Error bars represent standard deviation. OD450, optical density at 450 nm, the colorimetric read out of WST-1 assay.

FIG. 9. Characterization of cell lines overexpressing CLDN18, ARHGAP26, and CLDN18-ARHGAP26. (A) Antibodies to CLDN18 and ARHGAP26 detect CLDN18-ARHGAP26 fusion protein. MDCK cells expressing CLDN18-ARHGAP26 were immunostained with antibodies to CLDN18 and ARHGAP26. (B and C) Forced expression of CLDN18 in HeLa cells reverts to epithelial morphology as observed with immunofluorescence analysis of HeLa cells stably expressing CLDN18 and CLDN18-ARHGAP26 fusion gene using DAPI and antibodies to N-cadherin (B), β-catenin (C) and HA. (D) q-PCR analysis of non-transfected HeLa and stables expressing CLDN18 and CLDN18ΔP for N-cadherin, β-catenin and PAK1 levels. (E) Compensation effect of tight junction proteins in CLDN18-ARHGAP26 expressing MDCK cells observed via q-PCR analysis of tight junction proteins in MDCK stably expressing CLDN18, ARHGAP26 and CLDN18-ARHGAP26. Fold change were calculated relative to non-transfected MDCK cells. (F) MDCK stably expressing CLDN18, ARHGAP26 and CLDN18-ARHGAP26 fusion cells were fixed and immunostained with antibodies to ZO-1, HA or GFP.

FIG. 10. CLDN18-ARHGAP26 fusion expressing patient specimen and MDCK cells exhibit loss of epithelial phenotype and gain of cancer progression. (A) CLDN18 and (B) ARHGAP26 expression in normal and gastric tumor patient specimens. Immunofluorescence analysis of human normal (top) and tumor (bottom) stomach sections stained with antibodies to E-cadherin and DAPI as well as CLDN18 and ARHGAP26, respectively. (C) CLDN18-ARHGAP26 fusion expressing MDCK cells display fusiform and protrusive morphology. Phase contrast images of stable lines expressing CLDN18, ARHGAP26 and CLDN18-ARHGAP26 in MDCK cells obtained at sub-confluent levels. (D) Cell aggregation assay. MDCK non-transfected and stable lines expressing CLDN18, ARHGAP26 and CLDN18-ARHGAP26 fusion gene were plated as hanging-drops and phase contrast images were obtained the next day. (E) qPCR of EMT markers in MDCK cells stably expressing CLDN18, ARHGAP26 and CLDN18-ARHGAP26, respectively. (F) and (G) Western blot analysis of non-transfected HeLa and stables expressing CLDN18, ARHGAP26 and CLDN18-ARHGAP26 fusion gene by immunoblotting for antibodies to N-cadherin, β-catenin (F), Akt, pAkt, and PAK1 (G). Actin is used as loading control.

FIG. 11. CLDN18-ARHGAP26 expression results in reduced cell-ECM adhesion. (A) Top, cell-ECM adhesion assay. MDCK stable lines expressing CLDN18, ARHGAP26 and CLDN18-ARHGAP26 fusion gene were seeded on untreated plates and phase contrast images were obtained two hours after seeding. MDCK non-transfected cell were used as control. Bottom, quantification of cells that adhered to untreated, collagen type I and fibronectin-treated surfaces. 2×10⁴ cells were seeded on these surfaces, washed three times with PBS and fixed in PFA for 10 min. The number of cells per field was counted 3-4 times. The proportion of cells that adhered was quantified relative to non-transfected MDCK cells (100%). (B) MDCK stable lines expressing CLDN18, ARHGAP26 and CLDN18-ARHGAP26 fusion gene were fixed and immunostained with antibodies to activated FAK and HA or GFP. (C) Absence of Paxillin in free edge in CLDN18-ARHGAP26 expressing MDCK cells. MDCK stable lines expressing CLDN18, ARHGAP26 and CLDN18-ARHGAP26 fusion gene were fixed and immunostained with antibodies to Paxillin and HA or GFP. (D) Western blot analysis of focal adhesion molecule levels in MDCK non-transfected and stable lines expressing CLDN18, ARHGAP26 and CLDN18-ARHGAP26 fusion gene. GAPDH was used as loading control. (E) Reduced levels of focal adhesion molecules in CLDN18-ARHGAP26 expressing MDCK. qPCR analysis of MDCK stable lines expressing CLDN18, ARHGAP26 and CLDN18-ARHGAP26 for focal adhesion molecules. Fold changes were calculated relative to MDCK non-transfected cells. (F) Western blot analysis of non-transfected MDCK and stables expressing CLDN18, ARHGAP26 and CLDN18-ARHGAP26. Blots were probed to integrin β1 and β5 and tubulin was used as loading control. (G) Reduction in integrin subunit levels in CLDN18-ARHGAP26 fusion expressing MDCK. Integrin subunits qPCR analysis of MDCK-CLDN18, -ARHGAP26 and -CLDN18-ARHGAP26 stables. Fold changes were calculated relative to MDCK non-transfected cells. (H) MDCK stable lines expressing CLDN18, CLDN18 with inactivated C-terminal PDZ-binding motif (CLDN18ΔP), ARHGAP26, CLDN18-ARHGAP26 and non-transfected MDCK cells were seeded on Transwell inserts and TER values were measured over a period of 48 hours. Empty Transwell inserts were used as negative control. (I) Phase contrast images of non-transfected MDCK and stables expressing CLDN18, ARHGAP26 and CLDN18-ARHGAP26 at confluent levels.

FIG. 12. CLDN18-ARHGAP26 has a cell context specific impact on proliferation, invasion and wound closure. (A) Delayed cell proliferation rates in CLDN18-ARHGAP26 fusion expressing MDCK cells. MDCK stable lines expressing CLDN18, ARHGAP26 and CLDN18-ARHGAP26 were seeded at 800 cells in quadruplicate in 24 well plates. MDCK non-transfected cells were used as control. (B) Wound healing assay. MDCK stable lines expressing CLDN18, ARHGAP26 and CLDN18-ARHGAP26 were seeded on Ibidi culture insert in μ-Dish and the following day, the insert was peeled off to create a wound and monitored for closure. Prior to seeding the μ-Dish plates were treated with collagen type 1. Phase contrast images were obtained at the start of the experiments and at intervals. (C) HeLa cells stably expressing CLDN18, ARHGAP26 and CLDN18-ARHGAP26 fusion gene were seeded on Matrigel invasion chamber. Non-transfected HeLa cells were used as control. 5% FBS was added as chemoattractant at the basal media and incubated for 24 hours. Cells were fixed, washed and stained with crystal violet to obtain phase contrast images (left) and to quantitate (right) the number of cells that invaded the matrigel. (D) HeLa and HGC27 cells stably expressing CLDN18, ARHGAP26 and CLDN18-ARHGAP26 were seeded on soft agar, incubated for one month and imaged (left) and counted (right). Parental lines stably transfected with vector were used as control.

FIG. 13. CLDN18 and ARHGAP26 modulate epithelial phenotypes. (A) Actin cytoskeletal staining of MDCK cells expressing CLDN18, ARHGAP26 and CLDN18-ARHGAP26. Cells were immunostained with HA for CLDN18 and CLDN18-ARHGAP26 expressing cells and Phallodin conjugated with Alexa 594 fluorescence. Arrows indicate clearing of stress fibers in ARHGAP26 and CLDN18-ARHGAP26 expressing MDCK cells. (B) Western blot analysis of total RhoA in non-transfected MDCK and cells expressing CLDN18, ARHGAP26 and CLDN18-ARHGAP26. Cells were immunostained with RhoA antibody and GAPDH. (C) Active RhoA immunofluorescence analysis in MDCK cells expressing CLDN18, ARHGAP26 and CLDN18-ARHGAP26. MDCK stables cells were stained with an antibody to active RhoA and DAPI. (D) Reduced GAP activity in MDCK stables expressing ARHGAP26 and CLDN18-ARHGAP26. The GAP activity was analyzed in a pull-down assay (G-LISA, Cytoskeleton). The amount of endogenous active GTP-bound RhoA was determined in a 96-well plate coated with RDB domain of Rho-family effector proteins. The GTP form of Rho from cell lysates of the different stable lines bound to the plate was determined with RhoA primary antibody and secondary antibody conjugated to HRP. Luminescence values were calculated relative to non-transfected MDCK cells. (E) Live HeLa cells expressing CLDN18, ARHGAP26 and CLDN18-ARHGAP26 were incubated with Alexa 594 conjugated CTxB for 15 min at 37° C. followed by washing and fixation. Cells were immunostained with HA or GFP antibody and DAPI.

### DEFINITIONS

The following words and terms used herein shall have the meaning indicated:

As used herein, the term “prognosis” or grammatical variants thereof refers to a prediction of the probable course and outcome of a clinical condition or disease. A prognosis of a patient is usually made by evaluating factors or symptoms of a disease that are indicative of a favorable or unfavorable course or outcome of the disease. The term “prognosis” does not refer to the ability to predict the course or outcome of a condition with 100% accuracy. Instead, the term “prognosis” refers to an increased probability that a certain course or outcome will occur; that is, that a course or outcome is more likely to occur in a patient exhibiting a given condition, when compared to those individuals not exhibiting the condition. For example, the course or outcome of a condition may be predicted with 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55% and 50% accuracy.

An example of prognosis is testing a sample for the presence of a marker wherein the presence of the marker indicates a favourable or an unfavourable disease outcome. Another example of prognosis is testing a sample for the presence of a marker wherein the presence of the marker indicates that a patient is a candidate for a type of treatment.

As used herein, the term “differential treatment plan” refers to a tailored treatment plan specific to a patient or disease subtype. For example, presence of a cancer marker in a patient sample indicates that the patient is a candidate for a differential treatment plan, wherein the differential treatment plan is targeted cancer therapy.

The term “sample” or “biological sample” as used herein refers to a cell, tissue or fluid that has been obtained from, removed or isolated from the subject. An example of a sample is a tumour tissue biopsy. Samples may be frozen fresh tissue, paraffin embedded tissue or formalin fixed paraffin embedded (FFPE) tissue. Another example of a sample is a cell line. An example of fluid samples include but is not limited to blood, serum, saliva, urine, cerebrospinal fluid and bone marrow fluid.

The term “testing for the presence” in relation to a gene, fusion gene or protein product derived thereof refers to screening for the presence or absence of a gene, fusion gene or protein derived thereof in a sample. The term “testing for the presence” in relation to a gene, fusion gene or protein product derived thereof also refers to quantifying expression of the gene, fusion gene or protein product derived thereof in a sample. It will be understood that quantifying expression includes quantifying the absolute expression of the gene, fusion gene or protein product in a sample.

The term “fusion gene” as used herein refers to a hybrid gene formed from two or more separate genes. Full-length or fragments of the coding sequence, non-coding sequence or both may be fused. Fusion may occur by one or more of the processes of chromosomal rearrangement, including but not limited to chromosomal translocation, inversion, duplication or deletion. The two or more genes may be on the same chromosome, different chromosomes or a combination of both. The two or more fused genes may be fused in-frame or out of frame.

It will be understood that fusion genes may gain the functions of one of the original unfused genes, or lose the functions of one of the original unfused genes or both. It will also be understood that fusion genes may gain functions that are not present in any of the unfused genes. For illustration, a fusion gene that is fused from gene A and gene B may gain the function(s) of gene A only, and lose the function(s) of gene B. Alternatively, the fusion gene that is fused from gene A and gene B may gain functions not found in gene A or gene B.

It will therefore be understood that a cell with a fused gene may have properties not found in a cell without the fused gene.

As used herein, the term “cancer-associated fusion genes” refer to fusion genes that are associated with cancer. It will be understood that one or more fusion genes may be associated with a cancer. For example, the presence of one or more cancer-associated fusion genes in a patient sample may indicate that the subject has cancer or that the subject has an increased risk of cancer. The detection of one or more cancer-associated fusion genes in a patient sample may also indicate that the subject qualifies for a targeted cancer treatment plan. Examples of cancer-associated fusion genes include but are not limited to CLEC16A-EMP2, SNX2-PRDM6, MLL3-PRKAG2, DUS2L-PSKH1 and CLDN18-ARHGAP26. It will be understood that the fusion genes may be detected alone or in combination. Without being bound by theory, it is understood that the presence of a combination of more than one cancer-associated fusion genes is correlated with a poorer prognosis or disease outcome relative to the presence of a single cancer-associated fusion gene. As such, it will be understood that the presence of a combination of more than one cancer-associated fusion genes is predictive of disease outcome or prognosis. For example, the fusion genes may be selected from the group consisting of CLEC16A-EMP2, SNX2-PRDM6, MLL3-PRKAG2 and DUS2L-PSKH1 in combination with CLDN18-ARHGAP26. It will be understood that 0, 1, 2, 3, 4, 5 or more fusion genes may be detected in a sample. For example, CLEC16A-EMP2 may be detected in a sample, or CLEC16A-EMP2 in combination with CLDN18-ARHGAP26 may be detected in a sample. In one example, CLDN18-ARHGAP26 shows loss of CLDN18 function and gain of ARHGAP26 function.

It will be understood that variations may exist between nucleotide and amino acid sequences of fusion genes in different subject. These genetic variations may be due to mutation, polymorphism or splice variants. It will also be understood that genetic variations may result in a phenotypic change in a subject or sample or may have no change in phenotype.

Proteins derived from a fusion gene may be functional or non-functional. Proteins derived from a fusion gene may be elongated or truncated. As used herein, a “functional protein” refers to a polypeptide that has biological activity. It will be understood that the biological activity or property of a functional protein derived from a fusion gene may be the same as a functional protein derived from one of the original unfused genes. It will also be understood that the biological activity or property of a functional protein derived from a fusion gene may be different to the biological activity or property of the unfused gene.

As used herein, “truncated protein” refers to a protein or polypeptide that has a reduced number of amino acids than a full length, untruncated protein.

As used herein, “elongated protein” refers to a protein that has an increased number of amino acids than a full length, untruncated protein.

It will also be understood that a fusion gene may confer different a biological property to a cell. For example, a fusion gene may result in a cell having an enhanced migration rate, pro-metastatic feature or changes in cell shape. A fusion gene may also result in a cell losing its epithelial phenotype, having impaired epithelial barrier properties and impaired wound healing properties.

It will be understood to one of skill in the art that the presence of fusion genes may be detected by a variety of methods. Examples include but are not limited to polymerase chain reaction (PCR), quantitative PCR, microarray, RT-PCR, Southern blot, Northern blot, fluorescence in situ hybridization (FISH) and DNA sequencing. DNA sequencing includes but is not limited to DNA-Paired-end tags (DNA-PET) sequencing and Next-Generation sequencing, SOLiD™ sequencing.

It will also be understood to one of skill in the art that a variety of detection agents may be used to detect fusion genes. Examples of detection agents include but are not limited to primers, probes and complementary nucleic acid sequences that hybridise to the fusion gene.

The term “primer” is used herein to mean any single-stranded oligonucleotide sequence capable of being used as a primer in, for example, PCR technology. Thus, a “primer” according to the disclosure refers to a single-stranded oligonucleotide sequence that is capable of acting as a point of initiation for synthesis of a primer extension product that is substantially identical to the nucleic acid strand to be copied (for a forward primer) or substantially the reverse complement of the nucleic acid strand to be copied (for a reverse primer). A primer may be suitable for use in, for example, PCR technology.

The term “probe” as used herein refers to any nucleic acid fragment that hybridizes to a target sequence. A probe may be labeled with radioactive isotopes, fluorescent tags, antibodies or chemical labels to facilitate detection of the probe.

As used herein, “hybridise” means that the primer, probe or oligonucleotide forms a noncovalent interaction with the target nucleic acid molecule under standard stringency conditions. The hybridising primer or oligonucleotide may contain non-hybridising nucleotides that do not interfere with forming the noncovalent interaction, e.g., a 5′ tail or restriction enzyme recognition site to facilitate cloning.

Furthermore, as used herein, any “hybridisation” is performed under stringent conditions. The term “stringent conditions” means any hybridisation conditions which allow the primers to bind specifically to a nucleotide sequence within the allelic expansion, but not to any other nucleotide sequences. For example, specific hybridisation of a probe to a nucleic acid target region under “stringent” hybridisation conditions, include conditions such as 3×SSC, 0.1% SDS, at 50° C. It is within the ambit of the skilled person to vary the parameters of temperature, probe length and salt concentration such that specific hybridisation can be achieved. Hybridisation and wash conditions are well known in the art.

It will be understood to one of skill in the art that fusion proteins may be detected by a variety of methods. Examples of methods to detect fusion proteins include but are not limited to immunohistochemistry (IHC), immunofluorescence labelling, Western blot, ELISA and SDS-PAGE.

It will also be understood to one of skill in the art that there are a variety of detection agents to quantify fusion protein expression. Examples of detection agents include but are not limited to antibodies and ligands that specifically bind to the fusion protein.

As mentioned above, detection of one or more fusion genes in a sample obtained from a patient is indicative of cancer, or an increased risk of cancer.

As used herein, “increased risk of cancer” means that a subject has not been diagnosed to have cancer but has an increased probability of having cancer relative to a control or reference that does not have the one or more fusion genes.

The terms “reference”, “control” or “standard” as used herein refer to samples or subjects on which comparisons to determine prognosis be performed. Examples of a “reference”, “control” or “standard” include a non-cancerous sample obtained from the same subject, a sample obtained from a non-metastatic tumour, a sample obtained from a subject that does not have cancer or a sample obtained from a subject that has a different cancer subtype. The terms “reference”, “control” or “standard” as used herein may also refer to the average expression levels of a gene or protein in a patient cohort. The terms “reference”, “control” or “standard” as used herein may also refer to the presence or absence of a fusion gene or protein in a cell line or plurality of cell lines. The terms “reference”, “control” or “standard” as used herein may also refer to a subject who is not suffering from cancer or who is suffering from a different type of cancer. An example of a reference or control is a patient without any one or more of the cancer-associated fusion genes.

As used herein, “cancer” refers to an epithelial cancer. Examples of epithelial cancers include but are not limited to gastric cancer, lung cancer, breast cancer, urogenital cancer, colon cancer, prostate cancer and cervical cancer.

A fusion polypeptide may be obtained by inserting a fusion gene into an expression vector. As used herein, “expression vector” refers to a plasmid that is used to introduce a specific gene into a target cell. Expression vectors may be transient expression vectors or stable expression vectors.

It will be understood that a cell may be transformed with an expression vector. Methods for transforming a cell will be understood by one of skill in the art. For example, a cell may be transformed by electroporation, heat shock, chemical or viral transfection.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### DISCLOSURE OF OPTIONAL EMBODIMENTS

Exemplary, non-limiting embodiments of a method of determining or making of a prognosis if a patient has cancer or is at an increased risk of having cancer will now be disclosed.

The method comprises testing for the presence of one or more cancer-associated fusion genes, or proteins derived thereof, in a sample obtained from a patient, wherein said presence of one or more cancer-associated fusion genes in the sample indicates that said patient has cancer, or is at an increased risk of cancer, wherein the cancer-associated fusion genes are selected from the group consisting of CLEC16A-EMP2, SNX2-PRDM6, MLL3-PRKAG2 and DUS2L-PSKH1, or wherein the cancer-associated fusion genes are selected from the group consisting of CLEC16A-EMP2, SNX2-PRDM6, MLL3-PRKAG2 and DUS2L-PSKH1 in combination with CLDN18-ARHGAP26.

In one embodiment, the cancer-associated fusion gene is CLEC16A-EMP2, SNX2-PRDM6, MLL3-PRKAG2, DUS2L-PSKH1 or CLDN18-ARHGAP26. In a preferred embodiment, the cancer-associated fusion gene is CLEC16A-EMP2. In one embodiment, 2, 3 or 4 of the fusion genes are selected from the group consisting of CLEC16A-EMP2, SNX2-PRDM6, MLL3-PRKAG2 and DUS2L-PSKH1 in combination with CLDN18-ARHGAP26.

In one embodiment, CLEC16A-EMP2 is in combination with CLDN18-ARHGAP26. In one embodiment, SNX2-PRDM6 is in combination with CLDN18-ARHGAP26. In one embodiment, MLL3-PRKAG2 is in combination with CLDN18-ARHGAP26. In one embodiment, DUS2L-PSKH1 is in combination with CLDN18-ARHGAP26. In a preferred embodiment, CLEC16A-EMP2 is in combination with CLDN18-ARHGAP26. In a preferred embodiment, MLL3-PRKAG2 is in combination with CLDN18-ARHGAP26.

The method disclosed herein is suitable for determining or making a prognosis of cancer. The cancer may be a carcinoma, a sarcoma, leukaemia, lymphoma, myeloma or a cancer of the central nervous system.

In one embodiment the cancer is an epithelial cancer or carcinoma. The epithelial cancer is preferably selected from the group consisting of skin cancer, lung cancer, gastric cancer, breast cancer, urogenital cancer, colon cancer, prostate cancer, cervical cancer, skin cancer, ovarian cancer, liver cancer and renal cancer. In a preferred embodiment, the cancer is gastric cancer.

The method as described herein is suitable for use in a sample of fresh tissue, frozen tissue, paraffin-preserved tissue and/or ethanol preserved tissue. The sample may be a biological sample. Non-limiting examples of biological samples include whole blood or a component thereof (e.g. plasma, serum), urine, saliva lymph, bile fluid, sputum, tears, cerebrospinal fluid, bronchioalveolar lavage fluid, synovial fluid, semen, ascitic tumour fluid, breast milk and pus. In one embodiment, the sample is obtained from blood, amniotic fluid or a buccal smear. In a preferred embodiment, the sample is a tissue biopsy.

A biological sample as contemplated herein includes tissue samples, cultured biological materials, including a sample derived from cultured cells, such as culture medium collected from cultured cells or a cell pellet. Accordingly, a biological sample may refer to a lysate, homogenate or extract prepared from a whole organism or a subset of its tissues, cells or component parts, or a fraction or portion thereof. A biological sample may also be modified prior to use, for example, by purification of one or more components, dilution, and/or centrifugation.

Well-known extraction and purification procedures are available for the isolation of nucleic acid from a sample. The nucleic acid may be used directly following extraction from the sample or, more preferably, after a polynucleotide amplification step (e.g. PCR). The amplified polynucleotide is ‘derived’ from the sample.

Preferably, the nucleic acid sequence is denatured prior to amplification. In one embodiment, the denaturation comprises heat treatment. Preferably, the heat treatment is carried out at a temperature in the range selected from the group consisting of from about 70-110° C.; about 75-105° C.; about 80-100° C. and about 85-95° C. Preferably, the denaturation step is carried out at 94° C.

In another embodiment, the denaturation step is carried out for a period selected from the group consisting of from about 1-30 minutes; about 2-25 minutes and about 3-10 minutes. Preferably, the denaturation step is carried out for 3 minutes.

In a preferred embodiment, the amplification step comprises a polymerase chain reaction (PCR). Preferably, the PCR comprises 15 cycles at 94° C. for 20 seconds, 58° C. for 30 seconds and 68° C. for 10 minutes, and 20 cycles of 94° C. for 20 seconds, 55° C. for 30 seconds and 68° C. for 10 minutes and a final extension step at 68° C. for 15 minutes.

The one or more further amplicons may be analysed by capillary electrophoresis, melt curve analysis, on a DNA chip or next generation sequencing.

The primers according to the disclosure may additionally comprise a detectable label, enabling the probe to be detected. Examples of labels that may be used include: fluorescent markers or reporter dyes, for example, 6-carboxyfluorescein (6FAM™), NED™ (Applera Corporation), HEX™ or VIC™ (Applied Biosystems); TAMRA™ markers (Applied Biosystems, Calif., USA); chemiluminescent markers, for example Ruthenium probes.

Alternatively the label may be selected from the group consisting of electroluminescent tags, magnetic tags, affinity or binding tags, nucleotide sequence tags, position specific tags, and or tags with specific physical properties such as different size, mass, gyration, ionic strength, dielectric properties, polarisation or impedance.

Well-known extraction and purification procedures are available for the isolation of protein from a sample. The protein may be used directly following extraction from the sample. Protein extraction may be by physical cell disruption or detergent based cell lysis. Extracted proteins may be analysed by Western blot, Coomasie stain, Bradford assay and BCA assay.

The method disclosed herein is suitable for determining if a patient is a candidate for a differential treatment plan. A differential treatment plan may comprise of one or more types of treatment selected from the group consisting of chemotherapy, immunotherapy, radiation therapy, targeted therapy and transplantation. A differential treatment plan may also include a combination of one or more therapies. A differential treatment plan may comprise one or more therapies applied simultaneously or sequentially. In a preferred embodiment, the differential therapy is targeted therapy. In another preferred embodiment, the differential therapy is targeted therapy in combination with chemotherapy. In one embodiment, the differential treatment plan is transtuzumab or ramucirumab. In another embodiment, the differential treatment plan is transtuzumab or ramucirumab in combination with chemotherapy.

The method disclosed herein is suitable for determining or making of a prognosis if a person is at risk of cancer. As previously described, a person at risk of cancer has an increased probability of having cancer relative to a control or reference that does not have the one or more fusion genes. In one embodiment, a person or patient has a 10%, 15%, 20%, 25%, 30%, 35%, 40%, 45%, 50%, 55%, 60%, 65%, 70%, 75%, 80%, 85%, 90%, 95% or 99% increased risk of cancer.

The nucleotide sequence of the one or more fusion genes may be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%. 92%, 93%, 94%, 95%, 96%, 97%, 98%, 99% or 100% identical to a sequence selected from the group consisting of CLEC16A-EMP2 (SEQ ID NO.: 97, 99 or 101), SNX2-PRDM6 (SEQ ID NO. 115), MLL3 PRKAG2 (SEQ ID NO.: 121, 123 or 125), DUS2L-PSKH1 (SEQ ID NO.: 131 or 133) and CLDN18-ARHGAP26 (SEQ ID NO: 107). In one example, the nucleotide sequence of CLEC16A-EMP2 is 70% identical to SEQ ID NO.: 97. In another example, the nucleotide sequence of CLDN18-ARHGAP26 is 95% identical to SEQ ID NO: 107. In yet another example, wherein the cancer-associated fusion gene is CLEC16A-EMP2 in combination with CLDN18-ARHGAP26, CLEC16A-EMP2 is 80% identical to SEQ ID NO. 97 and CLDN18-ARHGAP26 is 85% identical to SEQ ID NO. 107.

There is also provided an expression vector comprising the coding sequence of any of the fusion genes disclosed herein. In one embodiment, the expression vector is a mammalian expression vector. Suitable expression vectors include but are not limited to pMXs-Puro, pVSVG, pEGFP and pCMVmyc.

There is also provided a cell transformed with an expression vector as disclosed herein. Transformation may be by electroporation, heat shock, chemical or viral transfection. In one embodiment, the cell is transformed by chemical transfection. In another embodiment, the chemical transfection is by Lipofectamine 2000. In another embodiment, transformation is by viral transfection. In yet another embodiment, viral transfection is lentiviral or retroviral transfection.

There is also provided a method for producing a polypeptide, comprising culturing the transformed cell in Eagle's Minimum Essential Medium or Dulbecco's Modified Eagle's Medium or RPMI with 10% bovine serum, 2 mM Glutamine, 1% non essential amino acids and 1% penicillin/streptomycin in a humidified chamber at 5% CO2 and 37° C. for polypeptide expression and collecting the amount of said polypeptide from the cell. It is within the ambit of the skilled person to vary the parameters of the culture conditions to optimize production and extraction of the polypeptide.

Also disclosed is a use of a cancer-associated fusion gene in the determination or prognosis of cancer in a patient, wherein the presence of one or more cancer-associated fusion genes in a sample obtained from the patient indicates that the patient has cancer or is at an increased risk of developing cancer.

### EXPERIMENTAL SECTION

Non-limiting examples of the invention and comparative examples will be further described in greater detail by reference to specific Examples, which should not be construed as in any way limiting the scope of the invention.

Materials and Methods

Clinical Tumor Samples

Patient samples and clinical information were obtained from patients who had undergone surgery for gastric cancer at the National University Hospital, Singapore, and Tan Tock Seng Hospital, Singapore. Informed consent was obtained from all subjects and the study was approved by the Institutional Review Board of the National University of Singapore (reference code 05-145) as well as the National Healthcare Group Domain Specific Review Board (reference code 2005/00440).

DNA/RNA Extraction from Samples

Genomic DNA and total RNA extraction from tissue samples was performed using Allprep DNA/RNA Mini Kit (Qiagen). Genomic DNA was extracted from blood samples with Blood & Cell Culture DNA kit (Qiagen).

Primers and Oligonucleotides

The primers and oligonucleotides used in this study are described in Table 1.

TABLE 1
Primers used in this study.
Primers for screening for
presence of the 5 fusion genes

| | | |
|---|---|---|
| CLDN18- | Forward | TTTCAACTACCAGGGGCTGT |
| ARHGAP26 | | (SEQ ID NO: 1) |
| | Reverse | GCCAGTCTTTCCGTTCAGAG |
| | | (SEQ ID NO: 2) |
| CLEC16A- | Forward | TAGTGGAGACCATCCGTTCC |
| EMP2 | | (SEQ ID NO: 3) |
| | Reverse | CCTTCTCTGGTCACGGGATA |
| | | (SEQ ID NO: 4) |
| DUS2L- | Forward | CAGTACGGTGTGTGGAGCTG |
| PSKH1 | | (SEQ ID NO: 5) |
| | Reverse | GGTGCAGGTTCTTCATGGAT |
| | | (SEQ ID NO: 6) |
| MLL3- | Forward | CCTTTCCAGAGAGCCAGAAA |
| PRKAG2 | | (SEQ ID NO: 7) |
| | Reverse | GCAAAACGTGACCCAGAGAC |
| | | (SEQ ID NO: 8) |
| SNX2- | Forward | TTCACCAGCACTGTCTCCAC |
| PRDM6 | | (SEQ ID NO: 9) |
| | Reverse | TTCGATTGATTCTGGGCTCT |
| | | (SEQ ID NO: 10) |

Primers for cloning gastric
fusion gene constructs

| | | |
|---|---|---|
| CLEC16A- | Forward | GGCGCGGATCCGCCGCCACC |
| EMP2 | | ATGTTTGGCCGCTCGCGGAG |
| | | (SEQ ID NO: 11) |
| | Reverse | TGATAGCGGCCGCTCATCAA |
| | | GCGTAATCTGGAACATCGTA |
| | | TGGGTACTCGAGTTTGCGCT |
| | | TCCTCAGTATCAG |
| | | (SEQ ID NO: 12) |
| CLDN18- | Forward | GGCGCGGATCCGCCGCCACC |
| ARHGAP26 | | ATGGCCGTGACTGCCTGTCA |
| | | (SEQ ID NO: 13) |
| | Reverse | GATAGCGGCCGCTCATCAAG |
| | | CGTAATCTGGAACATCGTAT |
| | | GGGTACTCGAGGAGGAACTC |
| | | CACGTAATTCTCA |
| | | (SEQ ID NO: 14) |
| SNX2- | Forward | GGCGCTTAATTAAGCCGCCA |
| PRDM6 | | CCATGGCGGCCGAGAGGGAA |
| | | CC |
| | | (SEQ ID NO: 15) |
| | Reverse | TGATAGCGGCCGCTCATCAA |
| | | GCGTAATCTGGAACATCGTA |
| | | TGGGTACTCGAGATCCACTT |
| | | CGATTGATTCTGG |
| | | (SEQ ID NO: 16) |
| DUS2L- | Forward | GGCGCGGATCCGCCGCCACC |
| PSKH1 | | ATGATTTTGAATAGCCTCTC |
| | | (SEQ ID NO: 17) |
| | Reverse | TGATAGCGGCCGCTCATCAA |
| | | GCGTAATCTGGAACATCGTA |
| | | TGGGTACTCGAGGCCATTGT |
| | | ATTGCTGCTGGTAG |
| | | (SEQ ID NO: 18) |

Canine primers for qPCR

| | | |
|---|---|---|
| EMT primers | | |
| E cadherin | Forward | AAAACCCACAGCCTCATGTC |
| | | (SEQ ID NO: 19) |
| | Reverse | CACCTGGTCCTTGTTCTGGT |
| | | (SEQ ID NO: 20) |
| Fibronectin | Forward | GGTTTCCCATTATGCCATTG |
| | | (SEQ ID NO: 21) |
| | Reverse | TTCCAAGACATGTGCAGCTC |
| | | (SEQ ID NO: 22) |
| Vimentin | Forward | CCGACAGGATGTTGACAATG |
| | | (SEQ ID NO: 23) |
| | Reverse | TCAGAGAGGTCGGCAAACTT |
| | | (SEQ ID NO: 24) |
| MMP-2 | Forward | GGATGCTGCCTTTAATTGGA |
| | | (SEQ ID NO: 25) |
| | Reverse | CGCACCCTTGAAGAAGTAGC |
| | | (SEQ ID NO: 26) |
| MMP-9 | Forward | CAAACTCTACGGCTTCTGCC |
| | | (SEQ ID NO: 27) |
| | Reverse | TGGCACCGATGAATGATCTA |
| | | (SEQ ID NO: 28) |
| Slug | Forward | AAGCAGTTGCACTGTGATGC |
| | | (SEQ ID NO: 29) |
| | Reverse | GCAGTGAGGGCAAGAAAAAG |
| | | (SEQ ID NO: 30) |
| Snail | Forward | CAAGGCCTTCAACTGCAAAT |
| | | (SEQ ID NO: 31) |
| | Reverse | AAGGTTCGGGAACAGGTCTT |
| | | (SEQ ID NO: 32) |
| TJ primers | | |
| Cingulin | Forward | CTGAAGTAGCTTCCCCAGG |
| | | (SEQ ID NO: 33) |
| | Reverse | TGTTGATGAGTGAGTCCACTG |
| | | (SEQ ID NO: 34) |
| Occludin | Forward | ACACGGATCCCAGAGCAGC |
| | | (SEQ ID NO: 35) |
| | Reverse | TGCAGCGATAAAACAAAAGGC |
| | | (SEQ ID NO: 36) |
| ZO1 | Forward | GCCCCTGCACCGTGG |
| | | (SEQ ID NO: 37) |
| | Reverse | TCTCTGACCCTCCAGCCAAT |
| | | (SEQ ID NO: 38) |
| ZO2 | Forward | GCGACGGTTCTTTCTAGGGA |
| | | (SEQ ID NO: 39) |
| | Reverse | TCCCCTTGAGGAAATGGGAG |
| | | (SEQ ID NO: 40) |
| ZO3 | Forward | CCAGGGACAGTCCCCCC |
| | | (SEQ ID NO: 41) |
| | Reverse | GCGTCGGGTTCCGAGAT |
| | | (SEQ ID NO: 42) |
| Cld2 | Forward | GGTGGGCATGAGATGCACT |
| | | (SEQ ID NO: 43) |
| | Reverse | CACCACCGCCAGTCTGTCTT |
| | | (SEQ ID NO: 44) |
| Cld3 | Forward | GAGGGCCTGTGGATGAACTG |
| | | (SEQ ID NO: 45) |
| | Reverse | AGTCGTACACCTTGCACTGCA |
| | | (SEQ ID NO: 46) |
| Focal | | |
| adhesion | | |
| primers | | |
| Paxillin | Forward | TCCACCACCTCGCATATCTCT |
| | | (SEQ ID NO: 47) |
| | Reverse | GCCATTTAGGGCCTCACTGGA |
| | | (SEQ ID NO: 48) |
| Talin1 | Forward | CCAGAAGGTTCCTTTGTGGA |
| | | (SEQ ID NO: 49) |
| | Reverse | GGCTGGTGTTTGACTTGGTT |
| | | (SEQ ID NO: 50) |
| Talin2 | Forward | GGTGGCCCTGTCCTTAAAG |
| | | (SEQ ID NO: 51) |
| | Reverse | CGTACCCGTCCCTTCCTCC |
| | | (SEQ ID NO: 52) |
| FAK | Forward | AAGTGTGCTCTGGGGTCAAG |
| | | (SEQ ID NO: 53) |
| | Reverse | AGCCTTTGTCCGTGAGGTAA |
| | | (SEQ ID NO: 54) |
| ILK1 | Forward | AGCTCAACTTTCTGGCGAAG |
| | | (SEQ ID NO: 55) |
| | Reverse | CTTCACGACGATGTCATTGC |
| | | (SEQ ID NO: 56) |
| Pinch 1 | Forward | CCATTTAAAGATCTCCG |
| | | (SEQ ID NO: 57) |
| | Reverse | CATTTGGAAGTCATGTTCG |
| | | (SEQ ID NO: 58) |
| Proteoglycan | | |
| primers | | |
| Syndecan | Forward | AGGACGAGGGGAGCTATGACC |
| | | (SEQ ID NO: 59) |
| | Reverse | GTGGGGGCCTTCTGATAAG |
| | | (SEQ ID NO: 60) |
| Integrin | | |
| subunits | | |
| primers | | |
| β1 | Forward | ATCCCAGAGGCTCCAAAGAT |
| | | (SEQ ID NO: 61) |
| | Reverse | GCTGGAGCTTCTCTGCTGTT |
| | | (SEQ ID NO: 62) |
| β3 | Forward | GACCTTTGAGTGTGGGGTGT |
| | | (SEQ ID NO: 63) |
| | Reverse | TCTTCCGAGCATTCACACTG |
| | | (SEQ ID NO: 64) |
| β4 | Forward | ACAGTCCCAAGAAACGGATG |
| | | (SEQ ID NO: 65) |
| | Reverse | CCTTCACCGTGTAGCGGTAT |
| | | (SEQ ID NO: 66) |
| β5 | Forward | AAGCCCATCTCCACACACTC |
| | | (SEQ ID NO: 67) |
| | Reverse | AGGAGAAGGGGCTCTCAGTC |
| | | (SEQ ID NO: 68) |
| β6 | Forward | TGAGACCAGGCAGTGAACAG |
| | | (SEQ ID NO: 69) |
| | Reverse | CCGAGAGGTCCATGAGGTAA |
| | | (SEQ ID NO: 70) |
| β8 | Forward | CGTGACTTCCGTCTTGGATT |
| | | (SEQ ID NO: 71) |
| | Reverse | CCTTTCTGGGTGGATGCTAA |
| | | (SEQ ID NO: 72) |
| α2 | Forward | ATTTGGAAACTGCCACAAGC |
| | | (SEQ ID NO: 73) |
| | Reverse | ATTTGGAAACTGCCACAAGC |
| | | (SEQ ID NO: 74) |
| α3 | Forward | CATCTACCACAGCAGCTCCA |
| | | (SEQ ID NO: 75) |
| | Reverse | CTCCTCCCCATGGATTACCT |
| | | (SEQ ID NO: 76) |
| α5 | Forward | GACGACACGGAGGACTTTGT |
| | | (SEQ ID NO: 77) |
| | Reverse | TGTCTGAGCCATTGAGGATG |
| | | (SEQ ID NO: 78) |
| α6 | Forward | AGTGGAGCTGTGGTTTTGCT |
| | | (SEQ ID NO: 79) |
| | Reverse | AGACCTTCCCCGTCAAAAAT |
| | | (SEQ ID NO: 80) |
| αV | Forward | TCCAGGTGGAGCTTCTTTTG |
| | | (SEQ ID NO: 81) |
| | Reverse | TTCTTAGAGTGACCTGGAGACC |
| | | (SEQ ID NO: 82) |
| GAPDH | Forward | AACATCATCCCTGCTTCCAC |
| | | (SEQ ID NO: 83) |
| | Reverse | GACCACCTGGTCCTCAGTGT |
| | | (SEQ ID NO: 84) |
| Human | | |
| Primers | | |
| for qPCR | | |
| N cadherin | Forward | ACAGTGGCCACCTACAAAGG |
| | | (SEQ ID NO: 85) |
| | Reverse | CCGAGATGGGGTTGATAATG |
| | | (SEQ ID NO: 86) |
| Beta | Forward | AAAATGGCAGTGCGTTTAG |
| catenin | | (SEQ ID NO: 87) |
| | Reverse | TTTGAAGGCAGTCTGTCGTA |
| | | (SEQ ID NO: 88) |
| PAK1 | Forward | CGTGGCTACATCTCCCATTT |
| | | (SEQ ID NO: 89) |
| | Reverse | TCCCTCATGACCAGGATCTC |
| | | (SEQ ID NO: 90) |
| GAPDH | Forward | GACCCCTTCATTGA |
| | | (SEQ ID NO: 91) |
| | Reverse | CTTCTCCATGGTGG |
| | | (SEQ ID NO: 92) |

Antibodies and Reagents

Primary and secondary commercial antibodies and reagents are described in Table 2.

TABLE 2
Primary and secondary commercial antibodies and reagents.

| Protein | Catalogue number | Vendor |
|---|---|---|
| ARHGAP26 | Prestige | Sigma-Aldrich |
| | #HPA035107 | |
| Vinculin | #V9131 | Sigma-Aldrich |
| CLDN18 | mid, # 388100 | Life Technologies |
| ZO-1 | #61-7300 | Life Technologies |
| Alpha Tubulin | # 32-2500 | Life Technologies |
| GAPDH | # 437000 | Life Technologies |
| CTxB conjugated to | #C-34777 | Life Technologies |
| Alexa Fluro ® 594 | | |
| E cadherin | #610182 | BD Biosciences |
| N cadherin | #610920 | BD Biosciences |
| Beta catenin | #610153 | BD Biosciences |
| Paxillin | #610051 | BD Biosciences |
| pFAK | #611722 | BD Biosciences |
| Integrin beta 1 | # 610467 | BD Biosciences |
| FAK | #ab40794 | Abcam |
| Integrin beta 5 | #ab15449 | Abcam |
| ILK1 | #52480 | Abcam |
| Pinch 1 | #ab108609 | Abcam |
| AKT | #4691 | CST |
| pAKT | #4060 | CST |
| PAK1 | #2602 | CST |
| Talin-1 | #4021 | CST |
| RhoA | #21175 | CST |
| Beta Pix | #AB3829 | Chemicon |
| Actin | #MAB1501R | Chemicon |
| Active RhoA | #26904 | NewEast |
| | | Bioscience |
| GIT1(kind gift from Ed Manser) | | |
| Secondary antibodies for Western | | Biorad |
| blots | | Laboratories and |
| | | Thermo Fisher |
| | | Scientific |
| Secondary for immunofluorescence | | Life Technologies |
| Rat Collagen type 1 | | BD Biosciences |
| Human Fibronectin | | R&D Biosystems |

RT-PCR Screen for the Presence of a Fusion Gene

1 μg of total RNA is reverse transcribed to cDNA using the SuperScript III kit (Invitrogen) according to the manufacturer's recommendations. JumpStart RED AccuTaq LA DNA Polymerase kit (Sigma) was used with the following protocol:

| Reagent | Final Concentration | |
|---|---|---|
| AccuTaq LA 10x Buffer (Sigma) | 1x | |
| dNTP mix (10 mM) | 500 | μM |
| Forward primer (100 μM) | 0.4 | μM |
| Reverse primer (100 μM) | 0.4 | μM |
| JumpStart RED AccuTaq LA DNA | 0.05 | units/μL |
| Polymerase (Sigma) | | |
| Water | To 25 | μL |

Cycling conditions are as follows: 94° C. for 3 min, (94° C. for 20 seconds, 58° C. for 30 seconds, 68° C. for 10 min)×15 cycles, (94° C. for 20 seconds, 55° C. for 30 seconds, 68° C. for 10 min)×20 cycles, 68° C. for 15 min.

Cell Culture Conditions and Transfections

MDCK II, HeLa, HGC27 and TMK1 cell lines were cultured according to standard conditions. Transient and stable transfections experiments were carried using JetPrimePolyPlus transfection kit according to manufacturer's instructions. Stable transfectants were generated with G418 selection.

DNA-PET Libraries Construction, Sequencing, Mapping and Data Analysis

DNA-PET library construction of 10 kb fragments of genomic DNA, sequencing, mapping and data analysis were performed with refined bioinformatics filtering. The short reads were aligned to the NCBI human reference genome build 36.3 (hg18) using Bioscope (Life Technologies). DNA-PET data of TMK1 and tumors 17, 26, 28 and 38 have been previously described (NCBI Gene Expression Omnibus (GEO) accession no. GSE26954) and of tumors 82 and 92 (NCBI GEO accession number GSE30833). The SOLID sequencing data of the eight additional tumor/normal pairs can be accessed at NCBI's Sequence Read Archive (SRA) under BioProject ID PRJNA234469. Procedures for the identification of recurrent genomic breakpoints of CLDN18-ARHGAP26, filtering of germline structural variations (SV) in cancer genomes and breakpoint distribution analyses are described as follows.

For 10 of the 15 GC samples, paired normal samples were available and the respective DNA-PET data was used to filter germline SVs from the SVs which were identified in the tumors. For this, extended mapping coordinates of the clusters of discordant paired-end tag (dPET) sequences which defined the SVs were searched for overlap with dPET clusters of the paired normal sample. In addition, and in particular for the tumors without paired normal samples (tumors 17, 26, 28 and 38) and TMK1, all SVs of the paired normal samples and of 16 unrelated non-cancer individuals were used for filtering. Further, simulations were performed in which paired sequence tags in a distance distribution of a representative library were randomly selected from the reference sequence and were mapped and processed by the pipeline. Resulting dPET clusters represented mapping artifacts and were used for SV filtering. Further, dPET clusters were compared with SVs in the database of genomic variants (http://dgv.tcag.ca/dgv/app/home), paired-end sequencing studies of non-cancer individuals when the larger SV overlapped by ≧80% with SVs identified in cancer genomes. The data processing by the standard pipeline resulted in a large number of small deletions for the blood sample of patient 82 due to the abnormal insert size distribution and all the deletions smaller than 12 kb were removed.

MCF-7 RNA Polymerase II ChIA-PET and GC DNA-PET Comparison

To investigate whether the two partner sites of germline and somatic SV of the study were enriched for loci which are in proximity of each other in the nucleus, overlap of SVs were tested with genome-wide chromatin interaction data sets derived from ChIA-PET sequencing of the breast cancer cell line MCF-7 with the rationale that some chromatin interactions might be conserved across different cell types.

Driver Fusion Gene Prediction

The potential driver fusion genes were predicted by in silico analysis as previously described. The in silico analysis is a network fusion centrality approach in which the position of a gene product within transcript networks is used to predict its importance for the network to function. The threshold value 0.37 was set for identifying the potential fusion drivers.

In-Frame Fusion Gene Confirmation and Screening by RT-PCR

One microgram of total RNA was reverse-transcribed to cDNA using SuperScript III First-Strand Synthesis System for RT-PCR (Invitrogen) according to the manufacturer's instruction. PCR was done with JumpStart™ REDAccuTaq LA DNA Polymerase (Sigma-Aldrich Inc.).

GC Fusion Gene Constructs and Retroviral Transfections

The GC fusion genes CLEC16A-EMP2, CLDN18-ARHGAP26, SNX2-PRDM6 and DUS2L-PSKH1 were amplified from tumor samples by PCR using 2× Phusion Mastermix with HF buffer (Thermo Scientific) and the following primers.

Open reading frame of the CLEC16A-EMP2 fusion was constructed with the FLAG peptide of pMXs-Puro in frame using forward primer

(SEQ ID NO. 11)
5′ GGCGCGGATCCGCCGCCACCATGTTTGGCCGCTCGCGGAG-3′

(BamHI, kozak sequence and start codon follow by the first coding nucleotides of CLEC16A) and reverse primer 5′-

(SEQ ID NO.: 12)
5′-TGATAGCGGCCGCTCATCAAGCGTAATCTGGAACATCGTATGGGTA
CTCGAGTTTGCGCTTCCTCAGTATCAG-3′

(NotI, stop codon, HA-tag and XhoI followed by the 3′ end of the coding sequence of EMP2).

Similarly, open reading frame of the CLDN18-ARHGAP26 fusion was constructed with forward primer 5′ GGCGCGGATCCGCCGCCACCATGGCCGTGACTGCCTGTCA-3′ (SEQ ID NO.: 13) (BamHI, kozak, start, CLDN18) and reverse primer

(SEQ ID NO.: 14)
5′-GATAGCGGCCGCTCATCAAGCGTAATCTGGAACATCGTATGGGTAC
TCGAGGAGGAACTCCACGTAATTCTCA-3′

(NotI, stop, HA-tag, XhoI, ARHGAP26).

Open reading frame of the SNX2-PRDM6 fusion was constructed using forward primer 5′-GGCGCTTAATTAAGCCGCCACCATGGCGGCCGAGAGGGAACC-3′ (SEQ ID NO.: 15) (PacI, kozak, start, SNX2) and reverse

(SEQ ID NO.: 16)
5′-TGATAGCGGCCGCTCATCAAGCGTAATCTGGAACATCGTATGGGTA
CTCGAGATCCACTTCGATTGATTCTGG-3′

(NotI, stop, HA-tag, XhoI PRDM6).

Open reading frame of the DUS2L-PSKH1 fusion was constructed using forward primer 5′-GGCGCGGATCCGCCGCCACCATGATTTTGAATAGCCTCTC-3′ (SEQ ID NO.: 17) (BamHI, kozak, start, DUS2L) and reverse primer

(SEQ ID NO.: 18)
5′-TGATAGCGGCCGCTCATCAAGCGTAATCTGGAACATCGTATGGGTA
CTCGAGGCCATTGTATTGCTGCTGGTAG-3′

(NotI, stop, HA-tag, XhoI, PSKH1).

MLL3-PRKAG2 was synthesized with the FLAG peptide of pMXs-Puro by the gBlock method (Integrated DNA Technologies, Inc). The PCR products or MLL3-PRKAG2 were cloned into pMXs-Puro retroviral vector (Cell biolabs, RTV-012). The pMXs-Puro retroviral vectors containing the fusion genes were co-transfected with pVSVG (pseudotyping construct) into GP2-293 cells using lipofectamine 2000 to produce virus. Both HGC27 and HeLa cells were then infected with the viral supernatant containing empty vector or the fusion genes. Stable transfectants were obtained and maintained under selection pressure by puromycin dihydrochloride (Sigma, P9620).

Construction of CLDN18 and ARHGAP26 Plasmids

Human CLDN18 cDNA was obtained from IMAGE consortium (http://www.imageconsortium.org/) and cloned with an N-terminal HA-tag into pcDNA3 vector. The last three amino acids (DYV) of CLDN18 which encodes PDZ-binding motif was mutated to alanines and referred to as CLDN18ΔP. The human ARHGAP26 (GRAF1 isoform 2) cDNA in pEGFP vector and pCMVmyc were kindly provided by Dr Richard Lundmark (Medical Biochemistry and Biophysics, Umeå University, 901 87 Umeå, Sweden).

Details of the ARHGAP26 isoform is as follows:

Transcript: ARHGAP26-008 ENST00000378004 (http://www.ensembl.org) (SEQ ID NO.: 135)

ATGGGGCTCCCAGCGCTCGAGTTCAGCGACTGCTGCCTCGATAGTCCGC
ACTTCCGAGAGACGCTCAAGTCGCACGAAGCAGAGCTGGACAAGACCAA
CAAATTCATCAAGGAGCTCATCAAGGACGGGAAGTCACTCATAAGCGCG
CTCAAGAATTTGTCTTCAGCGAAGCGGAAGTTTGCAGATTCCTTAAATG
AATTTAAATTTCAGTGCATAGGAGATGCAGAAACAGATGATGAGATGTG
TATAGCAAGATCTTTGCAGGAGTTTGCCACTGTCCTCAGGAATCTTGAA
GATGAACGGATACGGATGATTGAGAATGCCAGCGAGGTGCTCATCACTC
CCTTGGAGAAGTTTCGAAAGGAACAGATCGGGGCTGCCAAGGAAGCCAA
AAAGAAGTATGACAAAGAGACAGAAAAGTATTGTGGCATCTTAGAAAAA
CACTTGAATTTGTCTTCCAAAAAGAAAGAATCTCAGCTTCAGGAGGCAG
ACAGCCAAGTGGACCTGGTCCGGCAGCATTTCTATGAAGTATCCCTGGA
ATATGTCTTCAAGGTGCAGGAAGTCCAAGAGAGAAAGATGTTTGAGTTT
GTGGAGCCTCTGCTGGCCTTCCTGCAAGGACTCTTCACTTTCTATCACC
ATGGTTACGAACTGGCCAAGGATTTCGGGGACTTCAAGACACAGTTAAC
CATTAGCATACAGAACACAAGAAATCGCTTTGAAGGCACTAGATCAGAA
GTGGAATCACTGATGAAAAAGATGAAGGAGAATCCCCTTGAGCACAAGA
CCATCAGTCCCTACACCATGGAGGGATACCTCTACGTGCAGGAGAAACG
TCACTTTGGAACTTCTTGGGTGAAGCACTACTGTACATATCAACGGGAT
TCCAAACAAATCACCATGGTACCATTTGACCAAAAGTCAGGAGGAAAAG
GGGGAGAAGATGAATCAGTTATCCTCAAATCCTGCACACGGCGGAAAAC
AGACTCCATTGAGAAGAGGTTTTGCTTTGATGTGGAAGCAGTAGACAGG
CCAGGGGTTATCACCATGCAAGCTTTGTCGGAAGAGGACCGGAGGCTCT
GGATGGAAGCCATGGATGGCCGGGAACCTGTCTACAACTCGAACAAAGA
CAGCCAGAGTGAAGGGACTGCGCAGTTGGACAGCATTGGCTTCAGCATA
ATCAGGAAATGCATCCATGCTGTGGAAACCAGAGGGATCAACGAGCAAG
GGCTGTATCGAATTGTGGGTGTCAACTCCAGAGTGCAGAAGTTGCTGAG
TGTCCTGATGGACCCCAAGACTGCTTCTGAGACAGAAACAGATATCTGT
GCTGAATGGGAGATAAAGACCATCACTAGTGCTCTGAAGACCTACCTAA
GAATGCTTCCAGGACCACTCATGATGTACCAGTTTCAAAGAAGTTTCAT
CAAAGCAGCAAAACTGGAGAACCAGGAGTCTCGGGTCTCTGAAATCCAC
AGCCTTGTTCATCGGCTCCCAGAGAAAAATCGGCAGATGTTACAGCTGC
TCATGAACCACTTGGCAAATGTTGCTAACAACCACAAGCAGAATTTGAT
GACGGTGGCAAACCTTGGTGTGGTGTTTGGACCCACTCTGCTGAGGCCT
CAGGAAGAAACAGTAGCAGCCATCATGGACATCAAATTTCAGAACATTG
TCATTGAGATCCTAATAGAAAACCACGAAAAGATATTTAACACCGTGCC
CGATATGCCTCTCACCAATGCCCAGCTGCACCTGTCTCGGAAGAAGAGC
AGTGACTCCAAGCCCCCGTCCTGCAGCGAGAGGCCCCTGACGCTCTTCC
ACACCGTTCAGTCAACAGAGAAACAGGAACAAAGGAACAGCATCATCAA
CTCCAGTTTGGAATCTGTCTCATCAAATCCAAACAGCATCCTTAATTCC
AGCAGCAGCTTACAGCCCAACATGAACTCCAGTGACCCAGACCTGGCTG
TGGTCAAACCCACCCGGCCCAACTCACTCCCCCCGAATCCAAGCCCAAC
TTCACCCCTCTCGCCATCTTGGCCCATGTTCTCGGCGCCATCCAGCCCT
ATGCCCACCTCATCCACGTCCAGCGACTCATCCCCCGTCAGCACACCGT
TCCGGAAGGCAAAAGCCTTGTATGCCTGCAAAGCTGAACATGACTCAGA
ACTTTCGTTCACAGCAGGCACGGTCTTCGATAACGTTCACCCATCTCAG
GAGCCTGGCTGGTTGGAGGGGACTCTGAACGGAAAGACTGGCCTCATCC
CTGAGAATTACGTGGAGTTCCTC

followed in frame by HA-tag followed by stop codon. The human influenza hemagglutinin (HA)-tag has one of the following nucleotide sequences: 5′ TAC CCA TAC GAT GTT CCA GAT TAC GCT 3′ or 5′ TAT CCA TAT GAT GTT CCA GAT TAT GCT 3′. It will also be understood that the stop codon can be selected from any one of the following: TAG, TAA, or TGA.

Fusion Gene Recurrence Significance Test

The statistical significance of the observed frequency of fusion genes was assessed using a randomization framework. SV profiles were defined that mimic the type, number and size distributions of SVs identified in the samples sequenced by DNA-PET. The SVs of a 15 GCs test data set were simulated using the SV profiles and the frequency of recurrent SVs on a simulated validation set of 85 GC samples was assessed. Letting N=10,000 be the number of random simulations and eₛ the frequency in the validation data set of an SV s present in the test data set, P values (eₛ) were defined as p/N, where p is the number of simulations where a SV k exists with a frequency eₖ≧eₛ.

Cell Aggregation, Cell Adhesion and Wound Healing Assays

For cell aggregation assay, 20 μl of 1.2×10⁶/ml cells were plated on tissue culture dishes as hanging drops and phase contrast images were obtained the next day using Nikon Eclipse TE2000-S.

For cell adhesion assay, 24-well plates were either non-treated or treated with 1 mg/ml of fibronectin and 10 μg/ml of rat collagen type 1 for 2 hrs and blocked with 0.1% BSA. 2.5×10⁴/ml of cells were seeded and incubated at 37° C. for 2 hrs.

In detail, 24-well plates were treated with 1 mg/ml of fibronectin and 10 μg/ml of rat collagen type 1 for 2 hrs. The plates were subsequently washed and non-specific binding was prevented by treating the surfaces with 0.1% bovine serum albumin (BSA) for 20 mins. The surfaces were again washed with PBS and 2.5×10⁴/ml of cells were seeded and incubated at 37° C. for 2 hrs. Cells were also seeded on untreated 24-well as control. Cells were imaged with phase contrast microscopy. For quantification of cells adhered to the surfaces, the cells were gently washed with PBS three times and fixed in PFA and counted.

For wound healing assay, 70 ul of 7×10⁵ cells/ml were plated on culture insert in μ-Dish 35 mm (Ibidi). The following day, the insert was peeled off to create a wound and migration was imaged with Nikon Eclispe TE2000 until closure of the wound.

Cell Proliferation Assay

800 cells were seeded in quadruplicates for each condition in 24-well plates and readings were taken according to manufacturer's instructions (Cell Proliferation Reagent WST-1: Roche) for 7 days. Absorbance was measured using Infinite M200 Quad4 Monochromator (Tecan) at 450 nm using a reference wavelength of 650 nm.

Cell Invasion Migration Assay

0.5 ml of 1×10⁵ stably transfected HeLa and MDCK cells in RPMI serum free media were plated into the Biocoat Matrigel invasion chamber according to manufacturer's instructions (Corning) with 5% FBS in media added as chemoattractant to the wells of the Matrigel invasion chamber for 24 hr. Specifically, 0.5 ml of 1×10⁵ HeLa and MDCK cells stably transfected with CLDN18, ARHGAP26 and CLDN18-ARHGAP26 in RPMI serum free media were plated into the Biocoat Matrigel invasion chamber according to manufacturer's instructions (Corning). 5% FBS in media was added as chemoattractant to the wells of the Matrigel invasion chamber for 24 hr. The following day, the cells were fixed for 10 min in 3.7% PFA and the insert was washed with PBS. 0.1% of crystal violet was added to the insert for 10 min and washed twice with water. A cotton swap was used to remove any non-invading cells and washed again. The number invading cells were imaged using Nikon Eclipse TE2000-S and counted.

Transepithelial Epithelial Resistance (TER) Analysis

2×10⁵ stably transfected MDCK cells were seeded on 12 mm Transwell inserts (Corning) to obtain a polarized monolayer. The next day, the inserts were placed in CellZcope (nanoAnalytics) for TER measurements.

Soft Agar Colony Formation Assay

5000 cells of HeLa and HGC27 stable cell lines were added to 2 ml soft agar (0.35% Noble agar and 2×FBS media) and plated onto solidified base layers (0.7% Nobel agar with 2×FBS media) with triplicates set up for each experiment. 2-4 weeks later, colonies were counted.

Fusion Genes

5 fusion genes were used in this study as detailed in Table 3 below.

TABLE 3
Fusion genes

| Fusion Gene | Gene | Gene Bank ID | Entrez Gene |
|---|---|---|---|
| CLEC16A-EMP2 | CLEC16A | AB002348 | |
| | EMP2 | HSU52100 | |
| CLDN18- | CLDN18 | AF221069 | |
| ARHGAP26 | | | |
| | ARHGAP26 | AB014521 | |
| SNX2-PRDM6 | SNX2 | AF043453 | |
| | PRDM6 | AF272898 | |
| MLL3-PRKAG2 | MLL3 | AF264750 | |
| | PRKAG2 | AF087875 | |
| DUS2L-PSKH1 | DUS2L | | 54920 |
| | PSKH1 | M14504 | |

Details on the five recurrent fusion genes are mentioned below.

All genomic coordinates are based on the February 2009 human reference sequence (GRCh37 or hg19; http://genome.ucsc.edu/). Transcript IDs are based on Ensembl genome database (http://www.ensembl.org/). Shaded in yellow are the coding parts of the 5′ fusion partner genes as discovered in the initial screen and shaded in green are the 3′ fusion partner genes.

Fusion Gene #1: CLEC16A-EMP2

CLEC16A

Genomic PCR confirmed breakpoint—chr16: 11073471

RT-PCR confirmed RNA fusion point in exon 9—chr16: 11073239

EMP2

Genomic PCR confirmed breakpoint—chr16: 10666428

RT-PCR confirmed RNA fusion point in exon 2 (5′ UTR)—chr16: 10641534

Transcript: CLEC16A-001 ENST00000409790

cDNA sequence (SEQ ID NO. 93), coding part of
fusion gene shaded.
AACTGCATTTCCCAGCGCCCCACGCGGCGGCGGCCGTAAAGCGCGGCGG
TCGAACGGCCGGTTCCGGCTGAATGTCAGTGCTGGGCTGTGGGCCGGGG
AGGAAGGCGGCTCGCGGTTCCTCCACCGCCTCCGCCGCCGCATCCTCCG
CTTGTGCTACCGCCGCGGGCGCTGGGCCGCTCTGCTGGTCCGGCATGAG
ACCGTGAGACGAGAGACGGGTCGGGGCCGCCGACATGTTTGGCCGCTCG
CGGAGCTGGGTGGGCGGGGGCCATGGCAAGACTTCCCGCAACATCCACT
CCTTGGACCACCTCAAGTATCTGTACCACGTTTTGACCAAAAACACCAC
AGTCACAGAACAGAACCGGAACCTGCTAGTGGAGACCATCCGTTCCATC
ACTGAGATCCTGATCTGGGGAGATCAAAATGACAGCTCTGTATTTGACT
TCTTCCTGGAGAAGAATATGTTTGTTTTCTTCTTGAACATCTTGCGGCA
AAAGTCGGGCCGTTACGTGTGCGTTCAGCTGCTGCAGACCTTGAACATC
CTCTTTGAGAACATCAGTCACGAGACCTCACTTTATTATTTGCTCTCAA
ATAACTACGTAAATTCTATCATCGTTCATAAATTTGACTTTTCTGATGA
GGAGATTATGGCCTATTATATATCGTTCCTGAAAACACTTTCGTTAAAA
CTCAACAACCACACTGTCCATTTCTTTTATAATGAGCACACCAATGACT
TTGCCCTGTACACAGAAGCCATCAAGTTTTTCAACCACCCTGAAAGCAT
GGTTAGAATTGCTGTAAGAACCATAACTTTGAATGTCTATAAAGTGTCA
TTGGATAACCAGGCCATGCTGCACTACATCCGAGATAAAACTGCTGTTC
CTTACTTCTCCAATTTGGTCTGGTTCATTGGGAGCCATGTGATCGAACT
CGATGACTGCGTGCAGACTGATGAGGAGCATCGGAATCGGGGTAAACTG
AGTGATCTGGTGGCAGAGCACCTAGACCACCTGCACTATCTCAATGACA
TCCTGATCATCAACTGTGAGTTCCTCAACGATGTGCTCACTGACCACCT
GCTCAACAGGCTCTTCCTGCCCCTCTACGTGTACTCACTGGAGAACCAG
GACAAGGGAGGAGAACGGCCGAAAATTAGCCTGCCGGTGTCTCTTTATC
TTCTGTCACAGGTCTTCTTAATTATACATCATGCACCGCTGGTGAACTC
GTTAGCTGAAGTCATTCTGAATGGTGATCTGTCTGAGATGTACGCTAAG
ACTGAACAGGATATTCAGAGAAGTTCTGCCAAGCCCAGCATTCGGTGCT
TCATTAAACCCACCGAGACACTCGAGCGGTCCCTTGAGATGAACAAGCA
CAAGGGCAAGAGGCGGGTGCAAAAGAGACCCAACTACAAAAACGTTGGG
GAAGAAGAAGATGAGGAGAAAGGGCCCACCGAGGATGCCCAAGAAGACG
CCGAGAAGGCTAAAGGTACAGAGGGTGGTTCAAAAGGCATCAAGACGAG
TGGGGAGAGTGAAGAGATCGAGATGGTGATCATGGAGCGTAGCAAGCTC
TCAGAGCTGGCCGCCAGCACCTCCGTGCAGGAGCAGAACACCACGGACG
AGGAGAAAAGCGCCGCCGCCACCTGCTCTGAGAGCACGCAATGGAGCAG
ACCCTTCCTGGATATGGTGTACCACGCGCTGGACAGCCCGGATGATGAT
TACCATGCCCTGTTCGTGCTCTGCCTCCTCTATGCCATGTCTCATAATA
AAGGCATGGATCCTGAAAAATTAGAGCGAATCCAGCTCCCCGTGCCAAA
TGCGGCCGAGAAGACCACCTACAACCACCCGCTAGCTGAAAGACTCATC
AGGATCATGAACAACGCTGCCCAGCCAGATGGGAAGATCCGGCTGGCGA
CGCTGGAGCTGAGCTGCCTGCTTCTGAAGCAGCAAGTCCTGATGAGTGC
TGGCTGCATCATGAAGGACGTGCACCTGGCCTGCCTGGAGGGTGCGAGA
GAAGAAAGTGTTCACCTTGTACGACATTTTTATAAGGGAGAAGACATTT
TTTTGGACATGTTTGAAGATGAGTATAGGAGCATGACAATGAAGCCCAT
GAACGTGGAATATCTCATGATGGACGCCTCCATCCTGCTGCCCCCAACA
GGCACGCCACTGACGGGCATTGACTTCGTGAAGCGGCTGCCGTGTGGCG
ATGTGGAGAAGACCCGGCGGGCCATCCGGGTGTTCTTCATGCTGCGTTC
CCTGTCACTGCAATTGCGAGGGGAGCCTGAGACACAGTTGCCGCTGACT
CGGGAGGAGGACCTGATCAAGACTGATGATGTCCTGGATCTGAATAACA
GCGACTTGATTGCATGTACAGTGATCACCAAGGATGGCGGCATGGTCCA
GCGATTCCTGGCTGTGGATATTTACCAGATGAGTTTGGTGGAGCCTGAT
GTGTCCAGGCTTGGCTGGGGAGTGGTCAAGTTTGCAGGCCTATTGCAGG
ACATGCAGGTGACTGGCGTGGAGGACGACAGCCGTGCCCTGAACATCAC
CATCCACAAGCCTGCGTCCAGCCCCCATTCCAAGCCCTTCCCCATCCTC
CAGGCCACCTTCATCTTCTCAGACCACATCCGCTGCATCATCGCCAAGC
AGCGCCTGGCCAAAGGCCGCATCCAGGCAAGGCGCATGAAGATGCAGAG
AATAGCTGCCCTCCTGGACCTCCCAATCCAGCCCACCACTGAAGTCCTG
GGGTTTGGACTCGGCTCCTCCACCTCCACTCAGCACCTGCCTTTCCGCT
TCTACGACCAGGGGCGCCGGGGCAGCAGCGACCCCACAGTGCAGCGCTC
CGTGTTTGCATCGGTGGACAAGGTGCCAGGCTTCGCCGTGGCCCAGTGC
ATAAACCAGCACAGCTCCCCGTCCCTGTCCTCACAGTCGCCACCCTCCG
CCAGCGGGAGCCCCAGCGGCAGCGGGAGCACCAGCCACTGCGACTCTGG
AGGCACCAGCTCGTCCTCCACCCCCTCCACAGCCCAGAGTCCAGCAGAT
GCCCCCATGAGTCCAGAACTGCCTAAGCCTCACCTTCCTGACCAGTTGG
TAATCGTCAACGAAACGGAAGCAGACTCTAAGCCCAGCAAGAACGTGGC
CAGGAGCGCAGCCGTGGAGACAGCCAGCCTGTCCCCCAGCCTCGTCCCT
GCCCGGCAGCCCACCATTTCCCTGCTCTGCGAGGACACGGCTGACACGC
TGAGCGTCGAATCGCTGACCCTTGTCCCCCCAGTTGACCCCCACAGCCT
CCGCAGCCTCACCGGCATGCCCCCGCTGTCCACGCCGGCTGCCGCCTGC
ACAGAGCCCGTGGGCGAAGAGGCTGCATGTGCTGAGCCTGTGGGCACCG
CTGAGGACTGAGTCAGTGCCGGGGCCTCCCTTTGTGTGTGTGGCCCCGC
TGGTAGGGACCCCAGTGCCGCTGACTGGCAAGACACACTGGGAGCACCC
ACCATTCTGTGCGGCCCCCAGCAGCCATCTCAACCACCTATCCCTGCGC
TCCCTTGAATGGGAAGAAGCCCCACGTTGTCCTTGAATTCCTTTTTCAC
TTTGCATCTCTTCACGTGCAGGCTGGGACCAGCGGAGACACCGCGGCGA
ATGCAGATGACTGCACCGGCCACTCAGGGAGCTGCCTGGGCTCCGTGTC
TCTGAGCCCCGGGTGGCAGGACCCACCGGCACCTCTTTCTTCCTCTGTC
ATATGGCTCCTCTGTCACCAGCCCCAGTGTGCACAGAAGAATTGGACCA
GGTCACTGTACGTAGAAATTTGTAGAAAAGCAGACTTAGATAAACATCT
CCTTTGGATATTTATTTCCGCTTTTGGCAGCAGGTGAACATTTATTTTT
AAAACTTCTATTTAAAAGAAGTCCAAAAACATCAACACTAAGGTTTGAT
GTCATGTGAAAAGTGTAATAATAACAGTTAAGATTTCATGATCATTTTC
ACTGGACCTTTCCTGATATTTTGTTTCAGAGTTCTTAGTGTGGCTTTTT
CCATTTATTTAAGTGATTCTTTGTTACTCACTAACTCTGCAAGCCTGTG
GAATAATGAAGTACCTTCCTGGAAAGTTTGGATTATTTTTTAAACAAAA
ACAAGGGAGATACATGTATTCTCAGGTACACACAGAGCTGAGAGGGCTG
AATGGTTTTCTGCTATAGCAGCCGAGAGGCCTCCCATCATGGAAAGATT
TCTCCAGGAAAAGGAGGAATGTAGCCAGCTCCCCACTCAGGACGCTTCC
TCATTTCTCTTCACCAAAACCAAACAGAGACAGCTTCCAGCACCTTCTT
CAGTGTTACCATCTCTAAGAAGGAACCAGTTGGGACCGTGAAGACTCCC
GACCCTGTGGCCATGATGGAAATCAAAGGAAGACACCCTCTACGTCACC
TGCCCTCGACTGTGTGTGCCCACATGTGCCGAGAGATGGCCCAGAGCCA
GTTCCCCTCCAGCTGCAAGGGCATGGTGTCCCCAGAGCTCTGAGTCTGT
CACTCTCCCTCTGCTACTGCTGCTGATCTGAATATGGAAACCCCATGGT
TCCCTTCCCCATTCGGACTGGGTGTGTACAAGCAAGGACCCAGATGCAT
CAGACACAGCCCCCAAGATGTTCCTTTCTACTCGGCCAGCTCGGGAGCC
AGACACAGCACTCACAGCCCAGGCCGTGATCCACCCTCCCCAAGTCCAC
CAGGGCCAGCGGCCCCTCACCTCTCTGGTCACTGGTGAGACCTTCCACA
ACTTTCCTCCAGACCTGCCAGCAGATGTGCCCACCAGGGGCATTAGGTA
TCCGCCGGAGCCTGGCCATAGGGTAGTCTCGGGAGCCGCGCTGAGATCT
TTTGCCACCTGCATTTTAGAAGAACATGGTCTCTGTCTCCTCGGCCCAG
CCAGCTGTCCCGGCAAGGCCTGCCGAGGGCAGTTTTCAACCTCATGAAG
GAAACACAGTCCTGCCAAGGAGGGGGAGTGGCGCCCATGGGGACAGGCC
TCAGTCCTTAGAAGCCCTCTGGGTAGCTGTGCCCACCCAGCCTTCATGG
CTGCAGGTACAAGGACCTTTGCTTCCATAGAGAAAACGCACAGCTCAGA
AAGGGGGCCACATGGGCAGAAACCCAAAGGAAGGACAAACCACGACCAC
CGTGGCCATCTGCAGAATCCCTGGAAGAGAAGGAAGGCAGGGTGGAGCG
GGGGGAAGACCATCATGGAGAGAAGGACCACAGCATCAGGAGACGGGAC
ACGCCACACCCAGCAGGCAGCCTGTGTGTTGCTTAATTTTTTAAGAGCA
AGAGGGGTAGAGAGGATCAAGCTGGCCCTGGCTGGAGATGGCTAGCCCC
TGAGACATGCACTTCTGGTTTTGAAATGACTCTGTCTGTGGGGCAGCAG
AAACTAGAGAAGGCAAGTGGCTGCCCCACCCCAAGGCGTGACCAGGAGG
AACAGCCTGCAGCTCACTCCATGCCACACGGGTGGGCCACCAGCCTGCT
GTCAGAAGTCTCTGGGCTCCAACTGGTCTTGTAACCACTGAGCACTGAA
GGAGAGAGGTCTTGGTCAGGGCTGGACAGCATGCCCGGGAGGACCAGCA
GAGGATTAAAGGTGACTGGGAGGACCAGCGGAGGATAAAAGACACTGCT
CAGGGCAGGGCTTCTACCCTGCATCCCTGGCCAAGAAAAGGGCAGTCCC
CATGTGGGCTTGCAGGGTCACTCTCAGGGGCCTCTTTCAGCTGGGGCTG
GCAACTTGCGTCTGGGGGACACCTCCAGGTGTGTGGGGTGAGGATTTCC
TATAACCAGGGCTCCCAGAAGCTTTGCTTATGTAAGGAGGTCTGGGAGC
CAGCCCATTGGAGGCCACCAGCCATTTTGGCTTCAAAGGACCCCACCTC
ACCCAGGTCTCAGCGGCAGTGGGCACAGCTATGTCTTCAGGAGCTCCCG
TCAAACCTCATAGCTGGGGCGCTCCCAGACAGGCCAGTCCAGACAGGAC
ACGCTGGGCCCCTGGCATCCAGAGGAAGAGCCAGGAGTGTGGGAAGGCC
CACAGTGGGGGCTGTGGCTTCTGACACTCAGGTCATAGCCTCAGAGGTC
TGAGGTCAGCCCCCACAGACCCATCCGGCCCGCCCCCCAAGTCCCTGCA
GAGAGCACTTAGAGTTATGGCCCAGGCCCTGGTCCACCCTTCCCCTGTG
CACCTCCGGCTGGGTTTGCCAAGTCAGGGAGCAGGGCTGGCCGCAGGAA
CTCCCAAACCTTGGCTTTGAATATTGTTGTGGAGGTGTGCTCGTCCCTT
TCTGGACGTGCAAGGTACCTGTCCCAGCAGGTCAGATGGGGCCAGCTGA
GGCGCTCCCCCAGGCAGGAAGGGCCAGCCTTCACCATCGCGTGGGATTG
GGAGGAGGGGCCTCCGTGAGCAGCCCCTCCTCTGCCGCTGTCCCAGCCC
AGTCCCTCTCCCGGAGCCTTGGCAGCCTCCCACAACCCAGACACTTGCG
TTCACAAGCAACCTAAGGGGCAGGTGAAGAAGCGCAGCCCTGCCAGACG
CGCTAGATTCCTCTAAGGTCTCTGAGATGCACCGTTTTTTAAAAAGGCG
TGGGGTGAACTGATTTTGATCTTCTTGTCTAGATGCAATAAATAAATCT
GAAGCATTTAATGTAGTCATCTTGACATTGGGCCTACACTGTACGAGTT
CCTTATGTTTCCTTGAGCTAAAAATATGTAAATAATTTTTGTCCCAGTG
AGAACCGAGGGTTAGAAAACCTCGATGCCTCTGAGCCTCGGGACCGCTC
TAGGGAAGTACCTGCTTTCGCCAGCATGACTCATGCTTCGTGGGTACTG
AACACGAGGGTGGAAATGAAAACTGGAACTTCCTTGTAAATTTAAACTT
GGCAATAAAAGAGAAAAAAAGTTACCAAGAA

Transcript: CLEC16A-001 ENST00000409790

Protein sequence (SEQ ID NO.: 94), coding part of
fusion gene shaded.
MFGRSRSWVGGGHGKTSRNIHSLDHLKYLYHVLTKNTTVTEQNRNLLVE
TIRSITEILIWGDQNDSSVFDFFLEKNMFVFFLNILRQKSGRYVCVQLL
QTLNILFENISHETSLYYLLSNNYVNSIIVHKFDFSDEEIMAYYISFLK
TLSLKLNNHTVHFFYNEHTNDFALYTEAIKFFNHPESMVRIAVRTITLN
VYKVSLDNQAMLHYIRDKTAVPYFSNLVWFIGSHVIELDDCVQTDEEHR
NRGKLSDLVAEHLDHLHYLNDILIINCEFLNDVLTDHLLNRLFLPLYVY
SLENQDKGGERPKISLPVSLYLLSQVFLIIHHAPLVNSLAEVILNGDLS
EMYAKTEQDIQRSSAKPSIRCFIKPTETLERSLEMNKHKGKRRVQKRPN
YKNVGEEEDEEKGPTEDAQEDAEKAKGTEGGSKGIKTSGESEEIEMVIM
ERSKLSELAASTSVQEQNTTDEEKSAAATCSESTQWSRPFLDMVYHALD
SPDDDYHALFVLCLLYAMSHNKGMDPEKLERIQLPVPNAAEKTTYNHPL
AERLIRIMNNAAQPDGKIRLATLELSCLLLKQQVLMSAGCIMKDVHLAC
LEGAREESVHLVRHFYKGEDIFLDMFEDEYRSMTMKPMNVEYLMMDASI
LLPPTGTPLTGIDFVKRLPCGDVEKTRRAIRVFFMLRSLSLQLRGEPET
QLPLTREEDLIKTDDVLDLNNSDLIACTVITKDGGMVQRFLAVDIYQMS
LVEPDVSRLGWGVVKFAGLLQDMQVTGVEDDSRALNITIHKPASSPHSK
PFPILQATFIFSDHIRCIIAKQRLAKGRIQARRMKMQRIAALLDLPIQP
TTEVLGFGLGSSTSTQHLPFRFYDQGRRGSSDPTVQRSVFASVDKVPGF
AVAQCINQHSSPSLSSQSPPSASGSPSGSGSTSHCDSGGTSSSSTPSTA
QSPADAPMSPELPKPHLPDQLVIVNETEADSKPSKNVARSAAVETASLS
PSLVPARQPTISLLCEDTADTLSVESLTLVPPVDPHSLRSLTGMPPLST
PAAACTEPVGEEAACAEPVGTAED

Transcript: EMP2-001 ENST00000359543

cDNA sequence (SEQ ID NO.: 95), coding part of fusion gene shaded.
GGCGGGATCGGGGAAGGAGGGGCCCCGCCGCCTAGAGGGTGGAGGGAGGGCGCGCAGTCC
CAGCCCAGAGCTTCAAAACAGCCCGGCGGCCTCGCCTCGCACCCCCAGCCAGTCCGTCGA

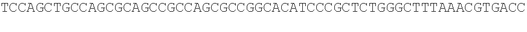

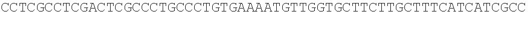

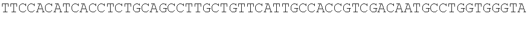

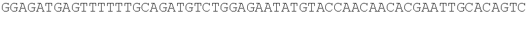

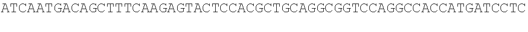

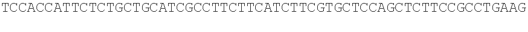

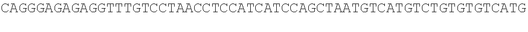

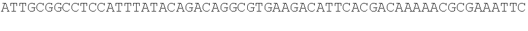

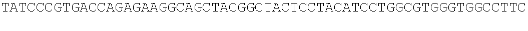

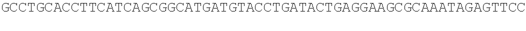

GGAGCTGGGTTGCTTCTGCTGCAGTACAGAATCCACATTCAGATAACCATTTTGTATATA
ATCATTATTTTTTGAGGTTTTTCTAGCAAACGTATTGTTTCCTTTAAAAGCCAAAAAAAA
AAAAAAAAAAAAAAAAAAAAGAAAAAAGAAAAAAAAAATCCAAAAGAGAGAAGAGTTTTT
GCATTCTTGAGATCAGAGAATAGACTATGAAGGCTGGTATTCAGAACTGCTGCCCACTCA
AAAGTCTCAACAAGACACAAGCAAAAATCCAGCAATGCTCAAATCCAAAAGCACTCGGCA
GGACATTTCTTAACCATGGGGCTGTGATGGGAGGAGAGGAGAGGCTGGGAAAGCCGGGTC
TCTGGGGACGTGCTTCCTATGGGTTTCAGCTGGCCCAAGCCCCTCCCGAATCTCTCTGCT
AGTGGTGGGTGGAAGAGGGTGAGGTGGGGTATAGGAGAAGAATGACAGCTTCCTGAGAGG
TTTCACCCAAGTTCCAAGTGAGAAGCAGGTGTAGTCCCTGGCATTCTGTCTGTATCCAAA
CCAGAGCCCAGCCATCCCTCCGGTATCGGGGTGGGTCAGAAAAAGTCTCACCTCAATTTG
CCGACAGTGTCACCTGCTTGCCTTAGGAATGGTCATCCTTAACCTGCGTGCCAGATTTAG
ACTCGTCTTTAGGCAAAACCTACAGCGCCCCCCCCCTCACCCCAGACCTACAGAATCAGA
GTCTTCAAGGGATGGGGCCAGGGAATCTGCATTTCTAACGCGCTCCCTGGGCAACGCTTC
AGATGCGTTGAAGTTGGGGACCACGGTGCCTGGGCCAGGTCAGCAGAGCTGCCTCGTAAA
TGCTGGGGTATCGTCATGTGGAGATGGGGAGGTGAATGCAACCCCCACAGCAGGCCAAAA
CCTTGGCCTCCATCGCCACAGCTGTCTACATCTAGGGCCCCAAAACTCCATTCCTGAGCC
ATGTGAACTCATAGACACCTTCAGGGTGTGGGGTACAGCCTCCTTCCCATCTTATCCCAG
AAGGCCTCTCCCTTCTTGTCCAGCCCTTCATGCTACACCTGGCTGGCCTCTCACCCCTAT
TTCTAGAGCCTCAGAGGACCCATCCACCATTCATTCATTCATTCATTCATTCATTCATTC
ATTCATTCATCAACATAAATCATAACTTGCATGCATGTGCCAGGCACAGGGGATACCCTC
TAGAGACAATCTCCTCCTAGGGCTCATGGCCTAGTGGAGGAGACAGATTAAAACTTAATT
AGAAAAACTGGCTGGGTACAGTGGCTCATGCTTGTAATCCCAGCACTTTGGGAGGCTGAG
GCGGGTGGATCACCTGAGGTCAGGAGTTCAAGACCAGCCTGGCCAAAATGGTAAAACCTG
TCTCTACTAAAAATACAAAAATGAGCTGGGCGTGGTGGTGCATGCCTGTAATCCCAGCTA
TCAGGTGGCTGAGGCAGGAGAATCACTTGAAATGGGAGGTGGAGGTTGCAGTGAGCCGAG
ACCGTGCCACTGCACTCCAGCCTGGGTGACAGAGTGAGACTCCATCTCAAAAAAAGAAAA
AAAAGAAAAGAAACTAATTACACACTGTGATGGAGGCTGCAAAGAACACCACTAAGAATT
CAAAATCAGCTGGGTGCGGTGGCTCACACCTGTAATCCCAGCACTTTGGGAGGCTGAGGC
AGGTGGATCACAAGGTCAGGAGTTCAAGACCAGCCTGGCCAACATGGTGAAACCCCGTCT
CTACCGAAAATACAACAAAATTAGCCCGGTGTGGTGGCAGGTGCCTGTAATCCCAGCTAC
TTAGGAGGCTGAGGCAGGAGAATCGCTTGAAACTGGGAGGCGGAGGTCGCAGTGAGCCGA
GATTCACCACTGCACTCCAGCCCAGGCGACAGTCTGAGACTCCGTCTCAAAAATAAAACG
ATTCAAAATCGAGGCCTGTGGCATGGTAGGGAGGCTGCTTTACGCGTGCCTATTATTAAA
TGCTCCTGGAGGCATTTAGGTATTTAGATCAGTCTAAATATAGCTCCATTCAGTTCGTGC
AGATGACAGTTATTGGGCAGTACCTGTCTGTGTAACACCCAGAAAACATGTCTGTGGAGG
GGCCCATGGTCCCGACAGTAAATGCGGTGAGAGGGTCCCATAGAGCTGGAGTTTTCAAGC
TTTAGGGGTTCCCGTGCTGCTTGGGACAGGCTGATTCAGAGGGTCTGGGTGAATGATTTC
CAGGTGATTTTAAGACTGTGCTGAGAAATAGGGCTTTTGGGGCCTTGTCCTTCAGGATCA
AAGCATGATGCTGTGTGGCAATGCAGACCACCCAGGAACCATCCCAGGAGATAAGCTCTT
TGCACCTCATTGTGTTTTTCTGCTTATGTTGGAGCAGGATGCTGGGGGCTGTCCTGGGAT
GGGGTGTGGGACCTCGTGCTATTTAAATACTTTTGCACTTGACCTTCTGCTGAGTGGAGT
GGTGGTTTGCCATCAGCTCAGTTCCAGTGGAGCTGAAGAGACATCTGGTTTGAGTAGTTT
TAGGGCCACCATGGATATCTCTTCAATGCAGGATTGGCTCTTTCCATCTGCTCTTTCATT
CATTTGTTTTTGACAGATAGTATTAAATGTTTACAATGTTCCAGGCACTGTGTGAGGCTC
TGAAAATACAGGGGTGAGCAAATCCAGATATCCTCCCTGCCATCATGAAGTTTGGAGTCT
ATGAGATAGGACCCCCTCCCTATGGAGAAGCCACCAATGCAGTACAGGGTGACCTGGGGC
CAGAGACAGGACAAATGTCACCTCCTGCCTCCATGAGATACTCTCACTAGTCATATTGTG
GGCAAGAATGTGGCTTACACCCCTAGGGTTAACAGGATGCTACCCAAGCTCATGGAGGAA
GTTGAATCTTAAGTTCCCTTGAAACTTTCTACCTTGGTGGCTTTTCTATAATTTTCTTTT
TTCTTTTTCTTTTTTTTTTTTTTTTTTGAGACTGAGTTTTGCTCTTGTTGCCCAGGCTGG
AGTGCAGTGGCACCATCTTGGCTCACCGCAACCTCTGCCTCCTGGGTTCAAGTGATTCTC
CTGCCTCAGCCTCCCGAGTAGCTGGGATTACAGGCATGTCCCACCATGCCCAGCTAATTT
TTGTATTTTTAGTAGAGATGGGGTTTCTCCATGTTGGTCAGGCTGGTTTCGAACTCCCAA
CCTCAGGTGATCCGCCCACCTCAGCCTTCCAAAGTGCTGGGATTACAGGCATGAGCCACT
GCGTCTGGCCTTCTATAATTTTCTGGTAGTCACGATGGAAACAAACAAAACACCTTAGAA
CCAGAGATCGACCCCCTCAAGCAATACATCAATTCCCTTCACAAGAAACGTCGGGGCTAC
ATGAGTATCTGTGTTGAATGCGGTCTGAAATGATCCTATGGATTTTCCCGGCTGGTTGCC
ACTGCTGTACAACATTCAGTGCCCACATCCACCTGTGCCATTAAGCTTTTTTGAGACATG
AGAGATGCCTCTTCCCTGCTGTATGACATGCATTTGGGAAGTTGGAAAGAAATGACAAAA
TCAGGGAGAAAACATCCAAGCTTCTTACCTGTAGATAGAATCAGCCCTCACTTGGTGCTT
ATTACCAGTTATTCAAGAACAATAACAACAACAAAATTAGTAGACATCCAAGAAGCACAT
ATTAGGACCAAAGATAGCATCAACTGTATTTGAAGGAACTGTAGTTTGCGCATTTTATGA
CATTTTTATAAAGTACTGTAATTCTTTCATTGAGGGGCTATGTGATGGAGACAGAGTAAC
TCATTTTGTTATTTGCATTAAAATTATTTTGGGTCTCTGTTCAAATGAGTTTGGAGAATG
CTTGACTTGTTGGTCTGTGTGAATGTGTATATATATATACCTGAATACAGGAACATCGGA
GACCTATTCACTCCCACACACTCTGCTATAGTTTGCGTGCTTTTGTGGACACCCCTCATG
AACAGGCTGGCGCTCTAGGACGCTCTGTGTTCACTGATGATGAAGAAACCTAGAACTCCA
AGCCTGTTTGTAAACACACTAAACACAGTGGCCTAGATAGAAACTGTATCGTAGTTTAAA
ATCTGCCTCGCGGGATGTTACTAAACTCGCTAATAGTTTAAAGGTTACTTACAATAGAGC
AAGTTGGACAATTTTGTGGTGTTGGGGAAATGTTAGGGCAAGGCCTAGAGGTTCATTTTG
AATCTTGGTTTGTGACTTTAGGGTAGTTAGAAACTTTCTACTTAATGTACCTTTAAAATA
GTCCATTTTCTATGTTTTGTATAATCTGAAACTGTACATGGAAAATAAAGTTTAAAACCA
GATTGCCCAGAGCAAGACTCTAATGTTCCCAACGGTGATGACATCTAGGGCAGAATGCTG
CCATTTTGAGGGGCAGGGGGTCAGCTGATTTCTCATCAAGATAATAATGTATGGTTTTTA
CACTAAGCAACTGATAAATGGACAATTTATCACTGGA

Transcript: EMP2-001 ENST00000359543

cDNA sequence

GGCGGGATCGGGGAAGGAGGGGCCCCGCCGCCTAGAGGGTGGAGGGAGGGCGCGCAGTCC
............................................................
CAGCCCAGAGCTTCAAAACAGCCCGGCGGCCTCGCCTCGCACCCCCAGCCAGTCCGTCGA
............................................................

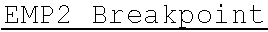

TCCAGCTGCCAGCGCAGCCGCCAGCGCCGGCACATCCCGCTCTGGGCTTTAAACGTGACC
............................................................
CCTCGCCTCGACTCGCCCTGCCCTGTGAAAATGTTGGTGCTTCTTGCTTTCATCATCGCC
..............................-M--L--V--L--L--A--F--I--I--A-
TTCCACATCACCTCTGCAGCCTTGCTGTTCATTGCCACCGTCGACAATGCCTGGTGGGTA
-F--H--I--T--S--A--A--L--L--F--I--A--T--V--D--N--A--W--W--V-
GGAGATGAGTTTTTTGCAGATGTCTGGAGAATATGTACCAACAACACGAATTGCAGAGTC
-G--D--E--F--F--A--D--V--W--R--I--C--T--N--N--T--N--C--T--V-
ATCAATGACAGCTTTCAAGAGTACTCCACGCTGCAGGCGGTCCAGGCCACCATGATCCTC
-I--N--D--S--F--Q--E--Y--S--T--L--Q--A--V--Q--A--T--M--I--L-
TCCACCATTCTCTGCTGCATCGCCTTCTTCATCTTCGTGCTCCAGCTCTTCCGCCTGAAG
-S--T--I--L--C--C--I--A--F--F--I--F--V--L--Q--L--F--R--L--K-
CAGGGAGAGAGGTTTGTCCTAACCTCCATCATCCAGCTAATGTCATGTCTGTGTGTCATG
-Q--G--E--R--F--V--L--T--S--I--I--Q--L--M--S--C--L--C--V--M-
ATTGCGGCCTCCATTTATACAGACAGGCGTGAAGACATTCACGACAAAAACGCGAAATTC
-I--A--A--S--I--Y--T--D--R--R--E--D--I--H--D--K--N--A--K--F-
TATCCCGTGACCAGAGAAGGCAGCTACGGCTACTCCTACATCCTGGCGTGGGTGGCCTIC
-Y--P--V--T--R--E--G--S--Y--G--Y--S--Y--I--L--A--W--V--A--F-
GCCTGCACCTTCATCAGCGGCATGATGTACCTGATACTGAGGAAGCGCAAATAGAGTTCC
-A--C--T--F--I--S--G--M--M--Y--L--I--L--R--K--R--K--*-......
GGAGCTGGGTTGCTTCTGCTGCAGTACAGAATCCACATTCAGATAACCATTTTGTATATA
............................................................
ATCATTATTTTTTGAGGTTTTTCTAGCAAACGTATTGTTTCCTTTAAAAGCCAAAAAAAA
............................................................
AAAAAAAAAAAAAAAAAAAAGAAAAAAGAAAAAAAAAATCCAAAAGAGAGAAGAGTTTTT
............................................................
GCATTCTTGAGATCAGAGAATAGACTATGAAGGCTGGTATTCAGAACTGCTGCCCACTCA
............................................................
AAAGTCTCAACAAGACACAAGCAAAAATCCAGCAATGCTCAAATCCAAAAGCACTCGGCA
............................................................
GGACATTTCTTAACCATGGGGCTGTGATGGGAGGAGAGGAGAGGCTGGGAAAGCCGGGTC
............................................................
TCTGGGGACGTGCTTCCTATGGGTTTCAGCTGGCCCAAGCCCCTCCCGAATCTCTCTGCT
............................................................
AGTGGTGGGTGGAAGAGGGTGAGGTGGGGTATAGGAGAAGAATGACAGCTTCCTGAGAGG
............................................................
TTTCACCCAAGTTCCAAGTGAGAAGCAGGTGTAGTCCCTGGCATTCTGTCTGTATCCAAA
............................................................
CCAGAGCCCAGCCATCCCTCCGGTATCGGGGTGGGTCAGAAAAAGTCTCACCTCAATTTG
............................................................
CCGACAGTGTCACCTGCTTGCCTTAGGAATGGTCATCCTTAACCTGCGTGCCAGATTTAG
............................................................
ACTCGTCTTTAGGCAAAACCTACAGCGCCCCCCCCCTCACCCCAGACCTACAGAATCAGA
............................................................
GTCTTCAAGGGATGGGGCCAGGGAATCTGCATTTCTAACGCGCTCCCTGGGCAACGCTTC
............................................................
AGATGCGTTGAAGTTGGGGACCACGGTGCCTGGGCCAGGTCAGCAGAGCTGCCTCGTAAA
............................................................
TGCTGGGGTATCGTCATGTGGAGATGGGGAGGTGAATGCAACCCCCACAGCAGGCCAAAA
............................................................
CCTTGGCCTCCATCGCCACAGCTGTCTACATCTAGGGCCCCAAAACTCCATTCCTGAGCC
............................................................
ATGTGAACTCATAGACACCTTCAGGGTGTGGGGTACAGCCTCCTTCCCATCTTATCCCAG
............................................................
AAGGCCTCTCCCTTCTTGTCCAGCCCTTCATGCTACACCTGGCTGGCCTCTCACCCCTAT
............................................................
TTCTAGAGCCTCAGAGGACCCATCCACCATTCATTCATTCATTCATTCATTCATTCATTC
............................................................
ATTCATTCATCAACATAAATCATAACTTGCATGCATGTGCCAGGCACAGGGGATACCCTC
............................................................
TAGAGACAATCTCCTCCTAGGGCTCATGGCCTAGTGGAGGAGACAGATTAAAACTTAATT
............................................................
AGAAAAACTGGCTGGGTACAGTGGCTCATGCTTGTAATCCCAGCACTTTGGGAGGCTGAG
............................................................
GCGGGTGGATCACCTGAGGTCAGGAGTTCAAGACCAGCCTGGCCAAAATGGTAAAACCTG
............................................................
TCTCTACTAAAAATACAAAAATGAGCTGGGCGTGGTGGTGCATGCCTGTAATCCCAGCTA
............................................................
TCAGGTGGCTGAGGCAGGAGAATCACTTGAAATGGGAGGTGGAGGTTGCAGTGAGCCGAG
............................................................
ACCGTGCCACTGCACTCCAGCCTGGGTGACAGAGTGAGACTCCATCTCAAAAAAAGAAAA
............................................................
AAAAGAAAAGAAACTAATTACACACTGTGATGGAGGCTGCAAAGAACACCACTAAGAATT
............................................................
CAAAATCAGCTGGGTGCGGTGGCTCACACCTGTAATCCCAGCACTTTGGGAGGCTGAGGC
............................................................
AGGTGGATCACAAGGTCAGGAGTTCAAGACCAGCCTGGCCAACATGGTGAAACCCCGTCT
............................................................
CTACCGAAAATACAACAAAATTAGCCCGGTGTGGTGGCAGGTGCCTGTAATCCCAGCTAC
............................................................
TTAGGAGGCTGAGGCAGGAGAATCGCTTGAAACTGGGAGGCGGAGGTCGCAGTGAGCCGA
............................................................
GATTCACCACTGCACTCCAGCCCAGGCGACAGTCTGAGACTCCGTCTCAAAAATAAAACG
............................................................
ATTCAAAATCGAGGCCTGTGGCATGGTAGGGAGGCTGCTTTACGCGTGCCTATTATTAAA
............................................................
TGCTCCTGGAGGCATTTAGGTATTTAGATCAGTCTAAATATAGCTCCATTCAGTTCGTGC
............................................................
AGATGACAGTTATTGGGCAGTACCTGTCTGTGTAACACCCAGAAAACATGTCTGTGGAGG
............................................................
GGCCCATGGTCCCGACAGTAAATGCGGTGAGAGGGTCCCATAGAGCTGGAGTTTTCAAGC
............................................................
TTTAGGGGTTCCCGTGCTGCTTGGGACAGGCTGATTCAGAGGGTCTGGGTGAATGATTTC
............................................................
CAGGTGATTTTAAGACTGTGCTGAGAAATAGGGCTTTTGGGGCCTTGTCCTTCAGGATCA
............................................................
AAGCATGATGCTGTGTGGCAATGCAGACCACCCAGGAACCATCCCAGGAGATAAGCTCTT
............................................................
TGCACCTCATTGTCTTTTTCTGCTTATGTTGGAGCAGGATGCTGGGGGCTGTCCTGGGAT
............................................................
GGGGTGTGGGACCTCGTGCTATTTAAATACTTTTGCACTTGACCTTCTGCTGAGTGGAGT
............................................................
GGTGGTTTGCCATCAGCTCAGTTCCAGTGGAGCTGAAGAGACATCTGGTTTGAGTAGTTT
............................................................
TAGGGCCACCATGGATATCTCTTCAATGCAGGATTGGCTCTTTCCATCTGCTCTTTCATT
............................................................
CATTTGTTTTTGACAGATAGTATTAAATGTTTACCATGTTCCAGGCACTGTGTGAGGCTC
............................................................
TGAAAATACAGGGGTGAGCAAATCCAGATATCCTCCCTGCCATCATGAAGTTTGGAGTCT
............................................................
ATGAGATAGGACCCCCTCCCTATGGAGAAGCCACCAATGCAGTACAGGGTGACCTGGGGC
............................................................
CAGAGACAGGACAAATGTCACCTCCTGCCTCCATGAGATACTCTCACTAGTCATATTGTG
............................................................
GGCAAGAATGTGGCTTACACCCCTAGGGTTAACAGGATGCTACCCAAGCTCATGGAGGAA
............................................................
GTTGAATCTTAAGTTCCCTTGAAACTTTCTACCTTGGTGGCTTTTCTATAATTTTCTTTT
............................................................
TTCTTTTTCTTTTTTTTTTTTTTTTTTGAGACTGAGTTTGCTCTTGTTGCCCAGGCTGG
............................................................
AGTGCAGTGGCACCATCTTGGCTCACCGCAACCTCTGCCTCCTGGGTTCAAGTGATTCTC
............................................................
CTGCCTCAGCCTCCCGAGTAGCTGGGATTACAGGCATGTCCCACCATGCCCAGCTAATTT
............................................................
TTGTATTTTTAGTAGAGATGGGGTTTCTCCATGTTGGTCAGGCTGGTTTCGAACTCCCAA
............................................................
CCTCAGGTGATCCGCCCACCTCAGCCTTCCAAAGTGCTGGGATTACAGGCATGAGCCACT
............................................................
GCGTCTGGCCTTCTATAATTTTCTGGTAGTCACGATGGAAACAAACAAAACACCTTAGAA
............................................................
CCAGAGATCGACCCCCTCAAGCAATACATCAATTCCCTTCACAAGAAACGTCGGGGCTAC
............................................................
ATGAGTATCTGTGTTGAATGCGGTCTGAAATGATCCTATGGATTTTCCCGGCTGGTTGCC
............................................................
ACTGCTGTACAACATTCAGTGCCCACATCCACCTGTGCCATTAAGCTTTTTTGAGACATG
............................................................
AGAGATGCCTCTTCCCTGCTGTATGACATGCATTTGGGAAGTTGGAAAGAAATGACAAAA
............................................................
TCAGGGAGAAAACATCCAAGCTTCTTACCTGTAGATAGAATCAGCCCTCACTTGGTGCTT
............................................................
ATTACCAGTTATTCAAGAACAATAACAACAACAAAATTAGTAGACATCCAAGAAGCACAT
............................................................
ATTAGGACCAAAGATAGCATCAACTGTATTTGAAGGAACTGTAGTTTGCGCATTTTATGA
............................................................
CATTTTTATAAAGTACTGTAATTCTTTCATTGAGGGGCTATGTGATGGAGACAGACTAAC
............................................................
TCATTTTGTTATTTGCATTAAAATTATTTTGGGTCTCTGTTCAAATGAGTTTGGAGAATG
............................................................
CTTGACTTGTTGGTCTGTGTGAATGTGTATATATATATACCTGAATACAGGAACATCGGA
............................................................
GACCTATTCACTCCCACACACTCTGCTATAGTTTGCGTGCTTTTGTGGACACCCCTCATG
............................................................
AACAGGCTGGCGCTCTAGGACGCTCTGTGTTCACTGATGATGAAGAAACCTAGAACTCCA
............................................................
AGCCTGTTTGTAAACACACTAAACACAGTGGCCTAGATAGAAACTGTATCGTAGTTTAAA
............................................................
ATCTGCCTCGCGGGATGTTACTAAACTCGCTAATAGTTTAAAGGTTACTTACAATAGAGC
............................................................
AAGTTGGACAATTTTGTGGTGTTGGGGAAATGTTAGGGCAAGGCCTAGAGGTTCATTTTG
............................................................
AATCTTGGTTTGTGACTTTAGGGTAGTTAGAAACTTTCTACTTAATGTACCTTTAAAATA
............................................................
GTCCATTTTCTATGTTTTGTATAATCTGAAACTGTACATGGAAAATAAAGTTTAAAACCA
............................................................
GATTGCCCAGAGCAAGACTCTAATGTTCCCAACGGTGATGACATCTAGGGCAGAATGCTG
............................................................
CCATTTTGAGGGGCAGGGGGTCAGCTGATTTCTCATCAAGATAATAATGTATGGTTTTTA
............................................................
CACTAAGCAACTGATAAATGGACAATTTATCACTGGA
.....................................

Transcript: EMP2-001 ENST00000359543

Protein sequence
(SEQ ID NO.: 96)
MLVLLAFIIAFHITSAALLFIATVDNAWWVGDEFFADVWRICTNNTNCT
VINDSFQEYSTLQAVQATMILSTILCCIAFFIFVLQLFRLKQGERFVLT
SIIQLMSCLCVMIAASIYTDRREDIHDKNAKFYPVTREGSYGYSYILAW
VAFACTFISGMMYLILRKRK

CLEC16A—EMP2 Fusion sequence exon 9 to exon 2 UTR

cDNA sequence (SEQ ID NO.: 97), EMP2 underlined.
ATGTTTGGCCGCTCGCGGAGCTGGGTGGGCGGGGGCCATGGCAAGACTTCCCGCAACATCCACTCCTTGGACCAC
CTCAAGTATCTGTACCACGTTTTGACCAAAAACACCACAGTCACAGAACAGAACCGGAACCTGCTAGTGGAGACC
ATCCGTTCCATCACTGAGATCCTGATCTGGGGAGATCAAAATGACAGCTCTGTATTTGACTTCTTCCTGGAGAAG
AATATGTTTGTTTTCTTCTTGAACATCTTGCGGCAAAAGTCGGGCCGTTACGTGTGCGTTCAGCTGCTGCAGACC
TTGAACATCCTCTTTGAGAACATCAGTCACGAGACCTCACTTTATTATTTGCTCTCAAATAACTACGTAAATTCT
ATCATCGTTCATAAATTTGACTTTTCTGATGAGGAGATTATGGCCTATTATATATCGTTCCTGAAAACACTTTCG
TTAAAACTCAACAACCACACTGTCCATTTCTTTTATAATGAGCACACCAATGACTTTGCCCTGTACACAGAAGCC
ATCAAGTTTTTCAACCACCCTGAAAGCATGGTTAGAATTGCTGTAAGAACCATAACTTTGAATGTCTATAAAGTG
TCATTGGATAACCAGGCCATGCTGCACTACATCCGAGATAAAACTGCTGTTCCTTACTTCTCCAATTTGGTCTGG
TTCATTGGGAGCCATGTGATCGAACTCGATGACTGCGTGCAGACTGATGAGGAGCATCGGAATCGGGGTAAACTG
AGTGATCTGGTGGCAGAGCACCTAGACCACCTGCACTATCTCAATGACATCCTGATCATCAACTGTGAGTTCCTC
AACGATGTGCTCACTGACCACCTGCTCAACAGGCTCTTCCTGCCCCTCTACGTGTACTCACTGGAGAACCAGGAC

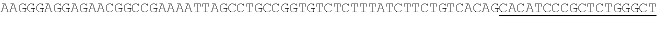

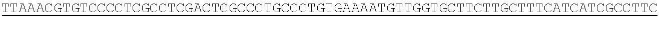

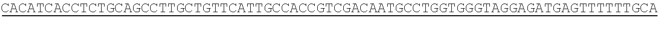

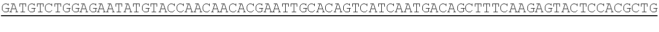

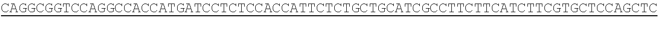

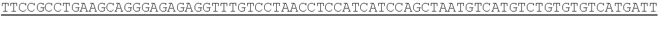

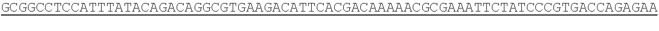

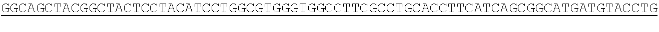

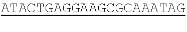

Protein sequence (SEQ ID NO.: 98), EMP2 underlined.
MFGRSRSWVGGGHGKTSRNIHSLDHLKYLYHVLTKNTTVTEQNRNLLVETIRSITEILIWGDQNDSSVFDFFLEK
NMFVFFLNILRQKSGRYVCVQLLQTLNILFENISHETSLYYLLSNNYVNSIIVHKFDFSDEEIMAYYISFLKTLS
LKLNNHTVHFFYNEHTNDFALYTEAIKFFNHPESMVRIAVRTITLNVYKVSLDNQAMLHYIRDKTAVPYFSNLVW
FIGSHVIELDDCVQTDEEHRNRGKLSDLVAEHLDHLHYLNDILIINCEFLNDVLTDHLLNRLFLPLYVYSLENQD

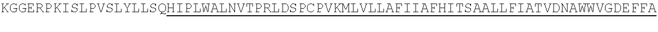

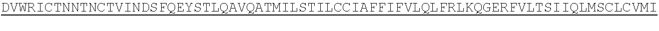

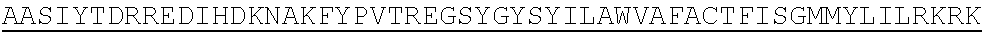

Protein Domain

Domains within the query sequence of 506 residues

| Name | Start | End |
|---|---|---|
| Transmembrane region | 341 | 363 |
| Transmembrane region | 400 | 422 |
| Transmembrane region | 434 | 456 |
| Transmembrane region | 480 | 502 |

CLEC16A—EMP2 Fusion sequence exon 4 to exon 2 UTR

cDNA sequence (SEQ ID NO.: 99), EMP2 underlined.
ATGTTTGGCCGCTCGCGGAGCTGGGTGGGCGGGGGCCATGGCAAGACTTCCCGCAACATCCACTCCTTGGACCAC
CTCAAGTATCTGTACCACGTTTTGACCAAAAACACCACAGTCACAGAACAGAACCGGAACCTGCTAGTGGAGACC
ATCCGTTCCATCACTGAGATCCTGATCTGGGGAGATCAAAATGACAGCTCTGTATTTGACTTCTTCCTGGAGAAG
AATATGTTTGTTTTCTTCTTGAACATCTTGCGGCAAAAGTCGGGCCGTTACGTGTGCGTTCAGCTGCTGCAGACC
TTGAACATCCTCTTTGAGAACATCAGTCACGAGACCTCACTTTATTATTTGCTCTCAAATAACTACGTAAATTCT
ATCATCGTTCATAAATTTGACTTTTCTGATGAGGAGATTATGGCCTATTATATATCGTTCCTGAAAACACTTTCG

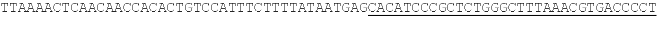

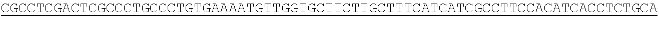

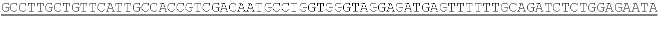

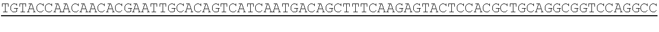

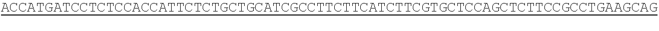

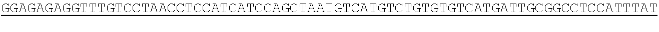

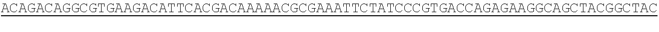

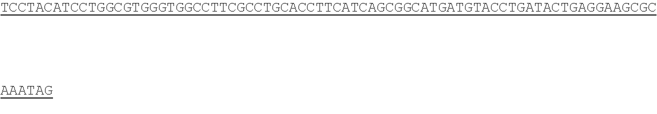

Protein sequence
(SEQ ID NO.: 100)

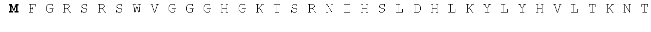

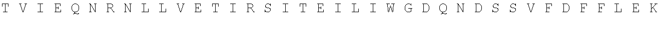

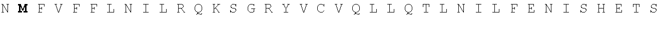

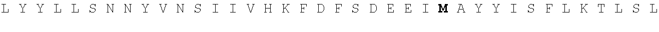

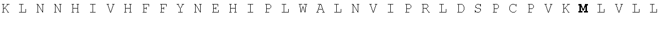

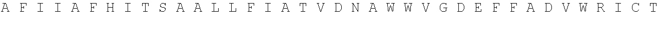

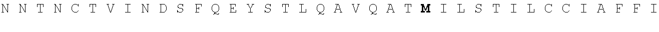

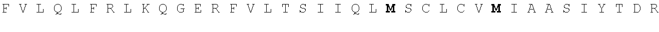

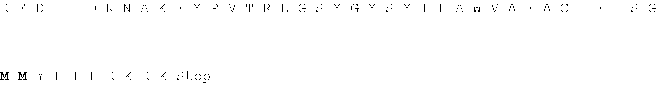

Protein Domain

Domains within the query sequence of 351 residues

| Name | Start | End |
|---|---|---|
| Transmembrane region | 186 | 208 |
| Transmembrane region | 245 | 267 |
| Transmembrane region | 279 | 301 |
| Transmembrane region | 325 | 347 |

CLEC16A—EMP2 Fusion sequence exon 10 to exon 2 UTR

cDNA sequence (SEQ ID NO.: 101), EMP2 underlined.
ATGTTTGGCCGCTCGCGGAGCTGGGTGGGCGGGGGCCATGGCAAGACTTCCCGCAACATCCACTCCTTGG
ACCACCTCAAGTATCTGTACCACGTTTTGACCAAAAACACCACAGTCACAGAACAGAACC
GGAACCTGCTAGTGGAGACCATCCGTTCCATCACTGAGATCCTGATCTGGGGAGATCAAA
ATGACAGCTCTGTATTTGACTTCTTCCTGGAGAAGAATATGTTTGTTTTCTTCTTGAACA
TCTTGCGGCAAAAGTCGGGCCGTTACGTGTGCGTTCAGCTGCTGCAGACCTTGAACATCC
TCTTTGAGAACATCAGTCACGAGACCTCACTTTATTATTTGCTCTCAAATAACTACGTAA
ATTCTATCATCGTTCATAAATTTGACTTTTCTGATGAGGAGATTATGGCCTATTATATAT
CGTTCCTGAAAACACTTTCGTTAAAACTCAACAACCACACTGTCCATTTCTTTTATAATG
AGCACACCAATGACTTTGCCCTGTACACAGAAGCCATCAAGTTTTTCAACCACCCTGAAA
GCATGGTTAGAATTGCTGTAAGAACCATAACTTTGAATGTCTATAAAGTGTCATTGGATA
ACCAGGCCATGCTGCACTACATCCGAGATAAAACTGCTGTTCCTTACTTCTCCAATTTGG
TCTGGTTCATTGGGAGCCATGTGATCGAACTCGATGACTGCGTGCAGACTGATGAGGAGC
ATCGGAATCGGGGTAAACTGAGTGATCTGGTGGCAGAGCACCTAGACCACCTGCACTATC
TCAATGACATCCTGATCATCAACTGTGAGTTCCTCAACGATGTGCTCACTGACCACCTGC
TCAACAGGCTCTTCCTGCCCCTCTACGTGTACTCACTGGAGAACCAGGACAAGGGAGGAG
AACGGCCGAAAATTAGCCTGCCGGTGTCTCTTTATCTTCTGTCACAGGTCTTCTTAATTA
TACATCATGCACCGCTGGTGAACTCGTTAGCTGAAGTCATTCTGAATGGTGATCTGTCTG

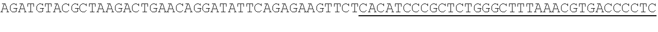

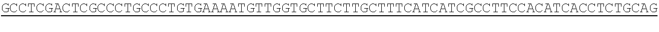

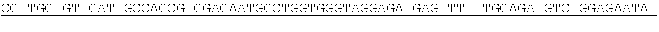

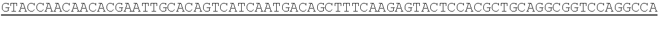

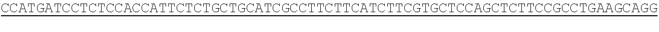

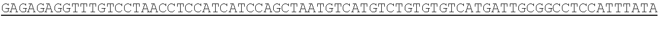

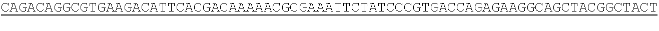

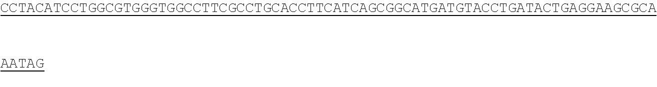

Protein sequence
(SEQ ID NO.: 102)

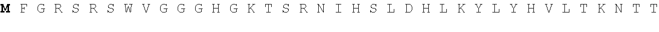

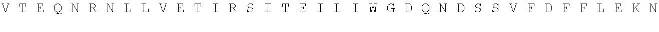

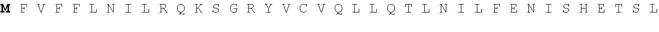

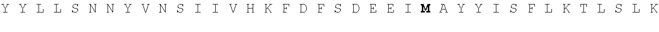

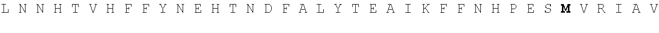

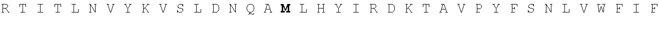

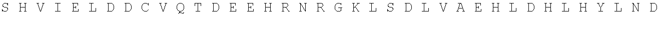

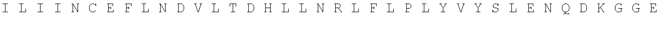

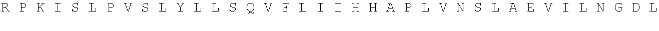

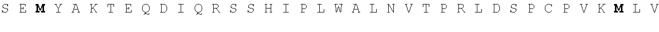

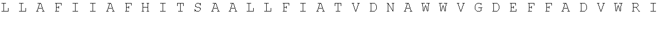

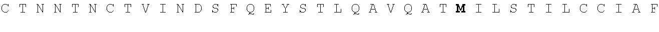

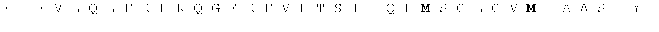

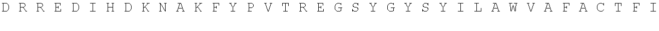

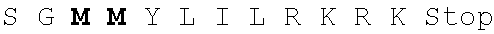

Protein Domain

Domains within the query sequence of 544 residues

| Name | Start | End |
|---|---|---|
| Transmembrane region | 379 | 401 |
| Transmembrane region | 438 | 460 |
| Transmembrane region | 472 | 494 |
| Transmembrane region | 518 | 540 |

Fusion Gene #2: CLDN18-ARHGAP26

CLDN18

Genomic PCR confirmed breakpoint in the discovery sample—chr3:137,752,065

RT-PCR confirmed RNA fusion point in exon 5—chr3: 137,749,947

ARHGAP26

Genomic PCR confirmed breakpoint in the discovery sample—chr5:142318274

RT-PCR confirmed RNA fusion point in exon 12—chr5: 142393645

Transcript: CLDN18-001 ENST00000343735

cDNA sequence (SEQ ID NO.: 103), coding part of
fusion gene shaded.
AACCGCCTCCATTACATGGTCCGTTCCTGACGTGTACACCAGCCTCTCA
GAGAAAACTCCATCCCTACACTCGGTAGTCTCAGAATTGCGCTGTCCAC
TTGTCGTGTGGCTCTGTGTCGACACTGTGCGCCACCATGGCCGTGACTG
CCTGTCAGGGCTTGGGGTTCGTGGTTTCACTGATTGGGATTGCGGGCAT
CATTGCTGCCACCTGCATGGACCAGTGGAGCACCCAAGACTTGTACAAC
AACCCCGTAACAGCTGTTTTCAACTACCAGGGGCTGTGGCGCTCCTGTG
TCCGAGAGAGCTCTGGCTTCACCGAGTGCCGGGGCTACTTCACCCTGCT
GGGGCTGCCAGCCATGCTGCAGGCAGTGCGAGCCCTGATGATCGTAGGC
ATCGTCCTGGGTGCCATTGGCCTCCTGGTATCCATCTTTGCCCTGAAAT
GCATCCGCATTGGCAGCATGGAGGACTCTGCCAAAGCCAACATGACACT
GACCTCCGGGATCATGTTCATTGTCTCAGGTCTTTGTGCAATTGCTGGA
GTGTCTGTGTTTGCCAACATGCTGGTGACTAACTTCTGGATGTCCACAG
CTAACATGTACACCGGCATGGGTGGGATGGTGCAGACTGTTCAGACCAG
GTACACATTTGGTGCGGCTCTGTTCGTGGGCTGGGTCGCTGGAGGCCTC
ACACTAATTGGGGGTGTGATGATGTGCATCGCCTGCCGGGGCCTGGCAC
CAGAAGAAACCAACTACAAAGCCGTTTCTTATCATGCCTCAGGCCACAG
TGTTGCCTACAAGCCTGGAGGCTTCAAGGCCAGCACTGGCTTTGGGTCC
AACACCAAAAACAAGAAGATATACGATGGAGGTGCCCGCACAGAGGACG
AGGTACAATCTTATCCTTCCAAGCACGACTATGTGTAATGCTCTAAGAC
CTCTCAGCACGGGCGGAAGAAACTCCCGGAGAGCTCACCCAAAAAACAA
GGAGATCCCATCTAGATTTCTTCTTGCTTTTGACTCACAGCTGGAAGTT
AGAAAAGCCTCGATTTCATCTTTGGAGAGGCCAAATGGTCTTAGCCTCA
GTCTCTGTCTCTAAATATTCCACCATAAAACAGCTGAGTTATTTATGAA
TTAGAGGCTATAGCTCACATTTTCAATCCTCTATTTCTTTITTTAAATA
TAACTITCTACTCTGATGAGAGAATGTGGTTTTAATCTCTCTCTCACAT
TTTGATGATTTAGACAGACTCCCCCTCTTCCTCCTAGTCAATAAACCCA
TTGATGATCTATTTCCCAGCTTATCCCCAAGAAAACTTTTGAAAGGAAA
GAGTAGACCCAAAGATGTTATTTTCTGCTGTTTGAATTTTGTCTCCCCA
CCCCCAACTTGGCTAGTAATAAACACTTACTGAAGAAGAAGCAATAAGA
GAAAGATATTTGTAATCTCTCCAGCCCATGATCTCGGTTTTCTTACACT
GTGATCTTAAAAGTTACCAAACCAAAGTCATTTTCAGTTTGAGGCAACC
AAACCTTTCTACTGCTGTTGACATCTTCTTATTACAGCAACACCATTCT
AGGAGTTTCCTGAGCTCTCCACTGGAGTCCTCTTTCTGTCGCGGGTCAG
AAATTGTCCCTAGATGAATGAGAAAATTATTTTTTTTAATTTAAGTCCT
AAATATAGTTAAAATAAATAATGTTTTAGTAAAATGATACACTATCTCT
GTGAAATAGCCTCACCCCTACATGTGGATAGAAGGAAATGAAAAAATAA
TTGCTTTGACATTGTCTATATGGTACTTTGTAAAGTCATGCTTAAGTAC
AAATTCCATGAAAAGCTCACTGATCCTAATTCTTTCCCTTTGAGGTCTC
TATGGCTCTGATTGTACATGATAGTAAGTGTAAGCCATGTAAAAAGTAA
ATAATGTCTGGGCACAGTGGCTCACGCCTGTAATCCTAGCACTTTGGGA
GGCTGAGGAGGAAGGATCACTTGAGCCCAGAAGTTCGAGACTAGCCTGG
GCAACATGGAGAAGCCCTGTCTCTACAAAATACAGAGAGAAAAAATCAG
CCAGTCATGGTGGCCTACACCTGTAGTCCCAGCATTCCGGGAGGCTGAG
GTGGGAGGATCACTTGAGCCCAGGGAGGTTGGGGCTGCAGTGAGCCATG
ATCACACCACTGCACTCCAGCCAGGTGACATAGCGAGATCCTGTCTAAA
AAAATAAAAAATAAATAATGGAACACAGCAAGTCCTAGGAAGTAGGTTA
AAACTAATTCTTTAAAAAAAAAAAAAAGTTGAGCCTGAATTAAATGTAA
TGTTTCGAAGTGACAGGTATCCACATTTGCATGGTTACAAGCCACTGCC
AGTTAGCAGTAGCACTTTCCTGGCACTGTGGTCGGTTTTGTTTTGTTTT
GCTTTGTTTAGAGACGGGGTCTCACTTTCCAGGCTGGCCTCAAACTCCT
GCACTCAAGCAATTCTTCTACCCTGGCCTCCCAAGTAGCTGGAATTACA
GGTGTGCGCCATCACAACTAGCTGGTGGTCAGTTTTGTTACTCTGAGAG
CTGTTCACTTCTCTGAATTCACCTAGAGTGGTTGGACCATCAGATGTTT
GGGCAAAACTGAAAGCTCTTTGCAACCACACACCTTCCCTGAGCTTACA
TCACTGCCCTTTTGAGCAGAAAGTCTAAATTCCTTCCAAGACAGTAGAA
TTCCATCCCAGTACCAAAGCCAGATAGGCCCCCTAGGAAACTGAGGTAA
GAGCAGTCTCTAAAAACTACCCACAGCAGCATTGGTGCAGGGGAACTTG
GCCATTAGGTTATTATTTGAGAGGAAAGTCCTCACATCAATAGTACATA
TGAAAGTGACCTCCAAGGGGATTGGTGAATACTCATAAGGATCTTCAGG
CTGAACAGACTATGTCTGGGGAAAGAACGGATTATGCCCCATTAAATAA
CAAGTTGTGTTCAAGAGTCAGAGCAGTGAGCTCAGAGGCCCTTCTCACT
GAGACAGCAACATTTAAACCAAACCAGAGGAAGTATTTGTGGAACTCAC
TGCCTCAGTTTGGGTAAAGGATGAGCAGACAAGTCAACTAAAGAAAAAA
GAAAAGCAAGGAGGAGGGTTGAGCAATCTAGAGCATGGAGTTTGTTAAG
TGCTCTCTGGATTTGAGTTGAAGAGCATCCATTTGAGTTGAAGGCCACA
GGGCACAATGAGCTCTCCCTTCTACCACCAGAAAGTCCCTGGTCAGGTC
TCAGGTAGTGCGGTGTGGCTCAGCTGGGTTTTTAATTAGCGCATTCTCT
ATCCAACATTTAATTGTTTGAAAGCCTCCATATAGTTAGATTGTGCTTT
GTAATTTTGTTGTTGTTGCTCTATCTTATTGTATATGCATTGAGTATTA
ACCTGAATGTTTTGTTACTTAAATATTAAAAACACTGTTATCCTAGAGT
T

Transcript: CLDN18-001 ENST00000343735

Protein sequence (SEQ ID NO.: 104), coding part
of fusion gene shaded.
MAVTACQGLGFVVSLIGIAGIIAATCMDQWSTQDLYNNPVTAVFNYQGL
WRSCVRESSGFTECRGYFTLLGLPAMLQAVRALMIVGIVLGAIGLLVSI
FALKCIRIGSMEDSAKANMTLTSGIMFIVSGLCAIAGVSVFANMLVTNF
WMSTANMYTGMGGMVQTVQTRYTFGAALFVGWVAGGLTLIGGVMMCIAC
RGLAPEETNYKAVSYHASGHSVAYKPGGFKASTGFGSNTKNKKIYDGGA
RTEDEVQSYPSKHDYV

Transcript: ARHGAP26-001 ENST00000274498

cDNA sequence (SEQ ID NO.: 105), coding part of fusion gene shaded.
GGCGGGGCGGCCGAGGCTGCTGTGAGAGGGCGCTCGAGGCTGCCGAGAGCTAGCTAGCGA
AGGAGGCGGGGAGGCGGCGTCTGCACTCGCTCGCCCGCTCGCTCGCTTCCCGGCGCCGCT
GCGGGTCCGCGCTGCGTTTCCTGCTCGCGATCCGCTCCGTTGCCCGCGCCCGGAACAGCA
GCACCTCGGCCGGGTCCGAGCTCGGTTCGGGAGTCTTGCGCGCCGGCGGACACCGCGCGC
GGAGTGAGCCAGCGCCACACCTGTGGAGCCGGCGGCCGTCGGGGGAGCCGGCCGGGGTCC
CGCCGCGTGAGTGCTCTGGGCGGCGGGCGGCCCGGGCCCCGGCGGAGGCGCGCCCCCCGG
CTGGGCGCCGCGCGCACCATGGGGCTCCCAGCGCTCGAGTTCAGCGACTGCTGCCTCGAT
AGTCCGCACTTCCGAGAGACGCTCAAGTCGCACGAAGCAGAGCTGGACAAGACCAACAAA
TTCATCAAGGAGCTCATCAAGGACGGGAAGTCACTCATAAGCGCGCTCAAGAATTTGTCT
TCAGCGAAGCGGAAGTTTGCAGATTCCTTAAATGAATTTAAATTTCAGTGCATAGGAGAT
GCAGAAACAGATGATGAGATGTGTATAGCAAGATCTTTGCAGGAGTTTGCCACTGTCCTC
AGGAATCTTGAAGATGAACGGATACGGATGATTGAGAATGCCAGCGAGGTGCTCATCACT
CCCTTGGAGAAGTTTCGAAAGGAACAGATCGGGGCTGCCAAGGAAGCCAAAAAGAAGTAT
GACAAAGAGACAGAAAAGTATTGTGGCATCTTAGAAAAACACTTGAATTTGTCTTCCAAA
AAGAAAGAATCTCAGCTTCAGGAGGCAGACAGCCAAGTGGACCTGGTCCGGCAGCATTTC
TATGAAGTATCCCTGGAATATGTCTTCAAGGTGCAGGAAGTCCAAGAGAGAAAGATGTTT
GAGTTTGTGGAGCCTCTGCTGGCCTTCCTGCAAGGACTCTTCACTTTCTATCACCATGGT
TACGAACTGGCCAAGGATTTCGGGGACTTCAAGACACAGTTAACCATTAGCATACAGAAC
ACAAGAAATCGCTTTGAAGGCACTAGATCAGAAGTGGAATCACTGATGAAAAAGATGAAG
GAGAATCCCCTTGAGCACAAGACCATCAGTCCCTACACCATGGAGGGATACCTCTACGTG
CAGGAGAAACGTCACTTTGGAACTTCTTGGGTGAAGCACTACTGTACATATCAACGGGAT
TCCAAACAAATCACCATGGTACCATTTGACCAAAAGTCAGGAGGAAAAGGGGGAGAAGAT
GAATCAGTTATCCTCAAATCCTGCACACGGCGGAAAACAGACTCCATTGAGAAGAGGTTT
TGCTTTGATGTGGAAGCAGTAGACAGGCCAGGGGTTATCACCATGCAAGCTTTGTCGGAA

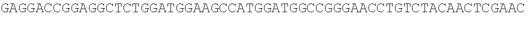

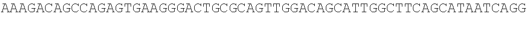

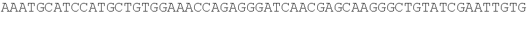

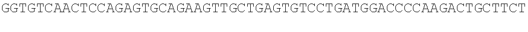

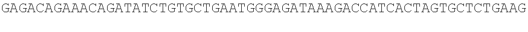

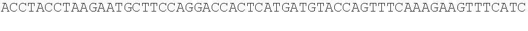

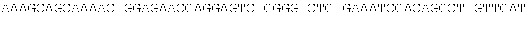

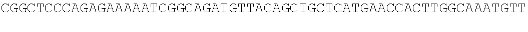

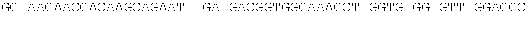

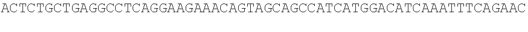

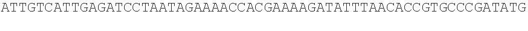

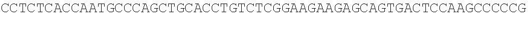

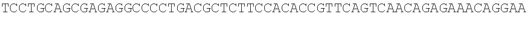

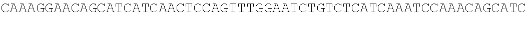

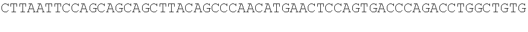

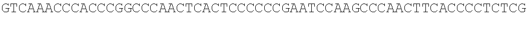

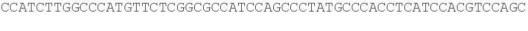

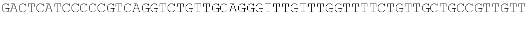

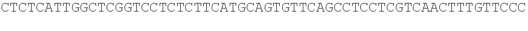

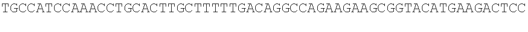

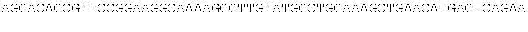

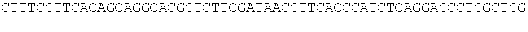

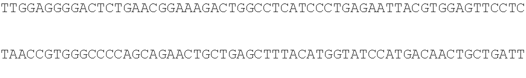

CCAGTGTCGAGGCCATTTCTCTTTGCCACTGAGAAATGCAGCGTGACTGACTCTGTTGCT
ACCTGTCAACATGAATGTTTCTGTGAGCTCTGGTGTCACTCATCTCCATGATCATCTCAG
CCAACATGCATCAGTACTGCAAGAAAAGAAGTCAATCAGCAGAGGAGAGCATTTGATAAC
TAAGAGGAAGACTTGCAAAGCCGTTTTCTCATGAGTACCCTGAATAGGGGGCACTCATTT
TGTTTCAACGGTCCAAACGCCCAACCTTCAGAAAGAGGAAGTCAGATAGAAATAGTCCCT
GAGAGCACACTGTGTAGCTAAGCCTGCTGGGGCTGGGTGAAGAAATTGGCGCTGAGATCC
AGGCTGGATCCATTGCTTTTGTTTACAATAGGCACTCTCTCTACCCCACCTCTCAGTACT
TGAGACTTAAAGTGCTACAGGCAGCTGGATCTGTTTGCATGCAGGATGAAGAGGGTTAAA
ACACTGTTTATATAAGATCCAATCTCTCACCATCTCTAAAGCAGCCGTTGGCCTGTCATC
AGTGAGATACAATCCAGTCTTCTCATGCACGGGAACACACACACCCTGCGTTTCTCCCTC
CCAGGCTAGGAACCTCTCTGCCACCAAGGGCTGCCATCCATCGCCTAGTAACCACGGCAA
CCCAACCTACTCTAAAACCAAACCAAAAAAATAAAATAACACATCCTCTTTGCATGACAC
ATTTTTTTTCTCCCCTTTTTGGTACACTTTTTTTGAATGGTTTTCTAACAACTTGAAGCA
CAGGATCAAGGAATTAGGGTGGTCTACTTGAGGCAGATGGGATAGTAGCTGGGAACTGTT
CCCTTTCTGATTAATTTCAGCAGCATCGGAATATATTTGGAGCACACCCTAGTAACCTCT
TGAGATTAAATTACATAGTCTTAATATTTCTGTTCCTCCATGCAACTGATGTTTGTTTTT
TAAAGGGTAAGATGCTGCCTCCCAATGGGTGATGCCATCTGACTGGTTTCCCCATGTCCT
CCCATTCACCCATCTCTGCTCCCACCCTTGCCTGCCTCTAACCCACCACTGGCCAGCCCC
CTTGCCCTACTCTGGGCTGCTGAACACTGGTGCTGTGGTGGTTTTCAAGGTTAATTCCTA
GGCTAACCGTATGGCCTATAGTTTAAAAGCACATCTATGTTCACTGCCACTCTGAAAAAG
GGAATTATTTCTCAGTCTTTCAAGGCTTGAGACTAATATAGGCCATTGTGATTCAGGAAG
AAACCCAAGGTTGGAGGGTGGGATGAGTACCCTCTGAAAAAGGGAATTTGCTGGTGAAAA
GAGGCTGGATCTTGTGGAAGACTGTCTTGGATGGGGAAGTACTACCTGGAGATTTCAAAT
TCACTTGGCCTGCAAACAACAGAGTTATCCGTATCTTCCACATGTGAATGTCATTGCAAG
GGTGACTCTAGACAAACTACAAACCGATGGACCGTCAAGCTCCCCAGGAGCCCCTTGGAT
GGCAGCGTTGCTTCAGAGTGTTTCCTGTTTCTGGAATTCCTTGTTAGGGAACTTTAAAGA
AGAAAAGAAAAACTTGAATTGTGTTGAATTACTGTATCTTTTACTTTTTTTTTTTTGAAA
AGATAAACTTGTAAATAGAGTGATTTGAAATACTATATGGCAAAGTTTTATATTTGATAT
TCTTTAAGTTAGTTGCTCACACACTTAGGCTTTGATTGCTGAAGAAGTATGTTTAAGAGG
GAGAGAGGGGAGGCAAAGCTGAAGAGAGTCAAGGTCACTGTCCCCGCTTCGGCCTGAAGG
AAAGAGAAGACATTTCTATGGCCTTGCTCTCTGCTGTCCTGTTGGTGGGCACGACACATC
AGTGGTGTTCAGTCTTTATGTGTTTTTAAGCATCCCTTGGGCTTTGGATTTGGAGATGGG
AAGAGCATCTCCAGGCAATGAGTTTTTCAAAGAATGCCTACTTAGTAGTAAGATGAAGCT
CAGGATTTAAATAAGTGGGGTCAGGCATTCCAGTTTTTGTCTTTCTTCTCAGGTGTATTT
CTTGGTACCCCCAAGATATCAGGCCAGAAAGAGATGAGTCAGTTGCTGTGCTCTTTACTT
CTTTTTCTCCACATCTTCTGAGGCTTTAGAAATGTGGACAAGCTAGTTTTCAAATTTTGT
GTGCGTCTGTAAGTTCTTAAAGAACCAGCTTCTTAGAATGTTCAGTTCTCAATGTGCTGC
TGCTTTCCCTTCTCCTAAACATTTTAAAACTCTTCCCTTTCACCTCCAATTCCCGTGATC
CCAAAAGAAGAGGAAGACTCCAGGAGGGGTATAGATTGTGCCGTCATAGCTTTACAGGTG
GTTTTAAAGTTAACAGGGGTTTGTCATGGTGATTCACTACTCAGTTTATCAGCTCAAGGA
TTATACAGCTCTTTTCCGGGAACTCACCCAGGAGCAAGCGAGACACTACCATTGAATCAG
GGAATGAGAATTAAGAATGGACAGGACCAAGACAGAACTCAAGAAAGCCACTGGGGAAAA
CTCGAGAAGAAAGGGAGTATACTAGTAGGTTAGATCTGTGAACCTGAGGACAAGAAGACC
TTGGGAAATGGAGGCCTCAGGGGATGTGCATTCACATACTATTACGCTTCTCAAAGAGAG
ACCAACATCATGCTTTTAACACATTTGATGAGGTTTTTTATTTGTGTTTTTGTTTGTTTT
TTGAGATGGAGTCTCACTCTGTGGCCCAGGCTGGAGTGCAGTGGCGCAATCTTGGCTCAC
TGCAACCTCCACCTCCCAGGTTCAAGTGATTCTCCTGTCTCAGCCTCCCAAGTAGCTGGG
ACTACAGGCATGAGCCATCACACCCAGCTAGTTTTTTGTATTTTTAGTAAAGATGGGGTT
TTGCCATGTTTGCCAGGCTGATCTCGAACTCCTGACCTCAAGTGATCTGCCCACTTCAGA
CCCCCAAAGTGCTGGGATTCCAGGTGTGAGCCGCTGCGGCCGACCACATTTGATGTTTGA
AGTTGTAATCTGTCCCATCATAAACTTACCTGGAGCTCATGTGGAGGAACAGAAGGCCAA
GATCCTTGCTTTGGGGGTGCCTCACGAAGCATCCCTGTAGACATTTGGCCCCAGCTTCAC
TGCTTGGAAGCATGTCCCTCCCTCTTGAGTTGGCTCTGATTTGAAATCGGGAGAAACAGA
GCTGCTGCCAATGGGATCTTTTAGGTAACTCCCTCCCTAGCTTCCGTGTGTCTGTGCAGT
GCCCATGAGCTGCTGCCAATGGGATCTTTCAGGTACCCCCTCCCCAGCTTCCCTGTGGCT
GTGCGGTGCCCTTGACAGATGGCTTCTCTGTTTCCCTTTGCCCAGCCAGGCTCCCCTCCT
TCCTATTAGCTACAAAACTGGATAAACTTCAGAATATGAGCCAATGAGTAGGAAGGAACT
TGAAGACTAAAGATTTTACTCTCTCCCCTATCCATGCCCCCTACCTCTGACTCTCTCTGT
GTGAACAGGAAACTTTAGGGCAGATGAGGAGAATGAATTGGTTATCAGAGTGGAAGACCA
TGGCCCAGGATCCCTGAGCTTTCCCAGTAGCCTCCAGTTTCCTTTGTAAGACCCAGGGAT
CACTTAGCCATAGCCTGAATCTTTTAGGGGTATTAAGGTCAGCCTCTCACTCTTCCTTCA
GGTTACTAACAAAATTTCGTAGCTAAAGAATGCCATGGCCGGGTGCAGTGGCTCACGCCT
ATAATCCCAGCACTTTGGGAGGCCGAGGCGGGCGGATCACGAGGTCAGGAGATTGAGACC
ATCCTGGCTACGACGGTGAAACCCCGTCTCTACTAAAAATACAAAAAATTAGCCGGGTGT
GGTGGCGGGCGCCTGTAGTCCCAGCTACTCTGGAGGCTGAGGCAGGAGAATGGCATGAAC
CCAGGAGGCAGAGATTGCAGTGAGCCAAGATCACGCCCCTGCACTCCAGCCTGGGTGACA
GAGCCAGACTCCGTCTCAAAGG

Transcript: ARHGAP26-001 ENST00000274498

Protein sequence (SEQ ID NO.: 106), coding part of fusion gene shaded.
MGLPALEFSDCCLDSPHFRETLKSHEAELDKTNKFIKELIKDGKSLISALKNLSSAKRKF
ADSLNEFKFQCIGDAETDDEMCIARSLQEFATVLRNLEDERIRMIENASEVLITPLEKFR
KEQIGAAKEAKKKYDKETEKYCGILEKHLNLSSKKKESQLQEADSQVDLVRQHFYEVSLE
YVFKVQEVQERKMFEFVEPLLAFLQGLFTFYHHGYELAKDFGDFKTQLTISIQNTRNRFE
GTRSEVESLMKKMKENPLEHKTISPYTMEGYLYVQEKRFFGTSWVKHYCTYQRDSKQITM
VPFDQKSGGKGGEDESVILKSCTRRKTDSIEKRFCFDVEAVDRPGVITMQALSEEDRRLW

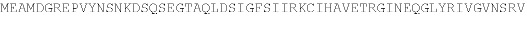

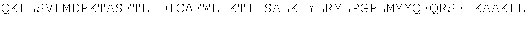

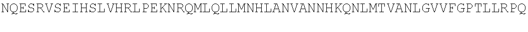

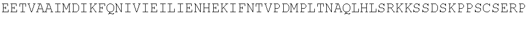

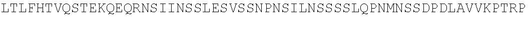

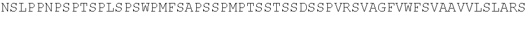

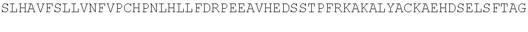

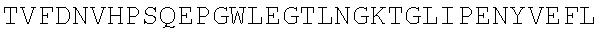

CLDN18-ARHGAP26 Fusion sequence

cDNA sequence (SEQ ID NO.: 107), ARHGAP26 underlined.
ATGGCCGTGACTGCCTGTCAGGGCTTGGGGTTCGTGGTTTCACTGATTGGGATTGCGGGCATCATTGCTGCCACC
TGCATGGACCAGTGGAGCACCCAAGACTTGTACAACAACCCCGTAACAGCTGTTTTCAACTACCAGGGGCTGTGG
CGCTCCTGTGTCCGAGAGAGCTCTGGCTTCACCGAGTGCCGGGGCTACTTCACCCTGCTGGGGCTGCCAGCCATG
CTGCAGGCAGTGCGAGCCCTGATGATCGTAGGCATCGTCCTGGGTGCCATTGGCCTCCTGGTATCCATCTTTGCC
CTGAAATGCATCCGCATTGGCAGCATGGAGGACTCTGCCAAAGCCAACATGACACTGACCTCCGGGATCATGTTC
ATTGTCTCAGGTCTTTGTGCAATTGCTGGAGTGTCTGTGTTTGCCAACATGCTGGTGACTAACTTCTGGATGTCC
ACAGCTAACATGTACACCGGCATGGGTGGGATGGTGCAGACTGTTCAGACCAGGTACACATTTGGTGCGGCTCTG
TTCGTGGGCTGGGTCGCTGGAGGCCTCACACTAATTGGGGGTGTGATGATGTGCATCGCCTGCCGGGGCCTGGCA
CCAGAAGAAACCAACTACAAAGCCGTTTCTTATCATGCCTCAGGCCACAGTGTTGCCTACAAGCCTGGAGGCTTC
AAGGCCAGCACTGGCTTTGGGTCCAACACCAAAAACAAGAAGATATACGATGGAGGTGCCCGCACAGAGGACGAG

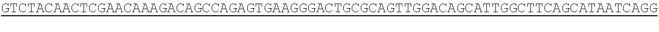

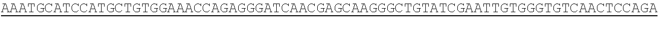

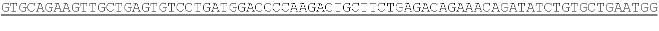

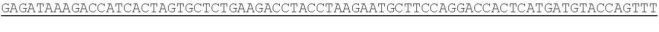

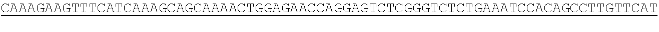

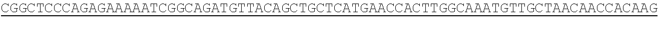

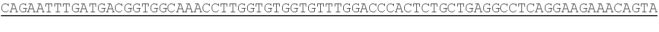

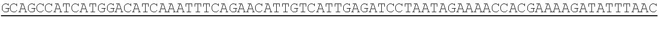

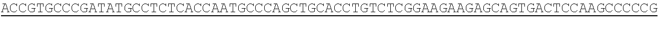

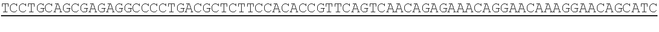

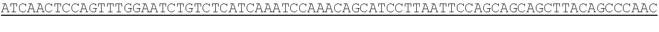

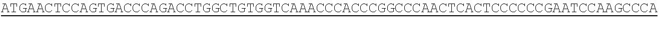

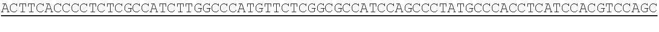

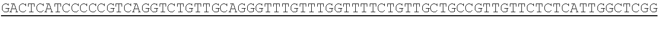

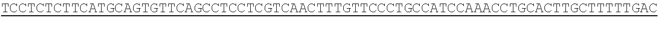

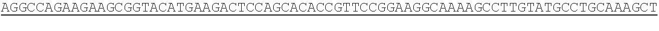

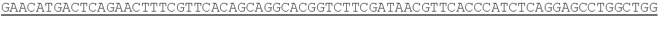

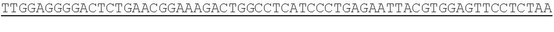

Protein sequence (SEQ ID NO.: 108), ARHGAP26 underlined.
MAVTACQGLGFVVSLIGIAGIIAATCMDQWSTQDLYNNPVTAVFNYQGLWRSCVRESSGFTECRGYFTLLGLPAM
LQAVRALMIVGIVLGAIGLLVSIFALKCIRIGSMEDSAKANMTLTSGIMFIVSGLCAIAGVSVFANMLVTNFWMS
TANMYTGMGGMVQTVQTRYTFGAALFVGWVAGGLTLIGGVMMCIACRGLAPEETNYKAVSYHASGHSVAYKPGGF

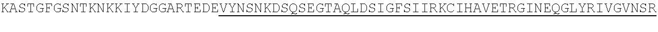

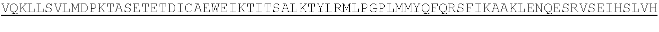

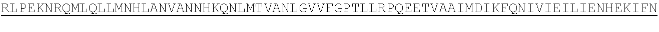

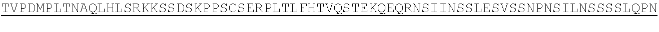

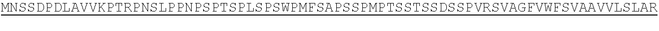

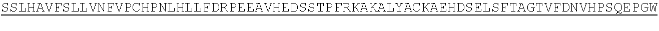

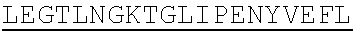

Protein Domain

Domains within the query sequence of 695 residues

| Name | Start | End |
|---|---|---|
| Transmembrane region | 4 | 26 |
| Transmembrane region | 84 | 106 |
| Transmembrane region | 126 | 148 |
| Transmembrane region | 169 | 191 |

Fusion Gene #3: SNX2-PRDM6

Confirmed genomic breakpoint for SNX2 on chr5:122162808 located in intron 12-13 of Transcript: SNX2-001 (ENST00000379516)

Confirmed genomic breakpoint for PRDM6 on chr5:122437347 located at intron 3-4 of Transcript: PRDM6-001 (ENST00000407847)

Transcript: SNX2-001 ENST00000379516

cDNA sequence (SEQ ID NO.: 109), coding part of
fusion gene shaded.
AGGCCGGCCGGGGGCGGGGAGGCTGGCGGGTCGGCGCGGGCCCAGCCGT
GCGTGCTCACGTGACGGGTCCGCGAGGCCCAGCTCGCGCAGTCGTTCGG
GTGAGCGAAGATGGCGGCCGAGAGGGAACCTCCTCCGCTGGGGGACGGG
AAGCCCACCGACTTTGAGGATCTGGAGGACGGAGAGGACCTGTTCACCA
GCACTGTCTCCACCCTAGAGTCAAGTCCATCATCTCCAGAACCAGCTAG
TCTTCCTGCAGAAGATATTAGTGCAAACTCCAATGGCCCAAAACCCACA
GAAGTTGTATTAGATGATGACAGAGAAGATCTTTTTGCAGAAGCCACAG
AAGAAGTTTCTTTGGACAGCCCTGAAAGGGAACCTATCCTATCCTCGGA
ACCTTCTCCTGCAGTCACACCTGTCACTCCTACTACACTCATTGCTCCT
AGAATTGAATCAAAGAGTATGTCTGCTCCCGTGATCTTTGATAGATCCA
GGGAAGAGATTGAAGAAGAAGCAAATGGAGACATTTTTGACATAGAAAT
TGGTGTATCAGATCCAGAAAAAGTTGGTGATGGCATGAATGCCTATATG
GCATATAGAGTAACAACAAAGACATCTCTTTCCATGTTCAGTAAGAGTG
AATTTTCAGTGAAAAGAAGATTCAGCGACTTTCTTGGTTTGCACAGCAA
ATTAGCAAGCAAATATTTACATGTTGGTTATATTGTGCCACCAGCTCCA
GAAAAGAGTATAGTAGGGATGACCAAGGTCAAAGTGGGTAAAGAAGACT
CATCATCCACTGAGTTTGTAGAAAAACGGAGAGCAGCTCTTGAAAGGTA
TCTTCAAAGAACAGTAAAACATCCAACTTTACTACAGGATCCTGATTTA
AGGCAGTTCTTGGAAAGTTCAGAGCTGCCTAGAGCAGTTAATACACAGG
CTCTGAGTGGAGCAGGAATATTGAGGATGGTGAACAAGGCTGCCGACGC
TGTCAACAAAATGACAATCAAGATGAATGAATCGGATGCATGGTTTGAA
GAAAAGCAGCAGCAATTTGAGAATCTGGATCAGCAACTTAGGAAACTTC
ATGTCAGTGTTGAAGCCTTGGTCTGTCATAGAAAAGAACTTTCAGCCAA
CACAGCTGCCTTTGCTAAAAGTGCTGCCATGTTAGGTAATTCTGAGGAT
CATACTGCTTTATCTAGAGCTTTGTCTCAGCTTGCAGAGGTTGAGGAGA
AGATAGACCAGTTACATCAAGAACAAGCTTTTGCTGACTTTTATATGTT
TTCAGAACTACTTAGTGACTACATTCGTCTTATTGCTGCAGTGAAAGGT
GTGTTTGACCATCGAATGAAGTGCTGGCAGAAATGGGAAGATGCTCAAA
TTACTTTGCTCAAAAAACGTGAAGCTGAAGCAAAAATGATGGTTGCTAA
CAAACCAGATAAAATACAGCAAGCTAAAAATGAAATAAGAGAGTGGGAG
GCGAAAGTGCAACAAGGGGAAAGAGATTTTGAACAGATATCTAAAACGA
TTCGAAAAGAAGTGGGAAGATTTGAGAAAGAACGAGTGAAGGATTTTAA
AACCGTTATCATCAAGTACTTAGAATCACTAGTTCAAACACAACAACAG
CTGATAAAATACTGGGAAGCATTCCTACCTGAAGCCAAAGCCATTGCCT
AGCAATAAGATTGTTGCCGTTAAGAAGACCTTGGATGTTGTTCCAGTTA
TGCTGGATTCCACAGTGAAATCATTTAAAACCATCTAAATAAACCACTA
TATATTTTATGAATTACATGTGGTTTTATATACACACACACACACACAC
ACACACACACACACACACTCTGACATTTTATTACAAGCTGCATGTCCTG
ACCCTCTTTGAATTAAGTGGACTGTGGCATGACATTCTGCAATACTTTG
CTGAATTGAACACTATTGTGTCTTAAATACTTGCACTAAATAGTGCACT
GCAAGACCAGAAAATTTTACAATATTTTTTCTTTACAATATGTTCTGTA
GTATGTTTACCCTCTTTATGAAGTGAATTACCAATGCTTTGAATAATGT
TCACTTATACATTCCTGTACAGAAATTACGATTTTGTGATTACAGTAAT
AAAATGATATTCCTTGTGAAA

Transcript: SNX2-001 ENST00000379516

Protein sequence (SEQ ID NO.: 110), coding part
of fusion gene shaded.
MAAEREPPPLGDGKPTDFEDLEDGEDLFTSTVSTLESSPSSPEPASLPAE
DISANSNGPKPTEVVLDDDREDLFAEATEEVSLDSPEREPILSSEPSPAV
TPVTPTTLIAPRIESKSMSAPVIFDRSREEIEEEANGDIFDIEIGVSDPE
KVGDGMNAYMAYRVTTKTSLSMFSKSEFSVKRRFSDFLGLHSKLASKYLH
VGYIVPPAPEKSIVGMTKVKVGKEDSSSTEFVEKRRAALERYLQRTVKHP
TLLQDPDLRQFLESSELPRAVNTQALSGAGILRMVNKAADAVNKMTIKMN
ESDAWFEEKQQQFENLDQQLRKLHVSVEALVCHRKELSANTAAFAKSAAM
LGNSEDHTALSRALSQLAEVEEKIDQLHQEQAFADFYMFSELLSDYIRLI
AAVKGVFDHRMKCWQKWEDAQITLLKKREAEAKMMVANKPDKIQQAKNEI
REWEAKVQQGERDFEQISKTIRKEVGRFEKERVKDFKTVIIKYLESLVQT
QQQLIKYWEAFLPEAKAIA

Transcript: PRDM6-001 ENST00000407847

cDNA sequence (SEQ ID NO: 111),
coding part of fusion gene shaded.

CTCTCTCACACACACACACACACACACACACACACACACACACACACACAC
ACACACACACACACACACACTCACTCTATTTTGTGCTGTCGTAAAACCCAC
GTGTCCAGCCGGGAAGCTGCCAGAGCGTGGAACCAAGGAGCCAGGACGCGG
CAGCGGCCAAGCGCAGCAGCCCACGGCGGTTGAGTCGGGCGCCCAGGTCCG
TCCGCACTCTCGCGCCCTCCGCGGGCCTCCCAATTTTCTCGCTTGCAGGTC
GGGAGGTTTCCGGGCGGCACAATCTCTAGGACTCTCCTCCCGCGCTGCTCA
GGGGCATGTAGCGCACGCAGGGCGCACACTCTCGCGCACCCGCACGCTCAC
CGAGACACCCGCACGCACCCACCGGCAGCACCGAGTTTTCAGTTCGAGGCG
CCGGACATGCTGAAGCCCGGAGACCCCGGCGGTTCGGCCTTCCTCAAAGTG
GACCCAGCCTACCTGCAGCACTGGCAGCAACTCTTCCCTCACGGAGGCGCA
GGCCCGCTCAAGGGCAGCGGCGCCGCGGGTCTCCTGAGCGCGCCGCAGCCT
CTTCAGCCGCCGCCGCCGCCCCCGCCCCCGGAGCGCGCTGAGCCTCCGCCG
GACAGCCTGCGCCCGCGGCCCGCCTCTCTCTCCTCCGCCTCGTCCACGCCG
GCTTCCTCTTCCACCTCCGCCTCCTCCGCCTCCTCCTGCGCTGCTGCGGCC
GCTGCCGCCGCGCTGGCTGGTCTCTCGGCCCTGCCGGTGTCGCAGCTGCCG
GTGTTCGCGCCTCTAGCCGCCGCTGCCGTCGCCGCCGAGCCGCTGCCCCCC
AAGGAACTGTGCCTCGGCGCCACCTCCGGCCCCGGGCCCGTCAAGTGCGGT
GGTGGTGGCGGCGGCGGCGGGGAGGGTCGCGGCGCCCCGCGCTTCCGCTGC
AGCGCAGAGGAGCTGGACTATTACCTGTATGGCCAGCAGCGCATGGAGATC
ATCCCGCTCAACCAGCACACCAGCGACCCCAACAACCGTTGCGACATGTGC
GCGGACAACCGCAACGGCGAGTGCCCTATGCATGGGCCACTGCACTCGCTG
CGCCGGCTTGTGGGCACCAGCAGCGCTGCGGCCGCCGCGCCCCCGCCGGAG
CTGCCGGAGTGGCTGCGGGACCTGCCTCGCGAGGTGTGCCTCTGCACCAGT
ACTGTGCCCGGCCTGGCCTACGGCATCTGCGCGGCGCAGAGGATCCAGCAA
GGCACCTGGATTGGACCTTTCCAAGGCGTGCTTCTGCCCCCAGAGAAGGTG

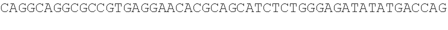

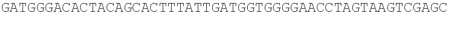

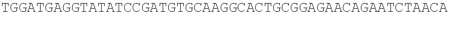

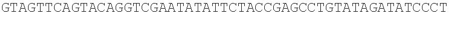

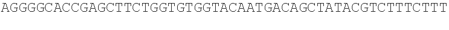

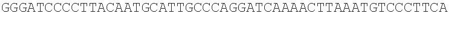

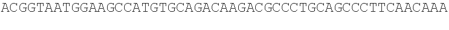

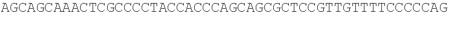

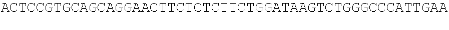

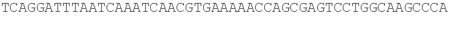

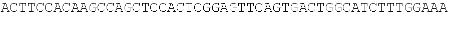

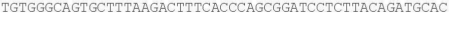

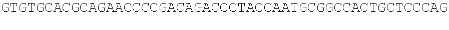

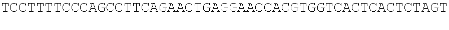

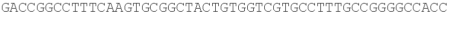

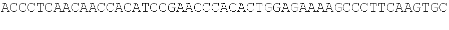

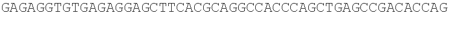

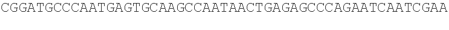

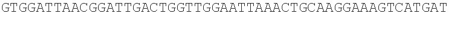

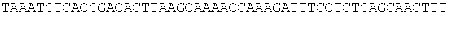

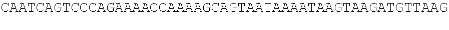

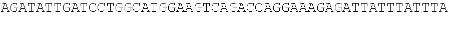

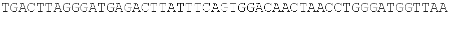

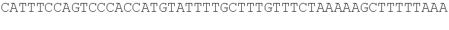

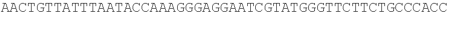

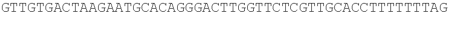

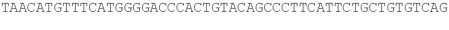

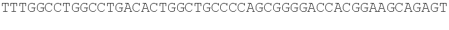

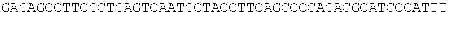

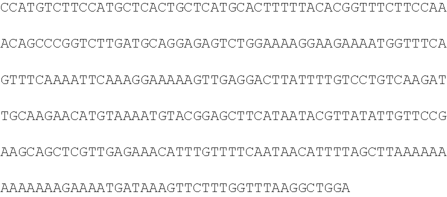

Transcript: PRDM6-001 ENST00000407847

Protein sequence (SEQ ID NO. :112). coding part of fusion gene shaded.
MLKPGDPGGSAFLKVDPAYLQHWQQLFPHGGAGPLKGSGAAGLLSAPQPLQPPPPPPPPE
RAEPPPDSLRPRPASLSSASSTPASSSTSASSASSCAAAAAAAALAGLSALPVSQLPVFA
PLAAAAVAAEPLPPKELCLGATSGPGPVKCGGGGGGGGEGRGAPRFRCSAEELDYYLYGQ
QRMEIIPLNQHTSDPNNRCDMCADNRNGECPMHGPLHSLRRLVGTSSAAAAAPPPELPEW
LRDLPREVCLCTSTVPGLAYGICAAQRIQQGTWIGPFQGVLLPPEKVQAGAVRNTQHLWE

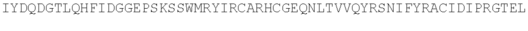

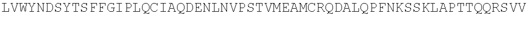

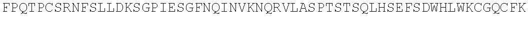

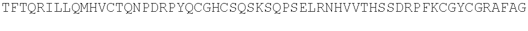

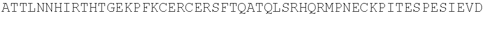

SNX2-PRDM6 Fusion sequence exon 12 to exon 4

cDNA sequence
(SEQ ID NO.: 113)
ATGGCGGCCGAGAGGGAACCTCCTCCGCTGGGGGACGGGAAGCCCACCGACTTTGAGGATCTGGAGGACGGAGAG
GACCTGTTCACCAGCACTGTCTCCACCCTAGAGTCAAGTCCATCATCTCCAGAACCAGCTAGTCTTCCTGCAGAA
GATATTAGTGCAAACTCCAATGGCCCAAAACCCACAGAAGTTGTATTAGATGATGACAGAGAAGATCTTTTTGCA
GAAGCCACAGAAGAAGTTTCTTTGGACAGCCCTGAAAGGGAACCTATCCTATCCTCGGAACCTTCTCCTGCAGTC
ACACCTGTCACTCCTACTACACTCATTGCTCCTAGAATTGAATCAAAGAGTATGTCTGCTCCCGTGATCTTTGAT
AGATCCAGGGAAGAGATTGAAGAAGAAGCAAATGGAGACATTTTTGACATAGAAATTGGTGTATCAGATCCAGAA
AAAGTTGGTGATGGCATGAATGCCTATATGGCATATAGAGTAACAACAAAGACATCTCTTTCCATGTTCAGTAAG
AGTGAATTTTCAGTGAAAAGAAGATTCAGCGACTTTCTTGGTTTGCACAGCAAATTAGCAAGCAAATATTTACAT
GTTGGTTATATTGTGCCACCAGCTCCAGAAAAGAGTATAGTAGGGATGACCAAGGTCAAAGTGGGTAAAGAAGAC
TCATCATCCACTGAGTTTGTAGAAAAACGGAGAGCAGCTCTTGAAAGGTATCTTCAAAGAACAGTAAAACATCCA
ACTTTACTACAGGATCCTGATTTAAGGCAGTTCTTGGAAAGTTCAGAGCTGCCTAGAGCAGTTAATACACAGGCT
CTGAGTGGAGCAGGAATATTGAGGATGGTGAACAAGGCTGCCGACGCTGTCAACAAAATGACAATCAAGATGAAT
GAATCGGATGCATGGTTTGAAGAAAAGCAGCAGCAATTTGAGAATCTGGATCAGCAACTTAGGAAACTTCATGTC
AGTGTTGAAGCCTTGGTCTGTCATAGAAAAGAACTTTCAGCCAACACAGCTGCCTTTGCTAAAAGTGCTGCCATG
TTAGGTAATTCTGAGGATCATACTGCTTTATCTAGAGCTTTGTCTCAGCTTGCAGAGGTTGAGGAGAAGATAGAC
CAGTTACATCAAGAACAAGCTTTTGCTGACTTTTATATGTTTTCAGAACTACTTAGTGACTACATTCGTCTTATT
GCTGCAGTGAAAGGTGTGTTTGACCATCGAATGAAGTGCTGGCAGAAATGGGAAGATGCTCAAATTACTTTGCTC
AAAAAACGTGAAGCTGAAGCAAAAATGATGGTTGCTAACAAACCAGATAAAATACAGCAAGCTAAAAATGAAATA

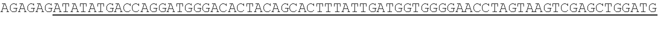

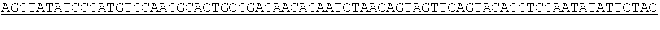

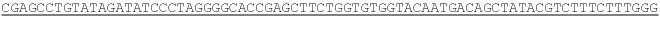

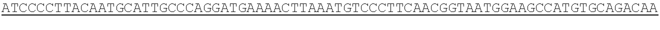

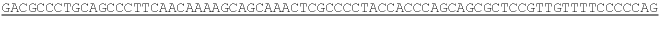

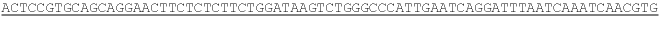

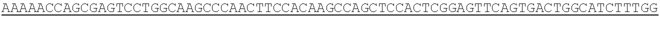

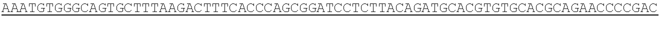

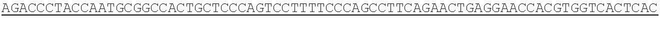

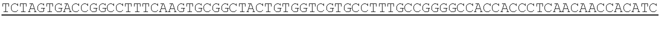

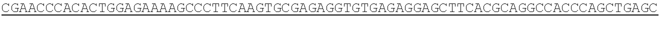

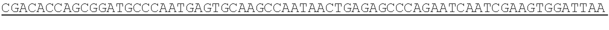

Protein sequence
(SEQ ID NO.: 114)
MAAEREPPPLGDGKPTDFEDLEDGEDLFTSTVSTLESSPSSPEPASLPAEDISANSNGPKPTEVVLDDDREDLFA
EATEEVSLDSPEREPILSSEPSPAVTPVTPTTLIAPRIESKSMSAPVIFDRSREEIEEEANGDIFDIEIGVSDPE
KVGDGMNAYMAYRVTTKTSLSMFSKSEFSVKRRFSDFLGLHSKLASKYLHVGYIVPPAPEKSIVGMTKVKVGKED
SSSTEFVEKRRAALERYLQRTVKHPTLLQDPDLRQFLESSELPRAVNTQALSGAGILRMVNKAADAVNKMTIKMN
ESDAWFEEKQQQFENLDQQLRKLHVSVEALVCHRKELSANTAAFAKSAAMLGNSEDHTALSRALSQLAEVEEKID
QLHQEQAFADFYMFSELLSDYIRLIAAVKGVFDHRMKCWQKWEDAQITLLKKREAEAKMMVANKPDKIQQAKNEI

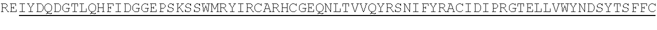

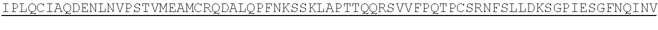

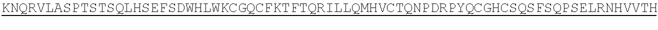

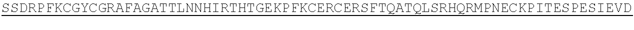

Protein Domains

No transmembrane domains.

SNX2-PRDM6 Fusion sequence exon 2 to exon 7

cDNA sequence
(SEQ ID NO.: 115)
ATGGCGGCCGAGAGGGAACCTCCTCCGCTGGGGGACGGGAAGCCCACCGACTTTGAGGATCTGGAGGACGGAGAG
GACCTGTTCACCAGCACTGTCTCCACCCTAGAGTCAAGTCCATCATCTCCAGAACCAGCTAGTCTTCCTGCAGAA
GATATTAGTGCAAACTCCAATGGCCCAAAACCCACAGAAGTTGTATTAGATGATGACAGAGAAGATCTTTTTGCA

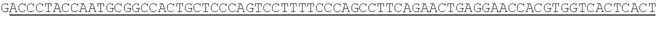

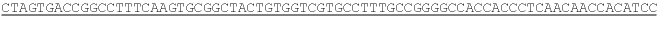

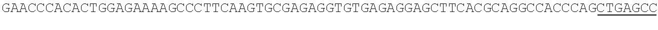

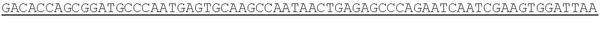

Protein sequence
(SEQ ID NO.: 116)
MAAEREPPPLGDGKPTDFEDLEDGEDLFTSTVSTLESSPSSPEPASLPAEDISANSNGPKPTEVVLDDDREDLFA

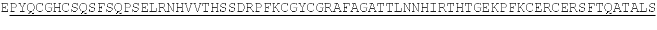

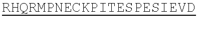

Protein Domains

No transmembrane domains.

Fusion Gene #4: MLL3-PRKAG2

Confirmed genomic breakpoint for MLL3 on chr7:151365906 (reference Transcript: MLL3-001 (ENST00000262189))

confirmed genomic breakpoint for PRKAG2 on chr7:151951997 (reference Transcript: PRKAG2-001 (ENST00000287878))

Transcript: MLL3-001 ENST00000262189

cDNA sequence (SEQ ID NO.: 117), part of fusion
gene is shaded.
GAGGTGCGCGCGCCCGCGCCGATGTGTGTGAGTGCGTGTCCTGCTCGCT
CCATGTTGCCGCCTCTCCCGGTACCTGCTGCTGCTCCCGGGGCTGCGGG
AAATGCGAGAGGCTGAGCCGGGGAGGAGGAACCCGAGCAGCAGCGGCGG
CGGCGGCGGCCGCGGCGGCGGGAGCCCCCCAGGAGGAGGACCGGGATCC
ATGTGTCTTTCCTGGTGACTAGGATGTCGTCGGAGGAGGACAAGAGCGT
GGAGCAGCCGCAGCCGCCGCCACCACCCCCCGAGGAGCCTGGAGCCCCG
GCCCCGAGCCCCGCAGCCGCAGACAAAAGACCTCGGGGCCGGCCTCGCA
AAGATGGCGCTTCCCCTTTCCAGAGAGCCAGAAAGAAACCTCGAAGTAG
GGGGAAAACTGCAGTGGAAGATGAGGACAGCATGGATGGGCTGGAGACA
ACAGAAACAGAAACGATTGTGGAAACAGAAATCAAAGAACAATCTGCAG
AAGAGGATGCTGAAGCAGAAGTGGATAACAGCAAACAGCTAATTCCAAC
TCTTCAGCGATCTGTGTCTGAGGAATCGGCAAACTCCCTGGTCTCTGTT
GGTGTAGAAGCCAAAATCAGTGAACAGCTCTGCGCTTTTTGTTACTGTG
GGGAAAAAAGTTCCTTAGGACAAGGAGACTTAAAACAATTCAGAATAAC
GCCTGGATTTATCTTGCCATGGAGAAACCAACCTTCTAACAAGAAGGAC
ATTGATGACAACAGCAATGGAACCTATGAGAAAATGCAAAACTCAGCAC
CACGAAAACAAAGAGGACAGAGAAAAGAACGATCTCCTCAGCAGAATAT
AGTATCTTGTGTAAGTGTAAGCACCCAGACAGCTTCAGATGATCAAGCT
GGTAAACTGTGGGATGAACTCAGTCTGGTTGGGCTTCCAGATGCCATTG
ATATCCAAGCCTTATTTGATTCTACAGGCACTTGTTGGGCTCATCACCG
TTGTGTGGAGTGGTCACTAGGAGTATGCCAGATGGAAGAACCATTGTTA
GTGAACGTGGACAAAGCTGTTGTCTCAGGGAGCACAGAACGATGTGCAT
TTTGTAAGCACCTTGGAGCCACTATCAAATGCTGTGAAGAGAAATGTAC
CCAGATGTATCATTATCCTTGTGCTGCAGGAGCCGGCACCTTTCAGGAT
TTCAGTCACATCTTCCTGCTTTGTCCAGAACACATTGACCAAGCTCCTG
AAAGATCGAAGGAAGATGCAAACTGTGCAGTGTGCGACAGCCCGGGAGA
CCTCTTAGATCAGTTCTTTTGTACTACTTGTGGTCAGCACTATCATGGA
ATGTGCCTGGATATAGCGGTTACTCCATTAAAACGTGCAGGTTGGCAAT
GTCCTGAGTGCAAAGTGTGCCAGAACTGCAAACAATCGGGAGAAGATAG
CAAGATGCTAGTGTGTGATACGTGTGACAAAGGGTATCATACTTTTTGT
CTTCAACCAGTTATGAAATCAGTACCAACCAATGGCTGGAAATGCAAAA
ATTGCAGAATATGTATAGAGTGTGGCACACGGTCTAGTTCTCAGTGGCA
CCACAATTGCCTGATATGTGACAATTGTTACCAACAGCAGGATAACTTA
TGTCCCTTCTGTGGGAAGTGTTATCATCCAGAATTGCAGAAAGACATGC
TTCATTGTAATATGTGCAAAAGGTGGGTTCACCTAGAGTGTGACAAACC
AACAGATCATGAACTGGATACTCAGCTCAAAGAAGAGTATATCTGCATG
TATTGTAAACACCTGGGAGCTGAGATGGATCGTTTACAGCCAGGTGAGG
AAGTGGAGATAGCTGAGCTCACTACAGATTATAACAATGAAATGGAAGT
TGAAGGCCCTGAAGATCAAATGGTATTCTCAGAGCAGGCAGCTAATAAA
GATGTCAACGGTCAGGAGTCCACTCCTGGAATTGTTCCAGATGCGGTTC
AAGTCCACACTGAAGAGCAACAGAAGAGTCATCCCTCAGAAAGTCTTGA
CACAGATAGTCTTCTTATTGCTGTATCATCCCAACATACAGTGAATACT
GAATTGGAAAAACAGATTTCTAATGAAGTTGATAGTGAAGACCTGAAAA
TGTCTTCTGAAGTGAAGCATATTTGTGGCGAAGATCAAATTGAAGATAA
AATGGAAGTGACAGAAAACATTGAAGTCGTTACACACCAGATCACTGTG
CAGCAAGAACAACTGCAGTTGTTAGAGGAACCTGAAACAGTGGTATCCA
GAGAAGAATCAAGGCCTCCAAAATTAGTCATGGAATCTGTCACTCTTCC
ACTAGAAACCTTAGTGTCCCCACATGAGGAAAGTATTTCATTATGTCCT
GAGGAACAGTTGGTTATAGAAAGGCTACAAGGAGAAAAGGAACAGAAAG
AAAATTCTGAACTTTCTACTGGATTGATGGACTCTGAAATGACTCCTAC
AATTGAGGGTTGTGTGAAAGATGTTTCATACCAAGGAGGCAAATCTATA
AAGTTATCATCTGAGACAGAGTCATCATTTTCATCATCAGCAGACATAA
GCAAGGCAGATGTGTCTTCCTCCCCAACACCTTCTTCAGACTTGCCTTC
GCATGACATGCTGCATAATTACCCTTCAGCTCTTAGTTCCTCTGCTGGA
AACATCATGCCAACAACTTACATCTCAGTCACTCCAAAAATTGGCATGG
GTAAACCAGCTATTACTAAGAGAAAATTTTCTCCTGGTAGACCTCGGTC
CAAACAGGGGGCTTGGAGTACCCATAATACAGTGAGCCCACCTTCCTGG
TCCCCAGACATTTCAGAAGGTCGGGAAATTTTTAAACCCAGGCAGCTTC
CTGGCAGTGCCATTTGGAGCATCAAAGTGGGCCGTGGGTCTGGATTTCC
AGGAAAGCGGAGACCTCGAGGTGCAGGACTGTCGGGGCGAGGTGGCCGA
GGCAGGTCAAAGCTGAAAAGTGGAATCGGAGCTGTTGTATTACCTGGGG
TGTCTACTGCAGATATTTCATCAAATAAGGATGATGAAGAAAACTCTAT
GCACAATACAGTTGTGTTGTTTTCTAGCAGTGACAAGTTCACTTTGAAT
CAGGATATGTGTGTAGTTTGTGGCAGTTTTGGCCAAGGAGCAGAAGGAA
GATTACTTGCCTGTTCTCAGTGTGGTCAGTGTTACCATCCATACTGTGT
CAGTATTAAGATCACTAAAGTGGTTCTTAGCAAAGGTTGGAGGTGTCTT
GAGTGCACTGTGTGTGAGGCCTGTGGGAAGGCAACTGACCCAGGAAGAC
TCCTGCTGTGTGATGACTGTGACATAAGTTATCACACCTACTGCCTAGA
CCCTCCATTGCAGACAGTTCCCAAAGGAGGCTGGAAGTGCAAATGGTGT
GTTTGGTGCAGACACTGTGGAGCAACATCTGCAGGTCTAAGATGTGAAT
GGCAGAACAATTACACACAGTGCGCTCCTTGTGCAAGCTTATCTTCCTG
TCCAGTCTGCTATCGAAACTATAGAGAAGAAGATCTTATTCTGCAATGT
AGACAATGTGATAGATGGATGCATGCAGTTTGTCAGAACTTAAATACTG
AGGAAGAAGTGGAAAATGTAGCAGACATTGGTTTTGATTGTAGCATGTG
CAGACCCTATATGCCTGCGTCTAATGTGCCTTCCTCAGACTGCTGTGAA
TCTTCACTTGTAGCACAAATTGTCACAAAAGTAAAAGAGCTAGACCCAC
CCAAGACTTATACCCAGGATGGTGTGTGTTTGACTGAATCAGGGATGAC
TCAGTTACAGAGCCTCACAGTTACAGTTCCAAGAAGAAAACGGTCAAAA
CCAAAATTGAAATTGAAGATTATAAATCAGAATAGCGTGGCCGTCCTTC
AGACCCCTCCAGACATCCAATCAGAGCATTCAAGGGATGGTGAAATGGA
TGATAGTCGAGAAGGAGAACTTATGGATTGTGATGGAAAATCAGAATCT
AGTCCTGAGCGGGAAGCTGTGGATGATGAAACTAAGGGAGTGGAAGGAA
CAGATGGTGTCAAAAAGAGAAAAAGGAAACCATACAGACCAGGTATTGG
TGGATTTATGGTGCGGCAAAGAAGTCGAACTGGGCAAGGGAAAACCAAA
AGATCTGTGATCAGAAAAGATTCCTCAGGCTCTATTTCCGAGCAGTTAC
CTTGCAGAGATGATGGCTGGAGTGAGCAGTTACCAGATACTTTAGTTGA
TGAATCTGTTTCTGTTACTGAAAGCACTGAAAAAATAAAGAAGAGATAC
CGAAAAAGGAAAAATAAGCTTGAAGAAACTTTCCCTGCCTATTTACAAG
AAGCTTTCTTTGGAAAAGATCTTCTAGATACAAGTAGACAAAGCAAGAT
AAGTTTAGATAATCTGTCAGAAGATGGAGCTCAGCTTTTATATAAAACA
AACATGAACACAGGTTTCTTGGATCCTTCCTTAGATCCACTACTTAGTT
CATCCTCGGCTCCAACAAAATCTGGAACTCACGGTCCTGCTGATGACCC
ATTAGCTGATATTTCTGAAGTTTTAAACACAGATGATGACATTCTTGGA
ATAATTTCAGATGATCTAGCAAAATCAGTTGATCATTCAGATATTGGTC
CTGTCACTGATGATCCTTCCTCTTTGCCTCAGCCAAATGTCAATCAGAG
TTCACGACCATTAAGTGAAGAACAGCTAGATGGGATCCTCAGTCCTGAA
CTAGACAAAATGGTCACAGATGGAGCAATTCTTGGAAAATTATATAAAA
TTCCAGAGCTTGGCGGAAAAGATGTTGAAGACTTATTTACAGCTGTACT
TAGTCCTGCGAACACTCAGCCAACTCCATTGCCACAGCCTCCCCCACCA
ACACAGCTGTTGCCAATACACAATCAGGATGCTTTTTCACGGATGCCTC
TCATGAATGGCCTTATTGGATCCAGTCCTCATCTCCCACATAATTCTTT
GCCACCTGGAAGCGGACTGGGAACTTTCTCTGCAATTGCACAATCCTCT
TATCCTGATGCCAGGGATAAAAATTCAGCCTTTAATCCAATGGCAAGTG
ATCCTAACAACTCTTGGACATCATCAGCTCCCACTGTGGAAGGAGAAAA
TGACACAATGTCGAATGCCCAGAGAAGCACGCTTAAGTGGGAGAAAGAG
GAGGCTCTGGGTGAAATGGCAACTGTTGCCCCAGTTCTCTACACCAATA
TTAATTTCCCCAACTTAAAGGAAGAATTCCCTGATTGGACTACTAGAGT
GAAGCAAATTGCCAAATTGTGGAGAAAAGCAAGCTCACAAGAAAGAGCA
CCATATGTGCAAAAAGCCAGAGATAACAGAGCTGCTTTACGCATTAATA
AAGTACAGATGTCAAATGATTCCATGAAAAGGCAGCAACAGCAAGATAG
CATTGATCCCAGCTCTCGTATTGATTCGGAGCTTTTTAAAGATCCTTTA
AAGCAAAGAGAATCAGAACATGAACAGGAATGGAAATTTAGACAGCAAA
TGCGTCAGAAAAGTAAGCAGCAAGCTAAAATTGAAGCCACACAGAAACT
TGAACAGGTGAAAAATGAGCAGCAGCAGCAGCAACAACAGCAATTTGGT
TCTCAGCATCTTCTGGTGCAGTCTGGTTCAGATACACCAAGTAGTGGGA
TACAGAGTCCCTTGACACCTCAGCCTGGCAATGGAAATATGTCTCCTGC
ACAGTCATTCCATAAAGAACTGTTTACAAAACAGCCACCCAGTACCCCT
ACGTCTACATCTTCAGATGATGTGTTTGTAAAGCCACAAGCTCCACCTC
CTCCTCCAGCCCCATCCCGGATTCCCATCCAGGATAGTCTTTCTCAGGC
TCAGACTTCTCAGCCACCCTCACCGCAAGTGTTTTCACCTGGGTCCTCT
AACTCACGACCACCATCTCCAATGGATCCATATGCAAAAATGGTTGGTA
CCCCTCGACCACCTCCTGTGGGCCATAGTTTTTCCAGAAGAAATTCTGC
TGCACCAGTGGAAAACTGTACACCTTTATCATCGGTATCTAGGCCCCTT
CAAATGAATGAGACAACAGCAAATAGGCCATCCCCTGTCAGAGATTTAT
GTTCTTCTTCCACGACAAATAATGACCCCTATGCAAAACCTCCAGACAC
ACCTAGGCCTGTGATGACAGATCAATTTCCCAAATCCTTGGGCCTATCC
CGGTCTCCTGTAGTTTCAGAACAAACTGCAAAAGGCCCTATAGCAGCTG
GAACCAGTGATCACTTTACTAAACCATCTCCTAGGGCAGATGTGTTTCA
AAGACAAAGGATACCTGACTCATATGCACGACCCTTGTTGACACCTGCA
CCTCTTGATAGTGGTCCTGGACCTTTTAAGACTCCAATGCAACCTCCTC
CATCCTCTCAGGATCCTTATGGATCAGTGTCACAGGCATCAAGGCGATT
GTCTGTTGACCCTTATGAAAGGCCTGCTTTGACACCAAGACCTATAGAT
AATTTTTCTCATAATCAGTCAAATGATCCATATAGTCAGCCTCCCCTTA
CCCCACATCCAGCAGTGAATGAATCTTTTGCCCATCCTTCAAGGGCTTT
TTCCCAGCCTGGAACCATATCAAGGCCAACATCTCAGGACCCATACTCC
CAACCCCCAGGAACTCCACGACCTGTTGTAGATTCTTATTCCCAATCTT
CAGGAACAGCTAGGTCCAATACAGACCCTTACTCTCAACCTCCTGGAAC
TCCCCGGCCTACTACTGTTGACCCATATAGTCAGCAGCCCCAAACCCCA
AGACCATCTACACAAACTGACTTGTTTGTTACACCTGTAACAAATCAGA
GGCATTCTGATCCATATGCTCATCCTCCTGGAACACCAAGACCTGGAAT
TTCTGTCCCTTACTCTCAGCCACCAGCAACACCAAGGCCAAGGATTTCA
GAGGGTTTTACTAGGTCCTCAATGACAAGACCAGTCCTCATGCCAAATC
AGGATCCTTTCCTGCAAGCAGCACAAAACCGAGGACCAGCTTTACCTGG
CCCGTTGGTAAGGCCACCTGATACATGTTCCCAGACACCTAGGCCCCCT
GGACCTGGTCTTTCAGACACATTTAGCCGTGTTTCCCCATCTGCTGCCC
GTGATCCCTATGATCAGTCTCCAATGACTCCAAGATCTCAGTCTGACTC
TTTTGGAACAAGTCAAACTGCCCATGATGTTGCTGATCAGCCAAGGCCT
GGATCAGAGGGGAGCTTCTGTGCATCTTCAAACTCTCCAATGCACTCCC
AAGGCCAGCAGTTCTCTGGTGTCTCCCAACTTCCTGGACCTGTGCCAAC
TTCAGGAGTAACTGATACACAGAATACTGTAAATATGGCCCAAGCAGAT
ACAGAGAAATTGAGACAGCGGCAGAAGTTACGTGAAATCATTCTCCAGC
AGCAACAGCAGAAGAAGATTGCAGGTCGACAGGAGAAGGGGTCACAGGA
CTCACCCGCAGTGCCTCATCCAGGGCCTCTTCAACACTGGCAACCAGAG
AATGTTAACCAGGCTTTCACCAGACCCCCACCTCCCTATCCTGGGAACA
TTAGGTCTCCTGTTGCCCCTCCTTTAGGACCTAGATATGCTGTTTTCCC
AAAAGATCAGCGTGGACCCTATCCTCCTGATGTTGCTAGTATGGGGATG
AGACCTCATGGATTTAGATTTGGATTTCCAGGAGGTAGTCATGGTACCA
TGCCGAGTCAAGAGCGCTTCCTTGTGCCTCCTCAGCAAATACAGGGATC
TGGAGTTTCTCCACAGCTAAGAAGATCAGTATCTGTAGATATGCCTAGG
CCTTTAAATAACTCACAAATGAATAATCCAGTTGGACTTCCTCAGCATT
TTTCACCACAGAGCTTGCCAGTTCAGCAGCACAACATACTGGGCCAAGC
ATATATTGAACTGAGACATAGGGCTCCTGACGGAAGGCAACGGCTGCCT
TTCAGTGCTCCACCTGGCAGCGTTGTAGAGGCATCTTCTAATCTGAGAC
ATGGAAACTTCATTCCCCGGCCAGACTTTCCGGGCCCTAGACACACAGA
CCCCATGCGACGACCTCCCCAGGGTCTACCTAATCAGCTACCTGTGCAC
CCAGATTTGGAACAAGTGCCACCATCTCAACAAGAGCAAGGTCATTCTG
TCCATTCATCTTCTATGGTCATGAGGACTCTGAACCATCCACTAGGTGG
TGAATTTTCAGAAGCTCCTTTGTCAACATCTGTACCGTCTGAAACAACG
TCTGATAATTTACAGATAACCACCCAGCCTTCTGATGGTCTAGAGGAAA
AACTTGATTCTGATGACCCTTCTGTGAAGGAACTGGATGTTAAAGACCT
TGAGGGGGTTGAAGTCAAAGACTTAGATGATGAAGATCTTGAAAACTTA
AATTTAGATACAGAGGATGGCAAGGTAGTTGAATTGGATACTTTAGATA
ATTTGGAAACTAATGATCCCAACCTGGATGACCTCTTAAGGTCAGGAGA
GTTTGATATCATTGCATATACAGATCCAGAACTTGACATGGGAGATAAG
AAAAGCATGTTTAATGAGGAACTAGACCTTCCAATTGATGATAAGTTAG
ATAATCAGTGTGTATCTGTTGAACCAAAAAAAAAGGAACAAGAAAACAA
AACTCTGGTTCTCTCTGATAAACATTCACCACAGAAAAAATCCACTGTT
ACCAATGAGGTAAAAACGGAAGTACTGTCTCCAAATTCTAAGGTGGAAT
CCAAATGTGAAACTGAAAAAAATGATGAGAATAAAGATAATGTTGACAC
TCCTTGCTCACAGGCTTCTGCTCACTCAGACCTAAATGATGGAGAAAAG
ACTTCTTTGCATCCTTGTGATCCAGATCTATTTGAGAAAAGAACCAATC
GAGAAACTGCTGGCCCCAGTGCAAATGTCATTCAGGCATCCACTCAACT
ACCTGCTCAAGATGTAATAAACTCTTGTGGCATAACTGGATCAACTCCA
GTTCTCTCAAGTTTACTTGCTAATGAGAAATCTGATAATTCAGACATTA
GGCCATCGGGGTCTCCACCACCACCAACTCTGCCGGCCTCCCCATCCAA
TCATGTGTCAAGTTTGCCTCCTTTCATAGCACCGCCTGGCCGTGTTTTG
GATAATGCCATGAATTCTAATGTGACAGTAGTCTCTAGGGTAAACCATG
TTTTTTCTCAGGGTGTGCAGGTAAACCCAGGGCTCATTCCAGGTCAATC
AACAGTTAACCACAGTCTGGGGACAGGAAAACCTGCAACTCAAACTGGG
CCTCAAACAAGTCAGTCTGGTACCAGTAGCATGTCTGGACCCCAACAGC
TAATGATTCCTCAAACATTAGCACAGCAGAATAGAGAGAGGCCCCTTCT
TCTAGAAGAACAGCCTCTACTTCTACAGGATCTTTTGGATCAAGAAAGG
CAAGAACAGCAGCAGCAAAGACAGATGCAAGCCATGATTCGTCAGCGAT
CAGAACCGTTCTTCCCTAATATTGATTTTGATGCAATTACAGATCCTAT
AATGAAAGCCAAAATGGTGGCCCTTAAAGGTATAAATAAAGTGATGGCA
CAAAACAATCTGGGCATGCCACCAATGGTGATGAGCAGGTTCCCTTTTA
TGGGCCAGGTGGTAACTGGAACACAGAACAGTGAAGGACAGAACCTTGG
ACCACAGGCCATTCCTCAGGATGGCAGTATAACACATCAGATTTCTAGG
CCTAATCCTCCAAATTTTGGTCCAGGCTTTGTCAATGATTCACAGCGTA
AGCAGTATGAAGAGTGGCTCCAGGAGACCCAACAGCTGCTTCAAATGCA
GCAGAAGTATCTTGAAGAACAAATTGGTGCTCACAGAAAATCTAAGAAG
GCCCTTTCAGCTAAACAACGTACTGCCAAGAAAGCTGGGCGTGAATTTC
CAGAGGAAGATGCAGAACAACTCAAGCATGTTACTGAACAGCAAAGCAT
GGTTCAGAAACAGCTAGAACAGATTCGTAAACAACAGAAAGAACATGCT
GAATTGATTGAAGATTATCGGATCAAACAGCAGCAGCAATGTGCAATGG
CCCCACCTACCATGATGCCCAGTGTCCAGCCCCAGCCACCCCTAATTCC
AGGTGCCACTCCACCCACCATGAGCCAACCCACCTTTCCCATGGTGCCA
CAGCAGCTTCAGCACCAGCAGCACACAACAGTTATTTCTGGCCATACTA
GCCCTGTTAGAATGCCCAGTTTACCTGGATGGCAACCCAACAGTGCTCC
TGCCCACCTGCCCCTCAATCCTCCTAGAATTCAGCCCCCAATTGCCCAG
TTACCAATAAAAACTTGTACACCAGCCCCAGGGACAGTCTCAAATGCAA
ATCCACAGAGTGGACCACCACCTCGGGTAGAATTTGATGACAACAATCC
CTTTAGTGAAAGTTTTCAAGAACGGGAACGTAAGGAACGTTTACGAGAA
CAGCAAGAGAGACAACGGATCCAACTCATGCAGGAGGTAGATAGACAAA
GAGCTTTGCAGCAGAGGATGGAAATGGAGCAGCATGGTATGGTGGGCTC
TGAGATAAGTAGTAGTAGGACATCTGTGTCCCAGATTCCCTTCTACAGT
TCCGACTTACCTTGTGATTTTATGCAACCTCTAGGACCCCTTCAGCAGT
CTCCACAACACCAACAGCAAATGGGGCAGGTTTTACAGCAGCAGAATAT
ACAACAAGGATCAATTAATTCACCCTCCACCCAAACTTTCATGCAGACT
AATGAGCGAAGGCAGGTAGGCCCTCCTTCATTTGTTCCTGATTCACCAT
CAATCCCTGTTGGAAGCCCAAATTTTTCTTCTGTGAAGCAGGGACATGG
AAATCTTTCTGGGACCAGCTTCCAGCAGTCCCCAGTGAGGCCTTCTTTT
ACACCTGCTTTACCAGCAGCACCTCCAGTAGCTAATAGCAGTCTCCCAT
GTGGCCAAGATTCTACTATAACCCATGGACACAGTTATCCGGGATCAAC
CCAATCGCTCATTCAGTTGTATTCTGATATAATCCCAGAGGAAAAAGGG
AAAAAGAAAAGAACAAGAAAGAAGAAAAGAGATGATGATGCAGAATCCA
CCAAGGCTCCATCAACTCCCCATTCAGATATAACTGCCCCACCGACTCC
AGGCATCTCAGAAACTACCTCTACTCCTGCAGTGAGCACACCCAGTGAG
CTTCCTCAACAAGCCGACCAAGAGTCGGTGGAACCAGTCGGCCCATCCA
CTCCCAATATGGCAGCAGGCCAGCTATGTACAGAATTAGAGAACAAACT
GCCCAATAGTGATTTCTCACAAGCAACTCCAAATCAACAGACGTATGCA
AATTCAGAAGTAGACAAGCTCTCCATGGAAACCCCTGCCAAAACAGAAG
AGATAAAACTGGAAAAGGCTGAGACAGAGTCCTGCCCAGGCCAAGAGGA
GCCTAAATTGGAGGAACAGAATGGTAGTAAGGTAGAAGGAAACGCTGTA
GCCTGTCCTGTCTCCTCAGCACAGAGTCCTCCCCATTCTGCTGGGGCCC
CTGCTGCCAAAGGAGACTCAGGGAATGAACTTCTGAAACACTTGTTGAA
AAATAAAAAGTCATCTTCTCTTTTGAATCAAAAACCTGAGGGCAGTATT
TGTTCAGAAGATGACTGTACAAAGGATAATAAACTAGTTGAGAAGCAGA
ACCCAGCTGAAGGACTGCAAACTTTGGGGGCTCAAATGCAAGGTGGTTT
TGGATGTGGCAACCAGTTGCCAAAAACAGATGGAGGAAGTGAAACCAAG
AAACAGCGAAGCAAACGGACTCAGAGGACGGGTGAGAAAGCAGCACCTC
GCTCAAAGAAAAGGAAAAAGGACGAAGAGGAGAAACAAGCTATGTACTC
TAGCACTGACACGTTTACCCACTTGAAACAGCAGAATAATTTAAGTAAT
CCTCCAACACCCCCTGCCTCTCTTCCTCCTACACCACCTCCTATGGCTT
GTCAGAAGATGGCCAATGGTTTTGCAACAACTGAAGAACTTGCTGGAAA
AGCCGGAGTGTTAGTGAGCCATGAAGTTACCAAAACTCTAGGACCTAAA
CCATTTCAGCTGCCCTTCAGACCCCAGGACGACTTGTTGGCCCGAGCTC
TTGCTCAGGGCCCCAAGACAGTTGATGTGCCAGCCTCCCTCCCAACACC
ACCTCATAACAATCAGGAAGAATTAAGGATACAGGATCACTGTGGTGAT
CGAGATACTCCTGACAGTTTTGTTCCCTCATCCTCTCCTGAGAGTGTGG
TTGGGGTAGAAGTGAGCAGGTATCCAGATCTGTCATTGGTCAAGGAGGA
GCCTCCAGAACCGGTGCCGTCCCCCATCATTCCAATTCTTCCTAGCACT
GCTGGGAAAAGTTCAGAATCAAGAAGGAATGACATCAAAACTGAGCCAG
GCACTTTATATTTTGCGTCACCTTTTGGTCCTTCCCCAAATGGTCCCAG
ATCAGGTCTTATATCTGTAGCAATTACTCTGCATCCTACAGCTGCTGAG
AACATTAGCAGTGTTGTGGCTGCATTTTCCGACCTTCTTCACGTCCGAA
TCCCTAACAGCTATGAGGTTAGCAGTGCTCCAGATGTCCCATCCATGGG
TTTGGTCAGTAGCCACAGAATCAACCCGGGTTTGGAGTATCGACAGCAT
TTACTTCTCCGTGGGCCTCCGCCAGGATCTGCAAACCCTCCCAGATTAG
TGAGCTCTTACCGGCTGAAGCAGCCTAATGTACCATTTCCTCCAACAAG
CAATGGTCTTTCTGGATATAAGGATTCTAGTCATGGTATTGCAGAAAGC
GCAGCACTCAGACCACAGTGGTGTTGTCATTGTAAAGTGGTTATTCTTG
GAAGTGGTGTGCGGAAATCTTTCAAAGATCTGACCCTTTTGAACAAGGA
TTCCCGAGAAAGCACCAAGAGGGTAGAGAAGGACATTGTCTTCTGTAGT
AATAACTGCTTTATTCTTTATTCATCAACTGCACAAGCGAAAAACTCAG
AAAACAAGGAATCCATTCCTTCATTGCCACAATCACCTATGAGAGAAAC
GCCTTCCAAAGCATTTCATCAGTACAGCAACAACATCTCCACTTTGGAT
GTGCACTGTCTCCCCCAGCTCCCAGAGAAAGCTTCTCCCCCTGCCTCAC
CACCCATCGCCTTCCCTCCTGCTTTTGAAGCAGCCCAAGTCGAGGCCAA
GCCAGATGAGCTGAAGGTGACAGTCAAGCTGAAGCCTCGGCTAAGAGCT
GTCCATGGTGGGTTTGAAGATTGCAGGCCGCTCAATAAAAAATGGAGAG
GAATGAAATGGAAGAAGTGGAGCATTCATATTGTAATCCCTAAGGGGAC
ATTTAAACCACCTTGTGAGGATGAAATAGATGAATTTCTAAAGAAATTG
GGCACTTCCCTTAAACCTGATCCTGTGCCCAAAGACTATCGGAAATGTT
GCTTTTGTCATGAAGAAGGTGATGGATTGACAGATGGACCAGCAAGGCT
ACTCAACCTTGACTTGGATCTGTGGGTCCACTTGAACTGCGCTCTGTGG
TCCACGGAGGTCTATGAGACTCAGGCTGGTGCCTTAATAAATGTGGAGC
TAGCTCTGAGGAGAGGCCTACAAATGAAATGTGTCTTCTGTCACAAGAC
GGGTGCCACTAGTGGATGCCACAGATTTCGATGCACCAACATTTATCAC
TTCACTTGCGCCATTAAAGCACAATGCATGTTTTTTAAGGACAAAACTA
TGCTTTGCCCCATGCACAAACCAAAGGGAATTCATGAGCAAGAATTAAG
TTACTTTGCAGTCTTCAGGAGGGTCTATGTTCAGCGTGATGAGGTGCGA
CAGATTGCTAGCATCGTGCAACGAGGAGAACGGGACCATACCTTTCGCG
TGGGTAGCCTCATCTTCCACACAATTGGTCAGCTGCTTCCACAGCAGAT
GCAAGCATTCCATTCTCCTAAAGCACTCTTCCCTGTGGGCTATGAAGCC
AGCCGGCTGTACTGGAGCACTCGCTATGCCAATAGGCGCTGCCGCTACC
TGTGCTCCATTGAGGAGAAGGATGGGCGCCCAGTGTTTGTCATCAGGAT
TGTGGAACAAGGCCATGAAGACCTGGTTCTAAGTGACATCTCACCTAAA
GGTGTCTGGGATAAGATTTTGGAGCCTGTGGCATGTGTGAGAAAAAAGT
CTGAAATGCTCCAGCTTTTCCCAGCGTATTTAAAAGGAGAGGATCTGTT
TGGCCTGACCGTCTCTGCAGTGGCACGCATAGCGGAATCACTTCCTGGG
GTTGAGGCATGTGAAAATTATACCTTCCGATACGGCCGAAATCCTCTCA
TGGAACTTCCTCTTGCCGTTAACCCCACAGGTTGTGCCCGTTCTGAACC
TAAAATGAGTGCCCATGTCAAGAGGTTTGTGTTAAGGCCTCACACCTTA
AACAGCACCAGCACCTCAAAGTCATTTCAGAGCACAGTCACTGGAGAAC
TGAACGCACCTTATAGTAAACAGTTTGTTCACTCCAAGTCATCGCAGTA
CCGGAAGATGAAAACTGAATGGAAATCCAATGTGTATCTGGCACGGTCT
CGGATTCAGGGGCTGGGCCTGTATGCTGCTCGAGACATTGAGAAACACA
CCATGGTCATTGAGTACATCGGGACTATCATTCGAAACGAAGTAGCCAA
CAGGAAAGAGAAGCTTTATGAGTCTCAGAACCGTGGTGTGTACATGTTC
CGCATGGATAACGACCATGTGATTGACGCGACGCTCACAGGAGGGCCCG
CAAGGTATATCAACCATTCGTGTGCACCTAATTGTGTGGCTGAAGTGGT
GACTTTTGAGAGAGGACACAAAATTATCATCAGCTCCAGTCGGAGAATC
CAGAAAGGAGAAGAGCTCTGCTATGACTATAAGTTTGACTTTGAAGATG
ACCAGCACAAGATTCCGTGTCACTGTGGAGCTGTGAACTGCCGGAAGTG
GATGAACTGAAATGCATTCCTTGCTAGCTCAGCGGGCGGCTTGTCCCTA
GGAAGAGGCGATTCAACACACCATTGGAATTTTGCAGACAGAAAGAGAT
TTTTGTTTTCTGTTTTATGACTTTTTGAAAAAGCTTCTGGGAGTTCTGA
TTTCCTCAGTCCTTTAGGTTAAAGCAGCGCCAGGAGGAAGCTGACAGAA
GCAGCGTTCCTGAAGTGGCCGAGGTTAAACGGAATCACAGAATGGTCCA
GCACTTTTGCTTTTTTTTCTTTTCCTTTTCTTTTTTTTTTGTTTGTTTT
TTGTTTTGTTTTTCCCTTGTGGGTGGGTTTCATTGTTTTGGTTTTCTAG
TCTCACTAAGGAGAAACTTTTACTGGGGCAAAGAGCCGATGGCTGCCCT
GCCCCGGGCAGGGGCCTTCCTATGAATGTAAGACTGAAATCACCAGCGA
GGGGGACAGAGAGTGCTGGCCACGGCCTTATTAAAAAGGGGCAGGCCCT
CTAACTTCAAAATGTTTTTAAATAAAGTAGACACCACTGAACAAGGAAT
GTACTGAAATGACTTCCTTAGGGATAGAGCTAAGGGATAATAACTTGCA
CTAAATACATTTAAATACTTGATTCCATGAGTCAGTTTATTGTAGTTTT
TGATTTCTGTAAAATAAGAGAAACTTTTGTATTTATTATTGAATAAGTG
AATGAAGCTATTTTTAAATAAAGTTAGAAGAAAGCCAAGCTGCTGCTGT
TACCTGCAGAACTAACAAACCCTGTTACTTTGTACAGATATGTAAATAT
TTTGAGAAAAAATACAGTATAAAAATAGTTATTGACCAAATGCTACCAG
GCTCTGCAGCAGCTCGGGGGCTTATAAAATGTTCATAGGGATGTTACAA
TATAATTTTGTGTTATAAAATATGCCATTATAATTATGTAATAACCAAA
ATTTCAACCTAGAGTGTTGGGGGTTTTTTGGAAACCGCAGTCTATTAGT
ACTCAATGGTTTTATACACCTTACTTCTGACAGAGCGGGGCGTATGCTA
CGACTACAACTTTTATAGCTGTTTTGGTAATTTAAACTAATTTTTTCAT
ATTATATTGTTGCATCCCTACTTCTTCAGTCAGGTTTTTTTGTGCTTAC
AATTTGTGATAACTGTGAATAACTGCTTAAAAATACACCCAAATGGAGG
CTGAATTTTTTCTTCAGCAAAAGTAGTTTTGATTAGAACTTTGTTTCAG
CCACAGAGAATCATGTAAACGTAATAGGATCATGTAGCAGAAACTTAAA
TCTAACCCTTTAGCCTTCTATTTAACACAAAAATTTGAAAAAGTTAAAA
AAAAAAAGGAGATGTGATTATGCTTACAGCTGCAGGACTCTGGCAATAG
GGTTTTTGGAAGATGTAATTTTAAAATGTGTTTGTATGAACTGTTTGTT
TACATTTCTTTAATAAAAAAAACACTGTTTTGTGTTTGCTTGTAGAAAC
TTAATCAGCATTTTGAACCAGGTTAGCTTTTTATTTTGTACTTAAAATT
CTGGTACTGACACTTCACAGGCTAAGTATAAAATGAAGTTTTGTGTGCA
CAATTCAAGTGGACTGTAAACTGTTGGTATATTCAGTGATGCAGTTCTG
AACTTGTATATGGCATGATGTATTTTTATCTTACAGAATAAATCAATTG
TATATATTTTTCTCTTGATAAATAGCTGTATGAAATTTGTTTCCTGAAT
ATTTTTCTTCTCTTGTACAATATCCTGACATCCTACCAGTATTTGTCCT
ACCGGGTTTTTGTTGTTTTCTGTTCTGTATAATAGTATCTAATGTTGGC
AAAAATTGAATTTTTTGAAGTATACAGAGTGTTATGGGTTTTGGAATTT
GTGGACACAGATTTAGAAGATCACCATTTACAAATAAAATATTTTACAT
CTATAA

Transcript: MLL3-001 ENST00000262189

Protein sequence (SEQ ID NO.: 118), part of fusion
gene is shaded.
MSSEEDKSVEQPQPPPPPPEEPGAPAPSPAAADKRPRGRPRKDGASPFQR
ARKKPRSRGKTAVEDEDSMDGLETTETETIVETEIKEQSAEEDAEAEVDN
SKQLIPTLQRSVSEESANSLVSVGVEAKISEQLCAFCYCGEKSSLGQGDL
KQFRITPGFILPWRNQPSNKKDIDDNSNGTYEKMQNSAPRKQRGQRKERS
PQQNIVSCVSVSTQTASDDQAGKLWDELSLVGLPDAIDIQALFDSTGTCW
AHHRCVEWSLGVCQMEEPLLVNVDKAVVSGSTERCAFCKHLGATIKCCEE
KCTQMYHYPCAAGAGTFQDFSHIFLLCPEHIDQAPERSKEDANCAVCDSP
GDLLDQFFCTTCGQHYHGMCLDIAVTPLKRAGWQCPECKVCQNCKQSGED
SKMLVCDTCDKGYHTFCLQPVMKSVPTNGWKCKNCRICIECGTRSSSQWH
HNCLICDNCYQQQDNLCPFCGKCYHPELQKDMLHCNMCKRWVHLECDKPT
DHELDTQLKEEYICMYCKHLGAEMDRLQPGEEVEIAELTTDYNNEMEVEG
PEDQMVFSEQAANKDVNGQESTPGIVPDAVQVHTEEQQKSHPSESLDTDS
LLIAVSSQHTVNTELEKQISNEVDSEDLKMSSEVKHICGEDQIEDKMEVT
ENIEVVTHQITVQQEQLQLLEEPETVVSREESRPPKLVMESVTLPLETLV
SPHEESISLCPEEQLVIERLQGEKEQKENSELSTGLMDSEMTPTIEGCVK
DVSYQGGKSIKLSSETESSFSSSADISKADVSSSPTPSSDLPSHDMLHNY
PSALSSSAGNIMPTTYISVTPKIGMGKPAITKRKFSPGRPRSKQGAWSTH
NTVSPPSWSPDISEGREIFKPRQLPGSAIWSIKVGRGSGFPGKRRPRGAG
LSGRGGRGRSKLKSGIGAVVLPGVSTADISSNKDDEENSMHNTVVLFSSS
DKFTLNQDMCVVCGSFGQGAEGRLLACSQCGQCYHPYCVSIKITKVVLSK
GWRCLECTVCEACGKATDPGRLLLCDDCDISYHTYCLDPPLQTVPKGGWK
CKWCVWCRHCGATSAGLRCEWQNNYTQCAPCASLSSCPVCYRNYREEDLI
LQCRQCDRWMHAVCQNLNTEEEVENVADIGFDCSMCRPYMPASNVPSSDC
CESSLVAQIVTKVKELDPPKTYTQDGVCLTESGMTQLQSLTVTVPRRKRS
KPKLKLKIINQNSVAVLQTPPDIQSEHSRDGEMDDSREGELMDCDGKSES
SPEREAVDDETKGVEGTDGVKKRKRKPYRPGIGGFMVRQRSRTGQGKTKR
SVIRKDSSGSISEQLPCRDDGWSEQLPDTLVDESVSVTESTEKIKKRYRK
RKNKLEETFPAYLQEAFFGKDLLDTSRQSKISLDNLSEDGAQLLYKTNMN
TGFLDPSLDPLLSSSSAPTKSGTHGPADDPLADISEVLNTDDDILGIISD
DLAKSVDHSDIGPVTDDPSSLPQPNVNQSSRPLSEEQLDGILSPELDKMV
TDGAILGKLYKIPELGGKDVEDLFTAVLSPANTQPTPLPQPPPPTQLLPI
HNQDAFSRMPLMNGLIGSSPHLPHNSLPPGSGLGTFSAIAQSSYPDARDK
NSAFNPMASDPNNSWTSSAPTVEGENDTMSNAQRSTLKWEKEEALGEMAT
VAPVLYTNINFPNLKEEFPDWTTRVKQIAKLWRKASSQERAPYVQKARDN
RAALRINKVQMSNDSMKRQQQQDSIDPSSRIDSELFKDPLKQRESEHEQE
WKFRQQMRQKSKQQAKIEATQKLEQVKNEQQQQQQQQFGSQHLLVQSGSD
TPSSGIQSPLTPQPGNGNMSPAQSFHKELFTKQPPSTPTSTSSDDVFVKP
QAPPPPPAPSRIPIQDSLSQAQTSQPPSPQVFSPGSSNSRPPSPMDPYAK
MVGTPRPPPVGHSFSRRNSAAPVENCTPLSSVSRPLQMNETTANRPSPVR
DLCSSSTTNNDPYAKPPDTPRPVMTDQFPKSLGLSRSPVVSEQTAKGPIA
AGTSDHFTKPSPRADVFQRQRIPDSYARPLLTPAPLDSGPGPFKTPMQPP
PSSQDPYGSVSQASRRLSVDPYERPALTPRPIDNFSHNQSNDPYSQPPLT
PHPAVNESFAHPSRAFSQPGTISRPTSQDPYSQPPGTPRPVVDSYSQSSG
TARSNTDPYSQPPGTPRPTTVDPYSQQPQTPRPSTQTDLFVTPVTNQRHS
DPYAHPPGTPRPGISVPYSQPPATPRPRISEGFTRSSMTRPVLMPNQDPF
LQAAQNRGPALPGPLVRPPDTCSQTPRPPGPGLSDTFSRVSPSAARDPYD
QSPMTPRSQSDSFGTSQTAHDVADQPRPGSEGSFCASSNSPMHSQGQQFS
GVSQLPGPVPTSGVTDTQNTVNMAQADTEKLRQRQKLREIILQQQQQKKI
AGRQEKGSQDSPAVPHPGPLQHWQPENVNQAFTRPPPPYPGNIRSPVAPP
LGPRYAVFPKDQRGPYPPDVASMGMRPHGFRFGFPGGSHGTMPSQERFLV
PPQQIQGSGVSPQLRRSVSVDMPRPLNNSQMNNPVGLPQHFSPQSLPVQQ
HNILGQAYIELRHRAPDGRQRLPFSAPPGSVVEASSNLRHGNFIPRPDFP
GPRHTDPMRRPPQGLPNQLPVHPDLEQVPPSQQEQGHSVHSSSMVMRTLN
HPLGGEFSEAPLSTSVPSETTSDNLQITTQPSDGLEEKLDSDDPSVKELD
VKDLEGVEVKDLDDEDLENLNLDTEDGKVVELDTLDNLETNDPNLDDLLR
SGEFDIIAYTDPELDMGDKKSMFNEELDLPIDDKLDNQCVSVEPKKKEQE
NKTLVLSDKHSPQKKSTVTNEVKTEVLSPNSKVESKCETEKNDENKDNVD
TPCSQASAHSDLNDGEKTSLHPCDPDLFEKRTNRETAGPSANVIQASTQL
PAQDVINSCGITGSTPVLSSLLANEKSDNSDIRPSGSPPPPTLPASPSNH
VSSLPPFIAPPGRVLDNAMNSNVTVVSRVNHVFSQGVQVNPGLIPGQSTV
NHSLGTGKPATQTGPQTSQSGTSSMSGPQQLMIPQTLAQQNRERPLLLEE
QPLLLQDLLDQERQEQQQQRQMQAMIRQRSEPFFPNIDFDAITDPIMKAK
MVALKGINKVMAQNNLGMPPMVMSRFPFMGQVVTGTQNSEGQNLGPQAIP
QDGSITHQISRPNPPNFGPGFVNDSQRKQYEEWLQETQQLLQMQQKYLEE
QIGAHRKSKKALSAKQRTAKKAGREFPEEDAEQLKHVTEQQSMVQKQLEQ
IRKQQKEHAELIEDYRIKQQQQCAMAPPTMMPSVQPQPPLIPGATPPTMS
QPTFPMVPQQLQHQQHTTVISGHTSPVRMPSLPGWQPNSAPAHLPLNPPR
IQPPIAQLPIKTCTPAPGTVSNANPQSGPPPRVEFDDNNPFSESFQERER
KERLREQQERQRIQLMQEVDRQRALQQRMEMEQHGMVGSEISSSRTSVSQ
IPFYSSDLPCDFMQPLGPLQQSPQHQQQMGQVLQQQNIQQGSINSPSTQT
FMQTNERRQVGPPSFVPDSPSIPVGSPNFSSVKQGHGNLSGTSFQQSPVR
PSFTPALPAAPPVANSSLPCGQDSTITHGHSYPGSTQSLIQLYSDIIPEE
KGKKKRTRKKKRDDDAESTKAPSTPHSDITAPPTPGISETTSTPAVSTPS
ELPQQADQESVEPVGPSTPNMAAGQLCTELENKLPNSDFSQATPNQQTYA
NSEVDKLSMETPAKTEEIKLEKAETESCPGQEEPKLEEQNGSKVEGNAVA
CPVSSAQSPPHSAGAPAAKGDSGNELLKHLLKNKKSSSLLNQKPEGSICS
EDDCTKDNKLVEKQNPAEGLQTLGAQMQGGFGCGNQLPKTDGGSETKKQR
SKRTQRTGEKAAPRSKKRKKDEEEKQAMYSSTDTFTHLKQQNNLSNPPTP
PASLPPTPPPMACQKMANGFATTEELAGKAGVLVSHEVTKTLGPKPFQLP
FRPQDDLLARALAQGPKTVDVPASLPTPPHNNQEELRIQDHCGDRDTPDS
FVPSSSPESVVGVEVSRYPDLSLVKEEPPEPVPSPIIPILPSTAGKSSES
RRNDIKTEPGTLYFASPFGPSPNGPRSGLISVAITLHPTAAENISSVVAA
FSDLLHVRIPNSYEVSSAPDVPSMGLVSSHRINPGLEYRQHLLLRGPPPG
SANPPRLVSSYRLKQPNVPFPPTSNGLSGYKDSSHGIAESAALRPQWCCH
CKVVILGSGVRKSFKDLTLLNKDSRESTKRVEKDIVFCSNNCFILYSSTA
QAKNSENKESIPSLPQSPMRETPSKAFHQYSNNISTLDVHCLPQLPEKAS
PPASPPIAFPPAFEAAQVEAKPDELKVTVKLKPRLRAVHGGFEDCRPLNK
KWRGMKWKKWSIHIVIPKGTFKPPCEDEIDEFLKKLGTSLKPDPVPKDYR
KCCFCHEEGDGLTDGPARLLNLDLDLWVHLNCALWSTEVYETQAGALINV
ELALRRGLQMKCVFCHKTGATSGCHRFRCTNIYHFTCAIKAQCMFFKDKT
MLCPMHKPKGIHEQELSYFAVFRRVYVQRDEVRQIASIVQRGERDHTFRV
GSLIFHTIGQLLPQQMQAFHSPKALFPVGYEASRLYWSTRYANRRCRYLC
SIEEKDGRPVFVIRIVEQGHEDLVLSDISPKGVWDKILEPVACVRKKSEM
LQLFPAYLKGEDLFGLTVSAVARIAESLPGVEACENYTFRYGRNPLMELP
LAVNPTGCARSEPKMSAHVKRFVLRPHTLNSTSTSKSFQSTVTGELNAPY
SKQFVHSKSSQYRKMKTEWKSNVYLARSRIQGLGLYAARDIEKHTMVIEY
IGTIIRNEVANRKEKLYESQNRGVYMFRMDNDHVIDATLTGGPARYINHS
CAPNCVAEVVTFERGHKIIISSSRRIQKGEELCYDYKFDFEDDQHKIPCH
CGAVNCRKWMN

Transcript: PRKAG2-001 ENST00000287878

cDNA sequence (SEQ ID NO.: 119). part of fusion gene is shaded.
GAGCTGGTTTATTCTGCGGCCGAGGATTACATTTATGCACGAACGGGCTTACTGGTTCCA
GATTCCCCACTTGGGCACAGGCATAGGAGGCTTGTTTTCCAAATTGCTGGTTTTAATTGC
ACCTGCCTTTCAGATTACCTCTGGGAATCTGTGGGAGGAGCCGAGAGGGTGGAAAATGTT
TCTTAGCTTTGCAAAAGGAAGAAAACTTTGTCACCCAGCGGGAGACCTCAGCCACGAGTA
ACCCGGGGAGACACCAGAACCGGGACGGGCTTTGACTGATTTGCCTACGAGGGTTCCGTA
GGAAAGGACGCTTGAATTCGGCGCTTCGGCGGCGGCGGCGGCCGCGCGAGTTCCCTGCTC
ACCCTCCCTCTCCGCGGAAGTCCCCACGAGGTGGCTTCAGGGTGTAACAGAGCGCGCGGC
TCCAGTCCGAAGGCAGCGGCCGGGGGAGGGAAGGAGGGGACCGAACCCCCGAGGAGTTTC
GCAGAATCAACTTCTGGTTAGAGTTATGGGAAGCGCGGTTATGGACACCAAGAAGAAAAA
AGATGTTTCCAGCCCCGGCGGGAGCGGCGGCAAGAAAAATGCCAGCCAGAAGAGGCGTTC
GCTGCGCGTGCACATTCCGGACCTGAGCTCCTTCGCCATGCCGCTCCTGGACGGAGACCT
GGAGGGTTCCGGAAAGCATTCCTCTCGAAAGGTGGACAGCCCCTTCGGCCCGGGCAGCCC
CTCCAAAGGGTTCTTCTCCAGAGGCCCCCAGCCCCGGCCCTCCAGCCCCATGTCTGCACC
TGTGAGGCCCAAGACCAGCCCCGGCTCTCCCAAAACCGTGTTCCCGTTCTCCTACCAGGA
GTCCCCGCCACGCTCCCCTCGACGCATGAGCTTCAGTGGGATCTTCCGCTCCTCCTCCAA
AGAGTCTTCCCCCAACTCCAACCCTGCTACCTCGCCCGGGGGCATCAGGTTTTTCTCCCG
CTCCAGAAAAACCTCCGGCCTCTCCTCCTCTCCGTCAACACCCACCCAAGTGACCAAGCA
GCACACGTTTCCCCTGGAATCCTATAAGCACGAGCCTGAACGGTTAGAGAATCGCATCTA
TGCCTCGTCTTCCCCCCCGGACACAGGGCAGAGGTTCTGCCCGTCTTCCTTCCAGAGCCC

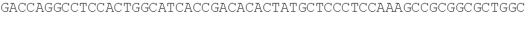

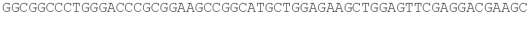

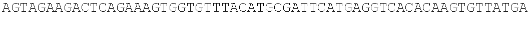

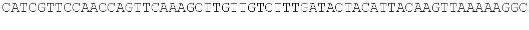

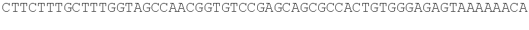

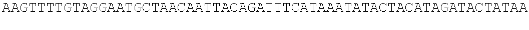

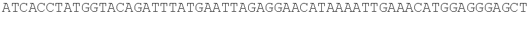

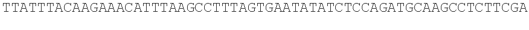

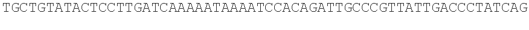

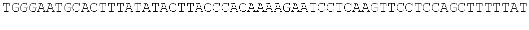

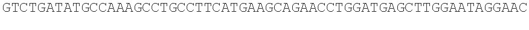

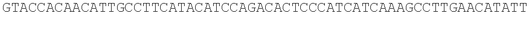

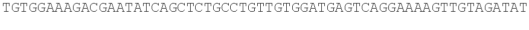

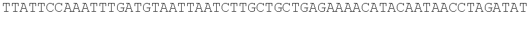

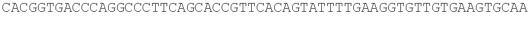

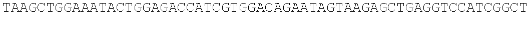

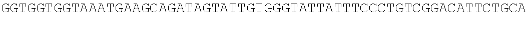

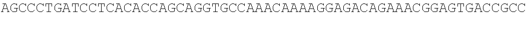

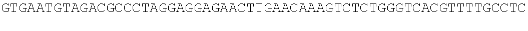

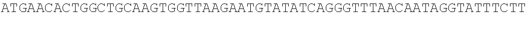

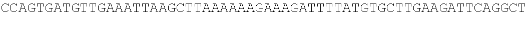

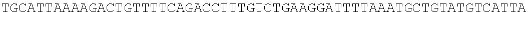

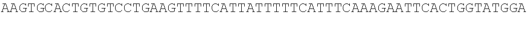

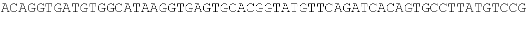

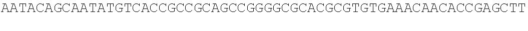

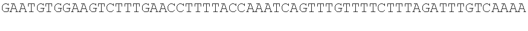

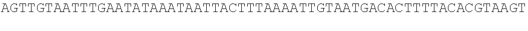

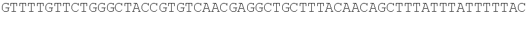

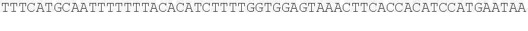

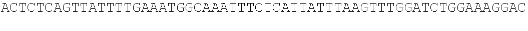

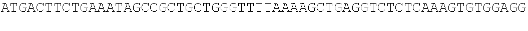

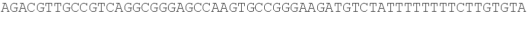

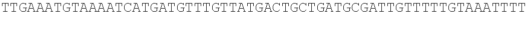

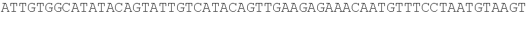

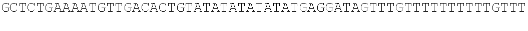

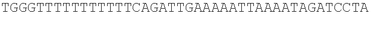

Transcript: PRKAG2-001 ENST00000287878

Protein sequence (SEQ ID NO.: 120), part of fusion gene is shaded.
MGSAVMDTKKKKDVSSPGGSGGKKNASQKRRSLRVHIPDLSSFAMPLLDGDLEGSGKHSS
RKVDSPFGPGSPSKGFFSRGPQPRPSSPMSAPVRPKTSPGSPKTVFPFSYQESPPRSPRR
MSFSGIFRSSSKESSPNSNPATSPGGIRFFSRSRKTSGLSSSPSTPTQVTKQHTFPLESY

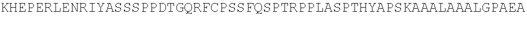

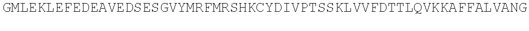

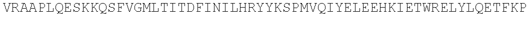

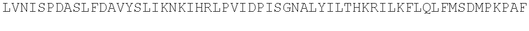

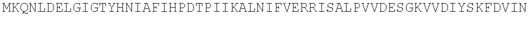

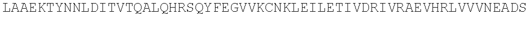

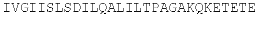

MLL3-PRKAG2 Fusion sequence exon 9 to exon 5

cDNA sequence (SEQ ID NO.: 121), PRKAG2 underlined.
ATGTCGTCGGAGGAGGACAAGAGCGTGGAGCAGCCGCAGCCGCCGCCACCACCCCCCGAGGAGCCTGGAGCCCCG
GCCCCGAGCCCCGCAGCCGCAGACAAAAGACCTCGGGGCCGGCCTCGCAAAGATGGCGCTTCCCCTTTCCAGAGA
GCCAGAAAGAAACCTCGAAGTAGGGGGAAAACTGCAGTGGAAGATGAGGACAGCATGGATGGGCTGGAGACAACA
GAAACAGAAACGATTGTGGAAACAGAAATCAAAGAACAATCTGCAGAAGAGGATGCTGAAGCAGAAGTGGATAAC
AGCAAACAGCTAATTCCAACTCTTCAGCGATCTGTGTCTGAGGAATCGGCAAACTCCCTGGTCTCTGTTGGTGTA
GAAGCCAAAATCAGTGAACAGCTCTGCGCTTTTTGTTACTGTGGGGAAAAAAGTTCCTTAGGACAAGGAGACTTA
AAACAATTCAGAATAACGCCTGGATTTATCTTGCCATGGAGAAACCAACCTTCTAACAAGAAGGACATTGATGAC
AACAGCAATGGAACCTATGAGAAAATGCAAAACTCAGCACCACGAAAACAAAGAGGACAGAGAAAAGAACGATCT
CCTCAGCAGAATATAGTATCTTGTGTAAGTGTAAGCACCCAGACAGCTTCAGATGATCAAGCTGGTAAACTGTGG
GATGAACTCAGTCTGGTTGGGCTTCCAGATGCCATTGATATCCAAGCCTTATTTGATTCTACAGGCACTTGTTGG
GCTCATCACCGTTGTGTGGAGTGGTCACTAGGAGTATGCCAGATGGAAGAACCATTGTTAGTGAACGTGGACAAA
GCTGTTGTCTCAGGGAGCACAGAACGATGTGCATTTTGTAAGCACCTTGGAGCCACTATCAAATGCTGTGAAGAG
AAATGTACCCAGATGTATCATTATCCTTGTGCTGCAGGAGCCGGCACCTTTCAGGATTTCAGTCACATCTTCCTG
CTTTGTCCAGAACACATTGACCAAGCTCCTGAAAGATCGAAGGAAGATGCAAACTGTGCAGTGTGCGACAGCCCG
GGAGACCTCTTAGATCAGTTCTTTTGTACTACTTGTGGTCAGCACTATCATGGAATGTGCCTGGATATAGCGGTT
ACTCCATTAAAACGTGCAGGTTGGCAATGTCCTGAGTGCAAAGTGTGCCAGAACTGCAAACAATCGGGAGAAGAT
AGCAAGATGCTAGTGTGTGATACGTGTGACAAAGGGTATCATACTTTTTGTCTTCAACCAGTTATGAAATCAGTA

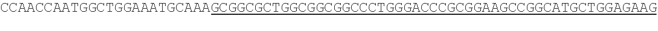

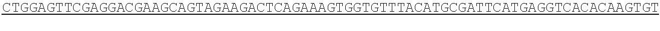

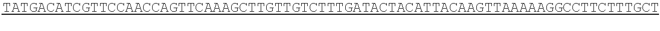

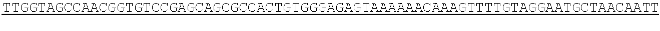

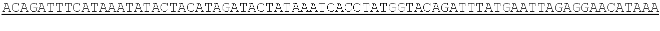

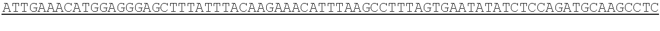

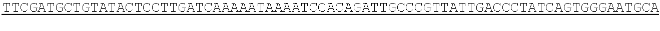

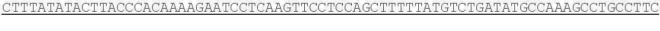

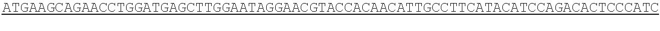

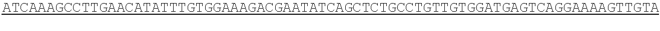

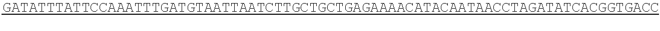

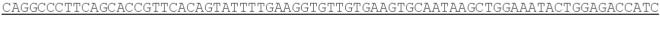

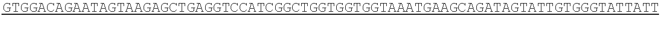

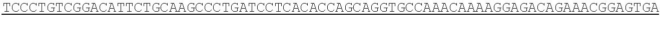

Protein sequence exon 9 to exon 5 (SEQ ID NO.: 122), PRKAG2 underlined.
MSSEEDKSVEQPQPPPPPPEEPGAPAPSPAAADKRPRGRPRKDGASPFQRARKKPRSRGKTAVEDEDSMDGLETT
ETETIVETEIKEQSAEEDAEAEVDNSKQLIPTLQRSVSEESANSLVSVGVEAKISEQLCAFCYCGEKSSLGQGDL
KQFRITPGFILPWRNQPSNKKDIDDNSNGTYEKMQNSAPRKQRGQRKERSPQQNIVSCVSVSTQTASDDQAGKLW
DELSLVGLPDAIDIQALFDSTGTCWAHHRCVEWSLGVCQMEEPLLVNVDKAVVSGSTERCAFCKHLGATIKCCEE
KCTQMYHYPCAAGAGTFQDFSHIFLLCPEHIDQAPERSKEDANCAVCDSPGDLLDQFFCTTCGQHYHGMCLDIAV

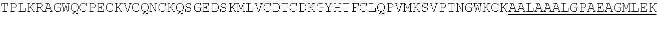

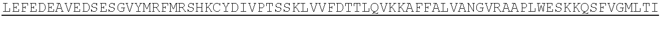

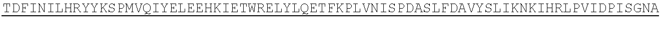

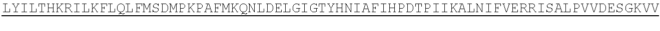

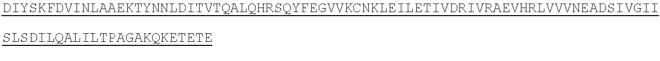

Protein Domain Exon 9 to Exon 5

Due to overlapping domains, there are 4 representations of the protein. No transmembrane domains.

MLL3-PRKAG2 Fusion sequence exon 6 to exon 7

cDNA sequence (SEQ ID NO.: 123), PRKAG2 underlined.
ATGTCGTCGGAGGAGGACAAGAGCGTGGAGCAGCCGCAGCCGCCGCCACCACCCCCCGAGGAGCCTGGAGCCCCG
GCCCCGAGCCCCGCAGCCGCAGACAAAAGACCTCGGGGCCGGCCTCGCAAAGATGGCGCTTCCCCTTTCCAGAGA
GCCAGAAAGAAACCTCGAAGTAGGGGGAAAACTGCAGTGGAAGATGAGGACAGCATGGATGGGCTGGAGACAACA
GAAACAGAAACGATTGTGGAAACAGAAATCAAAGAACAATCTGCAGAAGAGGATGCTGAAGCAGAAGTGGATAAC
AGCAAACAGCTAATTCCAACTCTTCAGCGATCTGTGTCTGAGGAATCGGCAAACTCCCTGGTCTCTGTTGGTGTA
GAAGCCAAAATCAGTGAACAGCTCTGCGCTTTTTGTTACTGTGGGGAAAAAAGTTCCTTAGGACAAGGAGACTTA
AAACAATTCAGAATAACGCCTGGATTTATCTTGCCATGGAGAAACCAACCTTCTAACAAGAAGGACATTGATGAC
AACAGCAATGGAACCTATGAGAAAATGCAAAACTCAGCACCACGAAAACAAAGAGGACAGAGAAAAGAACGATCT
CCTCAGCAGAATATAGTATCTTGTGTAAGTGTAAGCACCCAGACAGCTTCAGATGATCAAGCTGGTAAACTGTGG
GATGAACTCAGTCTGGTTGGGCTTCCAGATGCCATTGATATCCAAGCCTTATTTGATTCTACAGGCACTTGTTGG
GCTCATCACCGTTGTGTGGAGTGGTCACTAGGAGTATGCCAGATGGAAGAACCATTGTTAGTGAACGTGGACAAA

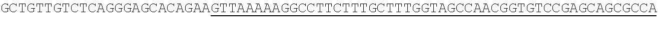

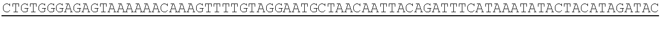

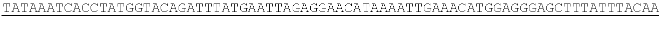

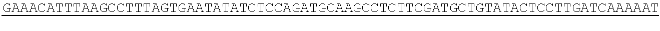

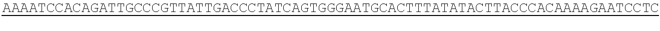

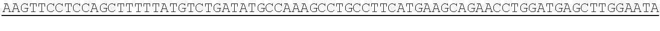

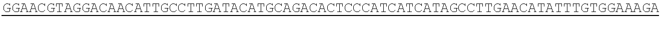

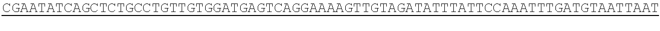

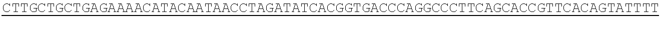

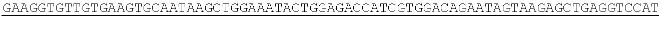

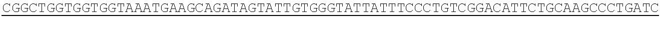

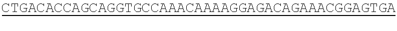

Protein sequence exon 6 to exon 7
(SEQ ID NO.: 124)

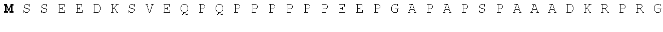

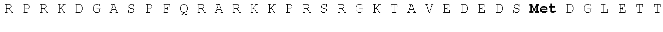

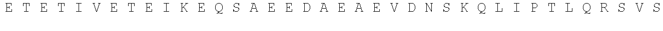

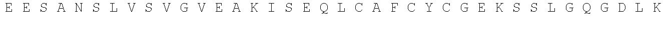

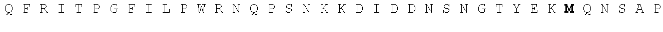

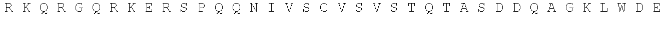

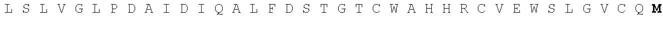

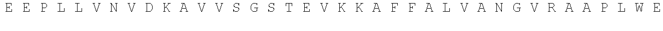

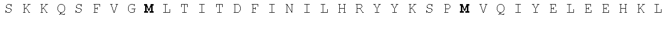

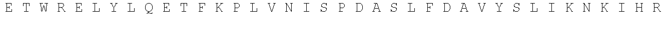

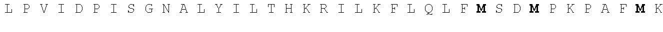

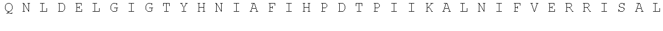

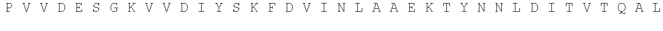

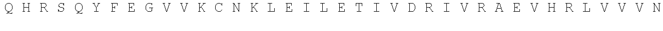

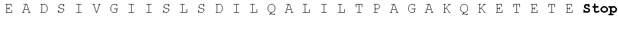

Protein Domain Exon 6 to Exon 7

No transmembrane domains within the query sequence of 566 residues.

MLL3-PRKAG2 Fusion sequence exon 23 to exon 6

cDNA sequence (SEQ ID NO.: 125), PRKAG2 underlined.
ATGTCGTCGGAGGAGGACAAGAGCGTGGAGCAGCCGCAGCCGCCGCCACCACCCCCCGAGGAGCCTGGAGCCCCG
GCCCCGAGCCCCGCAGCCGCAGACAAAAGACCTCGGGGCCGGCCTCGCAAAGATGGCGCTTCCCCTTTCCAGAGA
GCCAGAAAGAAACCTCGAAGTAGGGGGAAAACTGCAGTGGAAGATGAGGACAGCATGGATGGGCTGGAGACAACA
GAAACAGAAACGATTGTGGAAACAGAAATCAAAGAACAATCTGCAGAAGAGGATGCTGAAGCAGAAGTGGATAAC
AGCAAACAGCTAATTCCAACTCTTCAGCGATCTGTGTCTGAGGAATCGGCAAACTCCCTGGTCTCTGTTGGTGTA
GAAGCCAAAATCAGTGAACAGCTCTGCGCTTTTTGTTACTGTGGGGAAAAAAGTTCCTTAGGACAAGGAGACTTA
AAACAATTCAGAATAACGCCTGGATTTATCTTGCCATGGAGAAACCAACCTTCTAACAAGAAGGACATTGATGAC
AACAGCAATGGAACCTATGAGAAAATGCAAAACTCAGCACCACGAAAACAAAGAGGACAGAGAAAAGAACGATCT
CCTCAGCAGAATATAGTATCTTGTGTAAGTGTAAGCACCCAGACAGCTTCAGATGATCAAGCTGGTAAACTGTGG
GATGAACTCAGTCTGGTTGGGCTTCCAGATGCCATTGATATCCAAGCCTTATTTGATTCTACAGGCACTTGTTGG
GCTCATCACCGTTGTGTGGAGTGGTCACTAGGAGTATGCCAGATGGAAGAACCATTGTTAGTGAACGTGGACAAA
GCTGTTGTCTCAGGGAGCACAGAACGATGTGCATTTTGTAAGCACCTTGGAGCCACTATCAAATGCTGTGAAGAG
AAATGTACCCAGATGTATCATTATCCTTGTGCTGCAGGAGCCGGCACCTTTCAGGATTTCAGTCACATCTTCCTG
CTTTGTCCAGAACACATTGACCAAGCTCCTGAAAGATCGAAGGAAGATGCAAACTGTGCAGTGTGCGACAGCCCG
GGAGACCTCTTAGATCAGTTCTTTTGTACTACTTGTGGTCAGCACTATCATGGAATGTGCCTGGATATAGCGGTT
ACTCCATTAAAACGTGCAGGTTGGCAATGTCCTGAGTGCAAAGTGTGCCAGAACTGCAAACAATCGGGAGAAGAT
AGCAAGATGCTAGTGTGTGATACGTGTGACAAAGGGTATCATACTTTTTGTCTTCAACCAGTTATGAAATCAGTA
CCAACCAATGGCTGGAAATGCAAAAATTGCAGAATATGTATAGAGTGTGGCACACGGTCTAGTTCTCAGTGGCAC
CACAATTGCCTGATATGTGACAATTGTTACCAACAGCAGGATAACTTATGTCCCTTCTGTGGGAAGTGTTATCAT
CCAGAATTGCAGAAAGACATGCTTCATTGTAATATGTGCAAAAGGTGGGTTCACCTAGAGTGTGACAAACCAACA
GATCATGAACTGGATACTCAGCTCAAAGAAGAGTATATCTGCATGTATTGTAAACACCTGGGAGCTGAGATGGAT
CGTTTACAGCCAGGTGAGGAAGTGGAGATAGCTGAGCTCACTACAGATTATAACAATGAAATGGAAGTTGAAGGC
CCTGAAGATCAAATGGTATTCTCAGAGCAGGCAGCTAATAAAGATGTCAACGGTCAGGAGTCCACTCCTGGAATT
GTTCCAGATGCGGTTCAAGTCCACACTGAAGAGCAACAGAAGAGTCATCCCTCAGAAAGTCTTGACACAGATAGT
CTTCTTATTGCTGTATCATCCCAACATACAGTGAATACTGAATTGGAAAAACAGATTTCTAATGAAGTTGATAGT
GAAGACCTGAAAATGTCTTCTGAAGTGAAGCATATTTGTGGCGAAGATCAAATTGAAGATAAAATGGAAGTGACA
GAAAACATTGAAGTCGTTACACACCAGATCACTGTGCAGCAAGAACAACTGCAGTTGTTAGAGGAACCTGAAACA
GTGGTATCCAGAGAAGAATCAAGGCCTCCAAAATTAGTCATGGAATCTGTCACTCTTCCACTAGAAACCTTAGTG
TCCCCACATGAGGAAAGTATTTCATTATGTCCTGAGGAACAGTTGGTTATAGAAAGGCTACAAGGAGAAAAGGAA
CAGAAAGAAAATTCTGAACTTTCTACTGGATTGATGGACTCTGAAATGACTCCTACAATTGAGGGTTGTGTGAAA
GATGTTTCATACCAAGGAGGCAAATCTATAAAGTTATCATCTGAGACAGAGTCATCATTTTCATCATCAGCAGAC
ATAAGCAAGGCAGATGTGTCTTCCTCCCCAACACCTTCTTCAGACTTGCCTTCGCATGACATGCTGCATAATTAC
CCTTCAGCTCTTAGTTCCTCTGCTGGAAACATCATGCCAACAACTTACATCTCAGTCACTCCAAAAATTGGCATG
GGTAAACCAGCTATTACTAAGAGAAAATTTTCTCCTGGTAGACCTCGGTCCAAACAGGGGGCTTGGAGTACCCAT
AATACAGTGAGCCCACCTTCCTGGTCCCCAGACATTTCAGAAGGTCGGGAAATTTTTAAACCCAGGCAGCTTCCT
GGCAGTGCCATTTGGAGCATCAAAGTGGGCCGTGGGTCTGGATTTCCAGGAAAGCGGAGACCTCGAGGTGCAGGA
CTGTCGGGGCGAGGTGGCCGAGGCAGGTCAAAGCTGAAAAGTGGAATCGGAGCTGTTGTATTACCTGGGGTGTCT
ACTGCAGATATTTCATCAAATAAGGATGATGAAGAAAACTCTATGCACAATACAGTTGTGTTGTTTTCTAGCAGT
GACAAGTTCACTTTGAATCAGGATATGTGTGTAGTTTGTGGCAGTTTTGGCCAAGGAGCAGAAGGAAGATTACTT
GCCTGTTCTCAGTGTGGTCAGTGTTACCATCCATACTGTGTCAGTATTAAGATCACTAAAGTGGTTCTTAGCAAA
GGTTGGAGGTGTCTTGAGTGCACTGTGTGTGAGGCCTGTGGGAAGGCAACTGACCCAGGAAGACTCCTGCTGTGT
GATGACTGTGACATAAGTTATCACACCTACTGCCTAGACCCTCCATTGCAGACAGTTCCCAAAGGAGGCTGGAAG
TGCAAATGGTGTGTTTGGTGCAGACACTGTGGAGCAACATCTGCAGGTCTAAGATGTGAATGGCAGAACAATTAC
ACACAGTGCGCTCCTTGTGCAAGCTTATCTTCCTGTCCAGTCTGCTATCGAAACTATAGAGAAGAAGATCTTATT
CTGCAATGTAGACAATGTGATAGATGGATGCATGCAGTTTGTCAGAACTTAAATACTGAGGAAGAAGTGGAAAAT
GTAGCAGACATTGGTTTTGATTGTAGCATGTGCAGACCCTATATGCCTGCGTCTAATGTGCCTTCCTCAGACTGC
TGTGAATCTTCACTTGTAGCACAAATTGTCACAAAAGTAAAAGAGCTAGACCCACCCAAGACTTATACCCAGGAT
GGTGTGTGTTTGACTGAATCAGGGATGACTCAGTTACAGAGCCTCACAGTTACAGTTCCAAGAAGAAAACGGTCA
AAACCAAAATTGAAATTGAAGATTATAAATCAGAATAGCGTGGCCGTCCTTCAGACCCCTCCAGACATCCAATCA

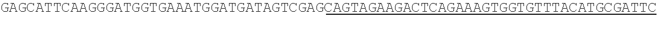

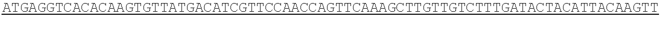

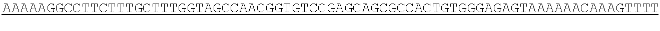

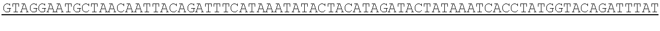

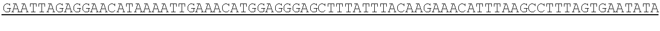

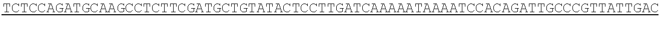

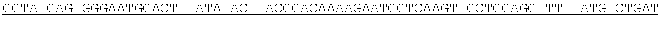

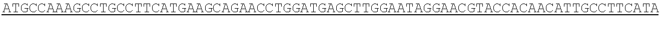

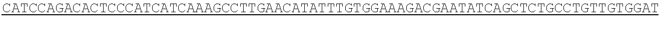

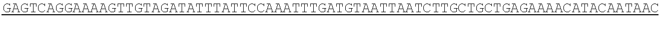

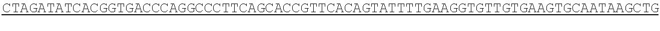

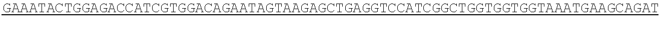

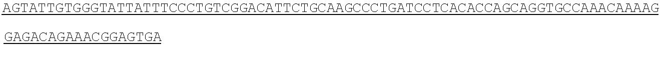

Protein sequence exon 23 to exon 6
(SEQ ID NO.: 126)

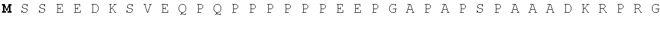

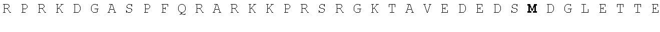

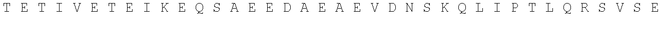

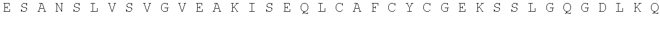

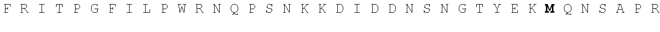

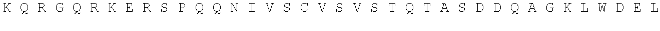

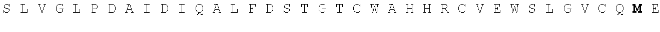

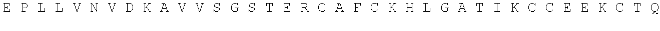

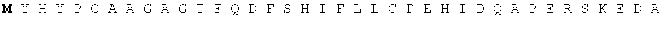

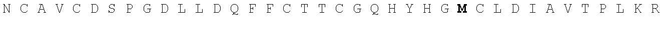

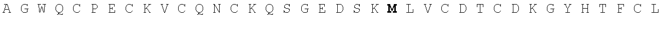

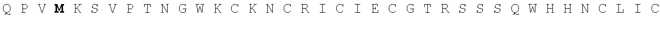

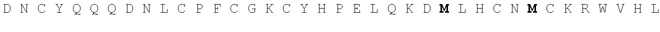

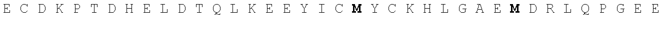

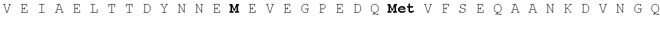

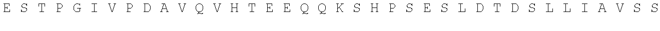

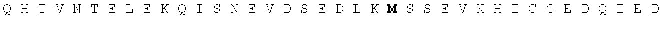

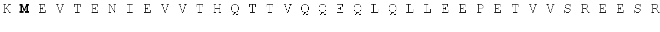

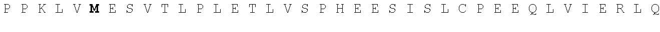

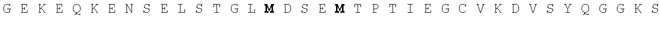

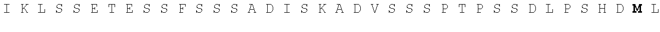

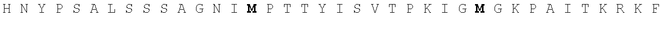

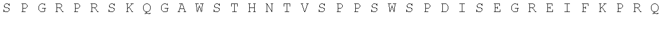

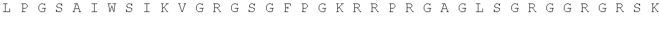

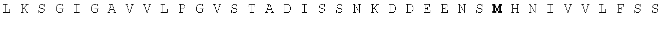

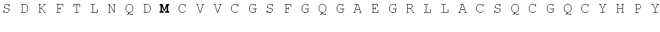

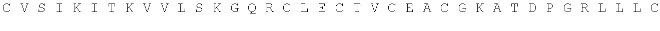

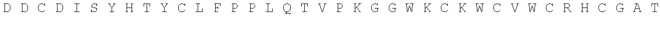

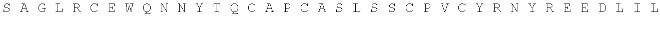

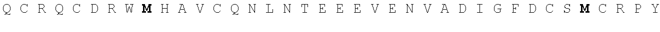

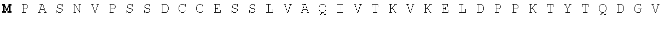

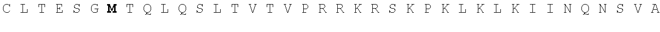

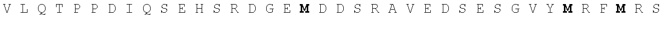

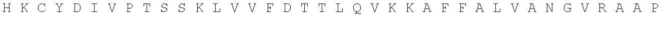

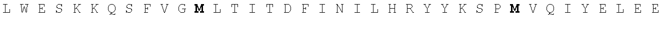

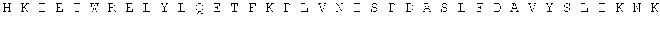

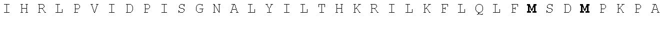

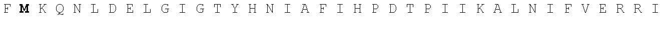

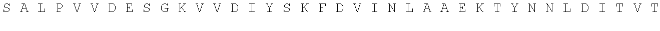

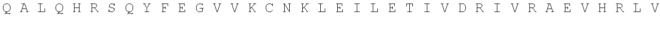

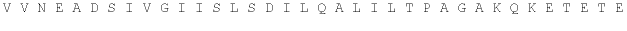

Stop

Protein Domain Exon 23 to Exon 6

Due to overlapping domains, there are 40 representation of the protein. No transmembrane domains.

Fusion Gene #5: DUS2L-PSKH1

Confirmed genomic breakpoints: DUS2L—chr16:67930935, PSKH1—chr16:68103638

Transcript: DUS2L-001 ENST00000565263

cDNA sequence (SEQ ID NO.: 127). part of fusion
gene shaded.
TGAGGCGCGCCGGCTGGTTCAACTCCGGCCGCCGCGCCGAAACCAGCAGC
GGTCCGGGTCGAACCAGCACCGGCCTCGGGAGGTTCCGCCGCCTGCTCTG
CCGCTGTTCCAACTGCCGCTGTAGAGCCACTGGGATGCGCACCACCGGCA
GGGGTTCGTCGGGACTGCGGACCGTGAGGCCCCGTCGCGGCGCCAGGAGC
AACCGAGTCACGAGGGAAAAGAGCCGCACCGGCCGCGTTAGAGCCATGTT
TCCCTTAGTGCGGGAGAAGCGCACATCAGTGACGTCACGGACGCGCCGCG
ACCTCGCGTACGGTGGCTGGCGAGGCTCAGTACGGTGTGTGGAGCTGGAG
CACCGTGAGGAAGAAGCGAGGTTCTTTTTAAGAGTTCAGCTGCGAGATAT
CAAACAAAGAATTACTCTGTACAAAGCCAGAACACATATATCAAAGTAAT
CCTGAAGTATCAGAACAAAATAATAGGCTGTAACAGAGGAGGAAATGATT
TTGAATAGCCTCTCTCTGTGTTACCATAATAAGCTAATCCTGGCCCCAAT
GGTTCGGGTAGGGACTCTTCCAATGAGGCTGCTGGCCCTGGATTATGGAG
CGGACATTGTTTACTGTGAGGAGCTGATCGACCTCAAGATGATTCAGTGC
AAGAGAGTTGTTAATGAGGTGCTCAGCACAGTGGACTTTGTCGCCCCTGA
TGATCGAGTTGTCTTCCGCACCTGTGAAAGAGAGCAGAACAGGGTGGTCT
TCCAGATGGGGACTTCAGACGCAGAGCGAGCCCTTGCTGTGGCCAGGCTT
GTAGAAAATGATGTGGCTGGTATTGATGTCAACATGGGCTGTCCAAAACA
ATATTCCACCAAGGGAGGAATGGGAGCTGCCCTGCTGTCAGACCCTGACA
AGATTGAGAAGATCCTCAGCACTCTTGTTAAAGGGACACGCAGACCTGTG
ACCTGCAAGATTCGCATCCTGCCATCGCTAGAAGATACCCTGAGCCTTGT
GAAGCGGATAGAGAGGACTGGCATTGCTGCCATCGCAGTTCATGGGAGGA
AGCGGGAGGAGCGACCTCAGCATCCTGTCAGCTGTGAAGTCATCAAAGCC
ATTGCTGATACCCTCTCCATTCCTGTCATAGCCAACGGAGGATCTCATGA
CCACATCCAACAGTATTCGGACATAGAGGACTTTCGACAAGCCACGGCAG
CCTCTTCCGTGATGGTGGCCCGAGCAGCCATGTGGAACCCATCTATCTTC
CTCAAGGAGGGTCTGCGGCCCCTGGAGGAGGTCATGCAGAAATACATCAG
ATACGCGGTGCAGTATGACAACCACTACACCAACACCAAGTACTGCTTGT
GCCAGATGCTACGAGAACAGCTGGAGTCGCCCCAGGGAAGGTTGCTCCAT
GCTGCCCAGTCTTCCCGGGAAATTTGTGAGGCCTTTGGCCTTGGTGCCTT
CTATGAGGAGACCACACAGGAGCTGGATGCCCAGCAGGCCAGGCTCTCAG
CCAAGACTTCAGAGCAGACAGGGGAGCCAGCTGAAGATACCTCTGGTGTC
ATTAAGATGGCTGTCAAGTTTGACCGGAGAGCATACCCAGCCCAGATCAC
CCCTAAGATGTGCCTACTAGAGTGGTGCCGGAGGGAGAAGTTGGCACAGC
CTGTGTATGAAACGGTTCAACGCCCTCTAGATCGCCTGTTCTCCTCTATT
GTCACCGTTGCTGAACAAAAGTATCAGTCTACCTTGTGGGACAAGTCCAA
GAAACTGGCGGAGCAGGCTGCAGCCATCGTCTGTCTGCGGAGCCAGGGCC
TCCCTGAGGGTCGGCTGGGTGAGGAGAGCCCTTCCTTGCACAAGCGAAAG
AGGGAGGCTCCTGACCAAGACCCTGGGGGCCCCAGAGCTCAGGAGCTAGC
ACAACCTGGGGATCTGTGCAAGAAGCCCTTTGTGGCCTTGGGAAGTGGTG
AAGAAAGCCCCCTGGAAGGCTGGTGACTACTCTTCCTGCCTTAGTCACCC
CTCCATGGGCCTGGTGCTAAGGTGGCTGTGGATGCCACAGCATGAACCAG
ATGCCGTTGAACAGTTTGCTGGTCTTGCCTGGCAGAAGTTAGATGTCCTG
GCAGGGGCCATCAGCCTAGAGCATGGACCAGGGGCCGCCCAGGGGTGGAT
CCTGGCCCCTTTGGTGGATCTGAGTGACAGGGTCAAGTTCTCTTTGAAAA
CAGGAGCTTTTCAGGTGGTAACTCCCCAACCTGACATTGGTACTGTGCAA
TAAAGACACCCCCTACCCTCACCCACGGCTGGCTGCTTCAGCCTTGGGCA
TCTTCATAAA

Transcript: DUS2L-001 ENST00000565263

cDNA sequence

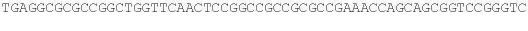

............................................................

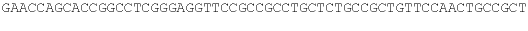

............................................................

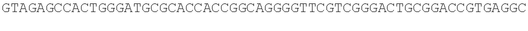

............................................................

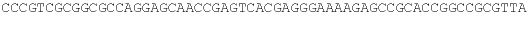

............................................................

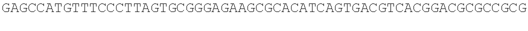

............................................................

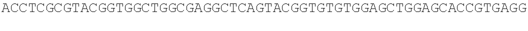

............................................................

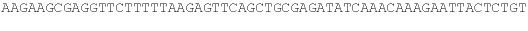

............................................................

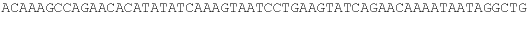

............................................................

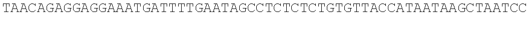

..............-M--I--L--N--S--L--S--L--C--Y--H--N--K--L--I--

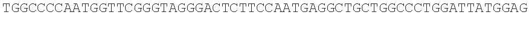

L--A--P--M--V--R--V--G--T--L--P--M--R--L--L--A--L--D--Y--G--

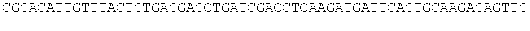

A--D--I--V--Y--C--E--E--L--I--D--L--K--M--I--Q--C--K--R--V--

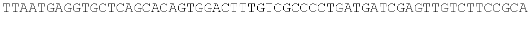

V--N--E--V--L--S--T--V--D--F--V--A--P--D--D--R--V--V--F--R--

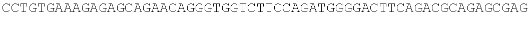

T--C--E--R--E--Q--N--R--V--V--F--Q--M--G--T--S--D--A--E--R--

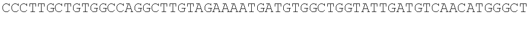

A--L--A--V--A--R--L--V--E--N--D--V--A--G--I--D--V--N--M--G--

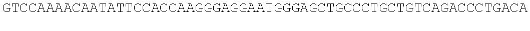

C--P--K--Q--Y--S--T--K--G--G--M--G--A--A--L--L--S--D--P--D--

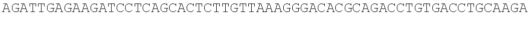

K--I--E--K--I--L--S--T--L--V--K--G--T--R--R--P--V--T--C--K--

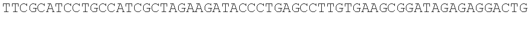

I--R--I--L--P--S--L--E--D--T--L--S--L--V--K--R--I--E--R--T--

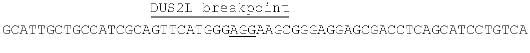

G--I--A--A--I--A--V--H--G--R--K--R--E--E--R--P--Q--H--P--V--

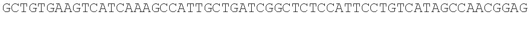

S--C--E--V--I--K--A--I--A--D--T--L--S--I--P--V--I--A--N--G--

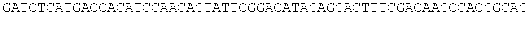

G--S--H--D--H--I--Q--Q--Y--S--D--I--E--D--F--R--Q--A--T--A--

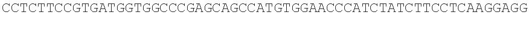

A--S--S--V--M--V--A--R--A--A--M--W--N--P--S--I--F--L--K--E--

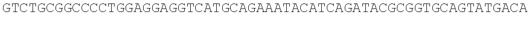

G--L--R--P--L--E--E--V--M--Q--K--Y--I--R--Y--A--V--Q--Y--D--

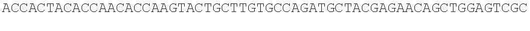

N--H--Y--T--N--T--K--Y--C--L--C--Q--M--L--R--E--Q--L--E--S--

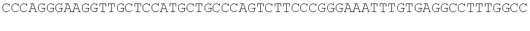

P--Q--G--R--L--L--H--A--A--Q--S--S--R--E--I--C--E--A--F--G--

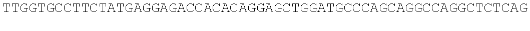

L--G--A--F--Y--E--E--T--T--Q--E--L--D--A--Q--Q--A--R--L--S--

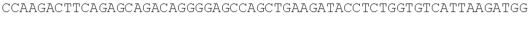

A--K--T--S--E--Q--T--G--E--P--A--E--D--T--S--G--V--I--K--M--

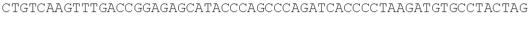

A--V--K--F--D--R--R--A--Y--P--A--Q--I--T--P--K--M--C--L--L--

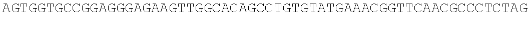

E--Q--C--R--R--E--K--L--A--Q--P--V--Y--E--T--V--Q--R--P--L--

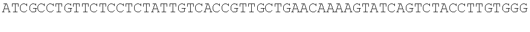

D--R--L--F--S--S--I--V--T--V--A--E--Q--K--Y--Q--S--T--L--W--

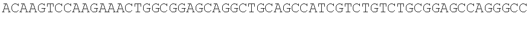

D--K--S--K--K--L--A--E--Q--A--A--A--I--V--C--L--R--S--Q--G--

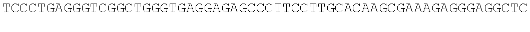

L--P--E--G--R--L--G--E--E--S--P--S--L--H--K--R--K--R--E--A--

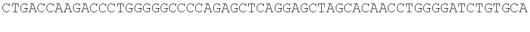

P--D--Q--D--P--G--G--P--R--A--Q--E--L--A--Q--P--G--D--L--C--

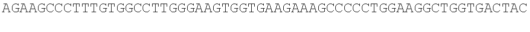

K--K--P--F--V--A--L--G--S--G--E--E--S--P--L--E--G--W--*-....

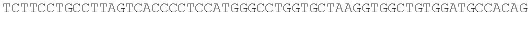

............................................................

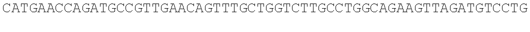

............................................................

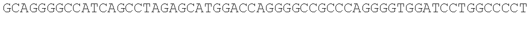

............................................................

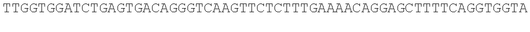

............................................................

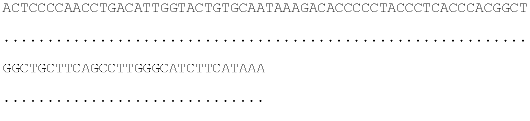

Transcript: DUS2L-001 ENST00000565263

Protein sequence (SEQ ID NO.: 128), parT of
fusion gene shaded.
MILNSLSLCYHNKLILAPMVRVGTLPMRLLALDYGADIVYCEELIDLKMI
QCKRVVNEVLSTVDFVAPDDRVVFRTCEREQNRVVFQMGTSDAERALAVA
RLVENDVAGIDVNMGCPKQYSTKGGMGAALLSDPDKIEKILSTLVKGTRR
PVTCKIRILPSLEDTLSLVKRIERTGIAAIAVHGRKREERPQHPVSCEVI
KAIADTLSIPVIANGGSHDHIQQYSDIEDFRQATAASSVMVARAAMWNPS
IFLKEGLRPLEEVMQKYIRYAVQYDNHYTNTKYCLCQMLREQLESPQGRL
LHAAQSSREICEAFGLGAFYEETTQELDAQQARLSAKTSEQTGEPAEDTS
GVIKMAVKFDRRAYPAQITPKMCLLEWCRREKLAQPVYETVQRPLDRLFS
SIVTVAEQKYQSTLWDKSKKLAEQAAAIVCLRSQGLPEGRLGEESPSLHK
RKREAPDQDPGGPRAQELAQPGDLCKKPFVALGSGEESPLEGW

Transcript: PSKH1-001 ENST00000291041

cDNA sequence (SEQ ID NO.: 129), part of fusion gene shaded.
GAGAATGGCGGCGGCGGCGGCGGCGGCGGCGGCCGCTGCCATTGCCCGGAGATGGCCGGC

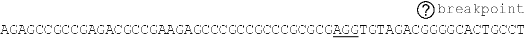

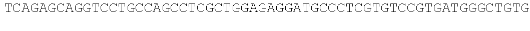

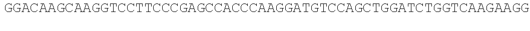

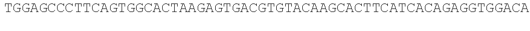

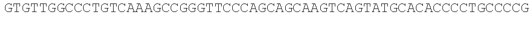

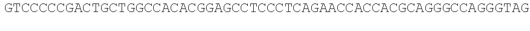

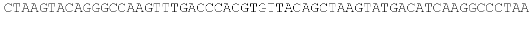

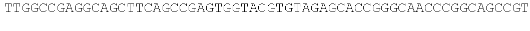

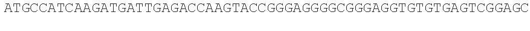

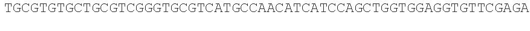

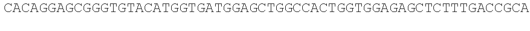

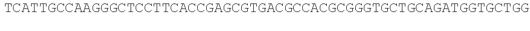

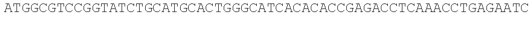

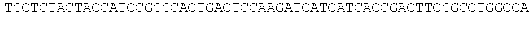

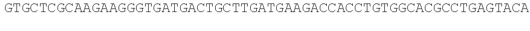

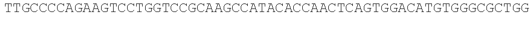

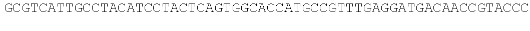

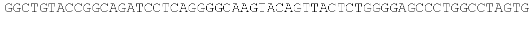

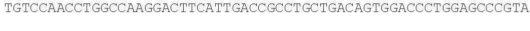

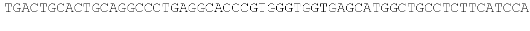

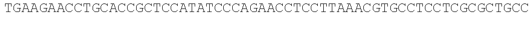

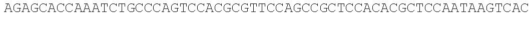

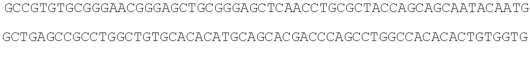

CCATCTGGGTCCGATGCCCTCTCTGGAGATAGGCCTATGTGGCCCACAGTAGGTGAAGAA
TGTCTGGCTCCAGCCCTTTCTCTGTGCCTTCAGCAGCCCCTGTCCTCACCATGGGCCTGG
GCCAGGTGTGACAGAGTAGAGGTAGCACAGGGGGCTGTGACTCCCCCTGAACTGGGAGCC
TGGCCTGGCACTGATACCCCTCTTGGTGGGCAGCTGCTCTGGTGGAGTTGGGAAGGGATA
GGACCTGGCCTTCACTGTCTCCCTTGCCCTTTGACTTTTCCCCAATCAAAGGGAACTGCA
GTGCTGGGTGGAGTGTCCTGTGGCCTCAGGACCCTTTGGGACAGTTACTTCTGGGACCCC
CTTTCCTCCACAGAGCCCTTCTCCCTGGTTTCACACATTCCCATGCATCCTGATCCTTAA
GATTATGCTCCAGTGGGAGACCCTGGTAGGCACAAAGCTTGTGCCTTGACTGGACCCGTA
GCCCCTGGCTAGGTCGAAACAGCCCTCCACCTCCCAGCCAAGATCTGTCTTCCTTCATGG
TGCCTCCAGGGAGCCTTCCTGGTCCCAGGACCTCTGGTGGAGGGCCATGGCGTGGACCTT
CACCCTTCTGGACTGTGTGGCCATGCTGGTCATCGGCTTGCCCAGGCTCCAGCCTCTCCA
GATTCTGAGGGGTCTCAGCCCACCGCCCTTGGTGCCTTCTTTGTAGAGCCCACCGCTACC
TCCCTCTCCCCGTTGGATGTCCATTCCATTCCCCAGGTGCCTCCTTCCCAACTGGGGGTG
GTTAAAGGGAGCCCCACTGCTGCTACCTGGGGAATGGGGCACCTGGGGGCCAAGGCAGAG
GGAAGGGGGTCCTCCCGATTAGGGTCGAGTGTCAGCCTGGGTTCTATCCTTTGGTGCAGC
CCCATTGCCTTTTCCCTTCAGGCTCTGTTGCTCCCTCCTCTGCAGCTGCACGAAGGCGCC
ATCTGGTGTCTGCATGGGTGTTGGCAGCCTGGGAGTGATCACTGCACGCCCATCGTGCAC
ACCTGCCCATCGTGCACACCCACCCATGGTGCACACCTGTAGTCCTCCATGAGGACATGG
GAAGGTAGGAGTTGCCGCCCTGGGGGAGGGTCCCGGGCTGCTCACCTCTCCCCTTCTGCT
GAGCTTCTGCGCACCCCTCCCTGGAACTTAGCCATACTGTGTGACCTGCCTCTGAAACCA
GGGTGCCAGGGGCACTGCCTTCTCACAGCTGGCCTTGCCCCGTCCACCCTGTGCTGCTTC
CCTTCACAGCATTAACCTTCCAGTCTGGGTCCCACTGAGCCTCAAGCTGGAAGGAGCCCC
TGCGGGAGGTGGGTGGGGTTGGGTGGCTGCTTTCCCAGAGGCCTGAGCCAGAACCATCCC
CATTTCTTTTGTGGTATCTCCCCCTACCACAAACCAGGCTGGAACCCAAGCCCCTTCCTC
CACAGCTGCCTTCAGTGGGTAGAATGGGGCCAGGGCCCAGCTTTGGCCTTAGCTTGACGG
CAGGGCCCCTGCCATTGCAGGAGGGTTTGGTTCCCACTCAGCTTCTGCCGGTCGGCAGCC
TGGGCCAGGCCCTTTTCCTGCATGTGCCACCTCCAGTGGGAAACAAAACTAAAGAGACCA
CTCTGTGCCAAGTCGACTATGCCTTAGACACATCCTCCTACCGTCCCCAATGCCCCCTGG
GCAGGAGGCAGTGGAGAACCAAGCCCCATGGCCTCAGAATTTCCCCCCAGTTCCCCAAGT
GTCTCTGGGGACCTGAAGCCCTGGGGCTTACGTTCTCTCTTGCCCAGGGTGGGCCTGGTC
CTGAGGGCAGGACAGGGGGTTTGGAGATGTGGGCCTTTGATAGACCCACTTGGGCCTTCA
TGCCATGGCCTGTGGATGGAGAATGTGCAGTTATTTATTATGCGTATTCAGTTTGTAAAC
GTATCCTCTGTATTCAGTAAACAGGCTGCCTCTCCAGGGAGGGCTGCCATTCATTCCAAC
AGTTCTGGCTTCTTGCTGTAGGACCAAGGGGTTGCCCTGGAGGAGGGGTGGGGGCCCCGG
CCTCGGCATGGCTACTCTAGGAAGAGCCACTGCTACTCAAGGAGTCACTCAGCCCCTTCT
GTGCCAGAAGTCCAAGTAGGGAGTCGGACCCTCAACAGCCTCTTCTTTCTCCTGAGCCAG
GAAGACAGACATGAATGCATGATGGGACAGGGCCTGGGTCTTTAATGGGTTGAGCTGGGG
AGGGCCTGTGGTGAGCTCAGTTGTAGGCTATGACCTGGTT

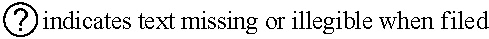

Transcript: PSKH1-001 ENST00000291041

cDNA sequence

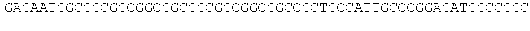

............................................................

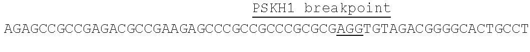

............................................................

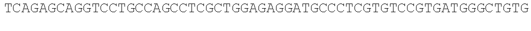

..................................................-M--G--C--

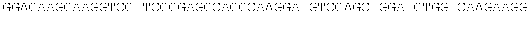

G--T--S--K--V--L--P--E--P--P--K--D--V--Q--L--D--L--V--K--K--

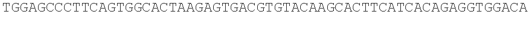

V--E--P--F--S--G--T--K--S--D--V--Y--K--H--F--I--T--E--V--D--

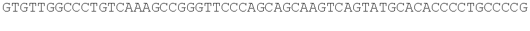

S--V--G--P--V--K--A--G--F--P--A--A--S--Q--Y--A--H--P--C--P--

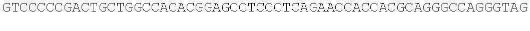

G--P--P--T--A--G--H--T--E--P--P--S--E--P--P--R--R--A--R--V--

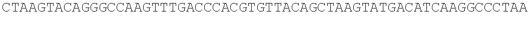

A--K--Y--R--A--K--F--D--P--R--V--T--A--K--Y--D--I--K--A--L--

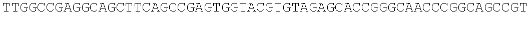

I--G--R--G--S--F--S--R--V--V--R--V--E--H--R--A--T--R--Q--P--

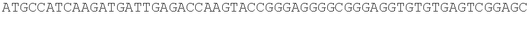

Y--A--I--K--M--I--E--T--K--Y--R--E--G--R--E--V--C--E--S--E--

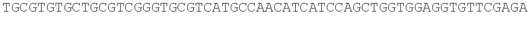

L--R--V--L--R--R--V--R--H--A--N--I--I--Q--L--V--E--V--F--E--

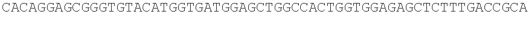

T--Q--E--R--V--Y--M--V--M--E--L--A--T--G--G--E--L--F--D--R--

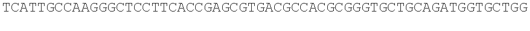

I--I--A--K--G--S--F--T--E--R--D--A--T--R--V--L--Q--M--V--L--

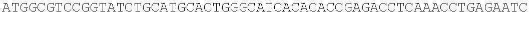

D--G--V--R--Y--L--H--A--L--G--I--T--H--R--D--L--K--P--E--N--

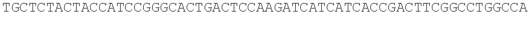

L--L--Y--Y--H--P--G--T--D--S--K--I--I--I--T--D--F--G--L--A--

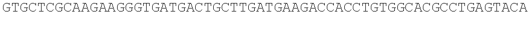

S--A--R--K--K--G--D--D--C--L--M--K--T--T--C--G--T--P--E--Y--

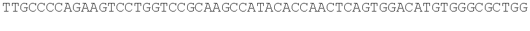

I--A--P--E--V--L--V--R--K--P--Y--T--N--S--V--D--M--W--A--L--

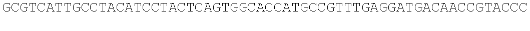

G--V--I--A--Y--I--L--L--S--G--T--M--P--F--E--D--D--N--R--T--

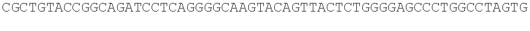

R--L--Y--R--Q--I--L--R--G--K--Y--S--Y--S--G--E--P--W--P--S--

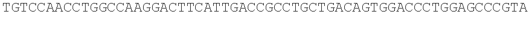

V--S--N--L--A--K--D--F--I--D--R--L--L--T--V--D--P--G--A--R--

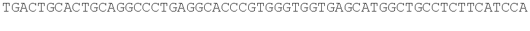

M--T--A--L--Q--A--L--R--H--P--W--V--V--S--M--A--A--S--S--S--

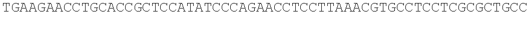

M--K--N--L--H--R--S--I--S--Q--N--L--L--K--R--A--S--S--R--C--

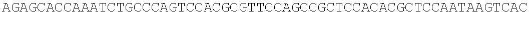

Q--S--T--K--S--A--Q--S--T--R--S--S--R--S--T--R--S--N--K--S--

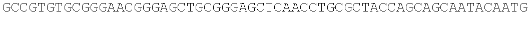

R--R--V--R--E--R--E--L--R--E--L--N--L--R--Y--Q--Q--Q--Y--N--

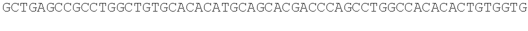

G--*-.......................................................

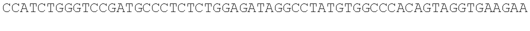

............................................................

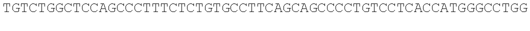

............................................................

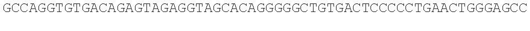

............................................................

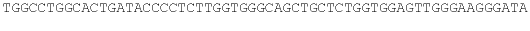

............................................................

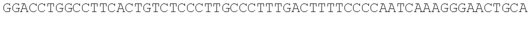

............................................................

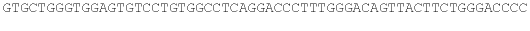

............................................................

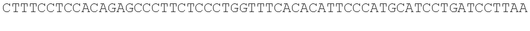

............................................................

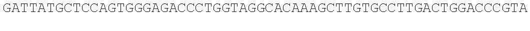

............................................................

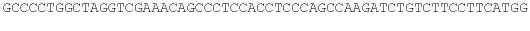

............................................................

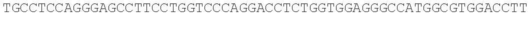

............................................................

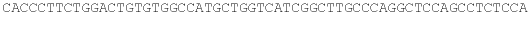

............................................................

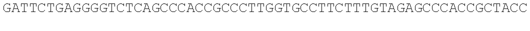

............................................................

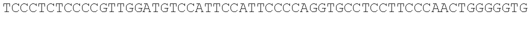

............................................................

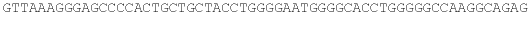

............................................................

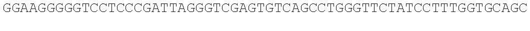

............................................................

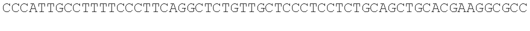

............................................................

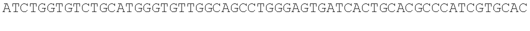

............................................................

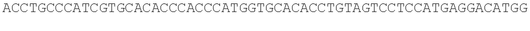

............................................................

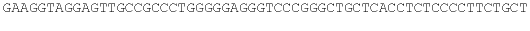

............................................................

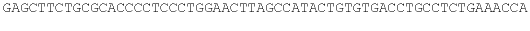

............................................................

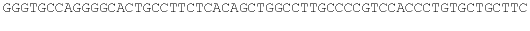

............................................................

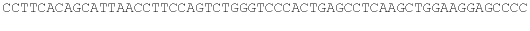

............................................................

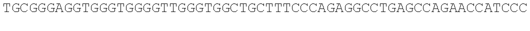

............................................................

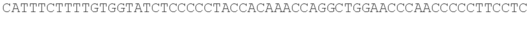

............................................................

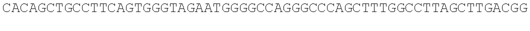

............................................................

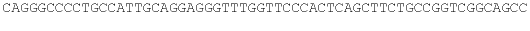

............................................................

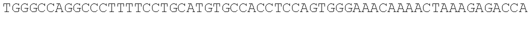

............................................................

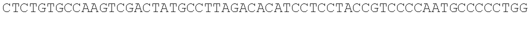

............................................................

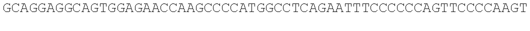

............................................................

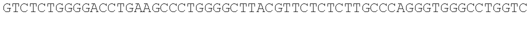

............................................................

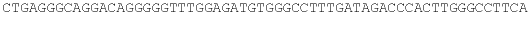

............................................................

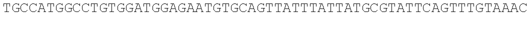

............................................................

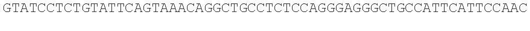

............................................................

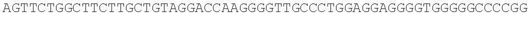

............................................................

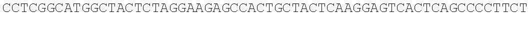

............................................................

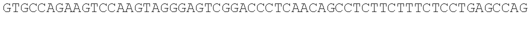

............................................................

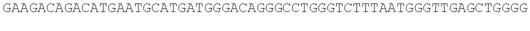

............................................................

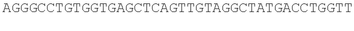

........................................

Transcript: PSKH1-001 ENST00000291041

Protein sequence (SEQ ID NO.: 130)
MGCGTSKVLPEPPKDVQLDLVKKVEPFSGTKSDVYKHFITEVDSVGPVKA
GFPAASQYAHPCPGPPTAGHTEPPSEPPRRARVAKYRAKFDPRVTAKYDI
KALIGRGSFSRVVRVEHRATRQPYAIKMIETKYREGREVCESELRVLRRV
RHANIIQLVEVFETQERVYMVMELATGGELFDRIIAKGSFTERDATRVLQ
MVLDGVRYLHALGITHRDLKPENLLYYHPGTDSKIIITDFGLASARKKGD
DCLMKTTCGTPEYIAPEVLVRKPYTNSVDMWALGVIAYILLSGTMPFEDD
NRTRLYRQILRGKYSYSGEPWPSVSNLAKDFIDRLLTVDPGARMTALQAL
RHPWVVSMAASSSMKNLHRSISQNLLKRASSRCQSTKSAQSTRSSRSTRS
NKSRRVRERELRELNLRYQQQYNG

DUS2L-PSKH1 Fusion sequence exon 10 to exon 2 UTR

cDNA sequence (SEQ ID NO.: 131). PSKH1 underlined.
ATGATTTTGAATAGCCTCTCTCTGTGTTACCATAATAAGCTAATCCTGGCCCCAATGGTTCGGGTAGGGACTCTT
CCAATGAGGCTGCTGGCCCTGGATTATGGAGCGGACATTGTTTACTGTGAGGAGCTGATCGACCTCAAGATGATT
CAGTGCAAGAGAGTTGTTAATGAGGTGCTCAGCACAGTGGACTTTGTCGCCCCTGATGATCGAGTTGTCTTCCGC
ACCTGTGAAAGAGAGCAGAACAGGGTGGTCTTCCAGATGGGGACTTCAGACGCAGAGCGAGCCCTTGCTGTGGCC
AGGCTTGTAGAAAATGATGTGGCTGGTATTGATGTCAACATGGGCTGTCCAAAACAATATTCCACCAAGGGAGGA
ATGGGAGCTGCCCTGCTGTCAGACCCTGACAAGATTGAGAAGATCCTCAGCACTCTTGTTAAAGGGACACGCAGA
CCTGTGACCTGCAAGATTCGCATCCTGCCATCGCTAGAAGATACCCTGAGCCTTGTGAAGCGGATAGAGAGGACT

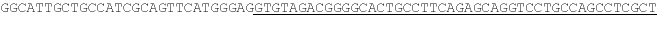

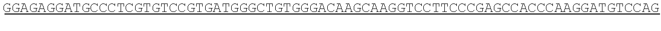

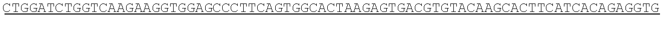

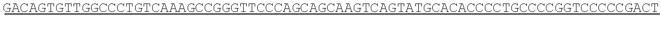

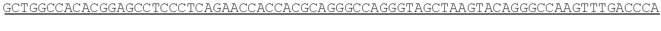

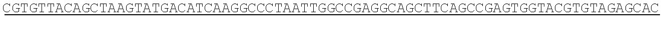

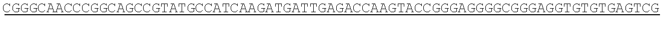

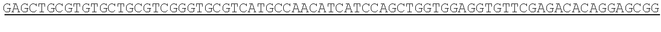

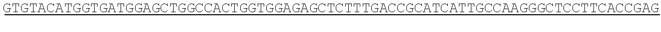

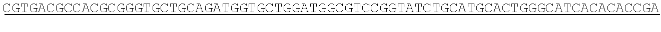

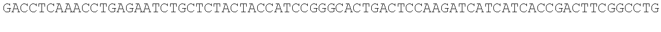

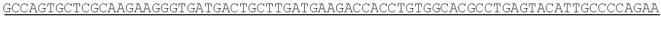

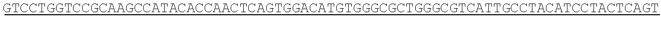

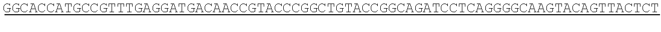

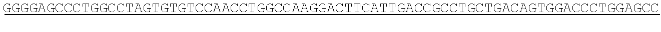

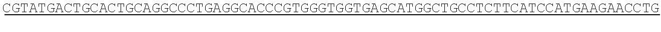

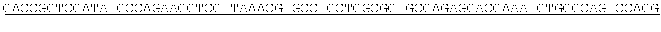

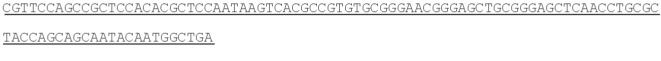

DUS2L-PSKH1 Fusion sequence exon 10 to exon 2 UTR

Protein sequence (SEQ ID NO.: 132), PSKH1 underlined.
MILNSLSLCYHNKLILAPMVRVGTLPMRLLALDYGADIVYCEELIDLKMIQCKRVVNEVLSTVDFVAPDDRVVFR
TCEREQNRVVFQMGTSDAERALAVARLVENDVAGIDVNMGCPKQYSTKGGMGAALLSDPDKIEKILSTLVKGTRR

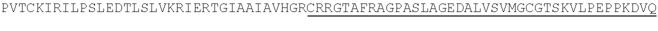

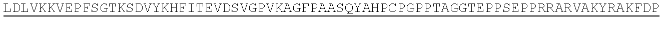

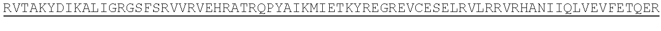

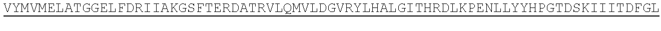

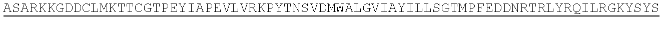

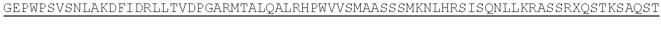

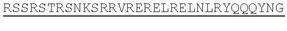

Protein Domain

No transmembrane domain.

DUS2L-PSKH1 Fusion sequence exon 3 to exon 2 UTR

cDNA sequence (SEQ ID NO.: 133), PSKH1 underlined.
ATGATTTTGAATAGCCTCTCTCTGTGTTACCATAATAAGCTAATCCTGGCCCCAATGGTTCGGGTAGGGACTCTT
CCAATGAGGCTGCTGGCCCTGGATTATGGAGCGGACATTGTTTACTGTGAGGAGCTGATCGACCTCAAGATGATT
CAGTGCAAGAGAGTTGTTAATGAGGTGCTCAGCACAGTGGACTTTGTCGCCCCTGATGATCGAGTTGTCTTCCGC

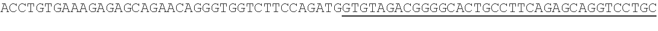

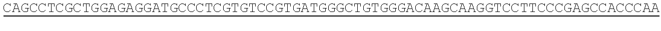

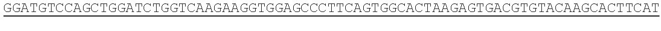

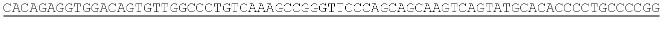

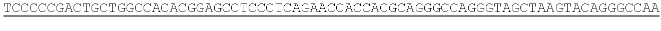

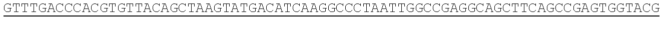

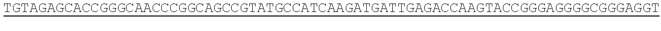

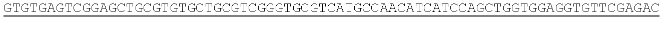

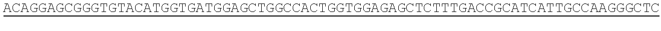

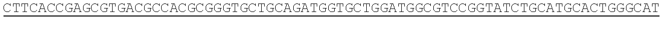

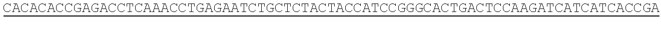

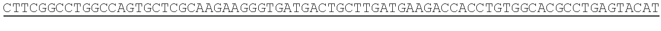

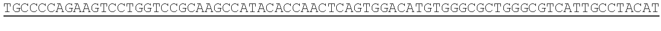

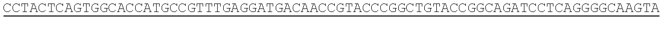

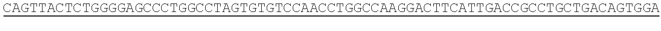

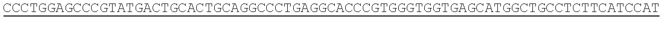

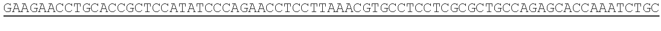

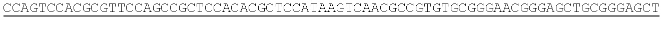

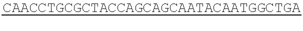

Protein sequence
(SEQ ID NO.: 134)

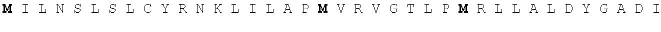

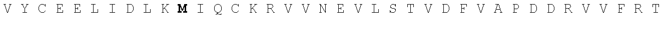

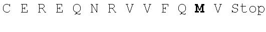

Protein Domain

No domains.

Genomic positions of the mRNA fusion points for each of the fusion genes in this study are presented in Table 4.

TABLE 4
Genomic locations corresponding to the mRNA fusion points of the five
recurrent fusion genes in this study.

| | RT-PCR breakpt Gene | | | RT-PCR breakpt Gene 2 | | | | |
|---|---|---|---|---|---|---|---|---|
| | 1 (5′) | | | (3′) | | | | |
| | | | Genomic | | | Genomic | | |
| Fusion | | | location | | | location | # of | Reading |
| gene | Chr | Exon | (hg19) | Chr | Exon | (hg19) | tumors | frame |
| CLEC16A- | 16 | 4 | 11,063,166 | 16 | 2 | 10,641,534 | 1 | In-frame |
| EMP2 | | | (+) | | (UTR) | (−) | | |
| | 16 | 9 | 11,073,239 | 16 | 2 | 10,641,534 | 2 | In-frame |
| | | | (+) | | (UTR) | (−) | | |
| | 16 | 10 | 11,076,848 | 16 | 2 | 10,641,534 | 2 | In-frame |
| | | | (+) | | (UTR) | (−) | | |
| CLDN18- | 3 | 5 | 137,749,947 | 5 | 12 | 142,393,645 | 3 | In-frame |
| ARHGAP26 | | | (+) | | | (+) | | |
| SNX2- | 5 | 12 | 122,161,888 | 5 | 4 | 122,491,578 | 1 | In-frame |
| PRDM6 | | | (+) | | | (+) | | |
| | 5 | 2 | 122,131,078 | 5 | 7 | 122,515,841 | 1 | Out-of- |
| | | | (+) | | | (+) | | frame |
| MLL3- | 7 | 6 | 152,007,051 | 7 | 7 | 151,273,538 | 1 | In-frame |
| PRKAG2 | | | (−) | | | (−) | | |
| | 7 | 9 | 151,960,101 | 7 | 5 | 151,329,224 | 1 | In-frame |
| | | | (−) | | | (−) | | |
| | 7 | 23 | 151,917,608 | 7 | 6 | 151,292,540 | 2 | In-frame |
| | | | (−) | | | (−) | | |
| DUS2L- | 16 | 3 | 68,072,052 | 16 | 2 | 67,942,583 | 1 | Out-of- |
| PSKH1 | | | (+) | | (UTR) | (+) | | frame |
| | 16 | 10 | 68,100,539 | 16 | 2 | 67,942,583 | 2 | In-frame |
| | | | (+) | | (UTR) | (+) | | |

### EXPERIMENTAL PROCEDURES

### Example 1

### Structural Variations (SVs) in Gastric Cancer (GC) Identified by Whole-Genome DNA-PET Sequencing

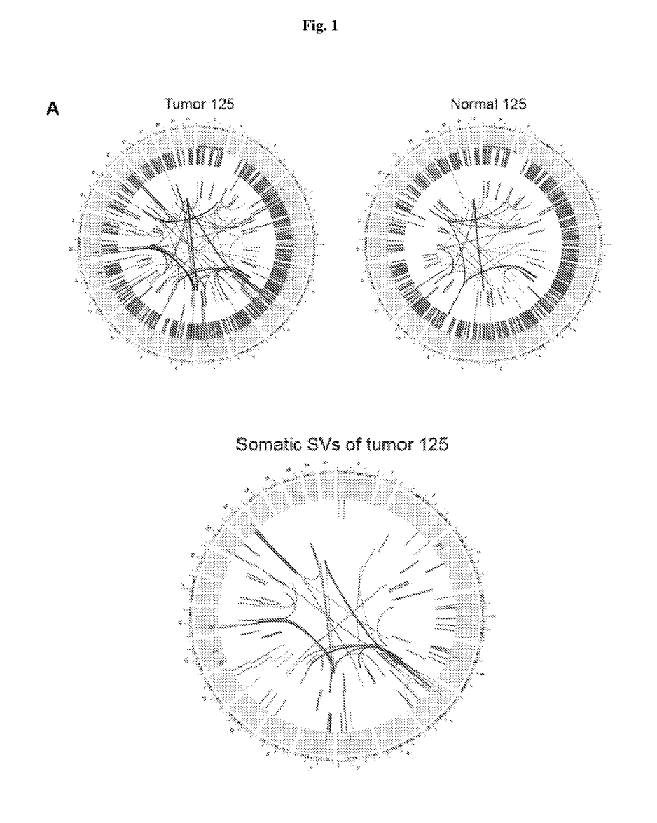

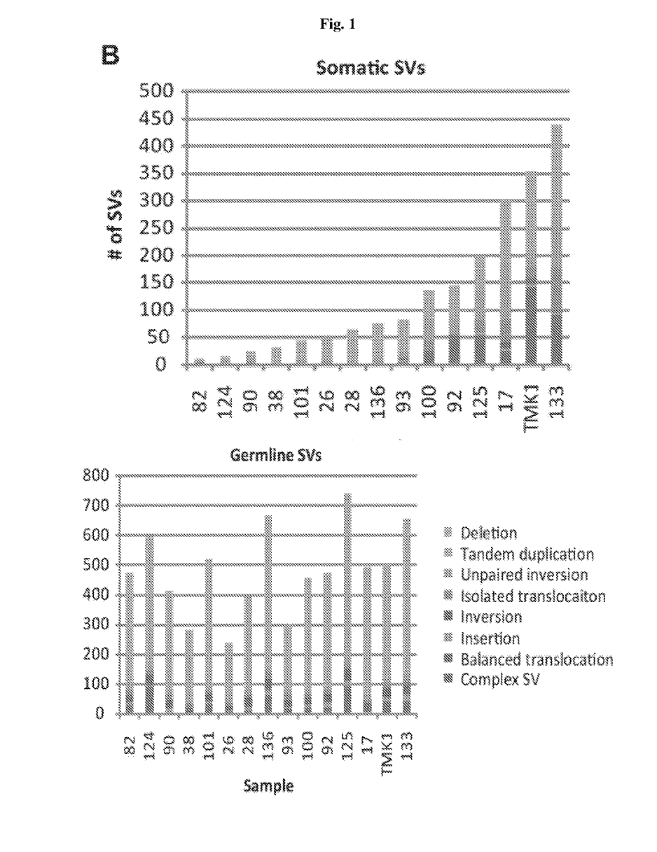

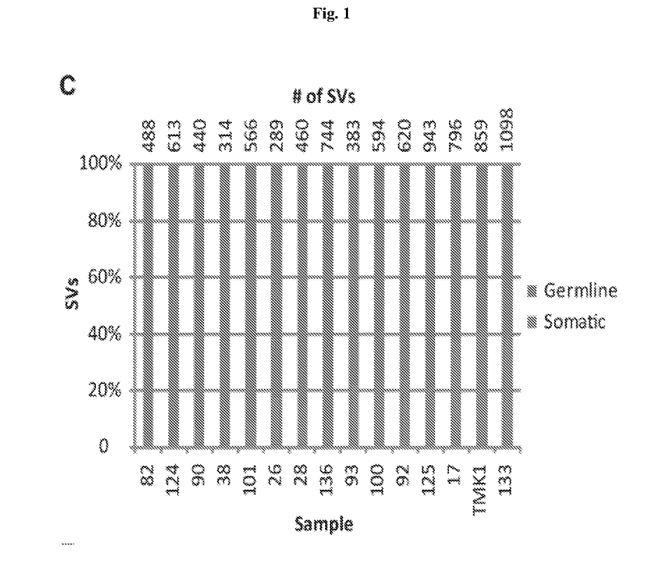

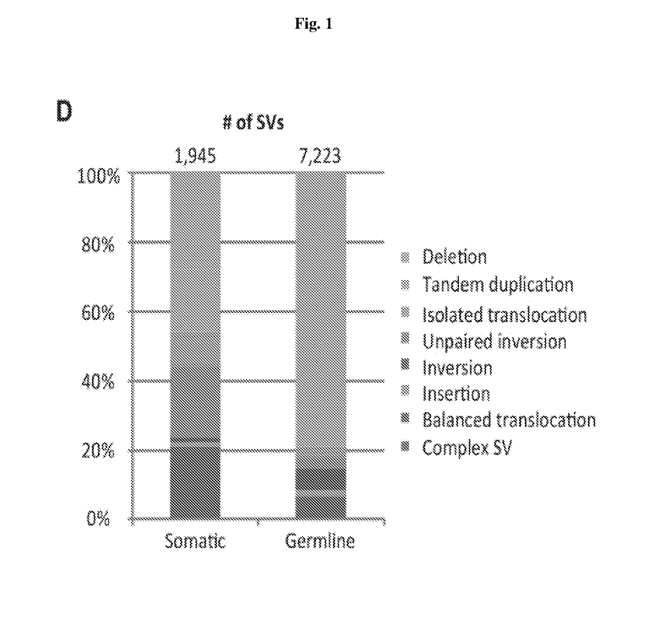

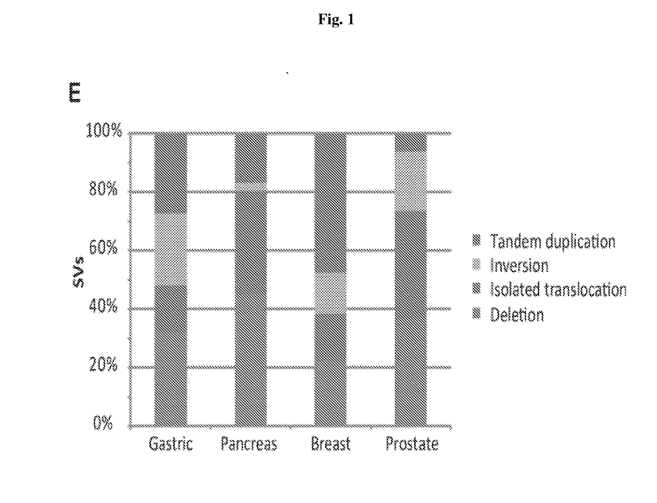

Genomic DNA was sequenced from 14 primary gastric tumors including ten paired normal samples and gastric cancer cell line TMK1 by DNA-PET. With approximately 2-fold by coverage and 200-fold physical coverage of the genome, 1,945 somatic SVs were identified (FIG. 1A-C) with significant differences in SV distributions between germline and somatic SVs (P=2.2×10⁻¹⁶, χ² tests, FIG. 1D) suggesting different mutational or selective mechanisms. Compared to other cancer types that have been analyzed for SVs in detail, GC showed a higher proportion of tandem duplications than prostate cancer and more inversions than pancreatic cancer (FIG. 1E), indicating that each cancer type bears its own rearrangement pattern.

### Example 2

### Characteristics of Somatic SVs in GC Provide Insight into Rearrangement Mechanisms

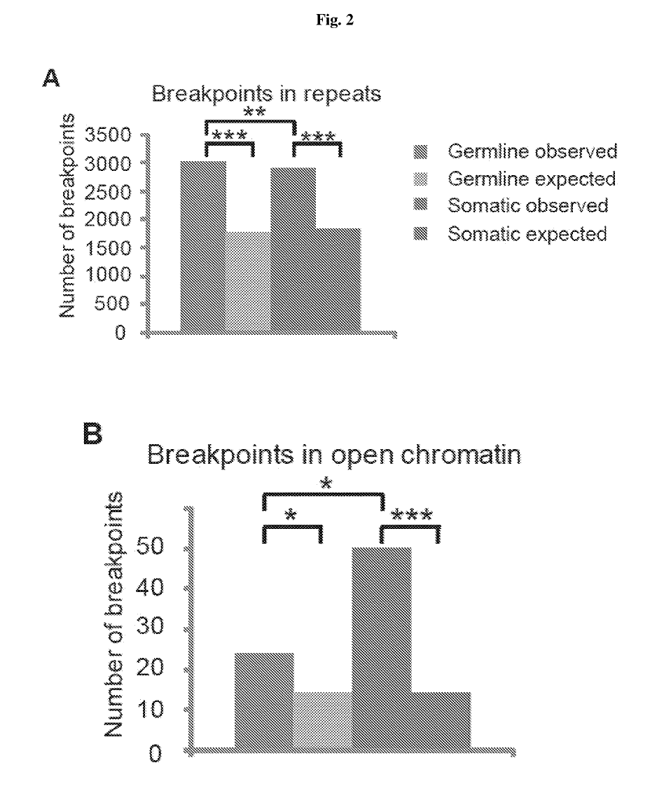

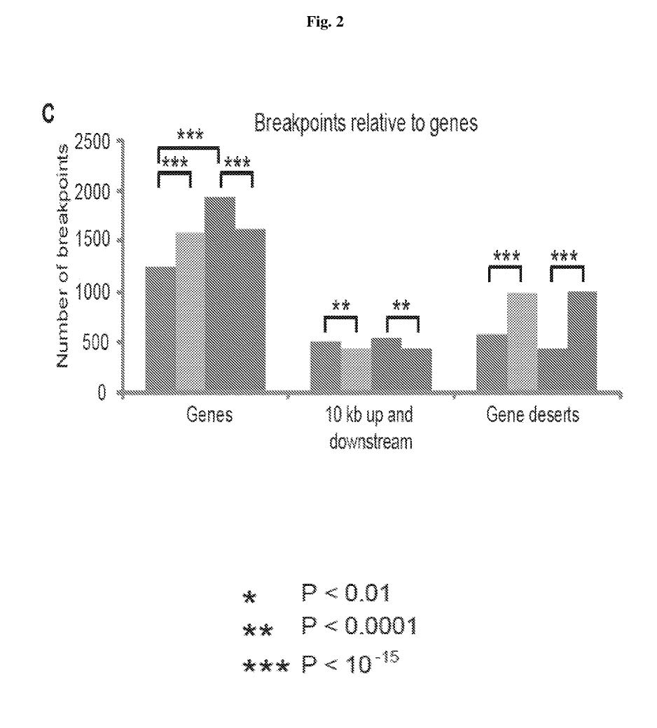

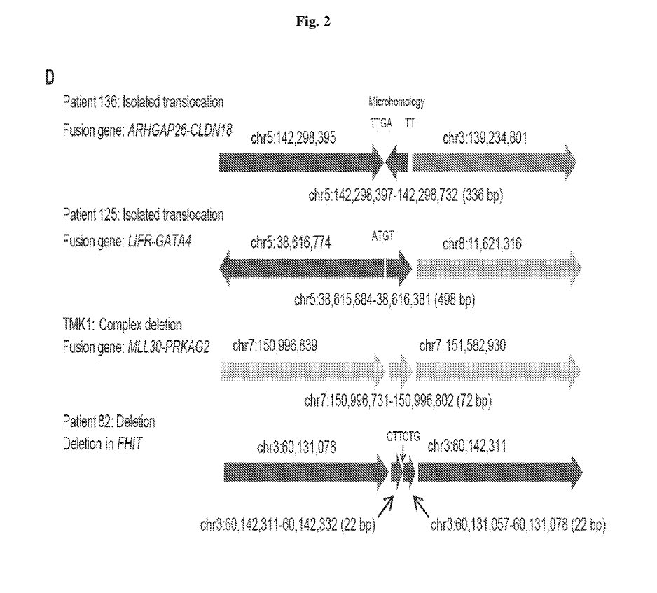

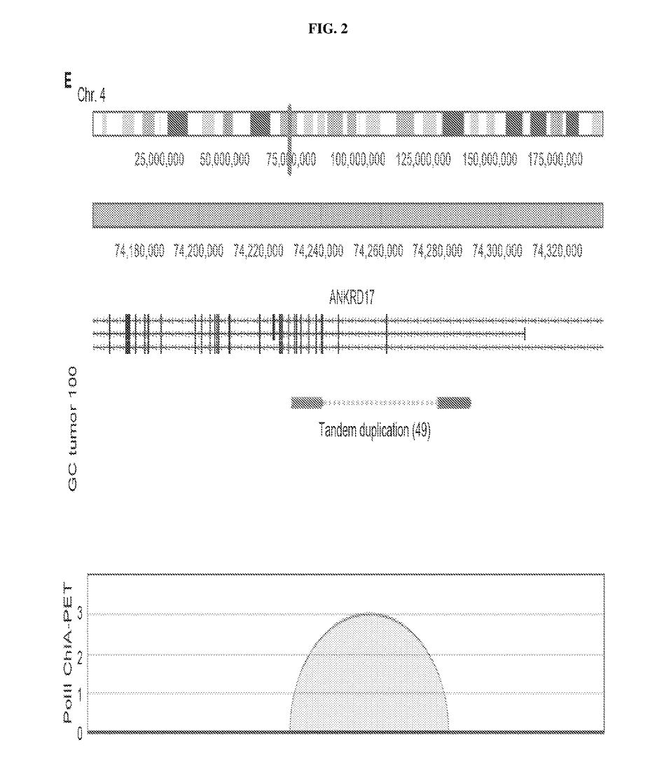

Both germline and somatic breakpoints were enriched in repeat regions (P<10⁻⁵ FIG. 2A) and open chromatin domains (P<10⁻²¹ χ² test; FIG. 2B) while only somatic breakpoints were enriched in genes (P<10⁻¹⁵ χ² test) and germline breakpoints were depleted in genes (P<10⁻¹⁵ χ² test, FIG. 2C), This may reflect the negative selection for gene-disruptive rearrangements in germline and, in contrast, the pro-cancer potential for somatic rearrangements altering gene structures. These observations suggest that transcriptionally active parts of the genome are more prone for somatic rearrangements in GC.

It was observed that 2% of validated fusion points have a characteristic pattern where the inserted sequence originated from a locus near the fusion point (FIG. 2D). Three of these cases created fusion genes (ARHGAP26-CLDN18, LIFR-GATA4, and MLL3-PRKAG2) The observation of these rearrangement features at the same locus may suggest a specific mechanism which might be transcription-coupled.

The possibility that the rearrangement partner sites of somatic SVs tend to be in spatial proximity within the nucleus was tested by searching for overlap between SVs and chromatin interaction analysis by paired-end-tag (ChIA-PET) sequencing data. As a proof of concept, cell line-derived (MCF-7 and K562) chromatin interactions and tumor derived somatic SVs for breast cancer and chronic myeloid leukemia (CML), respectively, were compared and significant overlap was observed.

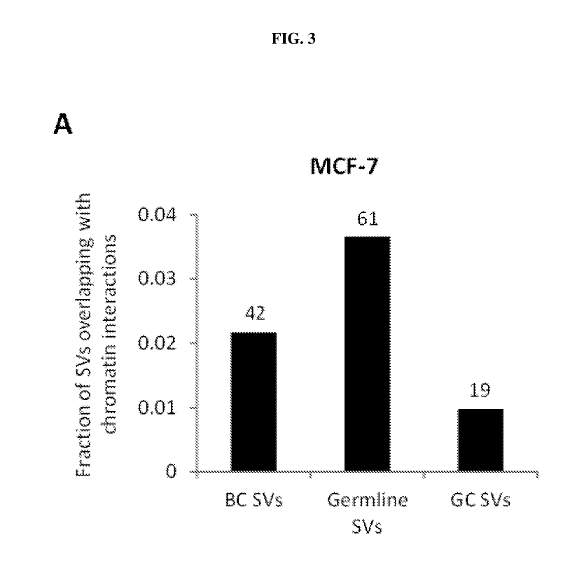

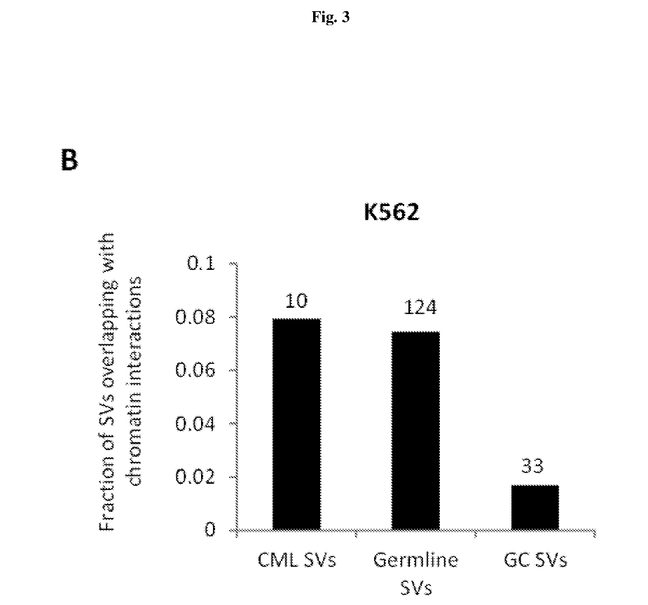

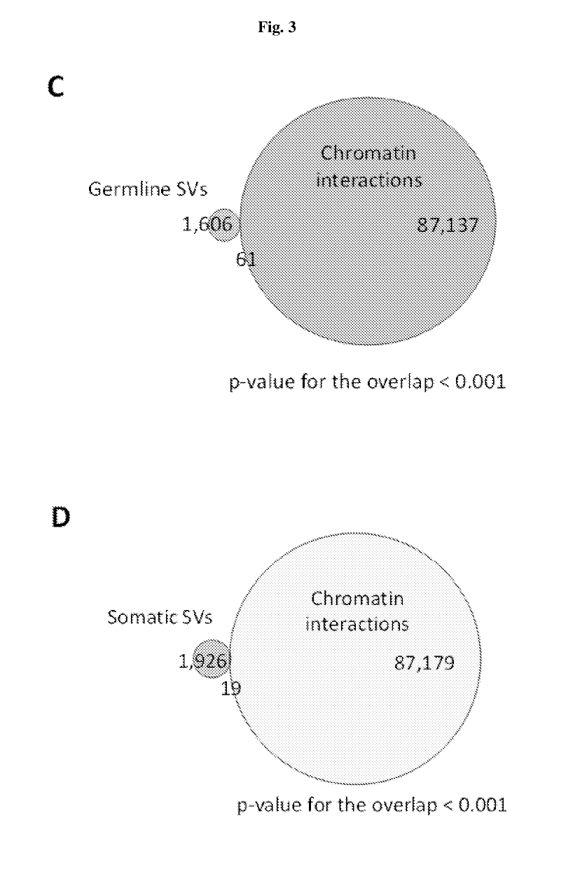

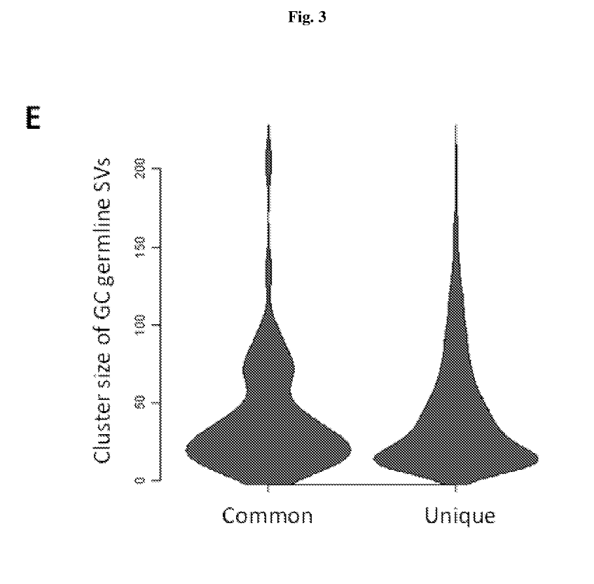

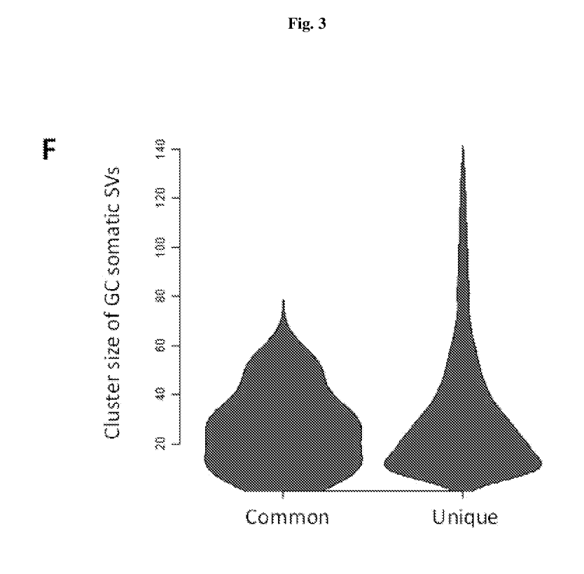

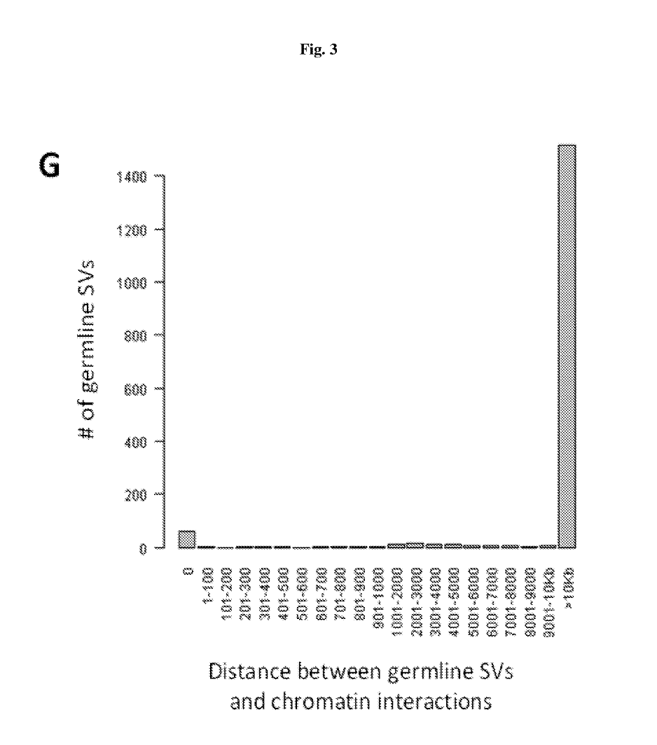

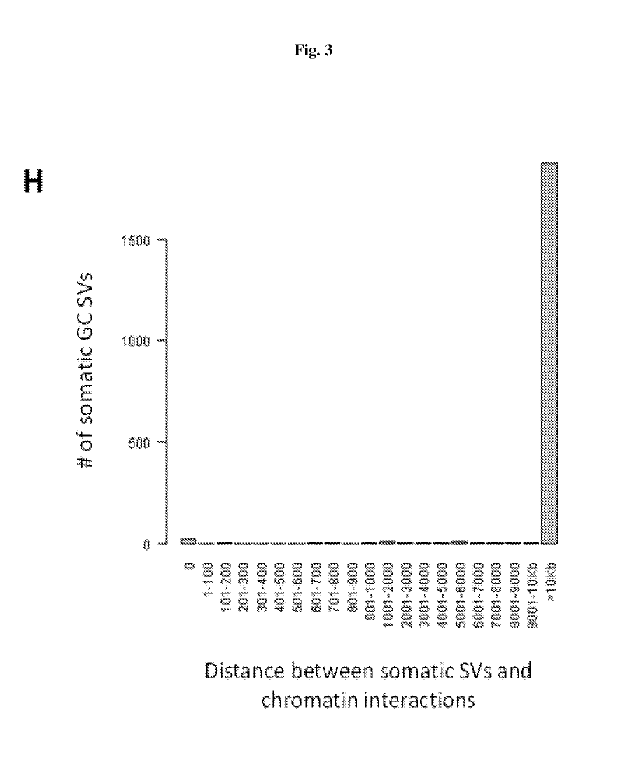

To investigate whether the two partner sites of germline and somatic SVs of the study were enriched for loci which are in proximity of each other in the nucleus, overlap of SVs were tested with genome-wide chromatin interaction data sets derived from ChIA-PET sequencing of the breast cancer cell line MCF-7 with the rationale that some chromatin interactions might be conserved across different cell types. (FIG. 3)

Since ChIA-PET data of a gastric cell line was not available, data from breast cancer cell line MCF-7 was used, with the assumption that some chromatin interactions are stable across different tissues. 1,667 germline and 1,945 somatic SVs of the 15 GCs were overlapped with 87,253 chromatin interactions of MCF-7 and 61 (3.7%) germline and 19 (1%) somatic SV overlaps were found, more than expected by chance (P<0.001, permutation based, FIG. 2E) indicating that chromatin interactions contribute to the shape of germline and somatic GC SVs.

### Example 3

### Rearrangement Hotspots in GC

14 recurrent somatic SVs were identified with stringent search criteria and an additional 173 were identified with relaxed search criteria. Recurrent rearrangements clustered in seven hotspots with FHIT, WWOX, MACROD2, PARK2, and PDE4D at known fragile sites and NAALADL2 and CCSER1 (FAM190A), at new hotspots. All recurrently rearranged genes were of relevance for cancer. Interestingly, tumor 17 and TMK1 which had the highest number of somatic SVs in the seven rearrangement hotspots (12 and 11, respectively), also ranged among the GCs with the largest number of somatic SVs (FIG. 1B), suggesting that either these rearrangement hotspots quickly accumulate rearrangements in tumors with genomic instability or that disruptions of the hotspot genes mechanistically contribute to genome instability. We also found recurrent tandem duplications at the MYC locus and recurrent deletions at the ATM locus, two key genes in cancer biology, further demonstrating that recurrent somatic SVs are likely of relevance to cancer biology.

### Example 4

### Recurrent Fusion Genes in GC

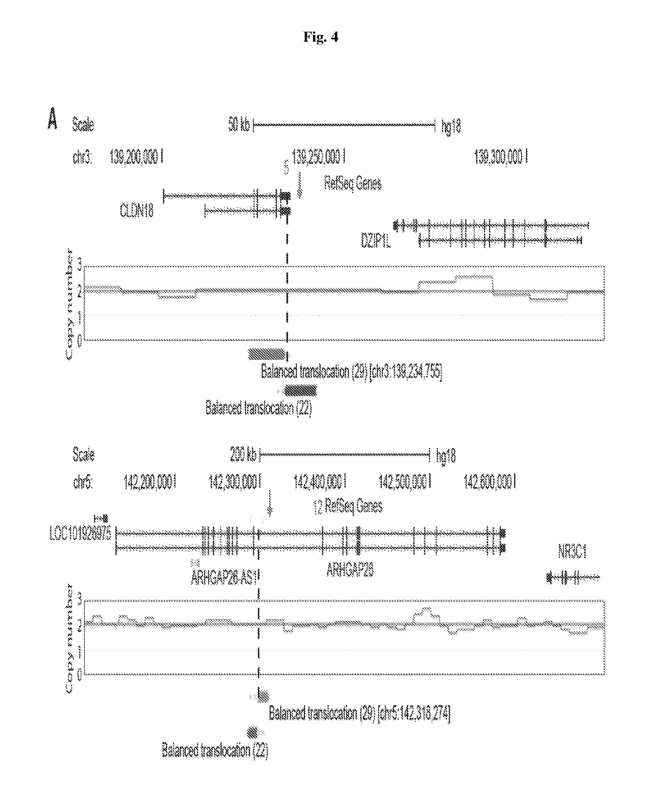

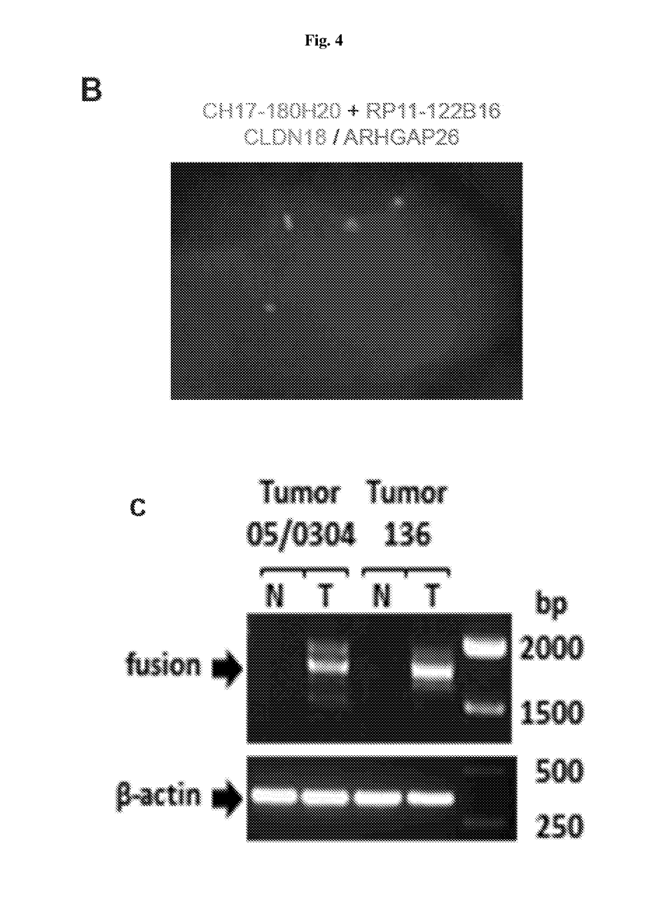

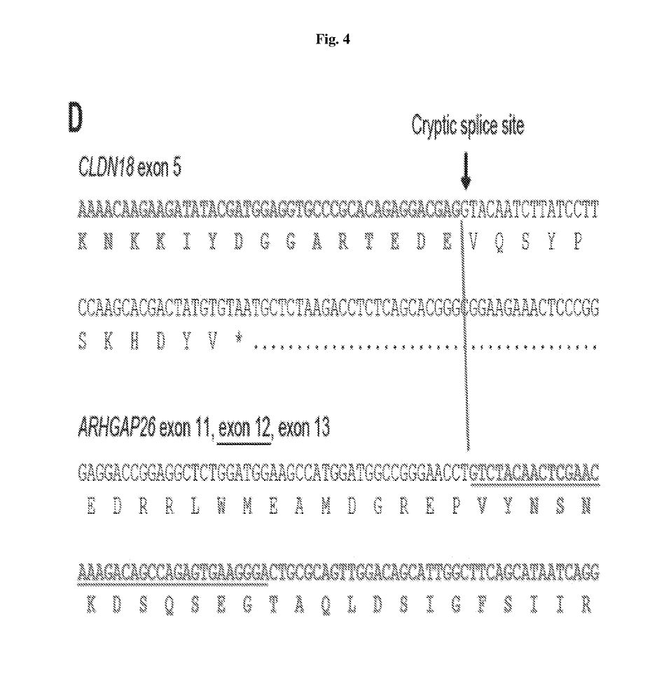

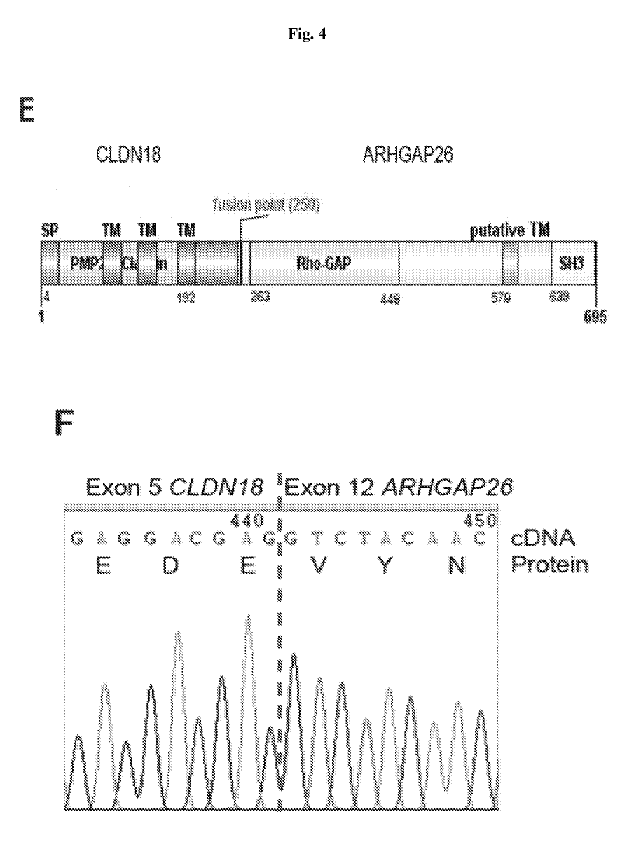

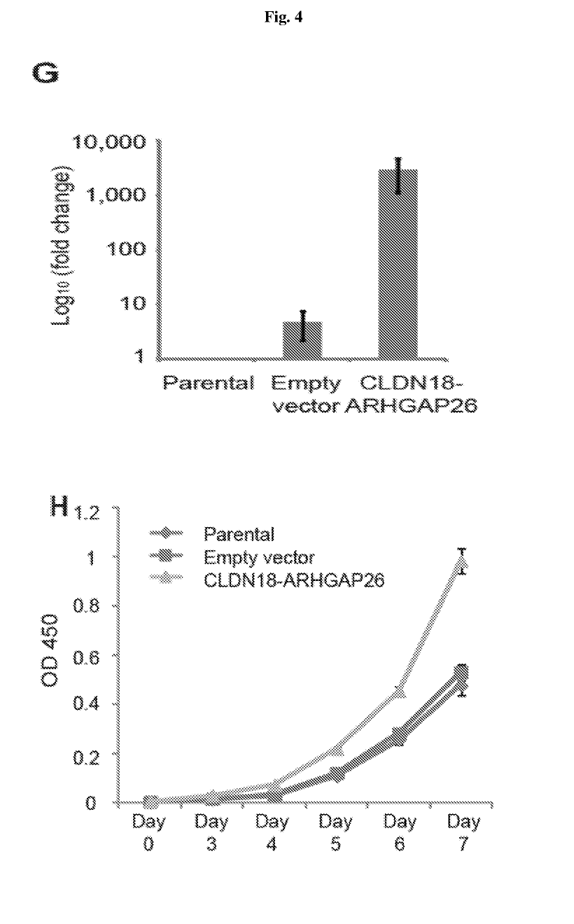

Using the somatic SVs of the 15 GCs, 136 fusion genes were predicted, 97 of them were validated by genomic PCR and Sanger sequencing, and the expression of 44 was confirmed by reverse transcription polymerase chain reaction (RT-PCR) in the respective tumours. Fifteen expressed fusion genes were in-frame. Since constitutively active oncogenic fusion genes are usually in-frame fusions, focus was placed on this category to screen an additional set of 85 GC tumor/normal pairs by RT-PCRs and found SNX2-PRDM6 in one additional tumor, CLDN18-ARHGAP26 and DUS2L-PSKH1 in two additional tumors, MLL3-PRKAG2 in three additional tumors, and CLEC16A-EMP2 in four additional tumors, giving overall frequencies of 2-5% (FIGS. 4A-C and 5 to 8). Statistical simulations were performed to assess the significance of such rates of recurrence. The statistical significance of the observed frequency of fusion genes was assessed using a randomization framework. 15 SV profiles were defined that mimic the type, number and size distributions of SVs identified in the samples sequenced by DNA-PET. The SVs of a 15 GCs test data set were simulated using the SV profiles and the frequency of recurrent SVs were assessed on a simulated validation set of 85 GC samples. Let N=10,000 be the number of random simulations and eₛ the frequency in the validation data set of an SV s present in the test data set, we define P values (eₛ) as p/N, where p is the number of simulations where a SV k exists with a frequency eₖ≧eₛ.

It was found that they were not expected by chance (P=0.00472), with higher levels of significance for two rediscoveries (P=9.98×10⁻⁵) and three rediscoveries (P=1.11×10⁻⁵). This suggests that these fusion genes are not randomly created but most likely by targeted rearrangement mechanisms and/or that the resulting fusion genes provide selective advantages,

### Example 5

### Effect of the Fusion Genes on Cell Proliferation

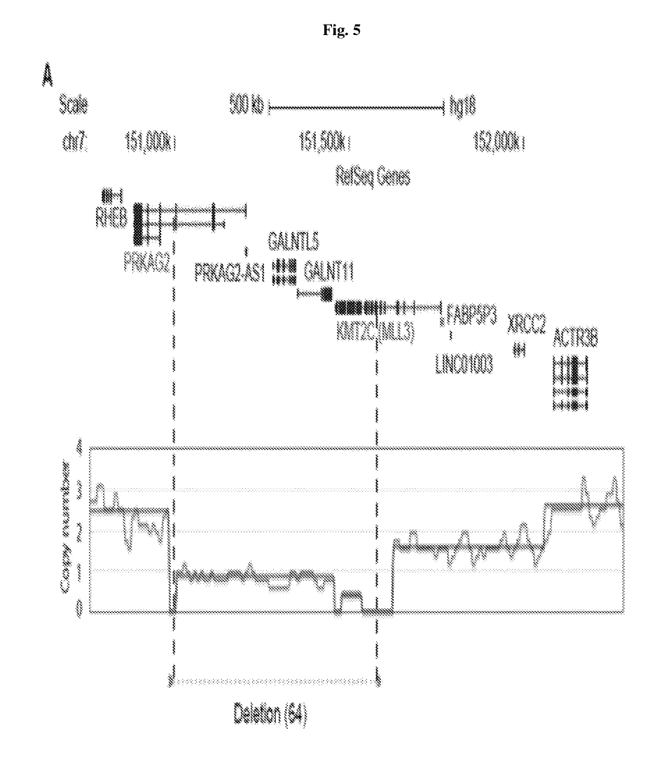

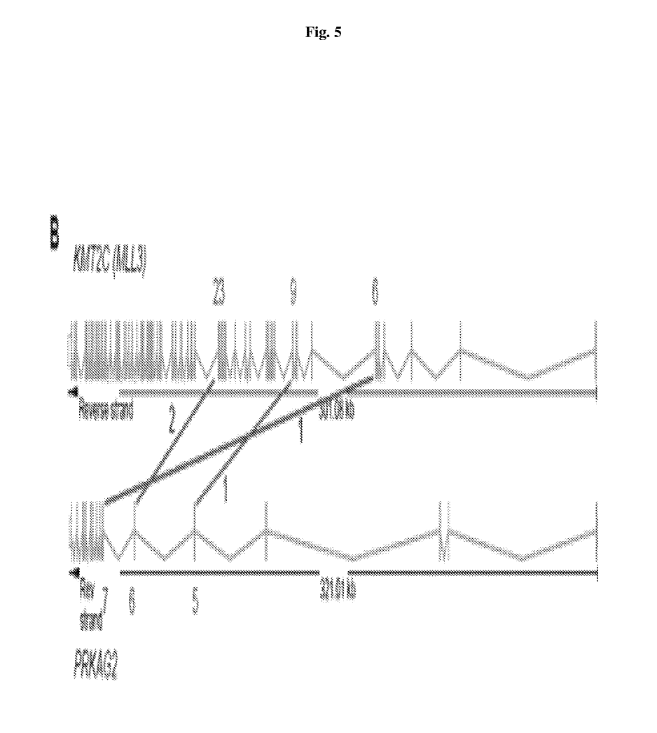

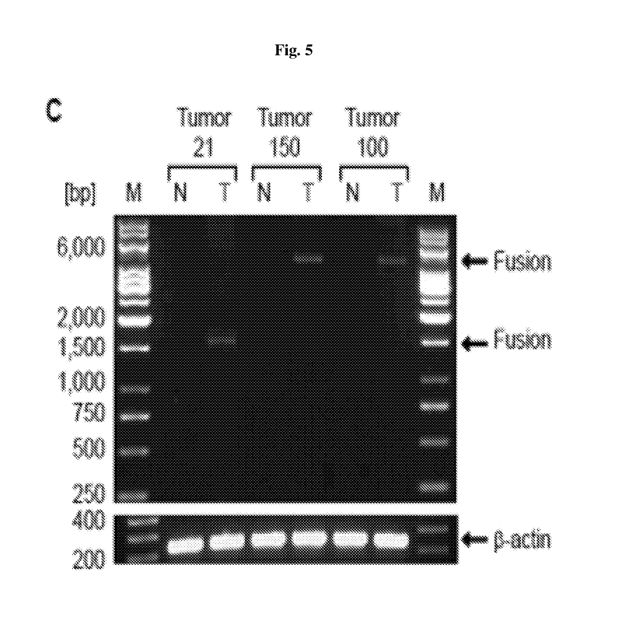

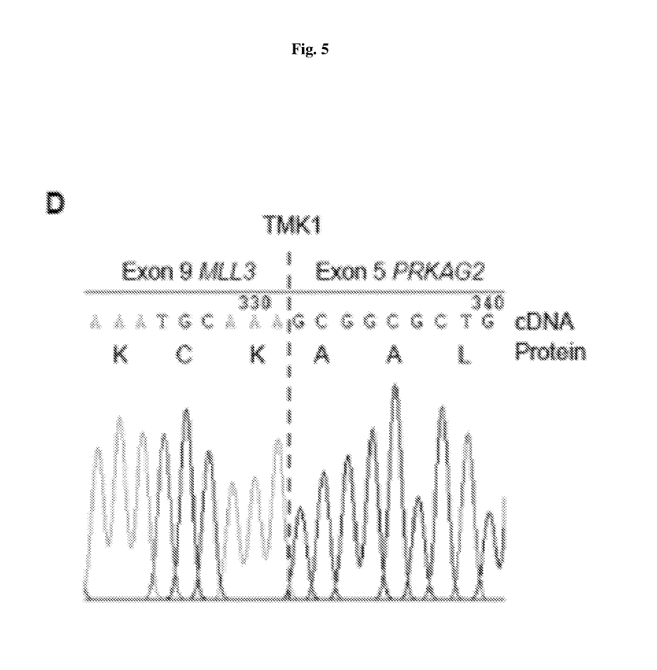

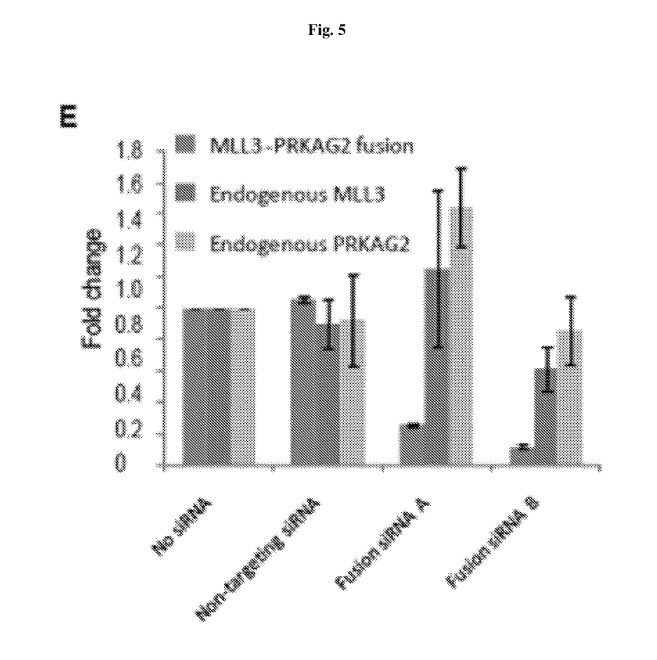

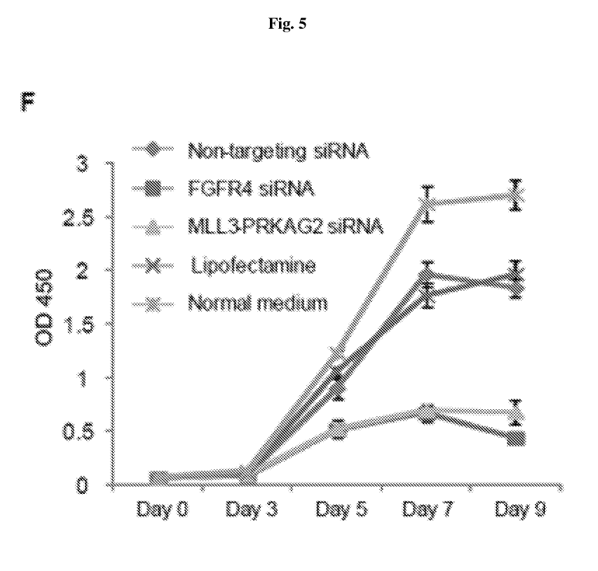

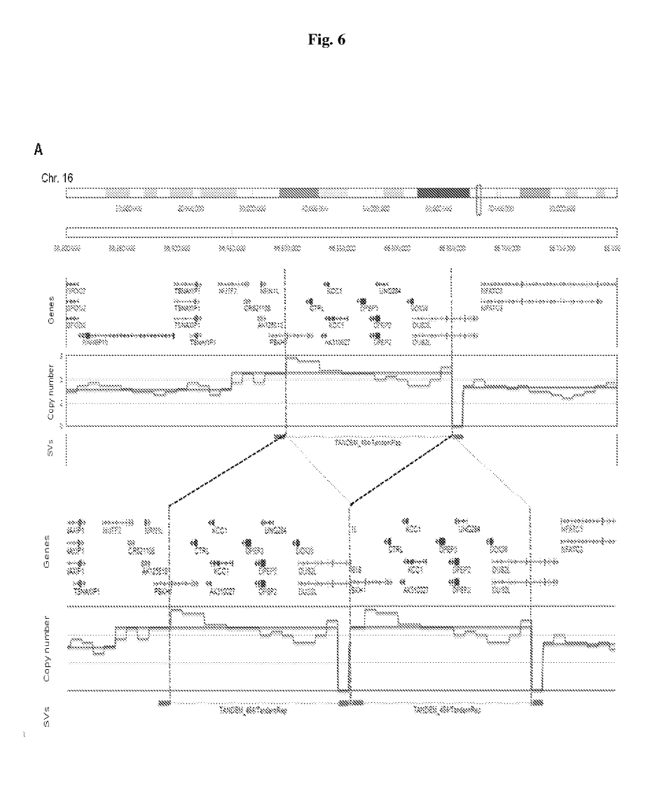

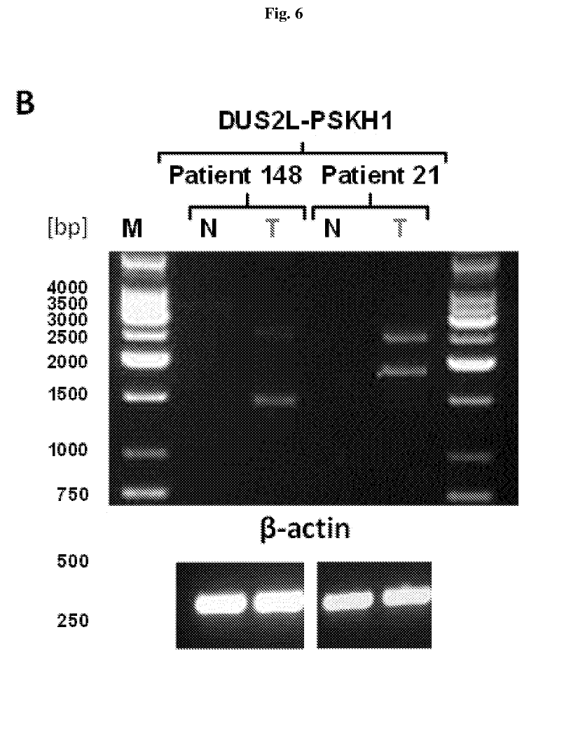

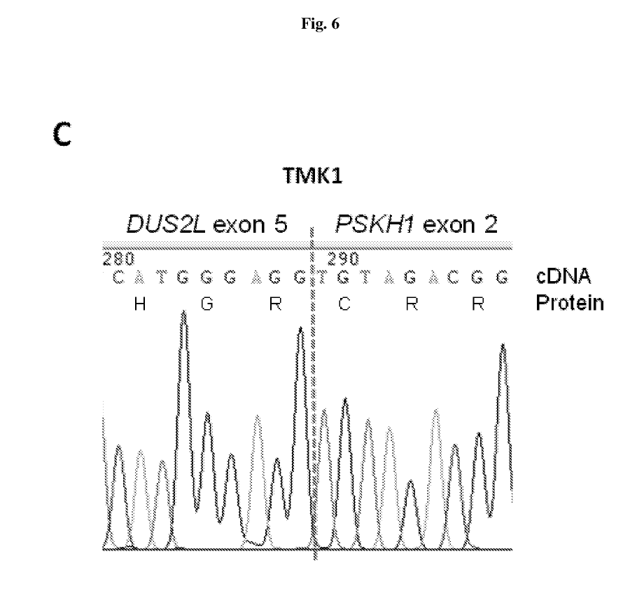

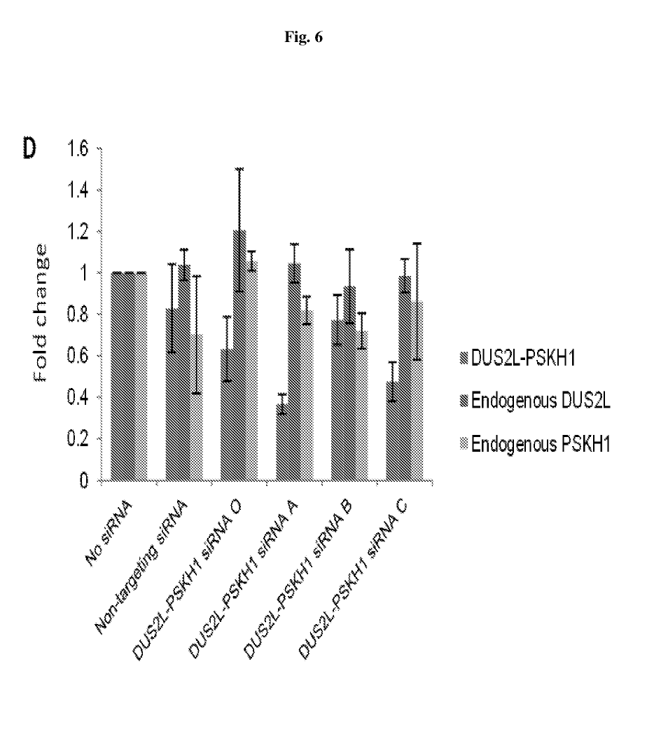

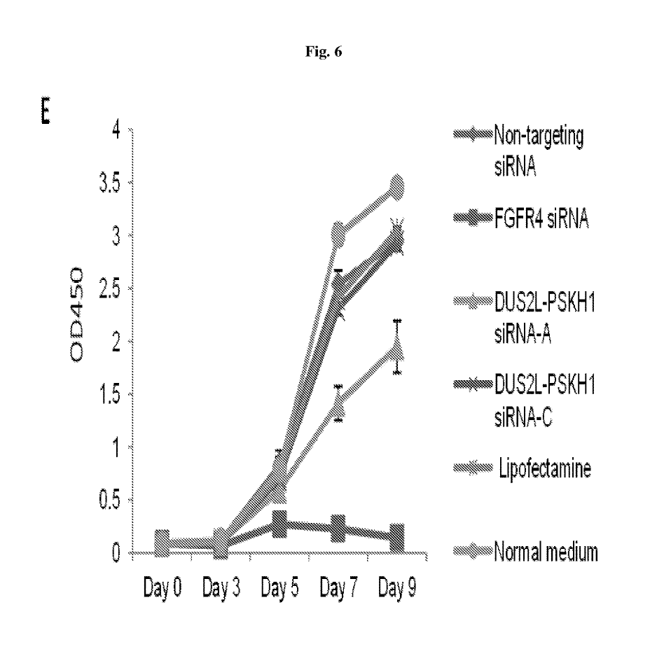

To explore if the fusion genes provided selective advantages, bioinformatics and cell biological approaches were used. In silico, a network fusion centrality analysis was used to predict driver fusion genes. Among the 136 fusion genes of this study, 38 were classified as potential driver fusion genes, including CLDN18-ARHGAP26, SNX2-PRDM6 and MLL3-PRKAG2 (Table 5). Since MLL3-PRKAG2 and DUS2L-PSKH1 in TMK1 were identified, short interfering RNA (siRNA) experiments specific for the fusion points of the MLL3-PRKAG2 and DUS2L-PSKH1 transcripts was performed. Reduced cell proliferation by 63% was observed when silencing MLL3-PRKAG2 (FIG. 5), but inconclusive changes were observed for DUS2L-PSKH1 knock-down cells (FIG. 6). Therefore, based on the frequency of 4% in GC, predicated driver properties, and the experimental evidence for a pro-proliferative effect, it is suggestive that MLL3-PRKAG2 is pro-carcinogenic for GC.

TABLE 5
Driver fusion gene prediction.

| | | | | All | All | | |
|---|---|---|---|---|---|---|---|
| | | | Fusion | Cancers | Cancers | Entrez | Entrez |
| | Partner | | Centrality | Citation # | Citation | gene1 | gene2 |
| Rank | Gene 1 | Partner Gene 2 | Score | Gene1 | # Gene2 | ID | ID |
| 1 | ROCK1 | ELF1 | 0.39152 | 44 | 7 | 6093 | 1997 |
| 2 | LIFR | GATA4 | 0.38719 | 8 | 17 | 3977 | 2626 |
| 3 | LOC96610 | BCR | 0.38562 | 1 | 156 | 96610 | 613 |
| 4 | GATAD2A | NCAN | 0.38272 | 2 | 3 | 54815 | 1463 |
| 5 | DGKD | INPP5D | 0.38268 | 4 | 18 | 8527 | 3635 |
| 6 | ZNF385D | EPHA3 | 0.38251 | 2 | 15 | 79750 | 2042 |
| 7 | ZBTB7C | SMAD2 | 0.38148 | 2 | 107 | 201501 | 4087 |
| 8 | PTPN11 | MYCBPAP | 0.38083 | 93 | 2 | 5781 | 84073 |
| 9 | ASPSCR1 | HGS | 0.38023 | 6 | 20 | 79058 | 9146 |
| 10 | CLDN18 | ARHGAP26 | 0.37873 | 8 | 2 | 51208 | 23092 |
| 11 | NRG1 | MTMR6 | 0.37836 | 45 | 6 | 3084 | 9107 |
| 12 | BCAS4 | PTPN1 | 0.37817 | 2 | 31 | 55653 | 5770 |
| 13 | RPL23A | NLK | 0.37731 | 2 | 6 | 6147 | 51701 |
| 14 | GHR | USH2A | 0.37657 | 24 | 1 | 2690 | 7399 |
| 15 | CRX | ANKRD24 | 0.37655 | 3 | 1 | 1406 | 170961 |
| 16 | MIR548W | TLK2 | 0.3759 | 0 | 2 | 0 | 11011 |
| 17 | MAP4 | SMARCC1 | 0.37561 | 4 | 20 | 4134 | 6599 |
| 18 | SLC20A2 | ANK1 | 0.37558 | 2 | 8 | 6575 | 286 |
| 19 | LUC7L | AXIN1 | 0.37535 | 4 | 42 | 55692 | 8312 |
| 20 | DTNA | PELI2 | 0.37527 | 2 | 2 | 1837 | 57161 |
| 21 | GRIN2D | GDF1 | 0.37513 | 6 | 1 | 2906 | 2657 |
| 22 | NCAM1 | OPCML | 0.3747 | 43 | 10 | 4684 | 4978 |
| 23 | CSNK1G2 | SCAMP4 | 0.37464 | 4 | 2 | 1455 | 113178 |
| 24 | CDKN2B | CDKN2A | 0.3738 | 76 | 670 | 1030 | 1029 |
| 25 | ZC3H15 | ITGAV | 0.37355 | 2 | 115 | 55854 | 3685 |
| 26 | TGIF1 | MYOM1 | 0.37341 | 9 | 1 | 7050 | 8736 |
| 27 | FLJ32810 | HLA-B | 0.37306 | 0 | 109 | 143872 | 3106 |
| 28 | HLA-B | FLJ32810 | 0.37306 | 109 | 0 | 3106 | 143872 |
| 29 | FLNC | FLJ45340 | 0.37253 | 6 | 0 | 2318 | 0 |
| 30 | SNX2 | PRDM6 | 0.37246 | 5 | 0 | 6643 | 93166 |
| 31 | PBX3 | RORB | 0.37142 | 6 | 3 | 5090 | 6096 |
| 32 | CDH22 | ADAMTSL4 | 0.37118 | 1 | 7 | 64405 | 54507 |
| 33 | C1ORF131 | RGS7 | 0.37108 | 1 | 3 | 128061 | 6000 |
| 34 | THRA | NR1D1 | 0.37086 | 26 | 2 | 7067 | 9572 |
| 35 | SMG1 | DCUN1D3 | 0.37083 | 6 | 2 | 23049 | 123879 |
| 36 | WDR88 | KIAA1303 | 0.37047 | 1 | 11 | 126248 | 57521 |
| 37 | SPATA17 | PTPN7 | 0.37042 | 2 | 9 | 128153 | 5778 |
| 38 | MLL3 | PRKAG2 | 0.37011 | 7 | 7 | 58508 | 51422 |
| 39 | KCNK2 | RNF2 | 0.36929 | 3 | 11 | 3776 | 6045 |
| 40 | EIF2C3 | STK40 | 0.36913 | 2 | 5 | 192669 | 83931 |
| 41 | PHF21A | CRY2 | 0.36909 | 3 | 7 | 51317 | 1408 |
| 42 | PILRB | PILRA | 0.36907 | 5 | 2 | 29990 | 29992 |
| 43 | KIRREL2 | SPTBN4 | 0.36876 | 2 | 3 | 84063 | 57731 |
| 44 | THAP4 | PARD3B | 0.36872 | 3 | 2 | 51078 | 117583 |
| 45 | YWHAB | BCAS1 | 0.36862 | 35 | 7 | 7529 | 8537 |
| 46 | DUS2L | PSKH1 | 0.3683 | 3 | 1 | 54920 | 5681 |
| 47 | NEK7 | TNFSF18 | 0.36809 | 0 | 6 | 140609 | 8995 |
| 48 | SMYD3 | MAST3 | 0.36783 | 12 | 1 | 64754 | 23031 |
| 49 | VDAC1 | CDKN2AIPNL | 0.36767 | 7 | 1 | 7416 | 91368 |
| 50 | SERF2 | PDIA3 | 0.3674 | 2 | 17 | 10169 | 2923 |
| 51 | CAT | CCAR1 | 0.36706 | 35 | 7 | 847 | 55749 |
| 52 | SLC19A2 | GATAD2B | 0.36671 | 6 | 4 | 10560 | 57459 |
| 53 | DAAM2 | RIMS1 | 0.36664 | 2 | 1 | 23500 | 22999 |
| 54 | LAMA3 | OSBPL1A | 0.36644 | 15 | 3 | 3909 | 114876 |
| 55 | MUC13 | MASP1 | 0.36589 | 1 | 4 | 56667 | 5648 |
| 56 | AP1M1 | LSM14A | 0.36577 | 7 | 1 | 8907 | 26065 |
| 57 | KIAA1529 | CTSL1 | 0.36428 | 1 | 21 | 57653 | 1514 |
| 58 | THBS4 | MSH3 | 0.36354 | 4 | 31 | 7060 | 4437 |
| 59 | STRBP | NDUFA8 | 0.3628 | 6 | 2 | 55342 | 4702 |
| 60 | DIRC3 | TNS1 | 0.36265 | 1 | 6 | 729582 | 7145 |
| 61 | RYR3 | APH1B | 0.36241 | 0 | 5 | 6263 | 83464 |
| 62 | MED13 | ABCA9 | 0.36239 | 7 | 3 | 9969 | 10350 |
| 63 | SOCS6 | TMX3 | 0.36181 | 4 | 0 | 9306 | 0 |
| 64 | EIF4G3 | ATPAF1 | 0.36162 | 8 | 1 | 8672 | 64756 |
| 65 | LOC100133991 | NMT1 | 0.36141 | 1 | 22 | 100133991 | 4836 |
| 66 | SOX5 | OVCH1 | 0.36134 | 9 | 0 | 6660 | 341350 |
| 67 | RNF138 | RNF125 | 0.36133 | 3 | 3 | 51444 | 54941 |
| 68 | TUT1 | IGHMBP2 | 0.36008 | 1 | 4 | 64852 | 3508 |
| 69 | OVCH1 | CCDC91 | 0.35958 | 0 | 2 | 341350 | 55297 |
| 70 | CAMTA1 | PRDM16 | 0.35942 | 6 | 12 | 23261 | 63976 |
| 71 | KIAA0999 | PCSK7 | 0.35923 | 3 | 9 | 23387 | 9159 |
| 72 | C18ORF1 | GABRB1 | 0.35905 | 2 | 2 | 753 | 2560 |
| 73 | TESC | FBXO21 | 0.35845 | 2 | 4 | 54997 | 23014 |
| 74 | TMEM49 | ACCN1 | 0.3584 | 7 | 2 | 81671 | 40 |
| 75 | SIPA1L3 | ZNF585A | 0.35823 | 3 | 1 | 23094 | 199704 |
| 76 | ZNF585A | SIPA1L3 | 0.35823 | 1 | 3 | 199704 | 23094 |
| 77 | KIAA0430 | NDE1 | 0.35797 | 1 | 4 | 9665 | 54820 |
| 78 | ALDH2 | MGAT4C | 0.35769 | 75 | 2 | 217 | 25834 |
| 79 | EMR3 | PEPD | 0.35768 | 1 | 8 | 84658 | 5184 |
| 80 | MYOM1 | LPIN2 | 0.35748 | 1 | 0 | 8736 | 9663 |
| 81 | INTS4 | RSF1 | 0.35725 | 1 | 8 | 92105 | 51773 |
| 82 | IMMP2L | DOCK4 | 0.35724 | 3 | 5 | 83943 | 9732 |
| 83 | C6ORF165 | RARS2 | 0.35711 | 3 | 2 | 154313 | 57038 |
| 84 | INTS9 | DCLK1 | 0.35685 | 2 | 4 | 55756 | 9201 |
| 85 | LOC729156 | GTF2IRD1 | 0.35662 | 0 | 3 | 0 | 9569 |
| 86 | CCNY | PCDH15 | 0.35661 | 1 | 1 | 219771 | 65217 |
| 87 | RABGAP1L | CACYBP | 0.35592 | 2 | 7 | 9910 | 27101 |
| 88 | MTMR2 | MAML2 | 0.3557 | 2 | 12 | 8898 | 84441 |
| 89 | SGCE | PEG10 | 0.35557 | 2 | 11 | 8910 | 23089 |
| 90 | FAM129C | PGLS | 0.35538 | 2 | 2 | 199786 | 25796 |
| 91 | GPI | KIAA0355 | 0.3552 | 19 | 2 | 2821 | 9710 |
| 92 | TFB2M | SMYD3 | 0.35463 | 2 | 12 | 64216 | 64754 |
| 93 | RNF157 | QRICH2 | 0.35461 | 1 | 2 | 114804 | 84074 |
| 94 | STOM | PALM2 | 0.35456 | 6 | 2 | 2040 | 114299 |
| 95 | MAP7 | RNF217 | 0.35449 | 6 | 2 | 9053 | 154214 |
| 96 | LOC401134 | CNGA1 | 0.35415 | 1 | 1 | 401134 | 1259 |
| 97 | RSL1D1 | BCAR4 | 0.35411 | 5 | 1 | 26156 | 400500 |
| 98 | COPG2 | AGBL3 | 0.35355 | 4 | 2 | 26958 | 340351 |
| 99 | CNN3 | SLC44A3 | 0.35319 | 3 | 3 | 1266 | 126969 |
| 100 | ADCY2 | OLFML2A | 0.35255 | 1 | 1 | 108 | 169611 |
| 101 | STARD10 | ODZ4 | 0.35244 | 4 | 1 | 10809 | 26011 |
| 102 | FBXO42 | CROCCL2 | 0.35224 | 2 | 1 | 54455 | 114819 |
| 103 | PHKB | GPT2 | 0.3521 | 2 | 1 | 5257 | 84706 |
| 104 | NAIF1 | CIZ1 | 0.35175 | 2 | 7 | 203245 | 25792 |
| 105 | C9ORF126 | MOBKL2B | 0.35143 | 2 | 4 | 286205 | 79817 |
| 106 | ST3GAL3 | KDM4A | 0.3505 | 3 | 0 | 6487 | 0 |
| 107 | DHDDS | FAM76A | 0.35028 | 1 | 3 | 79947 | 199870 |
| 108 | INSM2 | YTHDF3 | 0.34981 | 1 | 4 | 84684 | 253943 |
| 109 | KIAA1045 | CEP110 | 0.34943 | 2 | 5 | 23349 | 11064 |
| 110 | BSN | EGFEM1P | 0.34896 | 1 | 0 | 8927 | 0 |
| 111 | BAI3 | LMBRD1 | 0.34894 | 2 | 3 | 577 | 55788 |
| 112 | CDH13 | ACSS1 | 0.34886 | 36 | 1 | 1012 | 84532 |
| 113 | KCNK5 | CYP3A43 | 0.34871 | 1 | 7 | 8645 | 64816 |
| 114 | MPND | GLTSCR1 | 0.34864 | 1 | 4 | 84954 | 29998 |
| 115 | NIPBL | SPEF2 | 0.34842 | 3 | 2 | 25836 | 79925 |
| 116 | COL21A1 | C6ORF223 | 0.34825 | 2 | 1 | 81578 | 221416 |
| 117 | LOC644974 | DBR1 | 0.34767 | 1 | 2 | 644974 | 51163 |
| 118 | HARBI1 | AMBRA1 | 0.34766 | 2 | 2 | 283254 | 55626 |
| 119 | MOBKL2B | PCA3 | 0.34762 | 4 | 9 | 79817 | 50652 |
| 120 | SLC39A11 | SDK2 | 0.34738 | 1 | 1 | 201266 | 54549 |
| 121 | MTMR2 | SYVN1 | 0.34732 | 2 | 2 | 8898 | 84447 |
| 122 | NECAB1 | OTUD6B | 0.34658 | 1 | 1 | 64168 | 51633 |
| 123 | FAM65B | SPAG16 | 0.34618 | 2 | 1 | 9750 | 79582 |
| 124 | TMEM135 | MTMR2 | 0.34572 | 2 | 2 | 65084 | 8898 |
| 125 | C14ORF53 | ATP6V1D | 0.34565 | 1 | 3 | 440184 | 51382 |
| 126 | ACOXL | FBLN7 | 0.3455 | 2 | 1 | 55289 | 129804 |
| 127 | FRY | KIAA1328 | 0.34394 | 2 | 4 | 10129 | 57536 |
| 128 | MIR548W | TANC2 | 0.34288 | 0 | 1 | 0 | 26115 |
| 129 | KIAA0355 | GPATCH1 | 0.34217 | 2 | 1 | 9710 | 55094 |
| 130 | CLEC16A | EMP2 | 0.34199 | 1 | 6 | 23274 | 2013 |
| 131 | CCDC46 | CPD | 0.34004 | 1 | 5 | 201134 | 1362 |
| 132 | ABHD3 | KIAA1772 | 0.33999 | 2 | 1 | 171586 | 80000 |
| 133 | FHOD3 | CEP192 | 0.33888 | 3 | 6 | 80206 | 55125 |
| 134 | C19ORF26 | SBNO2 | 0.33591 | 2 | 1 | 255057 | 22904 |
| 135 | TMEM132B | TMEM132D | 0.33373 | 1 | 1 | 114795 | 121256 |
| 136 | LOC731220 | FAM160A1 | 0.3278 | 0 | 2 | 731220 | 729830 |

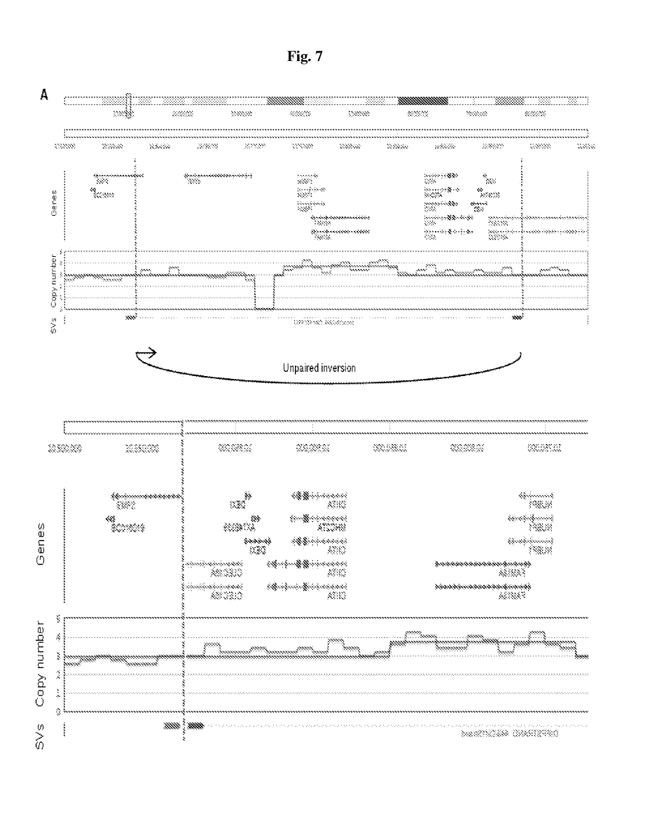

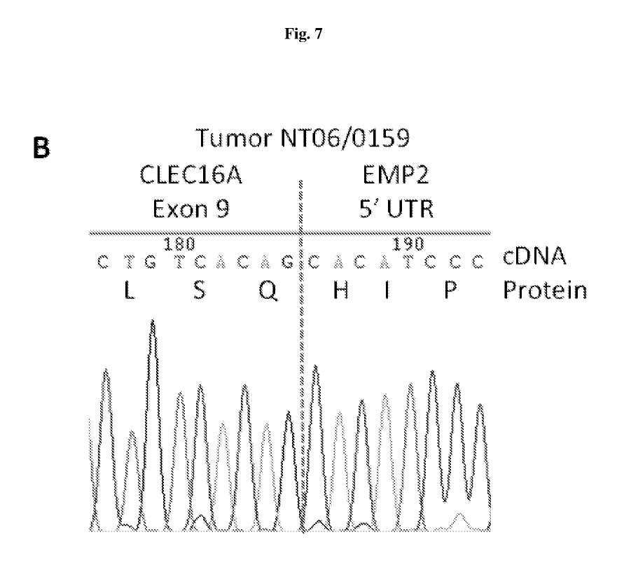

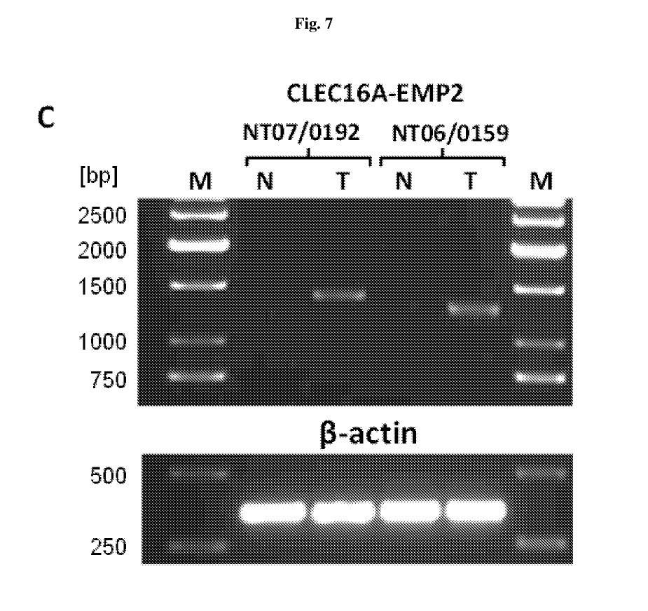

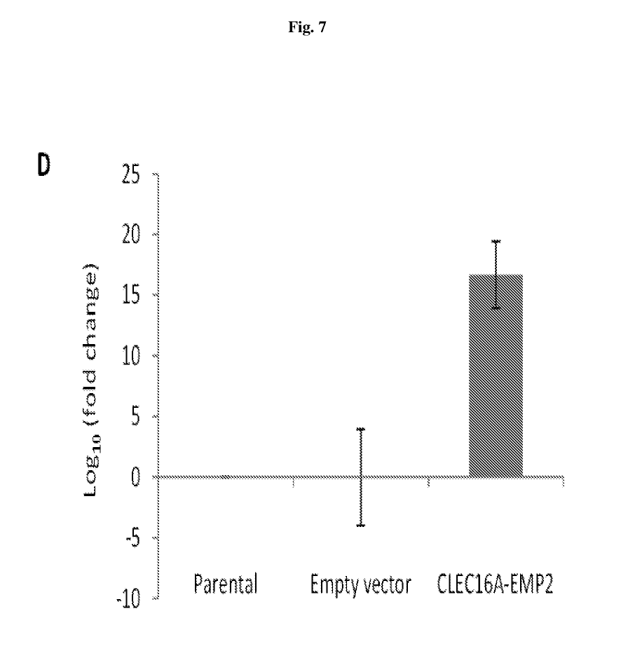

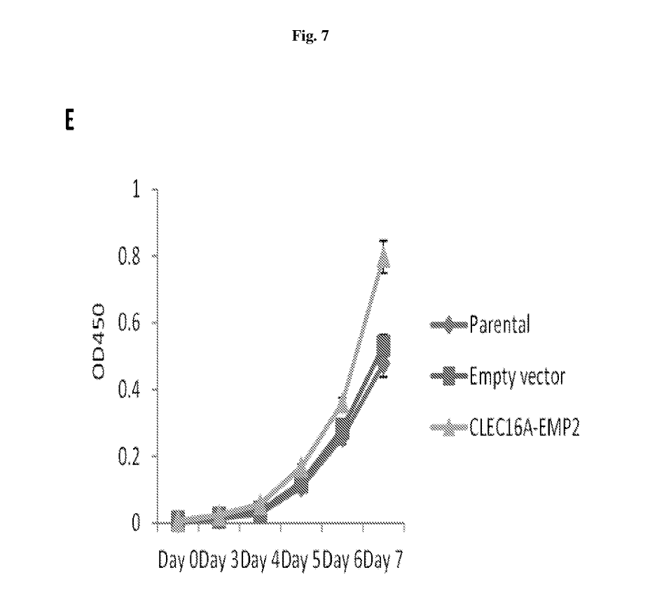

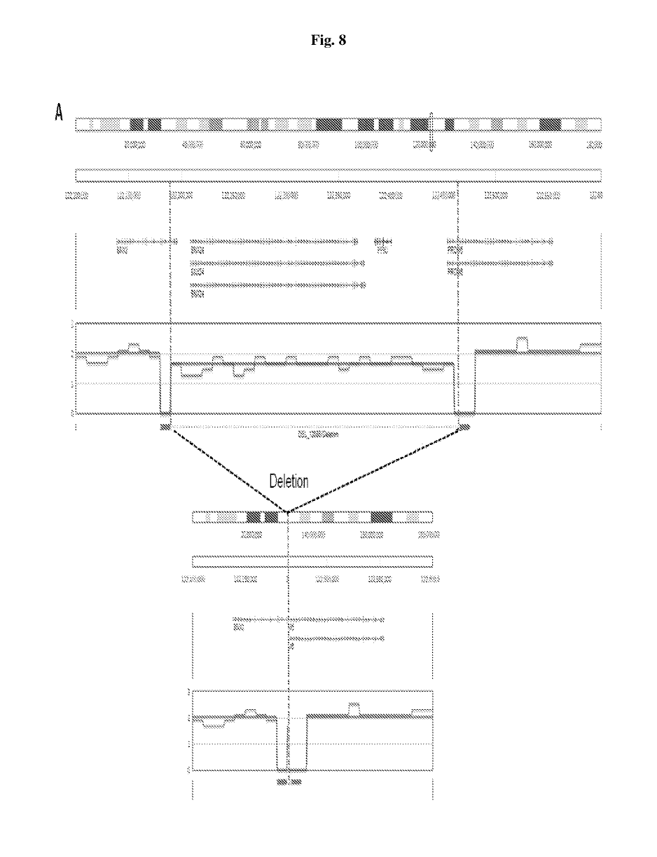

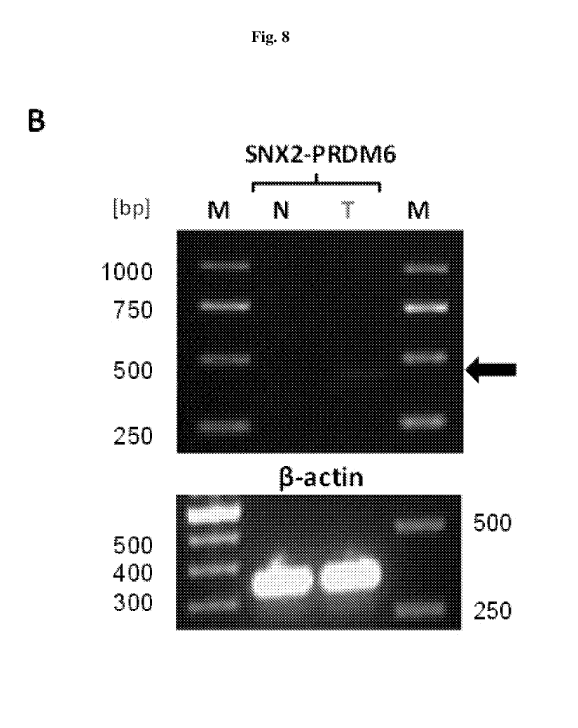

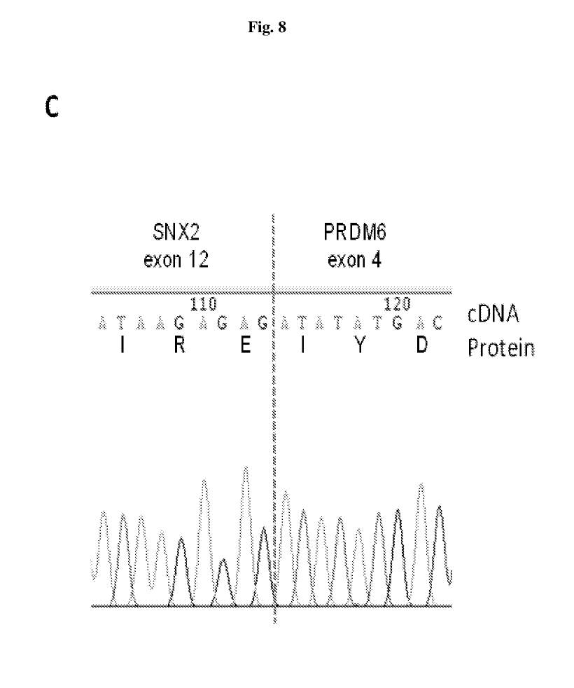

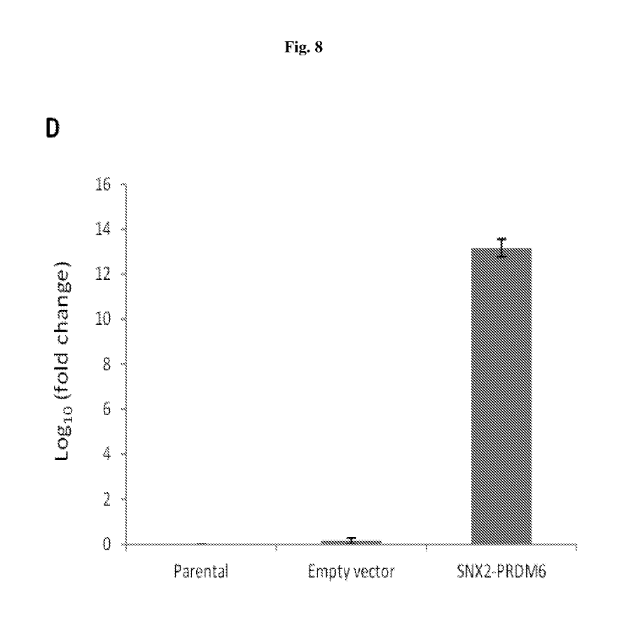

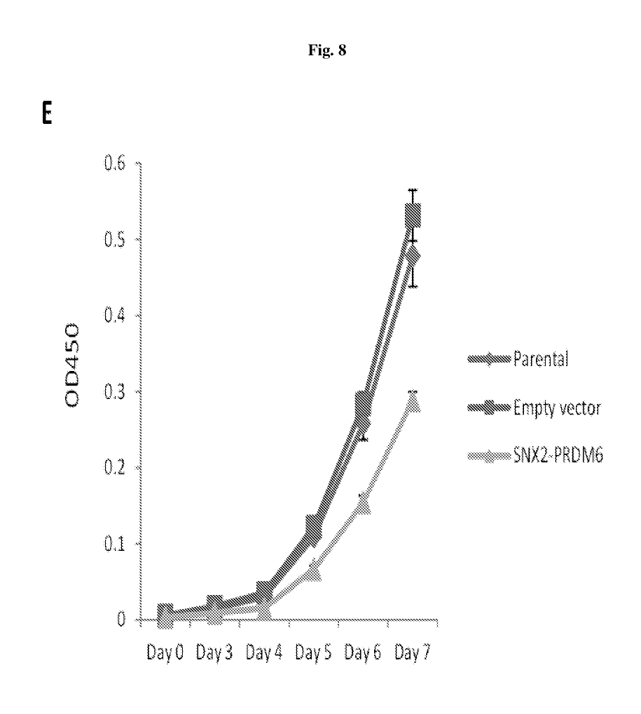

To investigate the function of CLDN18-ARHGAP26, CLEC16A-EMP2 and SNX2-PRDM6 in GC, stable overexpression was created in GC cell line HGC27, and showed increased cell proliferation rates for CLDN18-ARHGAP26 (85% increase, P=4.2×10⁻⁶, T-test FIGS. 4G, H) and CLEC16A-EMP2 (50% increase, P=7.9×10⁻⁵, T-test; FIG. 7) but a decreased proliferation rate for SNX2-PRDM6 (46% decrease, P=9×10⁻⁶, T-test; FIG. 8).

The high proliferation rate by overexpression of CLDN18-ARHGAP26 suggested an oncogenic role for this fusion gene, and further investigation of its function was performed. CLDN18-ARHGAP26 encodes a 75.6 kDa fusion protein containing all four transmembrane domains of CLDN18 and the RhoGAP domain of ARHGAP26, but lacking the C-terminal PDZ-binding motif of CLDN18 (FIG. 4E) that mediates interactions with zonula occludens scaffold proteins (ZO-1, ZO-2, ZO-3). CLDN18 belongs to the family of claudin proteins, which are components of the tight junctions (TJs). ARHGAP26 (GRAF1) binds to focal adhesion kinase (FAK), which modulates cell growth, proliferation, survival, adhesion and migration. ARHGAP26 can also negatively regulate the small GTP-binding protein RhoA, which is well known for its growth promoting effect in RAS-mediated malignant transformation.

In all three tumors with CLDN18-ARHGAP26 fusions, the transcripts were joined by a cryptic splice site within the coding region of exon 5 of CLDN18 and the regular splice site of exon 12 of ARHGAP26 (FIG. 4D). On the genomic level, we validated the CLDN18-ARHGAP26 rearrangement in tumor 136 by fluorescence in situ hybridization (FISH, FIG. 4B) and PCR/Sanger sequencing (FIG. 4C). Using custom capture sequencing, the genomic fusion points in tumor 07K611T were identified to 2,342 bp downstream of CLDN18 (FIG. 4A) indicating that the cryptic splice site mediates an in-frame fusion even when the breakpoint is downstream of the CLDN18 gene.

### Example 6

### Loss of Epithelial Phenotype in Patient Specimen and MDCK Cells Expressing CLDN18-ARHGAP26

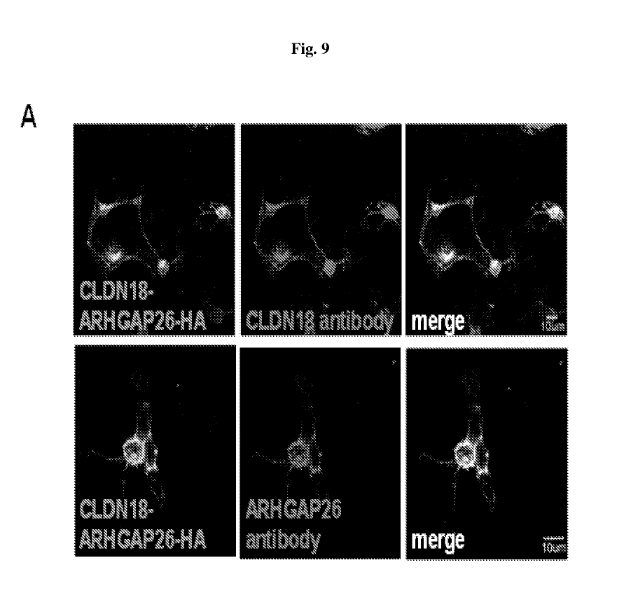

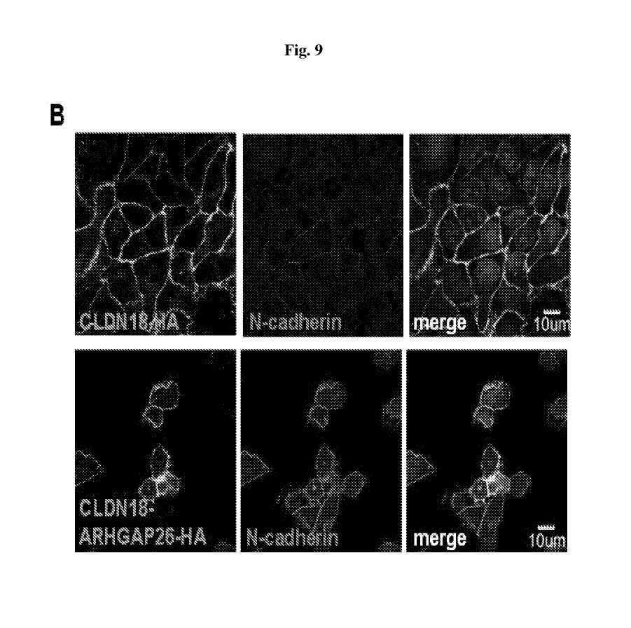

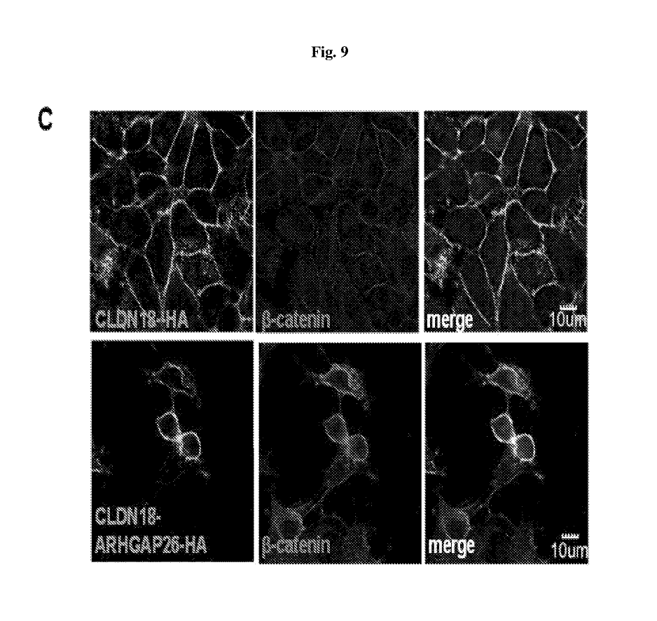

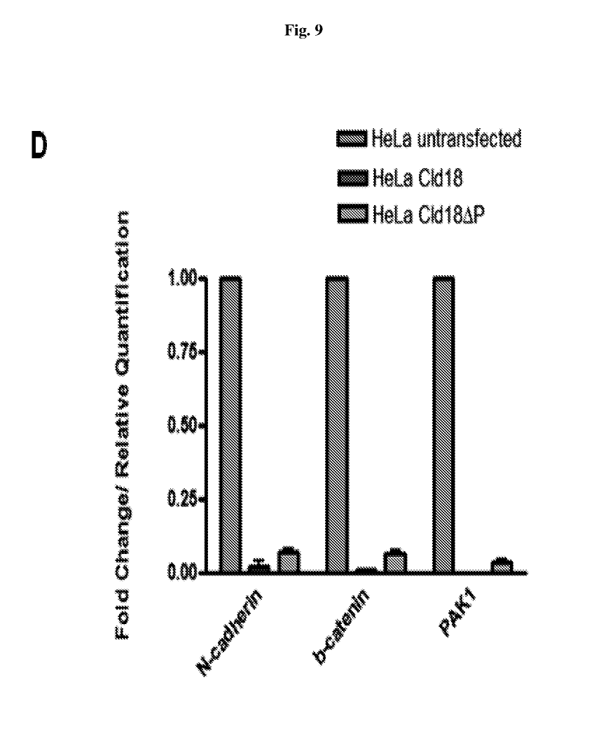

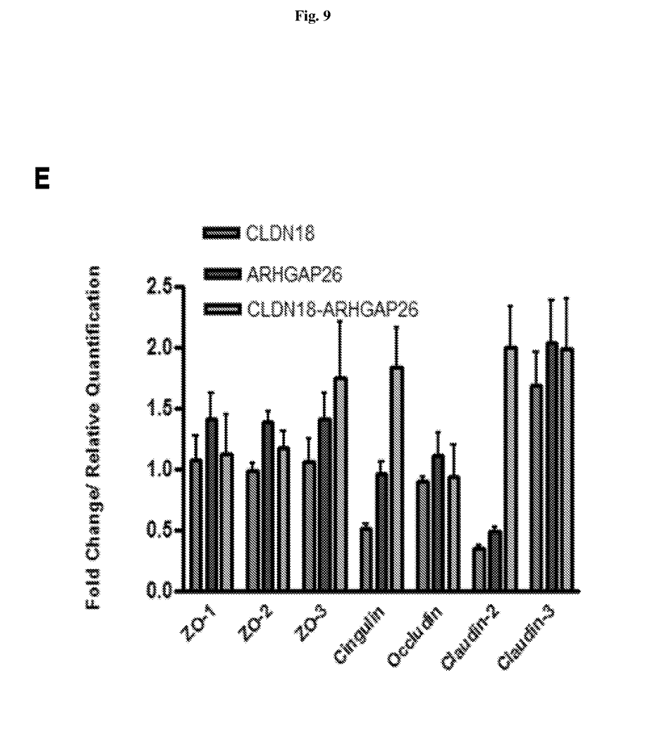

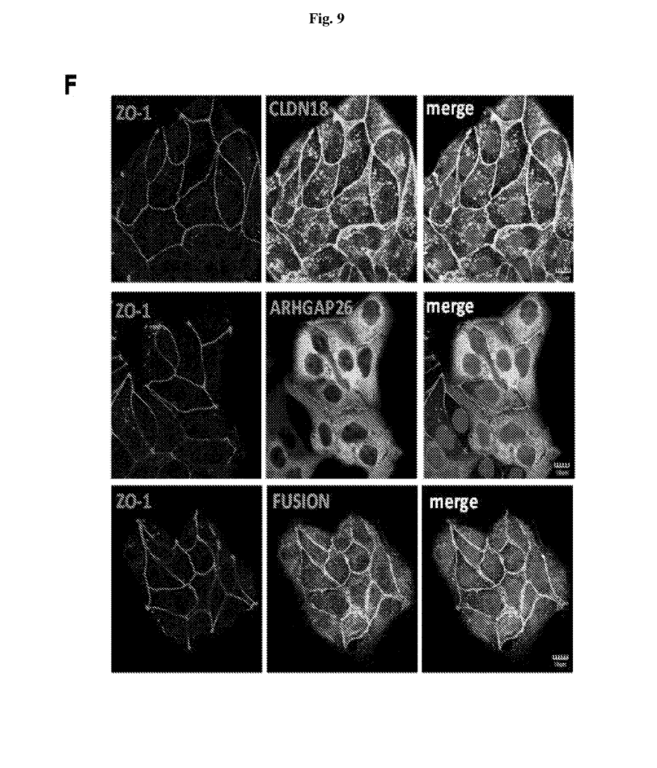

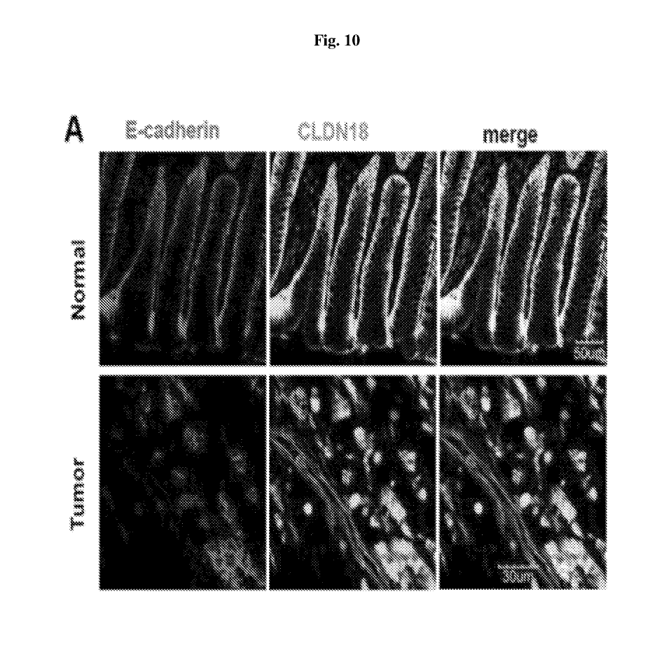

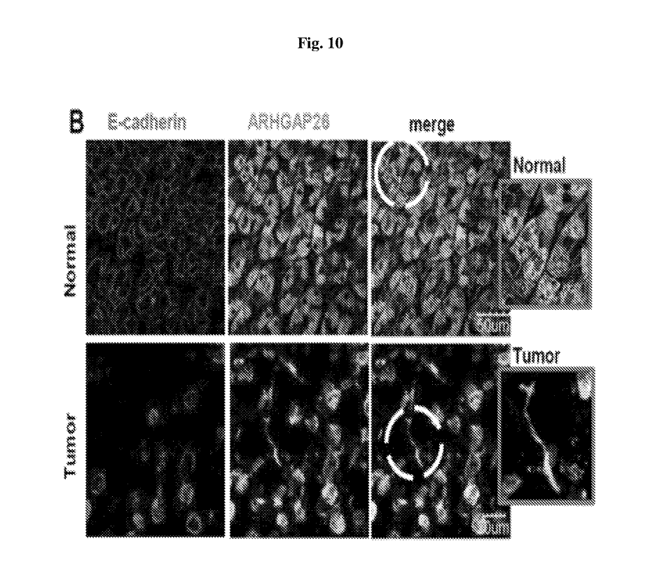

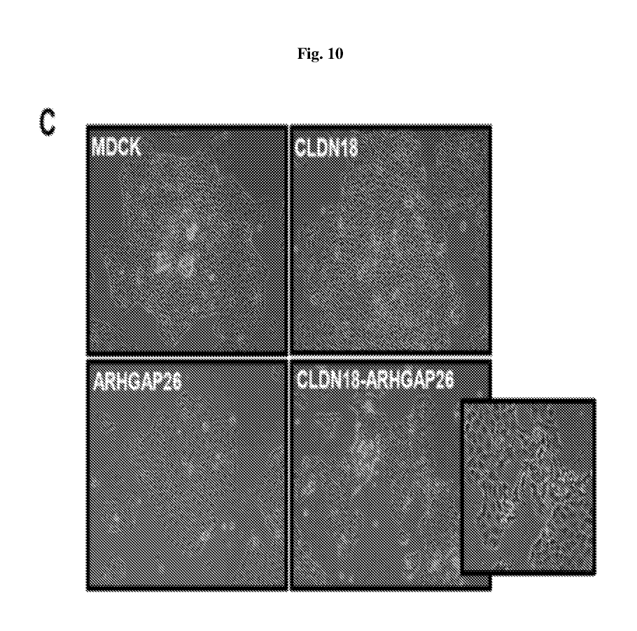

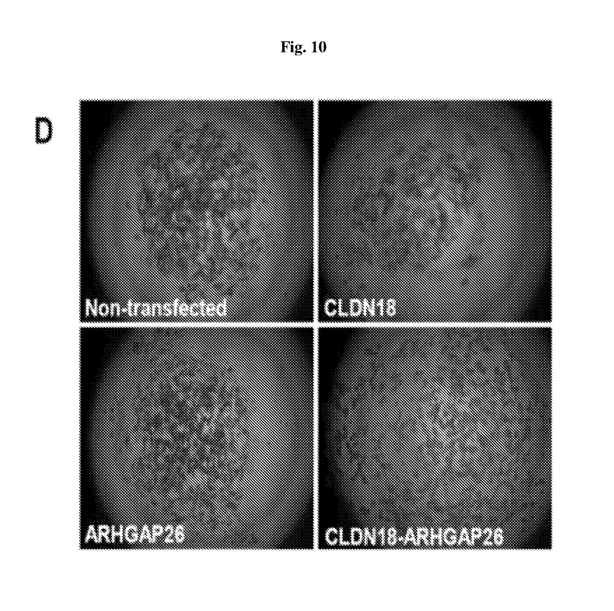

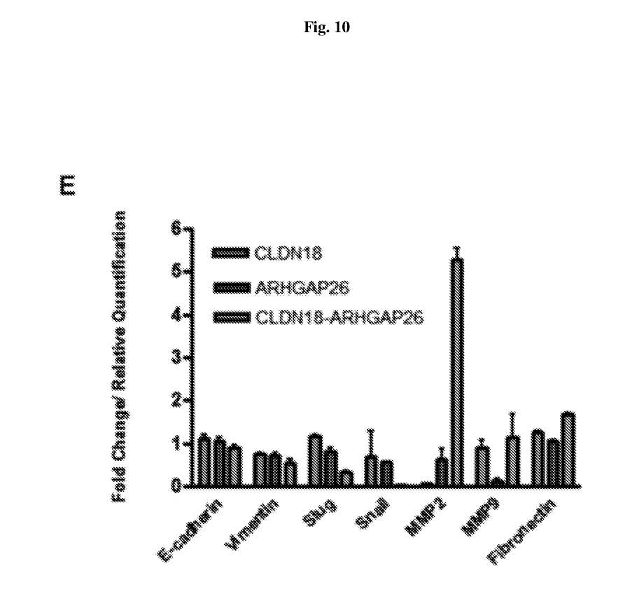

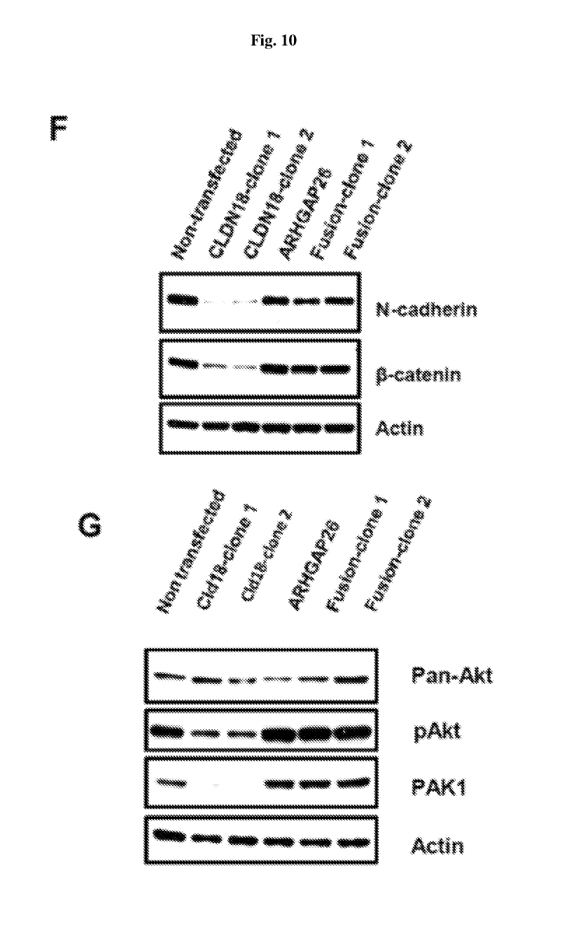

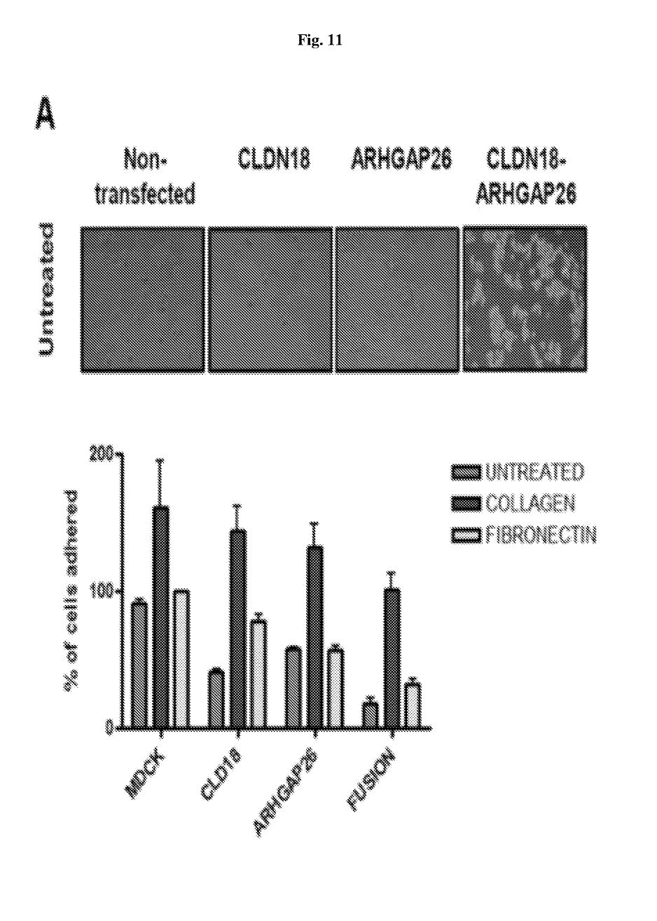

For immunofluorescence in tumor specimens, CLDN18 and ARHGAP26 antibodies were used which both were able to detect the CLDN18-ARHGAP26 fusion protein (FIG. 9A). In normal and fusion expressing tumor stomach specimens, CLDN18 protein was observed in the plasma membrane of epithelial cells lining the gastric pit region and at the base of the gastric glands (FIG. 10A). ARHGAP26 was previously detected on pleiomorphic tubular and punctate membrane structures in HeLa cells. In this study, ARHGAP26 was observed in normal stomach on vesicular structures throughout the gastric mucosa (FIG. 10B). In contrast to the well differentiated normal gastric epithelium, stomach tumor specimens expressing CLDN18-ARHGAP26 showed a disorganized structure. While the epithelial marker CDH1 (E-cadherin) was expressed at the membrane of epithelial cells in control tissues, it showed either an intracellular punctate distribution or was absent from cells in the tumor sample (FIG. 10A, B). CLDN18-ARHGAP26 was present in both E-cadherin positive and negative cells in the tumor sample, with the E-cadherin negative cells showing mesenchymal features (FIG. 10A, B), consistent with the fusion protein altering cell-cell adhesion leading to a loss of the epithelial phenotype. Overall, the fusion gene correlates with fatal impairment of gastric epithelial integrity.

To understand the contribution of the fusion protein to the observed changes in epithelial integrity in the tumor sample, CLDN18, ARHGAP26 or CLDN18-ARHGAP26 were stably expressed in non-transformed epithelial MDCK cells. Viewed by phase contrast, control and MDCK-CLDN18 cell cultures showed the characteristic epithelial morphology (FIG. 10C). While MDCK-ARHGAP26 cells were slightly more spindle-shaped and had short protrusions, MDCK-CLDN18-ARHGAP26 cells displayed a dramatic loss of epithelial phenotype and long protrusions, indicative of epithelial-mesenchymal transition (EMT) (FIG. 10C). Cell aggregation assays indicated poor aggregation for MDCK-CLDN18-ARHGAP26 cells (FIG. 10D) suggesting that indeed the fusion gene causes the observed epithelial changes Similar results were also obtained with HGC27 cells.

To evaluate if the phenotypic changes induced by CLDN18-ARHGAP26 reflected an EMT, the expression of various EMT markers was investigated using quantitative PCR (qPCR). While E-cadherin mRNA levels were unchanged in ARHGAP26 and CLDN18-ARHGAP26 expressing cells, mRNA of the master EMT regulators SNAI1 (Snail) and SNAI2 (Slug) were decreased (FIG. 10E). MDCK-CLDN18-ARHGAP26 showed a 5.2-fold increase in MMP2 (matrix metalloproteinase 2) mRNA levels relative to control MDCK cells (FIG. 10E), suggesting changes in extracellular matrix (ECM) adhesion induced by the fusion gene.

Interestingly, expression of CLDN18, but not the fusion protein, down-regulated N-cadherin and β-catenin expression was observed in transformed HeLa cells (FIGS. 10F and 9B-D), suggesting that CLDN18 can reverse the switch from an epithelial to a mesenchymal cadherin observed during EMT and suppress Wnt signaling, respectively. Wnt signaling is hyperactivated in many cancers, and N-cadherin expression activates AKT signaling, which is hyperactivated in many tumors. Indeed, pAKT protein levels, as well as those of the downstream effectors p21 activated kinase (PAK), were reduced in HeLa cells overexpressing CLDN18 as compared to controls (FIG. 10G). This suggests a role for CLDN18 as a tumor suppressor, by dampening AKT and Wnt signaling.

### Example 7

### CLDN18-ARHGAP26 Reduces Cell-Extracellular Matrix Adhesion

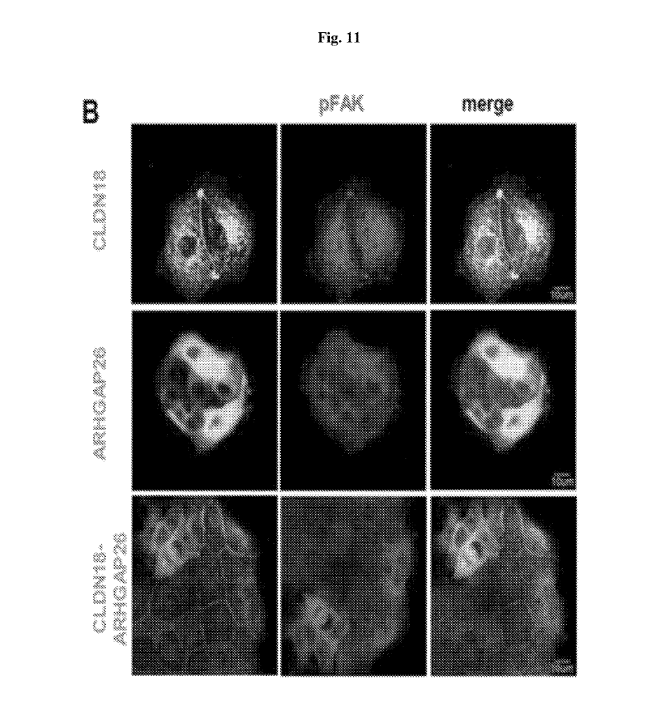

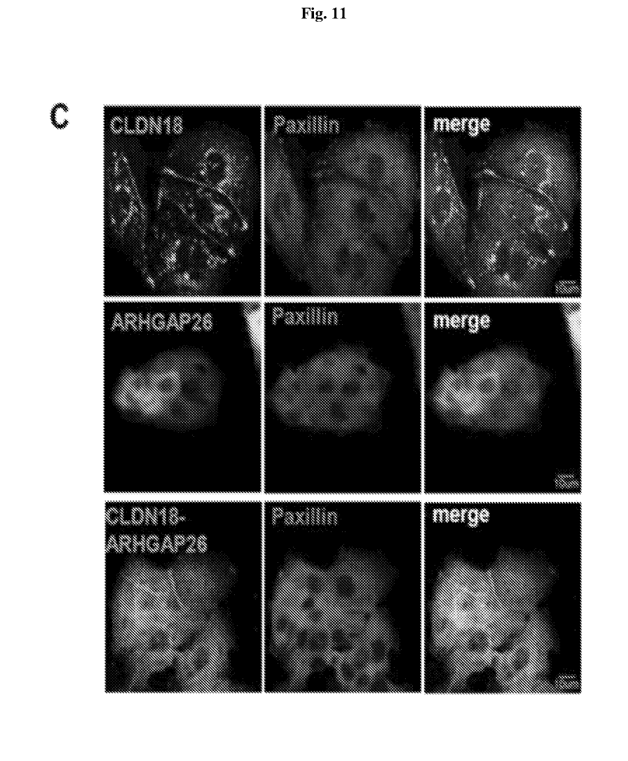

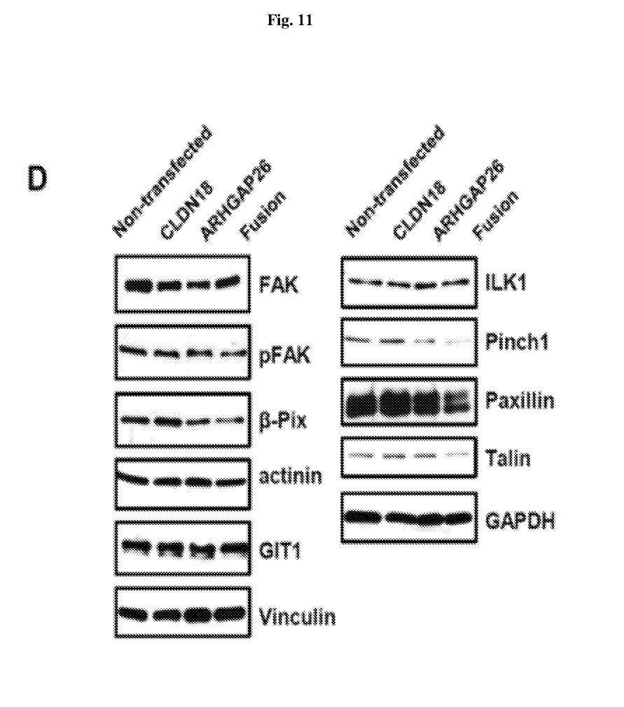

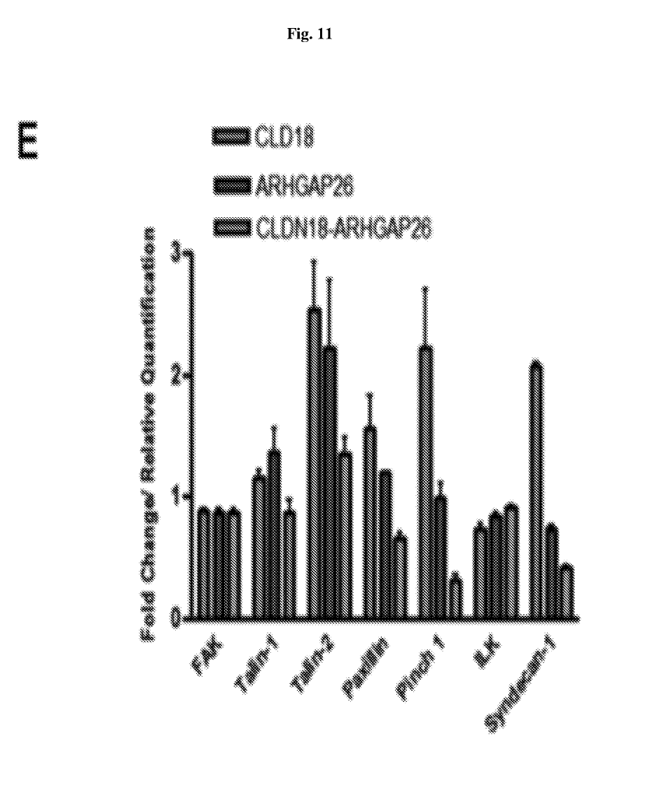

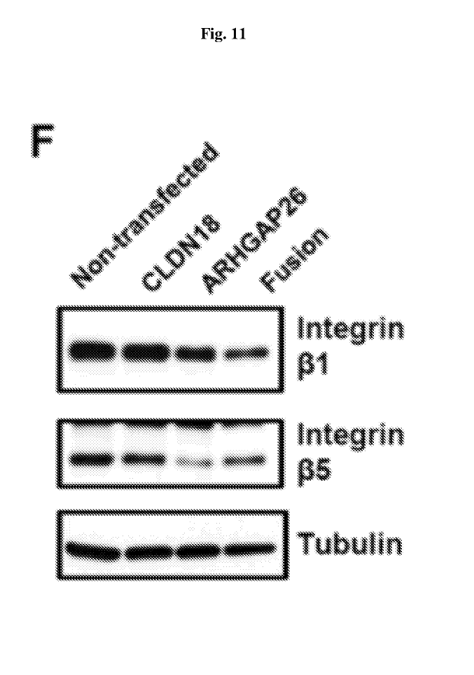

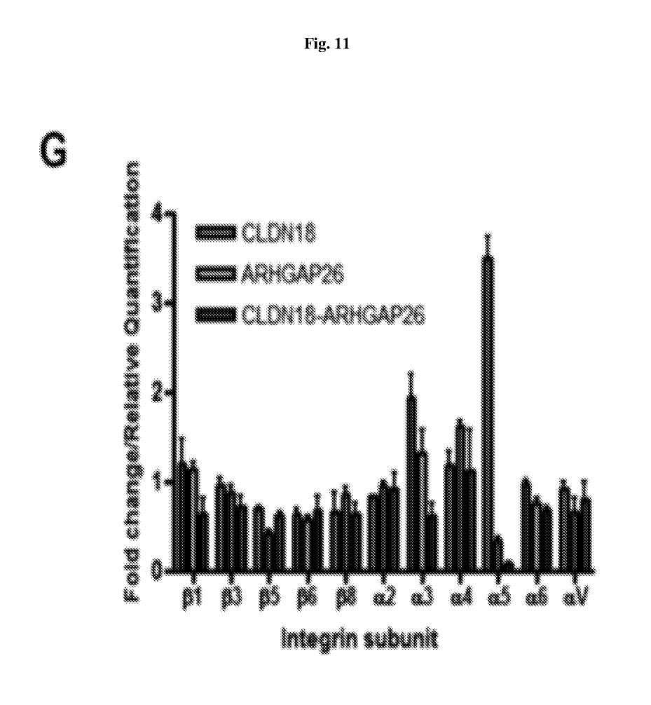

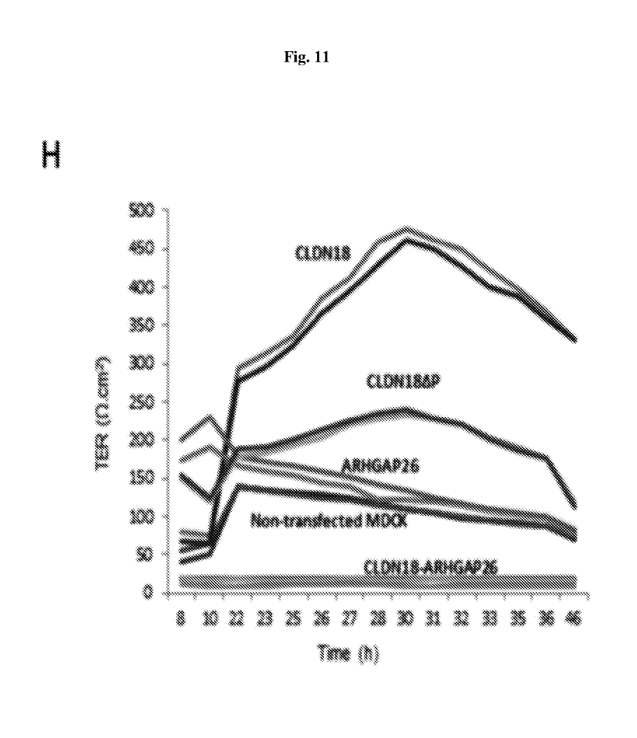

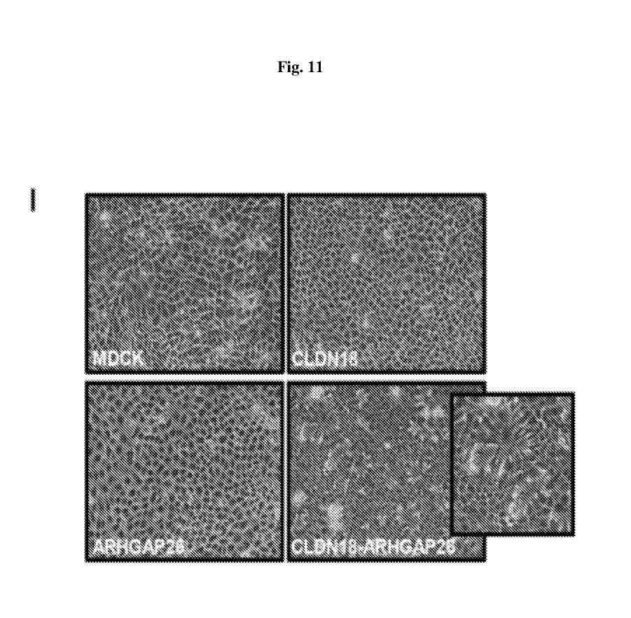

ARHGAP26 likely affects adhesion of cells to the ECM through its interaction with FAK and its regulation of RhoA, which in turn regulates focal adhesions. Adhesion assays showed that control and MDCK-CLDN18 cells attached and spread on either untreated or ECM-coated surfaces. Not only did ARHGAP26 and, even more so, CLDN18-ARHGAP26 expressing cells attach less efficiently to the surfaces (FIG. 11A), but the cells that did attach were still rounded-up two hours after seeding (FIG. 11A), showing that the fusion gene potentiates the effect of ARHGAP26 and strongly affects cell-ECM adhesive properties. The SH3 domain of ARHGAP26, present in the fusion protein, binds to the focal adhesion molecules, FAK and PXN (Paxillin). The effect of CLDN18-ARHGAP26 expression on focal adhesion proteins was therefore examined pFAK and Paxillin were detected at the free edge of MDCK-CLDN18 and MDCK-ARHGAP26, but were absent from this location in MDCK-CLDN18-ARHGAP26 cells (FIG. 11B, C). Western blot analysis for adhesion molecules associated with ARHGAP26 or focal adhesion complex proteins showed reduced levels for β-Pix, LIMS1 (PINCH1), and Paxillin in MDCK-ARHGAP26, and more pronounced so in MDCK-CLDN18-ARHGAP26 cells (FIG. 11D).

Mirroring the changes in protein levels, a significant decrease in levels of PINCH1 and Paxillin transcripts was observed in MDCK-ARHGAP26 and MDCK-CLDN18-ARHGAP26 cells by qPCR (FIG. 11E). A substantial decrease in Talin-1, Talin-2 and SDC1 (Syndecan 1) mRNA levels in cells expressing the fusion protein was also observed, a further indication of poor ECM-adhesion of CLDN18-ARHGAP26 cells (FIG. 11E).

In addition to the cytoplasmic components of focal adhesions, protein levels of integrin family members, which directly interact with the ECM components were analysed. Consistent with the poor attachment of MDCK-CLDN18-ARHGAP26 cells on collagen coated surfaces (FIG. 11A), these cells expressed reduced levels of ITGB1 (integrin β1) and ITGB5 (integrin β5) (FIG. 11F). Indeed, a decrease in transcript levels for a number of integrin subunits, in particular integrin α5, was observed in MDCK-CLDN18-ARHGAP26 cells (FIG. 11G). In summary, overexpression of ARHGAP26 and even more so of the fusion gene disrupt ECM adhesion.

### Example 8

### The Epithelial Barrier Promoted by CLDN18 is Compromised by CLDN18-ARHGAP26

Claudins are critical components of the paracellular epithelial barrier, including the protection of the gastric tissue from the acidic milieu in the lumen. Alterations of this barrier function might cause chronic inflammation, a risk factor for the development of GC. Therefore, the role of CLDN18 and the fusion protein in barrier formation was investigated. Overexpression of CLDN18, which is not endogenously expressed in MDCK cells, resulted in a dramatic increase in the transepithelial electrical resistance (TER) of MDCK-CLDN18 monolayers. While ARHGAP26 had no significant effect on the TER, CLDN18-ARHGAP26 completely abolished the TER (FIG. 11H). This effect did not simply reflect the lack of the C-terminal PDZ-binding motif, since a CLDN18 construct where this C-terminal PDZ-binding motif was inactivated (CLDN18ΔP) still increased the baseline TER of MDCK cells. Phase contrast images of confluent CLDN18-ARHGAP26 fusion expressing MDCK cells showed that these cells failed to form tight monolayers, explaining the loss of TER (FIG. 11I). While expression levels and subcellular localization of TJP1 (ZO-1), a scaffold protein that directly links claudins to the actin cytoskeleton, were not altered in MDCK cells expressing the fusion protein (FIG. 9E, F), the expression of several other TJ components was upregulated in MDCK-CLDN18-ARHGAP26, possibly as a compensatory mechanism (FIG. 9E).

### Example 9

### CLDN18-ARHGAP26 Exerts Cell Context Specific Effects on Cell Proliferation, Invasion and Migration

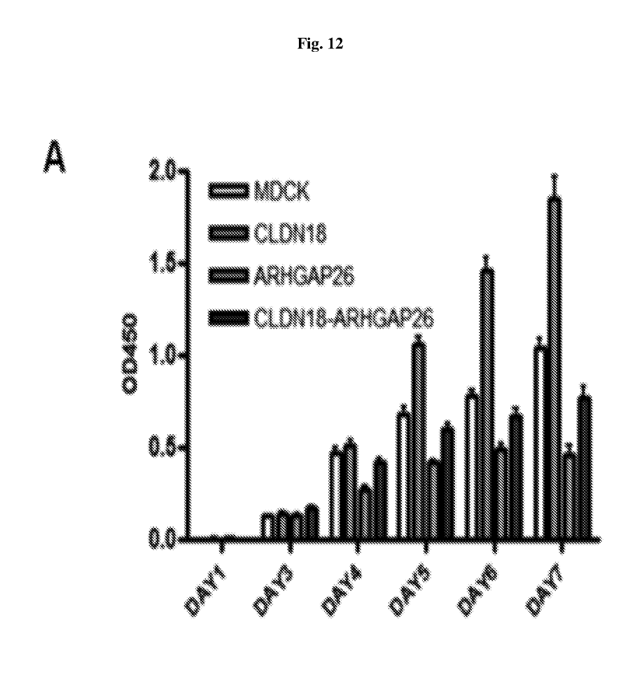

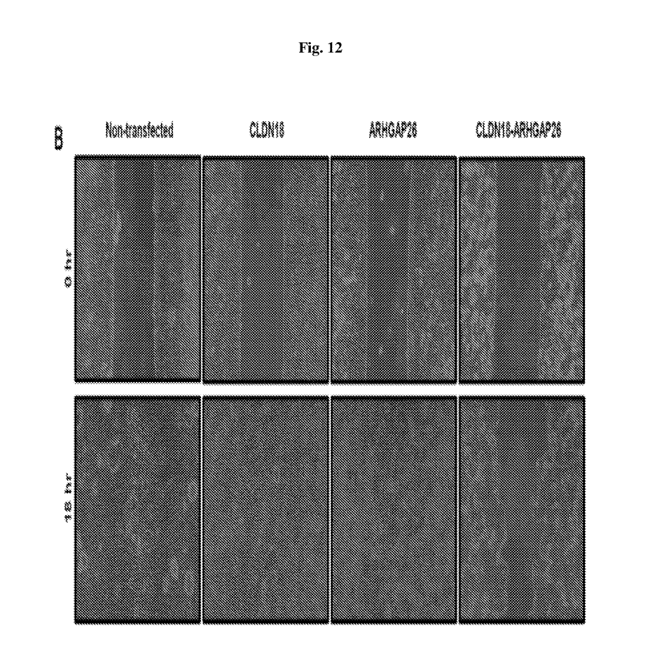

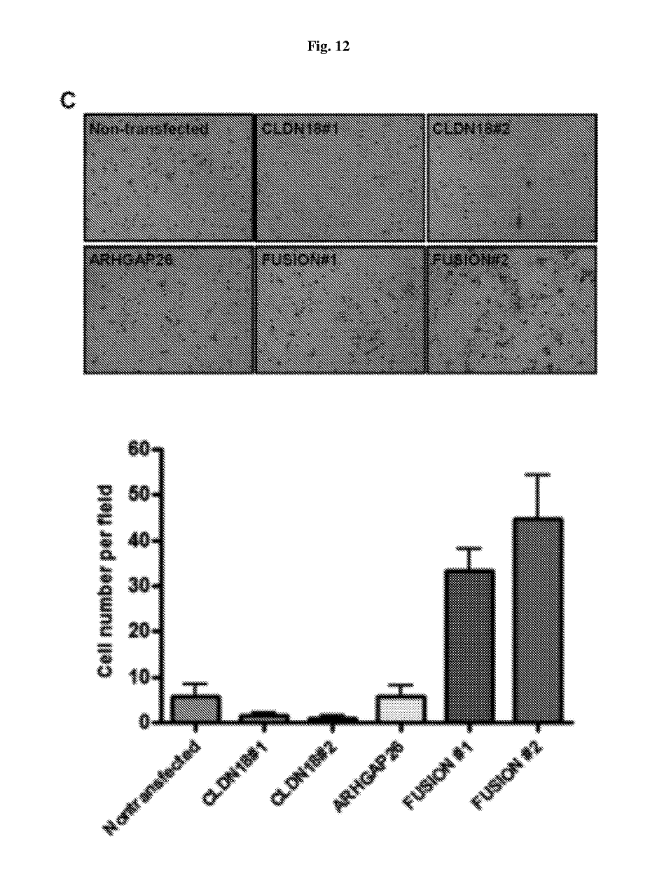

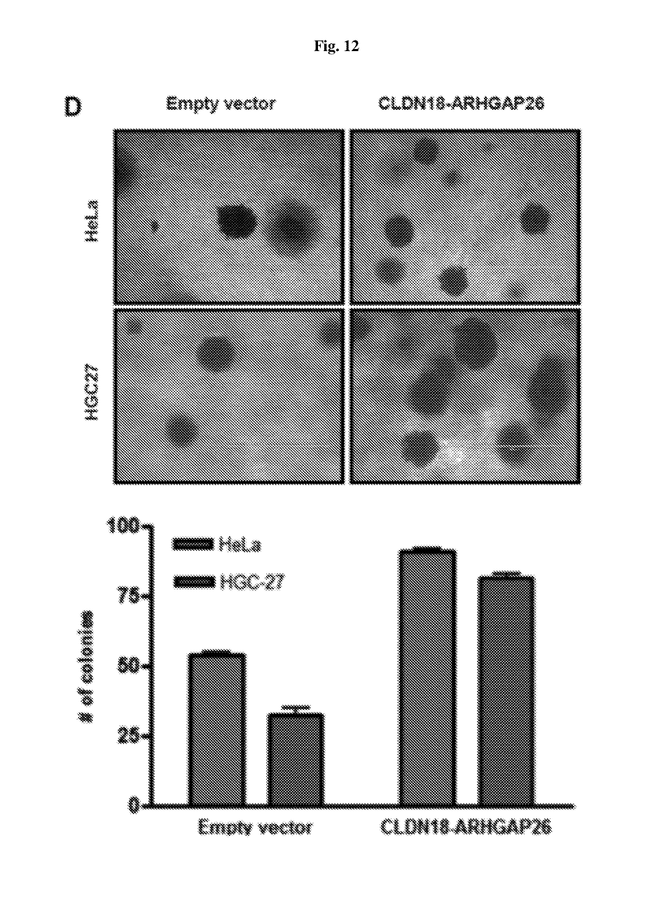

In GC cell line HGC27, CLDN18-ARHGAP26 induces a gain of proliferation (FIG. 4H). Interestingly however, in non-transformed MDCK cells, proliferation rates for MDCK-CLDN18-AHGAP26 cells were lower as compared to controls (FIG. 12A). While wound closure experiments showed a reduced cell migration of MDCK-CLDN18-ARHGAP26 cells compared to controls (FIG. 12B), expression of CLDN18-ARHGAP26 in MDCK cells had no effect on invasion and anchorage independent growth, which are features of cancer progression and metastasis. These processes were thus tested to determine if they were altered in cancer cell lines HGC27 and HeLa. Two independent HeLa cell lines stably expressing CLDN18-ARHGAP26 showed 3 to 4-fold increase in cell invasion (FIG. 12C) and HeLa and HGC27 cells stably expressing the fusion protein formed 30% more colonies in soft agar growth assays (FIG. 12D). These findings highlight different effects of the fusion protein on proliferation, invasion and anchorage independent growth in non-transformed and transformed cells, and suggest a role of the fusion protein driving late cancer events such as invasion and metastasis.

### Example 10

### Both ARHGAP26 and CLDN18-ARHGAP26 Inhibit RhoA and Stress Fiber Formation

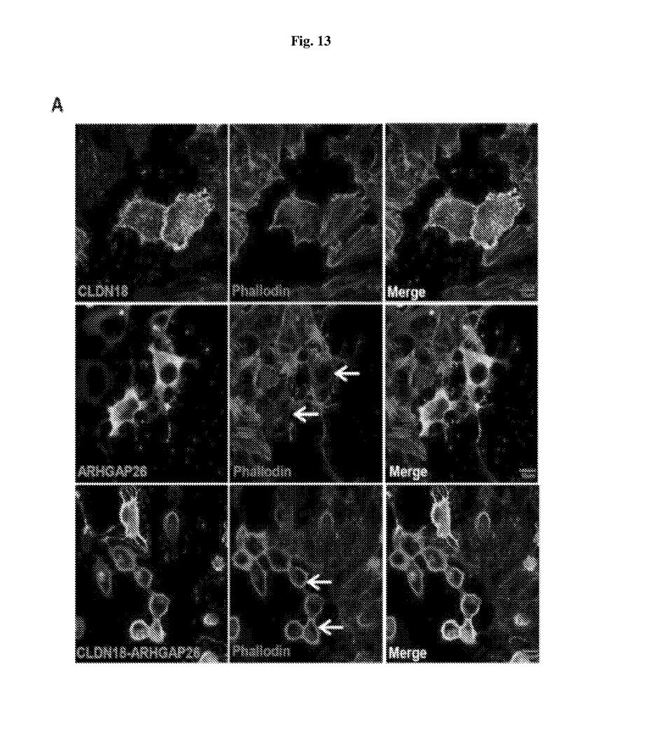

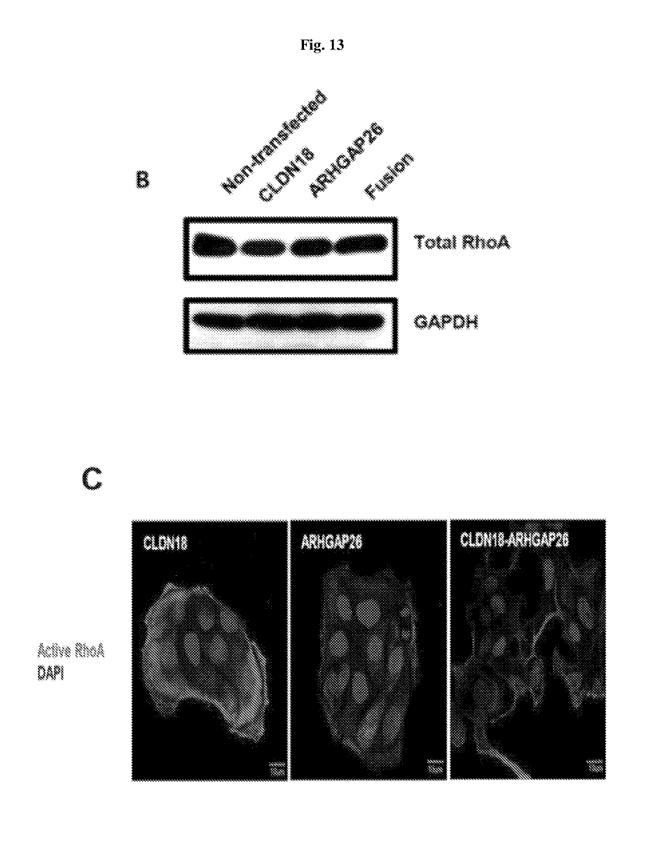

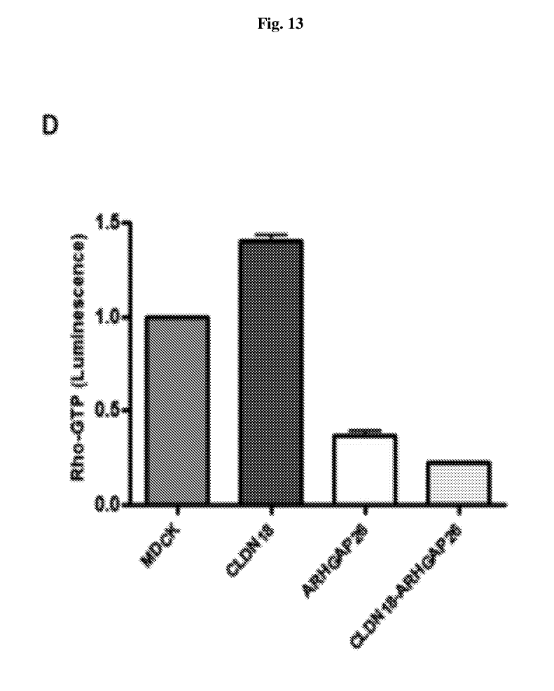

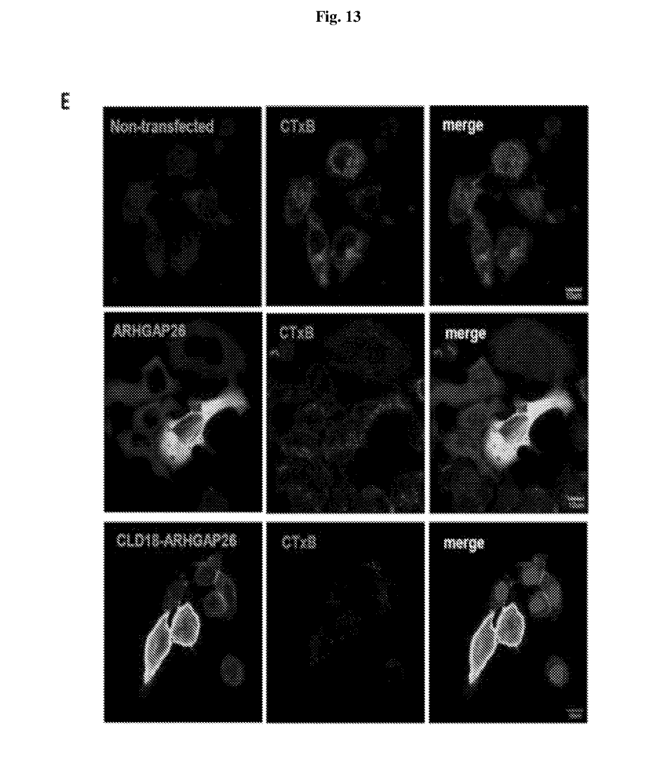

RhoA regulates many actin events like actin polymerization, contraction and stress fiber formation upon growth factor receptor or integrin binding to their respective ligands. ARHGAP26 stimulates, via its GAP domain, the GTPase activities of CDC42 and RhoA, resulting in their inactivation. Since the CLDN18-ARHGAP26 fusion protein retains the GAP domain of ARHGAP26, it may still be able to inactivate RhoA. To test this, the effect of CLDN18-ARHGAP26 expression on stress fiber formation and the presence and subcellular localization of active RhoA (e.g. GTP-bound RhoA) were analysed. In HeLa cells, stable overexpression of ARHGAP26 or CLDN18-ARHGAP26 induced cytoskeletal changes, notably a reduction in stress fibers indicative of RhoA inactivation (FIG. 13A). Labeling of stable cell lines with an antibody that specifically recognizes activated RhoA showed reduced labeling in ARHGAP26 and CLDN18-ARHGAP26 fusion protein expressing cells, while total RhoA levels remained unchanged (FIG. 13B, C). GLISA assay measuring levels of active RhoA further confirmed these results (FIG. 13D). These findings indicate that the GAP domain in the CLDN18-ARHGAP26 fusion protein retains its inhibitory activity on RhoA.

### Example 11

### CLDN18-ARHGAP26 Fusion Protein Suppresses Clathrin Independent Endocytosis

Changes in endocytosis can affect cell surface residence time and/or degradation of cell-ECM and cell-cell adhesion proteins as well as receptor tyrosine kinases (RTKs), thereby altering cell adhesion, migration and RTK signaling, which can drive carcinogenesis. In contrast to the other cell lines, HeLa cells expressing the CLDN18-ARHGAP26 fusion protein showed a significant reduction of endocytosis (FIG. 13E and Example 13), consistent with the absence of the BAR and PH domains, which are essential for endocytosis from the fusion protein.

### Example 12

### Biological Context of Recurrent Fusion Genes CLEC16A-EMP2, SNX2-PRDM6, MLL3-PRKAG2 and DUS2L-PSKH1

The fusion transcripts between DUS2L and PSKH1 were identified in the cancer cell line TMK1 and subsequently in two primary gastric tumors. However, in one tumor, the exon 3 of DUS2L was fused to the exon 2 (UTR region) of PSKH1 resulting in an out of frame fusion transcript (FIG. 6). In TMK1 and the second tumor, exon 10 of DUS2L was fused in frame to exon 2 of PSKH1. siRNA knock down of DUS2L in non-small cell lung carcinomas cells suppressed growth and association between high levels of DUS2L in tumors and poorer prognosis of lung cancer patients has been reported. PSKH1 was identified as a regulator of prostate cancer cell growth. Consistent proliferative effects for DUS2L-PSKH1 were not found (FIG. 6). However, proliferation is only one possible mechanism by which a (fusion) gene can contribute to tumorigenesis or progression and it remains possible that DUS2L-PSKH1 plays a role in GC.

Unpaired inversions created the fusion gene CLEC16A-EMP2 which were identified in five out of 100 GCs. Of CLEC16A, exon 4 (one tumor), exon 9 (two tumors) or exon 10 (two tumors) were fused to exon 2 of EMP2 (FIG. 7). The first 60 bp of EMP2 exon 2 are 5′ UTR and the fusion results in the inclusion of 20 amino acids in front of the canonical start methionine of EMP2. The predicted open reading frame codes for 328, 486 and 524 amino acids retaining the entire EMP2 protein with its functional domains Experiments in a B-cell lymphoma cell line suggest that EMP2 functions as a tumor suppressor. In contrast, EMP2 was found to be highly expressed in >70% of ovarian tumors antibodies against EMP2 significantly suppressed tumor growth and induced cell death in mouse xenografts with an ovarian cancer cell line. EMP2 therefore might be a drug target. Both studies suggest a role of EMP2 in cancer but the effect might be tissue specific. 14 of the 15 sequenced GCs were analysed by expression microarray and found high expression level of EMP2 in all GCs and the highest expression in tumor 113 which harbored the CLEC16A-EMP2 fusion (data not shown). This is in agreement with an oncogenic role of EMP2 as part of the fusion. Proliferation assays with HGC27 stably expressing the fusion gene (FIG. 7) further support that CLEC16A-EMP2 could have oncogenic properties.

SNX2-PRDM6 was found to be fused in frame in one gastric tumor (exon 12 of SNX2 fused to exon 4 of PRDM6) and out of frame in a second tumor (exon 2 of SNX2 fused to exon 7 of PRDM6, FIG. 8). SNX2 encodes a member of the sorting nexin family and members of this family are involved in intracellular trafficking. PRDM6 is likely to have a histone methyltransferase function and might act as a transcriptional repressor. Overexpression of PRDM6 in mouse embryonic endothelial cells induces apoptosis and reduced tube formation suggesting that PRDM6 may play a role in vasculature by chromatin modeling. A reduced proliferation rate for HGC27 stably expressing SNX2-PRDM6 was observed but a potentially oncogenic effect might be related to enhanced vasculature rather than proliferation.

### Example 13

### CLDN18-ARHGAP26 Fusion Protein Suppresses Clathrin Independent Endocytosis

ARHGAP26 is reported to be indispensable for clathrin independent endocytosis and many receptor tyrosine kinases (RTKs) can be internalized by both clathrin dependent and independent pathways. In order to evaluate the effect of the CLDN18-ARHGAP26 fusion protein on clathrin-independent endocytosis, fluorescein isothiocyanate (FITC) conjugated CTxB, a marker for clathrin-independent endocytosis, was incubated with live control HeLa cells or cells stably expressing CLDN18, ARHGAP26 or CLDN18-ARHAGP26 for 15 minutes. Cells were then fixed and internalized FITC-CTxB visualized by fluorescence microscopy. In contrast to the other cell lines, HeLa cells expressing the CLDN18-ARHGAP26 fusion protein showed a significant reduction in the amount of CTxB endocytosed (FIG. 13), consistent with the absence of the BAR and PH domains, which are essential for endocytosis, from the fusion protein.

Recurrent somatic SVs and recurrent fusion genes were observed in this study. The simulations show that the rate of recurrent fusion genes could not be explained by chance indicating that specific rearrangements are more likely to occur than others and/or that selective processes enrich for such rearrangements. By comparing the somatic SVs with a genome-wide view of chromatin interactions, significantly more overlaps of rearrangement sites with chromatin interactions were observed than expected by chance, suggesting that the chromatin structure contributes to recurrent fusions of distant loci in GC.

This is the first systematic correlation analysis between somatic SVs in cancer and chromatin interactions. Since the chromatin structure was profiled in a different cell type than GC, the actual rate of overlap between chromatin interactions and rearrangements may have been underestimated.

The validity, expression and reading frame characteristics of 136 fusion genes were evaluated, and five recurrent fusion genes were identified by an extended screen. CLDN18-ARHGAP26 was analysed in detail and functional properties promoting both, early cancer development and late disease progression were found. CLDN18 and ARHGAP26 are expressed in the gastric mucosa epithelium, where CLDN18 localizes to tight junctions (TJs) and ARHGAP26 to punctate tubular vesicular structures of epithelial cells. The CLDN18-ARHGAP26 fusion gene thus links functional protein domains of a regulator of RhoA to a TJ protein resulting in altered properties. These, as well as the aberrant localization of the GAP activity, result in changes to cellular functions that are associated with GC.

While CLDN18-ARHGAP26 was associated with increased proliferation, anchorage dependent growth and invasion in tumorigenic HeLa and HGC27 cells, such cellular processes were reduced (proliferation, wound closure) in non-transformed MDCK cells, suggesting that the degree of transformation influences some of the effects of the fusion protein, consistent with the multi-step model of carcinogenesis. In the relevant GC in situ as well as when over-expressed in MDCK cells, CLDN18-ARHGAP26 was linked to a loss of the epithelial phenotype.

1. A method of determining or making of a prognosis if a patient has cancer or is at an increased risk of having cancer, the method comprising testing for the presence of one or more cancer-associated fusion genes, or proteins derived thereof, in a sample obtained from a patient, wherein said presence of one or more cancer-associated fusion genes in the sample indicates that said patient has cancer, or is at an increased risk of cancer, wherein the cancer-associated fusion genes are selected from the group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH1 (SEQ ID NO.: 131 or 133), or wherein the cancer-associated fusion genes are selected from the group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH1 (SEQ ID NO.: 131 or 133) in combination with CLDN18-ARHGAP26 (SEQ ID NO: 107).

2. The method of claim 1, wherein the presence of one or more cancer-associated fusion genes in the sample indicates that the patient is a candidate for a differential treatment plan.

3. The method according to claim 1, wherein said cancer-associated fusion gene is 2, or 3, or 4 fusion genes selected from the group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH1 (SEQ ID NO.: 131 or 133), or wherein the cancer-associated fusion genes are selected from the group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125) and DUS2L-PSKH1 (SEQ ID NO.: 131 or 133) in combination with CLDN18-ARHGAP26 (SEQ ID NO: 107).

4. The method according to claim 1, wherein the cancer is an epithelial cancer.

5. The method according to claim 4, wherein the epithelial cancer is selected from the group consisting of gastric cancer, lung cancer, breast cancer, urogenital cancer, colon cancer, prostate cancer and cervical cancer.

6. The method according to claim 5, wherein said cancer is gastric cancer.

7. The method according to claim 1, wherein said cancer-associated fusion gene is CLEC16A-EMP2 (SEQ ID NO.: 97, 99 or 101) or CLEC16A-EMP2 (SEQ ID NO.: 97, 99 or 101) in combination with CLDN18-ARHGAP26 (SEQ ID NO: 107).

8. The method according to claim 7, wherein said cancer-associated fusion gene is CLEC16A-EMP2 (SEQ ID NO.: 97, 99 or 101).

9. The method according to claim 1, wherein the increased risk of cancer is determined in comparison to a sample from a patient without any one or more of the cancer-associated fusion genes.

10. The method according to claim 1, wherein the one or more fusion genes is at least 70% identical to a sequence selected from the group consisting of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125), DUS2L-PSKH1 (SEQ ID NO.: 131 or 133) and CLDN18-ARHGAP26 (SEQ ID NO: 107).

11. An expression vector comprising a nucleic acid sequence encoding any one of CLEC16A-EMP2 (SEQ ID NO.: 97, 99 or 101), SNX2-PRDM6 (SEQ ID NO.: 113 or 115), MLL3-PRKAG2 (SEQ ID NO.: 121, 123 or 125), DUS2L-PSKH1 (SEQ ID NO.: 131 or 133) or CLDN18-ARHGAP26 (SEQ ID NO: 107).

12. A cell transformed with the expression vector according to claim 11.

13. A method for producing a polypeptide, comprising culturing the transformed cell according to claim 12 under conditions suitable for polypeptide expression and collecting the amount of said polypeptide from the cell.

14.-21. (canceled)

22. A kit when used in the method according to claim 1, comprising:
a) a first primer selected from the group consisting of SEQ ID NO. 1, SEQ ID NO. 3, SEQ ID NO. 5, SEQ ID NO. 7 and SEQ ID NO. 9;
b) a second primer selected from the group consisting of SEQ ID NO. 2, SEQ ID NO. 4, SEQ ID NO. 6, SEQ ID NO. 8 and SEQ ID NO. 10; optionally together with instructions for use.

23. The kit according to claim 22, further comprising deoxyribonucleotide bases (dNTPs).

24. The kit according to claim 22, further comprising DNA polymerase.
